(19) United States
(12) Patent Application Publication
(10) Pub. No.: US 2023/0286925 A1
(43) Pub. Date: Sep. 14, 2023

(54) PHENYL TRIAZOLE MLL1-WDR5 PROTEIN-PROTEIN INTERACTION INHIBITOR

(71) Applicants: HUYABIO International, LLC, San Diego, CA (US); CHINA PHARMACEUTICAL UNIVERSITY, Nanjing (CN)

(72) Inventors: Qidong YOU, Nanjing (CN); Xiaoke GUO, Nanjing (CN); Farbod SHOJAEI, San Diego, CA (US); Xin CHEN, Nanjing (CN); J. Edward SEMPLE, Lake Forest, CA (US); Zhengyu JIANG, Nanjing (CN); Xiaoli XU, Nanjing (CN); Mireille GILLINGS, San Diego, CA (US)

(21) Appl. No.: 18/120,318

(22) Filed: Mar. 10, 2023

Related U.S. Application Data
(60) Provisional application No. 63/319,582, filed on Mar. 14, 2022.

(51) Int. Cl. C07D 249/06; C07D 401/12; C07D 403/10; C07D 403/12; C07D 405/14; C07D 401/14; C07D 405/12; C07D 403/14; C07D 413/14; C07D 409/14; C07D 239/48; C07D 487/04; C07D 417/14; C07D 417/12; A61P 35/02

(52) U.S. Cl. CPC C07D 249/06; C07D 401/12; C07D 403/10; C07D 403/12; C07D 405/14; C07D 401/14; C07D 405/12; C07D 403/14; C07D 413/14; C07D 409/14; C07D 239/48; C07D 487/04; C07D 417/14; C07D 417/12; A61P 35/02

(57) ABSTRACT

Described herein are a phenyl triazole MLL1-WDR5 protein-protein interaction inhibitors, pharmaceutical compositions and methods of use.

### CROSS-REFERENCE

This application claims the priority benefit of U.S. Provisional Application No. 63/319,582 filed Mar. 14, 2022, the contents of which are incorporated herein by reference in their entirety.

### FIELD OF THE DISCLOSURE

The present invention relates to the field of pharmaceutical chemistry, and more particularly to phenyl triazole compounds that inhibit MLL1-WDR5 protein-protein interactions, and preparation and medical uses thereof.

### BACKGROUND OF THE DISCLOSURE

Translocation and re-arrangement of the methyl transferase MLL1 gene for histone H3K4 can lead to mixed lineage leukemia (MLL1, acute myeloid leukemia and acute lymphoid leukemia). MLL1 gene rearrangement is found in about 10% of leukemia patients. Upon re-arrangement, MLL1 gene fuses with other chaperone genes to form fusion genes, and the carcinogenic MLL fusion protein is expressed. The fusion protein can interact with RNA polymerase II (Pol II) related elongation factors to form the super elongation complex (SEC). The complex can lead to abnormal expression of the Hox gene regulated by MLL1 through Pol II, which causes a series of serious consequences to induce MLL1 leukemia onset.

Chromosomal translocation of MLL1 gene is monoallelic and there is a wildtype MLL1. When the wildtype MLL1 allele is knocked out, the MLL1 fusion protein alone will not lead to leukemia, and the enzymatic activity of the wildtype MLL1 is necessary for the MLL1 fusion protein to induce leukemia. Thus, specific inhibition of the enzymatic activity of the wildtype MLL1 can achieve the effect of treating leukemia.

Catalytic activity on H3K4 methylation by MLL1 alone is very weak and can only result in monomethylation; the enzyme catalytic activity improves greatly upon the formation of the MLL1 core catalytic complex, especially the catalytic activity on H3K4me2. The MLL-C-terminal WIN motif moiety is capable of binding WDR5, RbBP5, Ash2L and DPY30 to form complexes. MLL1 interacts with WDR5 directly through the C-terminal WIN motif moiety, to mediate the interaction between the catalytic domain of MLLISET and other protein complexes. When WDR5 is knocked out, the level of H3K4me2/3 decreases and the Hox gene expression is downregulated.

Thus, use of small molecule inhibitors to interfere with the protein-protein interaction of MLL1-WDR5 is an effective method to inhibit MLL1 enzymatic activity and downregulate Hox and Meis-1 gene expression to block the progression of leukemia. MLL1-WDR5 protein-protein interaction inhibitors have been described in WO2019205687A1, which is herein incorporated by reference in its entirety. A need exists for additional MLL1-WDR5 protein-protein interaction inhibitors having additional and/or improved properties.

### SUMMARY

The foregoing and additional needs are satisfied by embodiments described herein. Provided herein are small molecule compounds that can regulate MLL1-WDR5 protein-protein interaction, and compositions and methods of using the compounds and compositions. Inhibitors of MLL1-WDR5 protein-protein interactions inhibit the enzyme catalytic activity of MLL1, and downregulate the methylation level of H3K4 and the gene expression levels of Hox and Meis-1 genes to induce the apoptosis of leukemia cells. Therefore, the compound and compositions described herein can be used to treat cancers such as, but not limited to leukemia.

Some embodiments described herein are phenyl triazole compounds, or pharmaceutically acceptable salt or solvates thereof. In some embodiments, the compounds are described in Table 1, Table 2, Table 3 or Table 4.

Some embodiments described herein are pharmaceutical compositions comprising a compound as described herein, or a pharmaceutically acceptable salt or solvate thereof, and one or more pharmaceutically acceptable carriers, diluents and excipients.

Some embodiments provided herein is a method for the treatment or prevention of acute leukemia in a patient in need thereof, comprising administering to the patient a therapeutically acceptable dose of the compound described herein, or a pharmaceutically acceptable salt or solvate thereof. In some embodiments provided herein is a method for the treatment or prevention of acute leukemia in a patient in need thereof, comprising administering to the patient a pharmaceutical composition as described herein. In some embodiments, the acute leukemia is acute leukemia with MLL1 gene rearrangement.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

### DETAILED DESCRIPTION

The phenyl triazole compounds and the aniline compounds as described herein have strong inhibitory activity against MLL1-WDR5 protein-protein interaction, can reduce the MLL1 catalytic activity of MLL1 at cellular level, downregulate the expression of Hox and Meis-1 genes and induce apoptosis of leukemia cells. Additionally, the compounds described herein exhibit good water solubility and pharmaceutical safety, and can be used for the treatment of cancers, such but not limited to leukemia.

### Compounds

In some embodiments, described herein is a compound of Formula (I), or a pharmaceutically acceptable salt or solvate thereof:

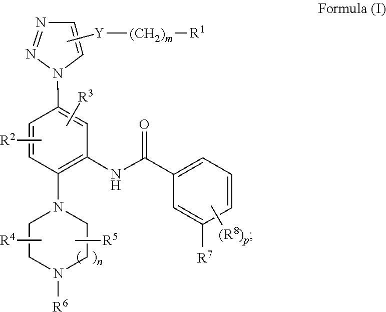

wherein:
Y is absent, —O—, —S—, —C(O)—, —CH₂O—, —(CO)O—, —O(CO)—, —NR¹¹—, —C(O)NR¹¹— or —NR¹²C(O)—,
   wherein
R¹⁰, R¹¹, and R¹² each independently is hydrogen, C₁-C₄ alkyl, C₁-C₄ haloalkyl, or substituted or unsubstituted phenyl {wherein the phenyl is substituted with one, two or three of halogen, amino, cyano, hydroxyl, trifluoro, —C₁-C₄ alkyl, C₁-C₄ alkoxy, carboxyl, or imidazolyl};
R¹ is hydrogen, hydroxyl, thiol, carboxyl, cyano, C₁-C₄ alkyl, substituted C₁-C₄ alkyl, substituted or unsubstituted phenyl, {wherein the substituent is C₁-C₄ alkoxy}, substituted or unsubstituted C₃-C₆ cycloalkyl, substituted or unsubstituted nitrogen- or oxygen-containing 3 to 7 membered heterocyclic ring, or substituted or unsubstituted 3 to 7 membered heterocyclic ring containing both nitrogen and oxygen, wherein substituents on the heterocyclic ring can optionally be on the hetero atom; unsaturated heterocycloalkyl containing nitrogen or oxygen or both, —NR¹³COR¹⁴, —OR¹⁷—, —C(O)O—R¹⁷, —O(CO)O—R¹⁷ —C(O)NR¹⁵R¹⁶ or —NR¹⁵R¹⁶, wherein
R¹³ is hydrogen, C₁-C₄ alkyl, C₁-C₄ haloalkyl, substituted or unsubstituted phenyl,
R¹⁴ is hydrogen, amino, hydroxyl, C₁-C₄ alkyl, C₁-C₄ alkoxy, substituted or unsubstituted phenyl, substituted or unsubstituted nitrogen- or oxygen-containing 3 to 7 membered heterocyclic ring, wherein substituents on the heterocyclic ring can be on the hetero atom;
R¹⁵ and R¹⁶ are each independently hydrogen, C₁-C₄ alkyl, substituted or unsubstituted phenyl, substituted or unsubstituted C₃-C₇ cycloalkyl, substituted or unsubstituted nitrogen- or oxygen- or both containing 3 to 7 membered heterocyclic ring {wherein the heterocyclic ring may be aryl, partially unsaturated, or fully saturate, wherein substituents on the heterocyclic ring can be on the hetero atom}, or
R¹⁵ and R¹⁶ together form: a nitrogen-, oxygen-, or nitrogen and oxygen- or nitrogen and nitrogen or oxygen and oxygen containing 3 to 7 membered heterocyclic ring, wherein the heterocyclic ring is optionally substituted with one, two or three substituents which are independently halogen, C₁-C₄ alkyl, C₁-C₄ alkoxy, amino, hydroxyl, thiol, carboxyl, cyano, trifluoromethyl or imidazolyl; wherein the substituents on the heterocyclic ring can be on the hetero atom;
R¹⁷ is C₁-C₄ alkyl, C₁-C₄ haloalkyl, substituted or unsubstituted phenyl, substituted or unsubstituted C₃-C₇ cycloalkyl, substituted or unsubstituted nitrogen- or oxygen- or both containing 3 to 7 membered heterocyclic ring; wherein substituents on the heterocyclic ring can be on the hetero atom;
R² and R³ are independently hydrogen, halogen, methyl, methoxy, difluoromethoxy, or trifluoromethoxy;
R⁴, R⁵ and R⁶ are each independently hydrogen, C₁-C₆ alkyl, or C₃-C₆ cycloalkyl;
each R⁷ and R⁸ is independently hydrogen, halogen, amino, cyano, hydroxy, thiol, nitro, C₁-C₆ alkyl, C₃-C₇ cycloalkyl, C₁-C₆ alkoxy, C₃-C₇ cycloalkoxy, trifluoromethyl, difluoromethyl, trifluoromethoxy, difluoromethoxy, C₁-C₆ alkylthio, C₁-C₆ alkylsulfinyl, C₁-C₆ alkylsulfonyl, hydroxy-(C₁-C₆)alkyl, amino-(C₁-C₆)alkyl, or —C(O)NR^{1A}R^{1B}, wherein R^{1A} and R^{1B} are each independently hydrogen or C₁-C₆ alkyl;
m is an integer from 0-5;
n is an integer from 0-2; and
p is an integer from 0-3.

In some embodiments, R⁷ is hydrogen, halogen, amino, thiol, C₁-C₆ alkyl, C₁-C₆ alkoxy, trifluoromethyl, difluoromethyl, trifluoromethoxy, difluoromethoxy, nitro or cyano. In some embodiments, each R⁷ and R⁸ is independently hydrogen, chloro, fluoro, bromo, amino, cyano, methyl, methoxy, trifluoromethyl, difluoromethyl, or trifluoromethyl. In some embodiments, R⁷ is —Cl, —F, —OH, —CF₃, —CH₃, —CF₃, or —OCH₃. In some embodiments, R⁷ is —NO₂ or —NH₂. In some embodiments, R⁷ is —NH₂. In some embodiments, R⁷ is —C(O)NH₂. In some embodiments, R⁷ is —OH, —SH, —OMe, —SMe, or —NMe.

In some embodiments, each R⁸ is independently hydrogen, halogen, C₁-C₆ alkyl, C₁-C₆ alkoxy, trifluoromethyl, difluoromethyl, trifluoromethoxy, difluoromethoxy, amino, nitro, or cyano. In some embodiments, each R⁸ is independently hydrogen, chloro, fluoro, bromo, amino, cyano, methyl, methoxy, trifluoromethyl, difluoromethyl, or trifluoromethyl. In some embodiments, each R⁸ is independently —Cl, —F, —OH, —CF₃, —CH₃, or —OCH₃. In some embodiments, each R⁸ is independently —CH₃, —Cl or —F. In some embodiments, each R⁹ is independently —OCF₃ or —CF₃. In some embodiments, each R⁸ is independently hydrogen. In some embodiments, each R⁸ is independently —C(O)NH₂. In some embodiments, each R⁷ is independently —OH, —SH, —OMe, —SMe, or —NMe,

In some embodiments, p is 1, 2, or 3. In some embodiments, p is 1 or 2. In some embodiments, m is 1. In some embodiments, p is 2. In some embodiments, p is 3. In some embodiments, p is 0.

In some embodiments, n is 1 or 2. In some embodiments, n is 1. In some embodiments, n is 2. In some embodiments, n is 0.

In some embodiments, the substituted phenyl is not 4-chloro-2-fluoro-3-methylaniline.

In some embodiments, the substituted phenyl is 4-chloro-2-fluoro-3-methylaniline.

Some embodiments of compounds of Formula (I) are inhibitors of the MLL1-WDR5 protein-protein interaction.

In some embodiments, the compound of Formula (I) has the structure of Formula (II), or a pharmaceutically acceptable salt or solvate thereof:

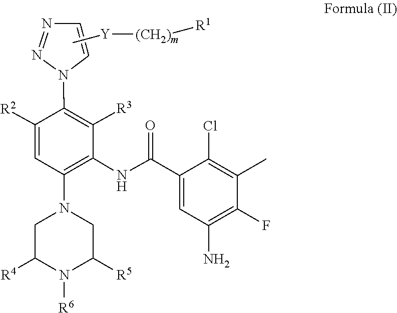

wherein, unless otherwise defined herein, the variable groups have the definitions provided in Formula (I).

In some embodiments, Y is —O—, —S—, —C(O)—, —CH₂O—, —NR¹⁰—, —C(O)NR¹¹— or —NR¹²C(O)—. In some embodiments, Y is —O— or —NR¹⁰—. In some embodiments, Y is —O— or —NR¹⁰—, wherein R¹⁰ is hydrogen or C₁-C₄ alkyl. In some embodiments, Y is —O—. In some embodiments, Y is —NR¹⁰—. In some embodiments, Y is —NH—. In some embodiments, Y is —NCH₃—. In some embodiments, Y is —S—. In some embodiments, Y is —C(O)—. In some embodiments, Y is —CH₂O—.

In some embodiments, Y is —C(O)NR¹¹. In some embodiments, Y is —C(O)NR¹¹—, wherein R¹¹ is hydrogen or C₁-C₄ alkyl. In some embodiments, Y is —C(O)NH—. In some embodiments, Y is —C(O)N(CH₃)—. In some embodiments, Y is —NR¹²C(O)—. In some embodiments, Y is —NR¹²C(O)—, wherein R¹¹ is hydrogen or C₁-C₄ alkyl. In some embodiments, Y is —NHC(O)—. In some embodiments, Y is —N(CH₃)C(O)—.

In some embodiments, Y is absent.

Some embodiments of compounds of Formula (II) are inhibitors of the MLL1-WDR5 protein-protein interaction.

In some embodiments, the compound has the structure of Formula (III), or a pharmaceutically acceptable salt or solvate thereof:

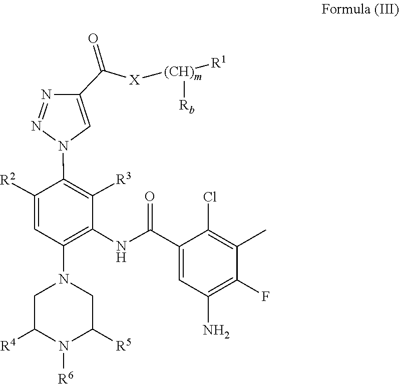

wherein:
X is —O— or —NR¹⁷—, wherein
R¹⁷ is hydrogen or C₁-C₆ alkyl.

In some embodiments, X is —O—. In some embodiments, X is —NR^{1A}—. In some embodiments, X is —NH—. In some embodiments, X is —N(CH₃)—.

In some embodiments, m is 0, 1, 2, 3, 4, or 5. In some embodiments, m is 1, 2, 3, or 4. In some embodiments, m is 1, 2, or 3. In some embodiments, m is 1. In some embodiments, m is 2. In some embodiments, m is 3. In some embodiments, m is 4. In some embodiments, m is 5. In some embodiments, m is 6.

In some embodiments, when m is greater than 1, R^{b} can be linked via any C atom in the link. In some embodiments, R^{b} is hydrogen, C₁-C₆ alkyl, substituted or unsubstituted C₃-C₆ heterocycloalkyl, {where the substituent is C₁-C₆ alkyl and wherein the substituent can be optionally present on the hetero atom}.

In some embodiments, R¹ is amino, hydroxyl, thiol, carboxyl, cyano, C₁-C₄ alkyl, C₁-C₆ alkoxy, substituted or unsubstituted phenyl, or a substituted or unsubstituted nitrogen- or oxygen-containing 3 to 7 membered heterocyclic ring. In some embodiments, R¹ is hydrogen. In some embodiments, R¹ is hydroxyl, thiol, carboxyl, cyano, C₁-C₄ alkyl, or C₁-C₆ alkoxy. In some embodiments, R¹ is —OH, —SH, —CN, —CH₃, or —OCH₃. In some embodiments, R¹ is phenyl.

In some embodiments, R¹ is a substituted or unsubstituted nitrogen- or oxygen-containing 3 to 7 membered heterocyclic ring. In some embodiments, the nitrogen- or oxygen-containing 3 to 7 membered heterocyclic ring is pyrrolidine, piperidine, piperazine, or morpholine. In some embodiments, the nitrogen- or oxygen-containing 3-7 membered heterocyclic ring is pyrrolidine. In some embodiments, the 3 to 7 membered ring is piperidine. In some embodiments, the 3 to 7 membered ring is piperazine. In some embodiments, the 3 to 7 membered ring is morpholine.

In some embodiments, R¹ is —NR¹³COR¹⁴, —C(O)NR¹⁵R¹⁶ or —NR¹⁵R¹⁶. In some embodiments, R¹ is —NR¹³COR¹⁴. In some embodiments, R¹ is —C(O)NR¹⁵R¹⁶. In some embodiments, R¹ is —NR¹⁵R¹⁶.

In some embodiments, R¹ is —NR¹⁵R¹⁶, wherein R¹⁵ and R¹⁶ are bonded together with the nitrogen to which they are attached to form a nitrogen- or oxygen-containing 3 to 7 membered heterocyclic ring. In some embodiments, the 3 to 7 membered ring is piperazine, or morpholine. In some embodiments, the 3 to 7 membered ring is piperazine. In some embodiments, the 3 to 7 membered ring is morpholine.

In some embodiments, R¹ is preferably —C(O)OR^{a}, where Ra is hydrogen, C₁-C₆ alkyl, substituted or unsubstituted C₃-C₆ cycloalkyl, {where the substituent is C₁-C₆ alkyl}, substituted or unsubstituted C₃-C₆ heterocycloalkyl, {where the substituent is C₁-C₆ alkyl and wherein the substituent can be optionally present on the hetero atom}.

In some embodiments, R⁴ and R⁵ are each independently C₃-C₆ cycloalkyl. In some embodiments, R⁴ and R⁵ are each independently cyclopropyl, cyclobutyl, cyclopentyl, or cyclohexyl.

In some embodiments, R⁴ and R⁵ are each independently hydrogen or C₁-C₆ alkyl. In some embodiments, R⁴ and R⁵ are each independently C₁-C₆ alkyl. In some embodiments, R⁴ and R⁵ are each independently methyl, ethyl, or iso-propyl. In some embodiments, R⁴ and R⁵ are each methyl. In some embodiments, R⁴ and R⁵ are each hydrogen.

In some embodiments, R⁴ is hydrogen; and R⁵ is C₃-C₆ cycloalkyl or C₁-C₆ alkyl. In some embodiments, R⁴ is hydrogen and R⁵ is C₁-C₆ alkyl. In some embodiments, R⁴ is hydrogen; and R⁵ is methyl, ethyl or isopropyl. In some embodiments, R⁴ is hydrogen; and R⁵ is methyl. In some embodiments, R⁴ is C₃-C₆ cycloalkyl or C₁-C₆ alkyl; and R⁵ is hydrogen. In some embodiments, R⁴ is C₁-C₆ alkyl; and R⁵ is hydrogen. In some embodiments, R⁴ is methyl, ethyl, or isopropyl; and R⁵ is hydrogen. In some embodiments, R⁴ is methyl; and R⁵ is hydrogen.

In some embodiments, R⁶ is C₃-C₆ cycloalkyl. In some embodiments, R⁶ is cyclopropyl, cyclobutyl, cyclopentyl, or cyclohexyl. In some embodiments, R⁶ is cyclopropyl. In some embodiments, R⁶ is cyclobutyl. In some embodiments, R⁶ is cyclopentyl. In some embodiments, R⁶ is cyclohexyl.

In some embodiments, R⁶ is hydrogen or C₁-C₆ alkyl. In some embodiments, R⁶ is C₁-C₆ alkyl. In some embodiments, R⁶ is methyl. In some embodiments, R⁶ is methyl, ethyl, propyl, isopropyl, sec-butyl, isobutyl or tert-butyl. In some embodiments, R⁶ is methyl. In some embodiments, R⁶ is ethyl. In some embodiments, R⁶ is tert-butyl. In some embodiments, R⁶ is hydrogen.

In some embodiments, R² and R³ are independently hydrogen, halogen, methyl, or methoxy. In some embodiments, R² and R³ are independently hydrogen, chloro, fluoro, bromo, iodo, methyl, or methoxy. In some embodiments, R² and R³ are independently hydrogen, chloro, fluoro, or methyl. In some embodiments, R² and R³ are independently difluoromethoxy or trifluoromethoxy.

In some embodiments, R² and R³ are each hydrogen, halogen, or methyl. In some embodiments, R² and R³ are each hydrogen. In some embodiments, R² and R³ are each halogen. In some embodiments, R² and R³ are each methyl.

In some embodiments, R² is halogen or methyl; and R³ is hydrogen. In some embodiments, R² is choro, fluoro, or methyl; and R³ is hydrogen. In some embodiments, R² is hydrogen; and R³ is halogen or methyl. In some embodiments, R² is hydrogen; and R³ is chloro, fluoro, or methyl.

Some embodiments of compounds of Formula (III) are inhibitors of the MLL1-WDR5 protein-protein interaction.

In some embodiments, the compound is not:
methyl 1-(3-(5-amino-2-chloro-4-fluoro-3-methylbenzoylamino)-4-(4-methylpiperazin-1-yl)phenyl)-1H-1,2,3-triazol-4-carboxylate;
1-(3-(5-amino-2-chloro-4-fluoro-3-methylbenzoylamino)-4-(4-methylpiperazin-1-yl)phenyl)-1H-1,2,3-triazol-4-carboxylic acid;
1-(3-(5-amino-2-chloro-4-fluoro-3-methylbenzoylamino)-4-(4-methylpiperazin-1-yl)phenyl)-N,N-dimethyl-1H-1,2,3-triazol-4-carboxamide;
5-amino-2-chloro-4-fluoro-3-methyl-N-(2-(4-methylpiperazin-1-yl)-5-(4-(morpholin-4-carbonyl)-1H-1,2,3-triazol-1-yl)phenyl)benzamide;
1-(3-(5-amino-2-chloro-4-fluoro-3-methylbenzoylamino)-4-(4-methylpiperazin-1-yl)phenyl)-N-(tetrahydro-2H-pyran-4-yl)-1H-1,2,3-triazol-4-carboxamide;
1-(3-(5-amino-2-chloro-4-fluoro-3-methylbenzoylamino)-4-(4-methylpiperazin-1-yl)phenyl)-N-(1-methylpiperidin-4-yl)-1H-1,2,3-triazol-4-carboxamide;
1-(3-(5-amino-2-chloro-4-fluoro-3-methylbenzoylamino)-4-(4-methylpiperazin-1-yl)phenyl)-N-(3-morpholinopropyl)-1H-1,2,3-triazol-4-carboxamide;
5-amino-2-chloro-4-fluoro-3-methyl-N-(2-(4-methylpiperazin-1-yl)-5-(4-(4-methylpiperazin-1-carbonyl)-1H-1,2,3-triazol-1-yl)phenyl)benzamide;
1-(3-(5-amino-2-chloro-4-fluoro-3-methylbenzoylamino)-4-(4-methylpiperazin-1-yl)phenyl)-N-(2-morpholinoethyl)-1H-1,2,3-triazol-4-carboxamide;
1-(3-(5-amino-2-chloro-4-fluoro-3-methylbenzoylamino)-4-(4-methylpiperazin-1-yl)phenyl)-N-(3-aminopropyl)-1H-1,2,3-triazol-4-carboxamide;
Tert-butyl(1-(3-(5-amino-2-chloro-4-fluoro-3-methylbenzoylamino)-4-(4-methylpiperazin-1-yl)phenyl)-1H-1,2,3-triazol-4-yl)carbamate;
N-(1-(3-(5-amino-2-chloro-4-fluoro-3-methylbenzoylamino)-4-(4-methylpiperazin-1-yl)phenyl)-1H-1,2,3-triazolopyridin-4-yl)-1-methylpiperidin-4-carboxamide;
N-(1-(3-(5-amino-2-chloro-4-fluoro-3-methylbenzoylamino)-4-(4-methylpiperazin-1-yl)phenyl)-1H-1,2,3-triazolopyridin-4-yl)piperidin-4-carboxamide;
5-amino-N-(5-(4-(4-aminobutyrylamino)-1H-1,2,3-triazol-1-yl)-2-(4-methylpiperazin-1-yl)phenyl)-2-chloro-4-fluoro-3-methylbenzamide; or
   or a pharmaceutically acceptable salt or solvate thereof.

In some embodiments, the compound has the structure of Formula (IV), or a pharmaceutically acceptable salt or solvate thereof:

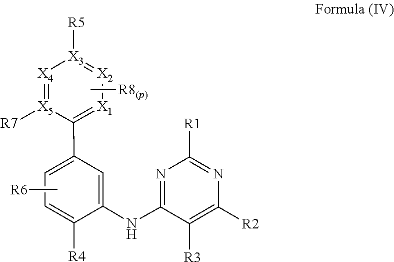

where X₁-X₅ are independently C, CH, or N;
R¹, R² and R³ are the same or different from each other, and are each independently selected from hydrogen, C₁-C₆ alkyl, C₃-C₆ cycloalkyl, C₁-C₆ alkoxy-substituted C₁-C₆ alkyl, nitro, halogen, cyano, aldehyde, hydroxyl, or amino;
R⁴ represents unsubstituted, mono-substituted, di-substituted or tri-substituted morpholinyl, unsubstituted, mono-substituted, di-substituted or tri-substituted piperazinyl, or unsubstituted, mono-substituted, di-substituted or tri-substituted homopiperazinyl, wherein each mono-, di- or tri-substituted morpholinyl, piperazinyl or homopiperazinyl is substituted by one, two or three substituents, each of which is independently C₁-C₄ alkyl, 3-7-membered cycloalkyl, hydroxyalkyl, or phenyl; unsubstituted, mono-substituted, di-substituted or tri substituted-N— or/and —O— containing 3-7 membered hetrocyclic ring; each of the substituent is independently C₁-C₄ alkyl, 3-7-membered cycloalkyl, hydroxyalkyl, or phenyl;
R⁵ is absent or R⁵ is: hydrogen; C₁-C₆ alkyl; substituted C₁-C₆ alkyl {wherein each substituent is a 3-7-membered cycloalkyl, a 3-7-membered heterocyclic ring containing nitrogen or oxygen, or a 3-7-membered heterocyclic ring containing both nitrogen and oxygen}; unsubstituted, mono-substituted, di-substituted or tri-substituted 3-7-membered heterocyclic ring containing nitrogen, oxygen or both {wherein the heterocyclic ring is aromatic, partially unsaturated or fully saturated and each substituent is independently C₁-C₆ alkyl, (e.g., —CH₃, —CH₂CH₃, or —CH(CH₃)₂) or NR¹⁰R¹¹}; —NHCOR⁹; —CONR¹⁰R¹¹; —COR²; or —OR¹⁵; wherein:
R⁹ is: hydrogen; C₁-C₆ alkyl; 3-7-membered cycloalkyl; 3-7-membered heterocycloalkyl containing nitrogen, oxygen or both; or substituted C₁-C₆ alkyl {wherein the substituent is 3-7-membered cycloalkyl, 3-7-membered heterocyclic ring containing nitrogen or oxygen, or 3-7-membered heterocyclic ring containing both nitrogen and oxygen};
R¹⁰, R¹¹ each independently is: hydrogen; C₁-C₆ alkyl; phenyl or substituted phenyl; substituted or unsubstituted 3-7-membered heterocyclic ring containing nitrogen, oxygen or both; substituted C₁-C₆ alkyl {wherein each substituent on R¹⁰ and R¹¹ is independently a 3-7-membered cycloalkyl, a 3-7-membered heterocyclic ring containing nitrogen or oxygen, or a 3-7-membered heterocyclic ring containing both nitrogen and oxygen};
R¹² is: C₁-C₆ alkyl; C₁-C₆ substituted alkyl (wherein the substituent is C₃-C₆ cycloalkyl); or NR¹⁰R¹¹ (wherein R¹⁰ and R¹¹ are defined in the immediate foregoing paragraph);
R¹⁵ is: C₁-C₆ alkyl; C₁-C₆ substituted alkyl (wherein the substituent is C₃-C₆ cycloalkyl);
R⁶ represents hydrogen, halogen, methyl, trifluoromethyl, amino, substituted amino, [wherein the substituent is C₁-C₄ alkyl, or allyl] or [—OR¹² where R¹² is a C₁-C₆ alkyl, or substituted C₁-C₆ alkyl, where the substituent is C₃-C₆ cycloalkyl];
R⁷ absent or R⁷ is hydrogen, halogen, methyl, trifluoromethyl, amino, substituted amino, and the substituent is C₁-C₄ alkyl, or allyl;
R⁸ is absent or represents: hydrogen; C₁-C₆ alkyl; C₁-C₆ substituted alkyl {wherein each alkyl substituent is NR¹³R¹⁴ [wherein each of R¹³ and R¹⁴ independently represents hydrogen, C₁-C₆ alkyl, C₃-C₆ cycloalkyl, or a 5-6-membered aromatic heterocyclic ring containing oxygen or nitrogen, or R¹³ and R¹⁴ are linked and together with N form an optionally substituted 5-7 membered heterocyclic ring containing nitrogen, oxygen, or both nitrogen and oxygen (wherein the optional substituents may be one, two or three C₁-C₄ alkyl (e.g., —CH₃, —CH₂CH₃ or —CH(CH₃)₂)]; 3-7-membered cycloalkyl; 3-7-membered heterocyclic ring containing nitrogen or oxygen, or 3-7-membered heterocyclic ring containing both nitrogen and oxygen}; —OR¹² {wherein R¹² is a C₁-C₆ alkyl, substituted C₁-C₆ alkyl (wherein the substituent is C₃-C₆ cycloalkyl)}; —NR¹³R¹⁴ {wherein each of R¹³ and R¹⁴ independently represents hydrogen, C₁-C₆ alkyl, C₃-C₆ cycloalkyl, 5-6-membered aromatic heterocyclic ring containing oxygen or nitrogen or both, or R¹³ and R¹⁴ are linked and together with N form an optionally substituted 5-7-membered heterocyclic group containing nitrogen, oxygen, or both (wherein the optional substituents may be one, two or three C₁-C₄ alkyl (e.g., —CH₃, —CH₂CH₃ or —CH(CH₃)₂)}; substituted 5-6-membered aromatic heterocyclic ring containing oxygen or nitrogen {wherein the substituent is C₁-C₄ alkyl, alkylamino, substituted alkyl amino [where the substitutent is C₁-C₆ alkyl, C₃-C₆ cycloalkyl, C₃-C₆ heterocycloalkyl containing N or O or both]}; unsubstituted or substituted 3-6-membered heterocyclic ring containing oxygen or nitrogen or both, {wherein the substituent is C₁-C₄ alkyl};
where p is an integer from 0-4.

In some embodiments of the compound of Formula (IV) R¹ is H, F or CH₃ (more preferably H), R² is halo (preferably F, Cl or Br, more preferably Cl), and R³ is NH₂, NHCH₃ or N(CH₃)₂ (more preferably NH₂).

In some embodiments of the compound of Formula (IV), R⁴ or R¹ is mono-, di- or tri-substituted piperazinly, wherein each substituent is C₁-C₃ alkyl (preferably methyl). In some embodiments, R⁴ is 4-methyl piperazinyl, 3,4-dimethyl piperazinyl, 3,4,5-trimethyl piperazinyl, 2,4-dimethylpiperazinyl, or 2,4,6-trimethyl piperazinyl. In some embodiments, R⁴ is mono-, di- or tri-substituted homopiperazinly, wherein each substituent is C₁-C₃ alkyl (preferably methyl). In some embodiments, R⁴ is N-methyl homopiperazinyl, 3,4-dimethyl homopiperazinyl, 3,4,5-trimethyl homopiperazinyl, 3,4,6-trimethyl homopiperazinyl, 3,4,7-trimethyl homopiperazinyl, 2,4-dimethylhomopiperazinyl, 2,4,6-trimethyl homopiperazinyl, or 2,4,7-trimethylhomopiperazinyl. In some embodiments, R⁴ is N-linked morpholino, 2-methyl morpholino, 3-methyl morpholino, 2,6-dimethylmorpholino or 3,5-dimethylmorpholino.

Some embodiments of compounds of Formula (IV) are inhibitors of the MLL1-WDR5 protein-protein interaction.

In some embodiments, the compound has the structure of Formula (V), or a pharmaceutically acceptable salt or solvate thereof:

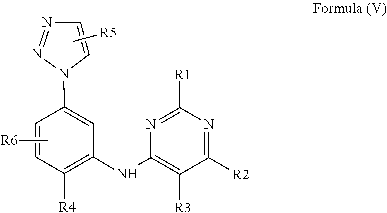

where R¹, R² and R³ are the same or different from each other, and are each independently selected from hydrogen, C₁-C₆ alkyl, C₃-C₆ cycloalkyl, C₁-C₆ alkoxy-substituted C₁-C₆ alkyl, nitro, halogen, cyano, aldehyde, hydroxyl;
R⁴ represents substituted or unsubstituted morpholinyl, substituted or unsubstituted piperazinyl, 4-substituted piperazinyl, 4-substituted homopiperazinyl, 3-substituted piperazinyl or 2-substituted piperazinyl, the substituent is C₁-C₄ alkyl, 3-7-membered cycloalkyl, hydroxyalkyl, or phenyl;
R⁵ is absent or R⁵ is nitro, amino, phenyl, substituted phenyl, 5-6-membered aromatic heterocyclic ring containing oxygen or nitrogen, substituted 5-6-membered aromatic heterocyclic ring containing oxygen or nitrogen, —CONR¹³R¹⁴, where R¹³, R¹⁴ represents hydrogen, C₁-C₆ alkyl, phenyl or substituted phenyl, substituted or unsubstituted 3-7-membered heterocyclic ring containing nitrogen, oxygen or both, or R¹³ and R¹⁴ together, form a 3-7-membered heterocyclic ring containing nitrogen, oxygen or both formed by linking R¹³ and R¹⁴;
R⁶ represents hydrogen, halogen, methyl, trifluoromethyl, amino, substituted amino, and the substituent is C₁-C₄ alkyl, or allyl.

In some embodiments of the compound of Formula (V) R¹ is H, F or CH₃ (preferably H), R² is halo (preferably F, Cl or Br, more preferably Cl), and R³ is —NH₂, —NHCH₃ or —N(CH₃)₂ (preferably —NH₂).

Some embodiments of compounds of Formula (V) are inhibitors of the MLL1-WDR5 protein-protein interaction.

In some embodiments, the compound has the structure of Formula (VI), or a pharmaceutically acceptable salt or solvate thereof:

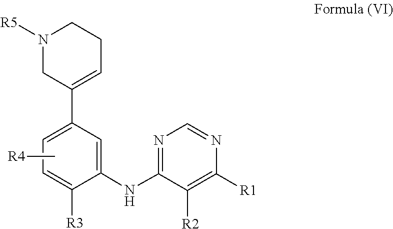

wherein R¹, R² and R⁴ are the same or different from each other, and are each independently selected from, Cl, F, Br, I or —NH₂;
R³ represents substituted or unsubstituted morpholinyl, substituted or unsubstituted piperazinyl, 4-substituted piperazinyl, 4-substituted homopiperazinyl, 3-substituted piperazinyl or 2-substituted piperazinyl, {where the substituent is C₁-C₄ alkyl, 3-7-membered cycloalkyl, hydroxyalkyl, or phenyl};
R⁵ is substituted 5-6-membered aromatic heterocyclic ring containing oxygen, nitrogen or both, {where each substituent is independently C₁-C₄ alkyl, substituted C₁-C₄ alkyl {wherein the substituent is —NR⁶R⁷, (wherein R⁶ and R⁷ are independently C₁-C₄ alkyl or 3-7-membered heterocyclic ring containing nitrogen or oxygen or both), or (R⁶ and R⁷ are linked together to form a 3-7-membered heterocyclic ring containing nitrogen, oxygen or both)}.

In some embodiments of the compound of Formula (VI) R¹ is H, F or CH₃ (preferably H), R² is halo (preferably F, Cl or Br, more preferably Cl), and R³ is NH₂, NHCH₃ or N(CH₃)₂ (preferably NH₂).

Some embodiments of compounds of Formula (VI) are inhibitors of the MLL1-WDR5 protein-protein interaction.

Any combination of the groups described above for the various variables is contemplated herein. Throughout the specification, groups and substituents thereof are chosen by one skilled in the field to provide stable moieties and compounds.

In some embodiments, compounds described herein include, but are not limited to the compound of Table 1, Table 2, Table 3 or Table 4, or a pharmaceutically acceptable salt or solvate thereof.

In some embodiments, compounds described herein do not include compounds of Formula A or DDO-2093 and Formula B or DDO-2213, or a pharmaceutically acceptable salt or solvate thereof, represented by Formula III and Formula IV respectively. In some embodiments, compounds of Formula I, Formula II, Formula III, and/or Formula V exclude the compound of Formula A (DDO-2093). In some embodiments, compounds of Formula IV exclude the compound of Formula B (DDO-2213).

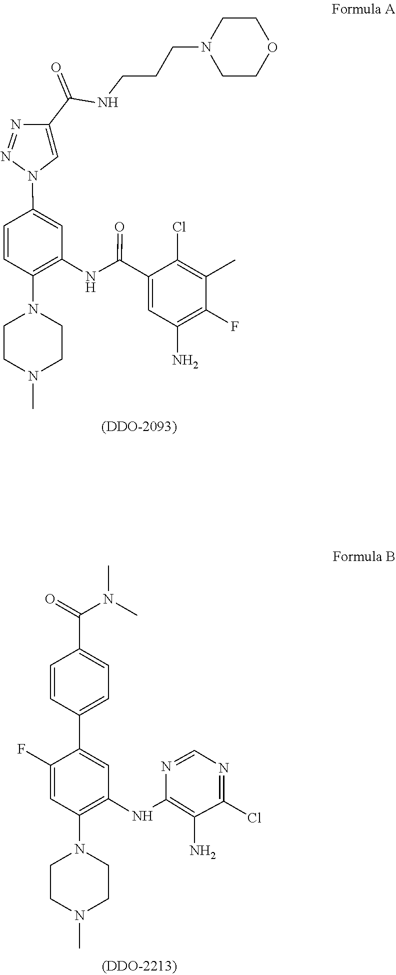 [SMILES: Cc1c(F)c(N)cc(C(=O)Nc2cc(-n3cc(C(=O)NCCCN4CCOCC4)nn3)ccc2N2CCN(C)CC2)c1Cl ; CN1CCN(c2cc(F)c(-c3ccc(C(=O)N(C)C)cc3)cc2Nc2ncnc(Cl)c2N)CC1]

TABLE 1
Compounds of the disclosure.

| Cmpd. No. | Structure |
|---|---|
| 1 (HYBI_002) | 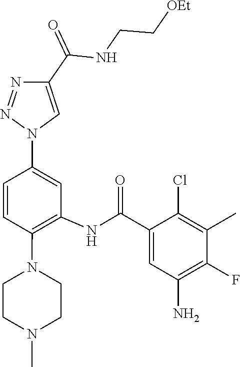 [SMILES: CCOCCNC(=O)c1cn(-c2ccc(N3CCN(C)CC3)c(NC(=O)c3cc(N)c(F)c(C)c3Cl)c2)nn1] |
| 2 (HYBI_003) | 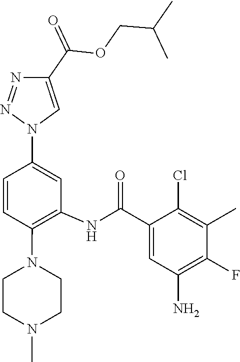 [SMILES: Cc1c(F)c(N)cc(C(=O)Nc2cc(-n3cc(C(=O)OCC(C)C)nn3)ccc2N2CCN(C)CC2)c1Cl] |
| 3 (HYBI_010) | 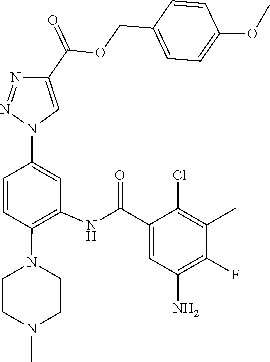 [SMILES: COc1ccc(COC(=O)c2cn(-c3ccc(N4CCN(C)CC4)c(NC(=O)c4cc(N)c(F)c(C)c4Cl)c3)nn2)cc1] |
| 4 (HYBI_013) | 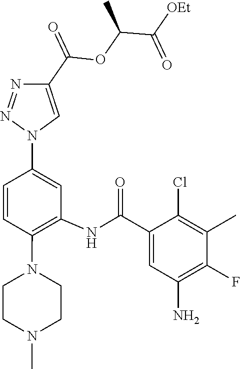 [SMILES: CCOC(=O)[C@H](C)OC(=O)c1cn(-c2ccc(N3CCN(C)CC3)c(NC(=O)c3cc(N)c(F)c(C)c3Cl)c2)nn1] |
| 5 (HYBI_014) | 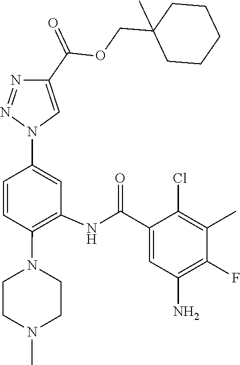 [SMILES: Cc1c(F)c(N)cc(C(=O)Nc2cc(-n3cc(C(=O)OCC4(C)CCCCC4)nn3)ccc2N2CCN(C)CC2)c1Cl] |
| 6 (HYBI_016) | 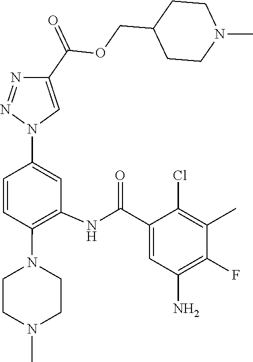 [SMILES: Cc1c(F)c(N)cc(C(=O)Nc2cc(-n3cc(C(=O)OCC4CCN(C)CC4)nn3)ccc2N2CCN(C)CC2)c1Cl] |
| 7 (HYBI_018) | 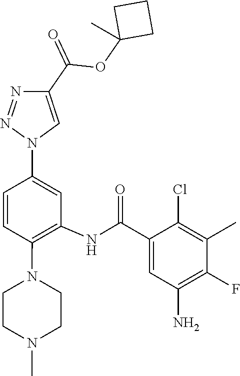 [SMILES: Cc1c(F)c(N)cc(C(=O)Nc2cc(-n3cc(C(=O)OC4(C)CCC4)nn3)ccc2N2CCN(C)CC2)c1Cl] |
| 8 (HYBI_019) | 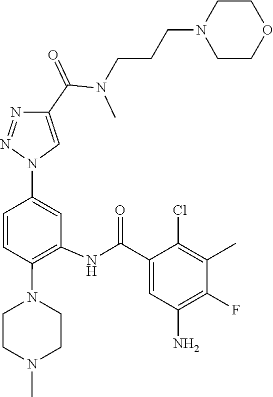 [SMILES: Cc1c(F)c(N)cc(C(=O)Nc2cc(-n3cc(C(=O)N(C)CCCN4CCOCC4)nn3)ccc2N2CCN(C)CC2)c1Cl] |
| 9 (HYBI_019A) | 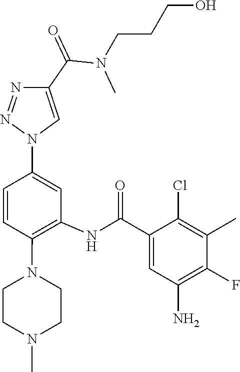 [SMILES: Cc1c(F)c(N)cc(C(=O)Nc2cc(-n3cc(C(=O)N(C)CCCO)nn3)ccc2N2CCN(C)CC2)c1Cl] |
| 10 (HYBI_020) | 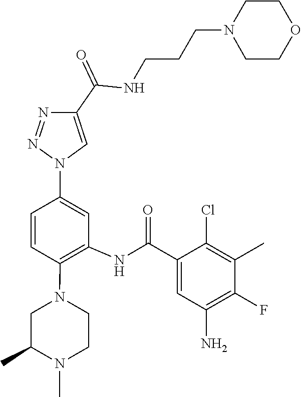 [SMILES: Cc1c(F)c(N)cc(C(=O)Nc2cc(-n3cc(C(=O)NCCCN4CCOCC4)nn3)ccc2N2CCN(C)[C@@H](C)C2)c1Cl] |
| 11 (HYBI_021) | 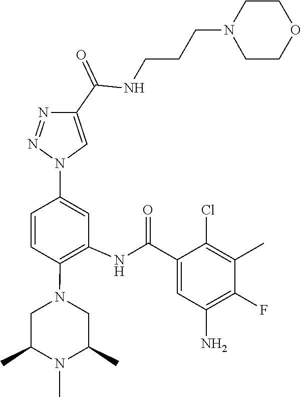 [SMILES: Cc1c(F)c(N)cc(C(=O)Nc2cc(-n3cc(C(=O)NCCCN4CCOCC4)nn3)ccc2N2C[C@@H](C)N(C)[C@@H](C)C2)c1Cl] |
| 12 (HYBI_022) | 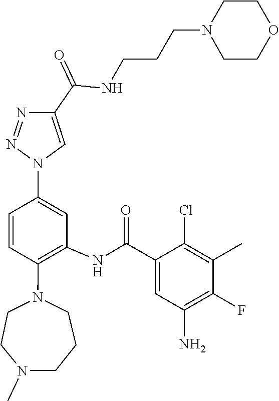 [SMILES: Cc1c(F)c(N)cc(C(=O)Nc2cc(-n3cc(C(=O)NCCCN4CCOCC4)nn3)ccc2N2CCCN(C)CC2)c1Cl] |
| 13 (HYBI_023) | 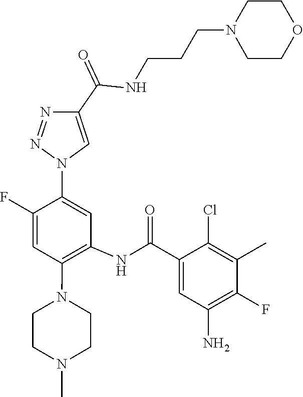 [SMILES: Cc1c(F)c(N)cc(C(=O)Nc2cc(-n3cc(C(=O)NCCCN4CCOCC4)nn3)c(F)cc2N2CCN(C)CC2)c1Cl] |
| 14 (HYBI_027) | 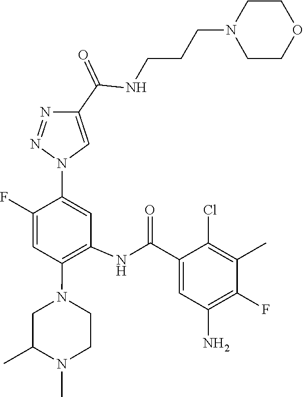 [SMILES: Cc1c(F)c(N)cc(C(=O)Nc2cc(-n3cc(C(=O)NCCCN4CCOCC4)nn3)c(F)cc2N2CCN(C)C(C)C2)c1Cl] |
| 15 (HYBI_028) | 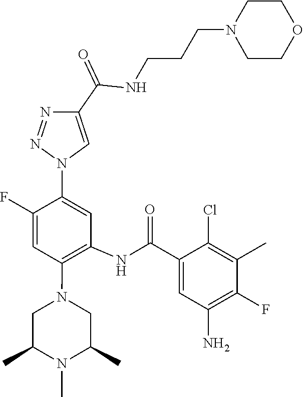 [SMILES: Cc1c(F)c(N)cc(C(=O)Nc2cc(-n3cc(C(=O)NCCCN4CCOCC4)nn3)c(F)cc2N2C[C@@H](C)N(C)[C@@H](C)C2)c1Cl] |
| 16 (HYBI_037) | 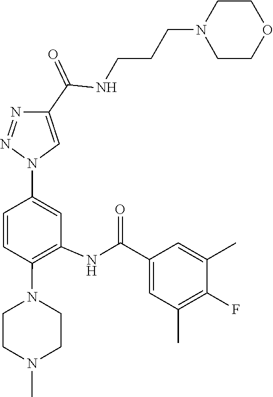 [SMILES: Cc1cc(C(=O)Nc2cc(-n3cc(C(=O)NCCCN4CCOCC4)nn3)ccc2N2CCN(C)CC2)cc(C)c1F] |
| 17 (HYBI_038) | 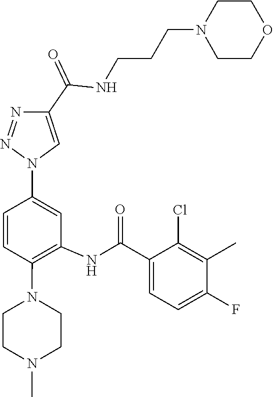 [SMILES: Cc1c(F)ccc(C(=O)Nc2cc(-n3cc(C(=O)NCCCN4CCOCC4)nn3)ccc2N2CCN(C)CC2)c1Cl] |
| 18 (HYBI_039) | 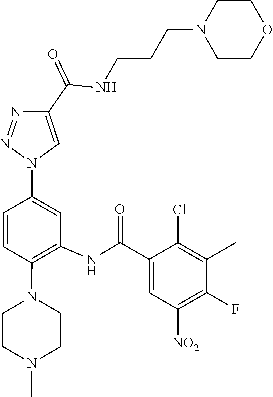 [SMILES: Cc1c(F)c([N+](=O)[O-])cc(C(=O)Nc2cc(-n3cc(C(=O)NCCCN4CCOCC4)nn3)ccc2N2CCN(C)CC2)c1Cl] |
| 19 (HYBI_040) | 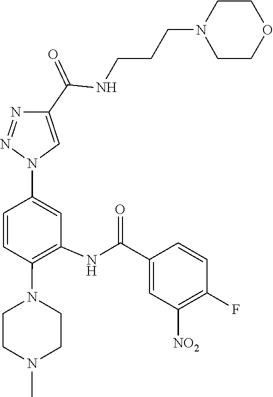 [SMILES: CN1CCN(c2ccc(-n3cc(C(=O)NCCCN4CCOCC4)nn3)cc2NC(=O)c2ccc(F)c([N+](=O)[O-])c2)CC1] |
| 20 (HYBI_041) | 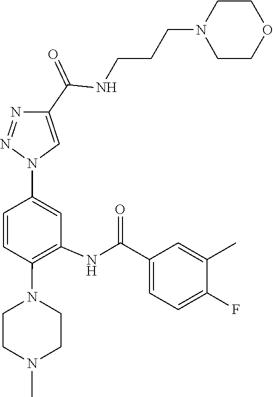 [SMILES: Cc1cc(C(=O)Nc2cc(-n3cc(C(=O)NCCCN4CCOCC4)nn3)ccc2N2CCN(C)CC2)ccc1F] |
| 21 (HYBI_042) | 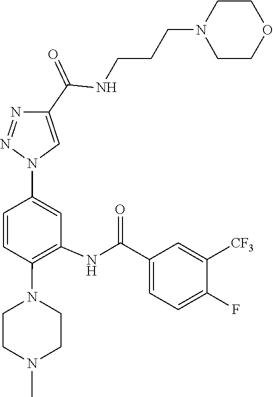 [SMILES: CN1CCN(c2ccc(-n3cc(C(=O)NCCCN4CCOCC4)nn3)cc2NC(=O)c2ccc(F)c(C(F)(F)F)c2)CC1] |
| 22 (HYBI_043) | 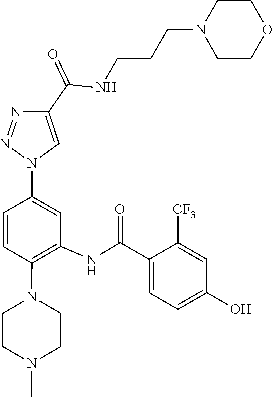 [SMILES: CN1CCN(c2ccc(-n3cc(C(=O)NCCCN4CCOCC4)nn3)cc2NC(=O)c2ccc(O)cc2C(F)(F)F)CC1] |
| 23 (HYBI_044) | 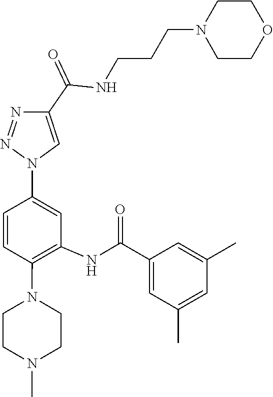 [SMILES: Cc1cc(C)cc(C(=O)Nc2cc(-n3cc(C(=O)NCCCN4CCOCC4)nn3)ccc2N2CCN(C)CC2)c1] |
| 24 (HYBI_045) | 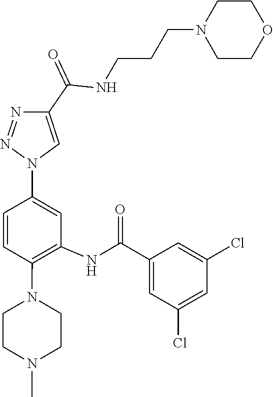 [SMILES: CN1CCN(c2ccc(-n3cc(C(=O)NCCCN4CCOCC4)nn3)cc2NC(=O)c2cc(Cl)cc(Cl)c2)CC1] |
| 25 (HYBI_046) | 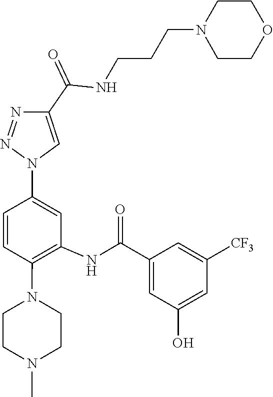 [SMILES: CN1CCN(c2ccc(-n3cc(C(=O)NCCCN4CCOCC4)nn3)cc2NC(=O)c2cc(O)cc(C(F)(F)F)c2)CC1] |
| 26 (HYBI_047) | 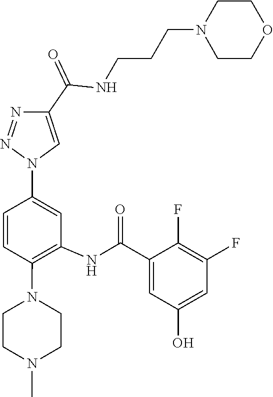 [SMILES: CN1CCN(c2ccc(-n3cc(C(=O)NCCCN4CCOCC4)nn3)cc2NC(=O)c2cc(O)cc(F)c2F)CC1] |
| 27 (HYBI_050) | 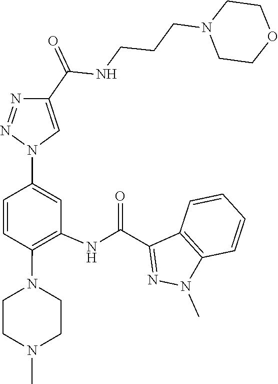 [SMILES: CN1CCN(c2ccc(-n3cc(C(=O)NCCCN4CCOCC4)nn3)cc2NC(=O)c2nn(C)c3ccccc23)CC1] |
| 28 | 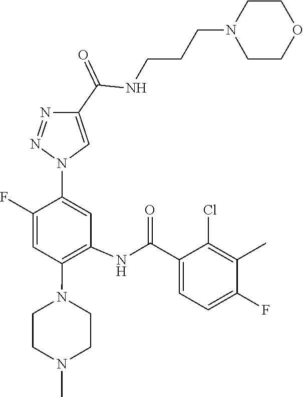 [SMILES: Cc1c(F)ccc(C(=O)Nc2cc(-n3cc(C(=O)NCCCN4CCOCC4)nn3)c(F)cc2N2CCN(C)CC2)c1Cl] |
| 29 | 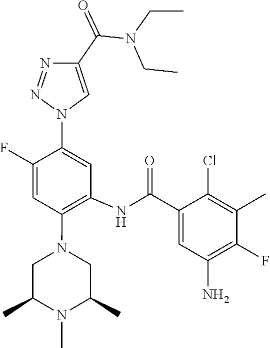 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1] |
| 30 (HYBI_083) | 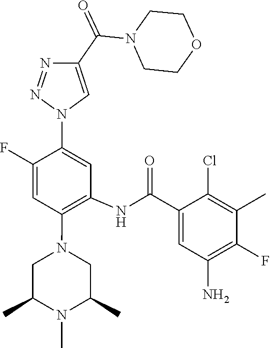 [SMILES: Cc1c(F)c(N)cc(C(=O)Nc2cc(-n3cc(C(=O)N4CCOCC4)nn3)c(F)cc2N2C[C@@H](C)N(C)[C@@H](C)C2)c1Cl] |
| 31 (HYBI_084) | 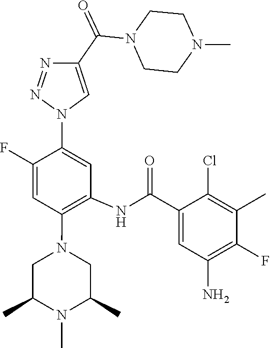 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl] |
| 32 (HYBI_085) | 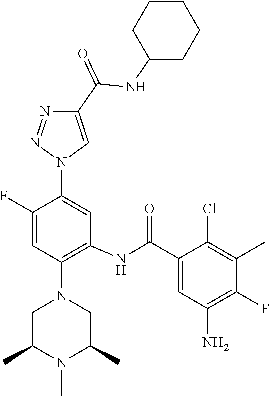 [SMILES: Cc1c(F)c(N)cc(C(=O)Nc2cc(-n3cc(C(=O)NC4CCCCC4)nn3)c(F)cc2N2C[C@@H](C)N(C)[C@@H](C)C2)c1Cl] |
| 33 (HYBI_086) | 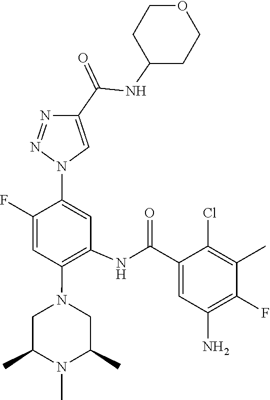 [SMILES: Cc1c(F)c(N)cc(C(=O)Nc2cc(-n3cc(C(=O)NC4CCOCC4)nn3)c(F)cc2N2C[C@@H](C)N(C)[C@@H](C)C2)c1Cl] |
| 34 (HYBI_087) | 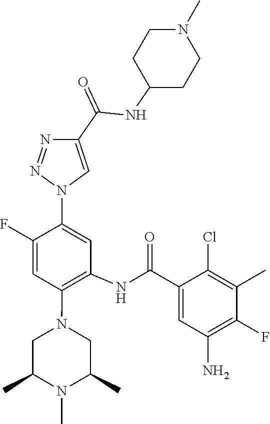 [SMILES: Cc1c(F)c(N)cc(C(=O)Nc2cc(-n3cc(C(=O)NC4CCN(C)CC4)nn3)c(F)cc2N2C[C@@H](C)N(C)[C@@H](C)C2)c1Cl] |
| 35 (HYBI_101) | 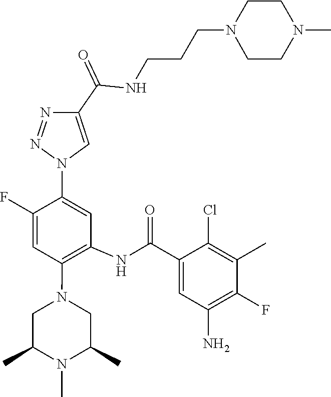 |
| 36 (HYBI_110) | 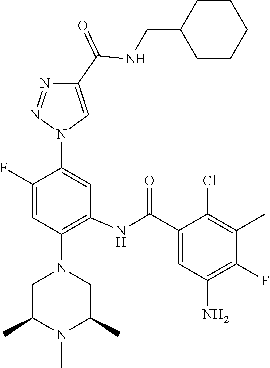 [SMILES: Cc1c(F)c(N)cc(C(=O)Nc2cc(-n3cc(C(=O)NCC4CCCCC4)nn3)c(F)cc2N2C[C@@H](C)N(C)[C@@H](C)C2)c1Cl] |
| 37 (DDO-2306) | 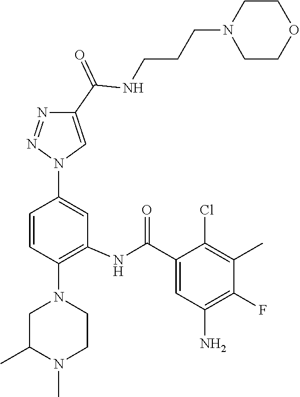 [SMILES: Cc1c(F)c(N)cc(C(=O)Nc2cc(-n3cc(C(=O)NCCCN4CCOCC4)nn3)ccc2N2CCN(C)C(C)C2)c1Cl] |
| 38 (DDO-2308) | 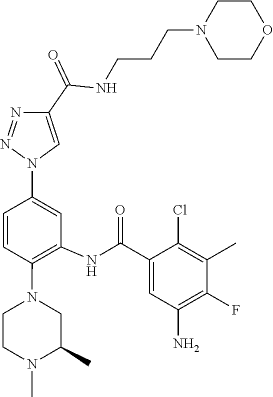 [SMILES: Cc1c(F)c(N)cc(C(=O)Nc2cc(-n3cc(C(=O)NCCCN4CCOCC4)nn3)ccc2N2CCN(C)[C@H](C)C2)c1Cl] |
| 39 (DDO-2312) | 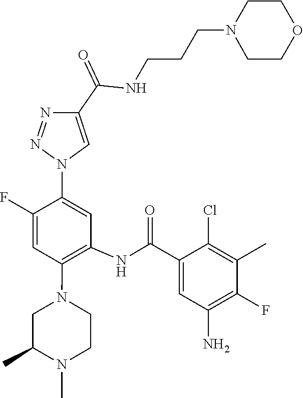 [SMILES: Cc1c(F)c(N)cc(C(=O)Nc2cc(-n3cc(C(=O)NCCCN4CCOCC4)nn3)c(F)cc2N2CCN(C)[C@@H](C)C2)c1Cl] |
| 40 (DDO-2313) | 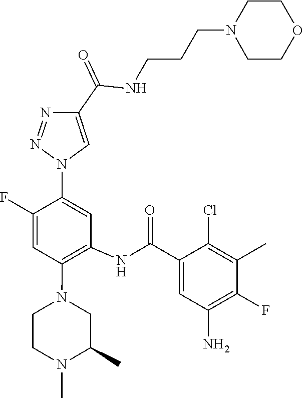 [SMILES: Cc1c(F)c(N)cc(C(=O)Nc2cc(-n3cc(C(=O)NCCCN4CCOCC4)nn3)c(F)cc2N2CCN(C)[C@H](C)C2)c1Cl] |
| 41 (DDO-2316) | 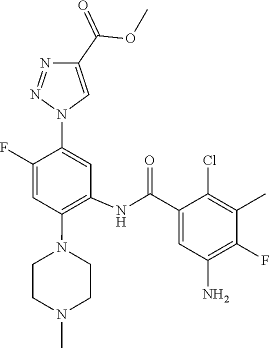 [SMILES: COC(=O)c1cn(-c2cc(NC(=O)c3cc(N)c(F)c(C)c3Cl)c(N3CCN(C)CC3)cc2F)nn1] |
| 42 (DDO-2315) | 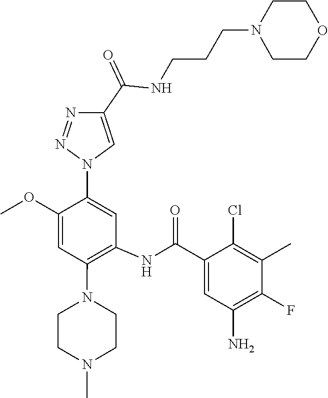 [SMILES: COc1cc(N2CCN(C)CC2)c(NC(=O)c2cc(N)c(F)c(C)c2Cl)cc1-n1cc(C(=O)NCCCN2CCOCC2)nn1] |

TABLE 2
Compounds of the disclosure.

| Cpd. No. | Structure |
|---|---|
| 44 | 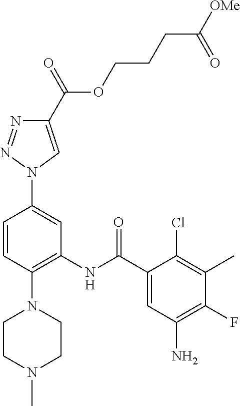 [SMILES: COC(=O)CCCOC(=O)c1cn(-c2ccc(N3CCN(C)CC3)c(NC(=O)c3cc(N)c(F)c(C)c3Cl)c2)nn1] |
| 45 | 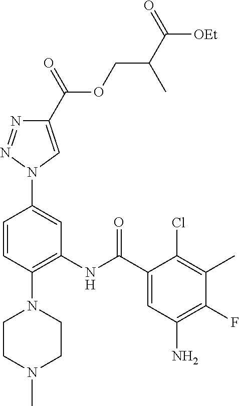 [SMILES: CCOC(=O)C(C)COC(=O)c1cn(-c2ccc(N3CCN(C)CC3)c(NC(=O)c3cc(N)c(F)c(C)c3Cl)c2)nn1] |
| 46 | 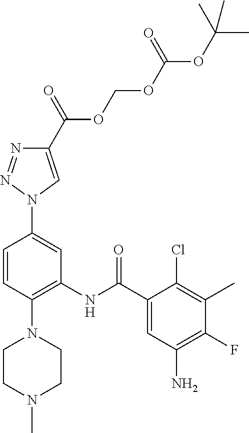 [SMILES: Cc1c(F)c(N)cc(C(=O)Nc2cc(-n3cc(C(=O)OCOC(=O)OC(C)(C)C)nn3)ccc2N2CCN(C)CC2)c1Cl] |
| 47 | 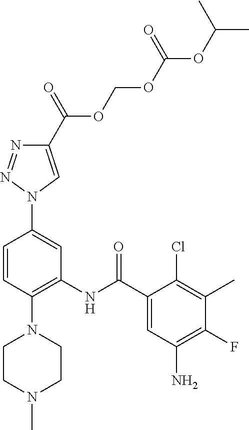 [SMILES: Cc1c(F)c(N)cc(C(=O)Nc2cc(-n3cc(C(=O)OCOC(=O)OC(C)C)nn3)ccc2N2CCN(C)CC2)c1Cl] |
| 48 | 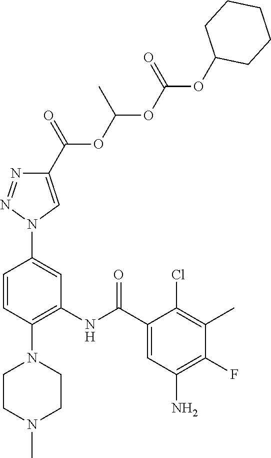 [SMILES: Cc1c(F)c(N)cc(C(=O)Nc2cc(-n3cc(C(=O)OC(C)OC(=O)OC4CCCCC4)nn3)ccc2N2CCN(C)CC2)c1Cl] |
| 49 | 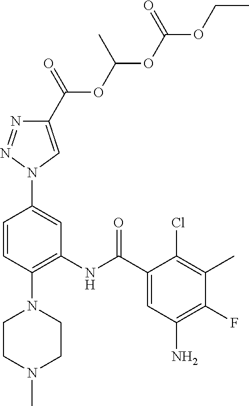 [SMILES: CCOC(=O)OC(C)OC(=O)c1cn(-c2ccc(N3CCN(C)CC3)c(NC(=O)c3cc(N)c(F)c(C)c3Cl)c2)nn1] |
| 50 | 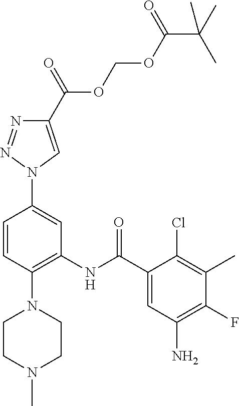 [SMILES: Cc1c(F)c(N)cc(C(=O)Nc2cc(-n3cc(C(=O)OCOC(=O)C(C)(C)C)nn3)ccc2N2CCN(C)CC2)c1Cl] |
| 51 | 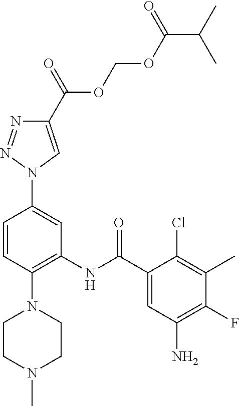 [SMILES: Cc1c(F)c(N)cc(C(=O)Nc2cc(-n3cc(C(=O)OCOC(=O)C(C)C)nn3)ccc2N2CCN(C)CC2)c1Cl] |
| 52 | 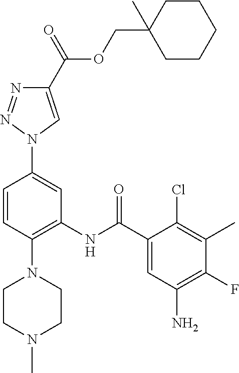 [SMILES: Cc1c(F)c(N)cc(C(=O)Nc2cc(-n3cc(C(=O)OCC4(C)CCCCC4)nn3)ccc2N2CCN(C)CC2)c1Cl] |
| 53 | 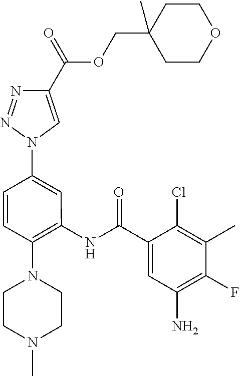 [SMILES: Cc1c(F)c(N)cc(C(=O)Nc2cc(-n3cc(C(=O)OCC4(C)CCOCC4)nn3)ccc2N2CCN(C)CC2)c1Cl] |
| 54 | 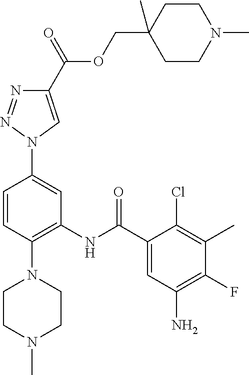 [SMILES: Cc1c(F)c(N)cc(C(=O)Nc2cc(-n3cc(C(=O)OCC4(C)CCN(C)CC4)nn3)ccc2N2CCN(C)CC2)c1Cl] |
| 55 | 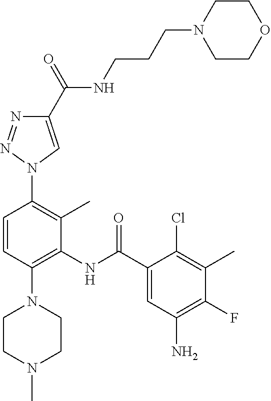 [SMILES: Cc1c(F)c(N)cc(C(=O)Nc2c(N3CCN(C)CC3)ccc(-n3cc(C(=O)NCCCN4CCOCC4)nn3)c2C)c1Cl] |
| 56 | 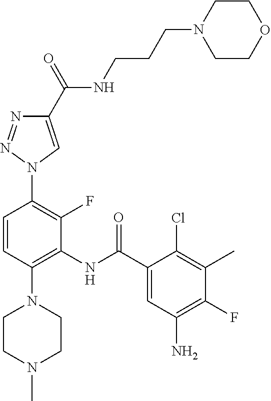 [SMILES: Cc1c(F)c(N)cc(C(=O)Nc2c(N3CCN(C)CC3)ccc(-n3cc(C(=O)NCCCN4CCOCC4)nn3)c2F)c1Cl] |
| 57 | 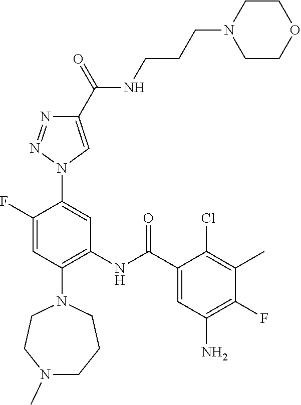 [SMILES: Cc1c(F)c(N)cc(C(=O)Nc2cc(-n3cc(C(=O)NCCCN4CCOCC4)nn3)c(F)cc2N2CCCN(C)CC2)c1Cl] |
| 58 | 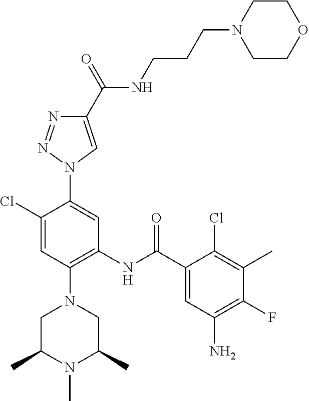 [SMILES: Cc1c(F)c(N)cc(C(=O)Nc2cc(-n3cc(C(=O)NCCCN4CCOCC4)nn3)c(Cl)cc2N2C[C@@H](C)N(C)[C@@H](C)C2)c1Cl] |
| 59 | 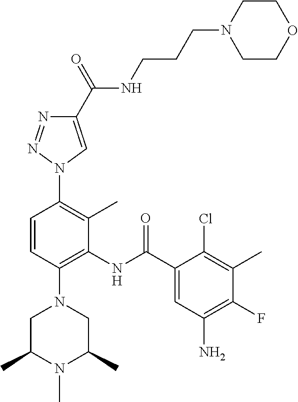 [SMILES: Cc1c(F)c(N)cc(C(=O)Nc2c(N3C[C@@H](C)N(C)[C@@H](C)C3)ccc(-n3cc(C(=O)NCCCN4CCOCC4)nn3)c2C)c1Cl] |
| 60 | 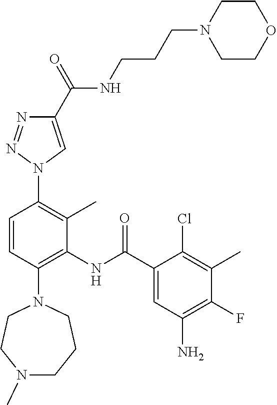 [SMILES: Cc1c(F)c(N)cc(C(=O)Nc2c(N3CCCN(C)CC3)ccc(-n3cc(C(=O)NCCCN4CCOCC4)nn3)c2C)c1Cl] |
| 61 | 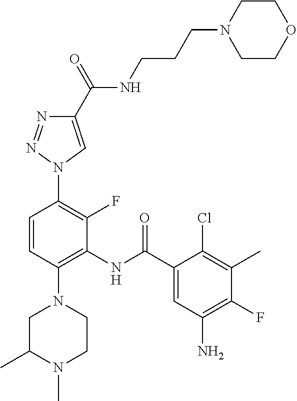 [SMILES: Cc1c(F)c(N)cc(C(=O)Nc2c(N3CCN(C)C(C)C3)ccc(-n3cc(C(=O)NCCCN4CCOCC4)nn3)c2F)c1Cl] |
| 62 | 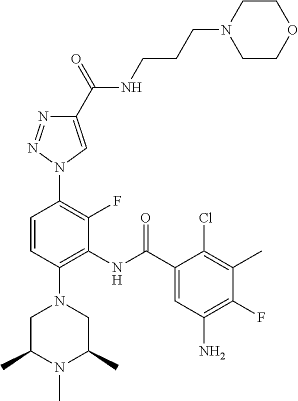 [SMILES: Cc1c(F)c(N)cc(C(=O)Nc2c(N3C[C@@H](C)N(C)[C@@H](C)C3)ccc(-n3cc(C(=O)NCCCN4CCOCC4)nn3)c2F)c1Cl] |
| 63 | 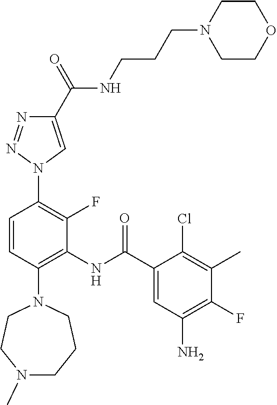 [SMILES: Cc1c(F)c(N)cc(C(=O)Nc2c(N3CCCN(C)CC3)ccc(-n3cc(C(=O)NCCCN4CCOCC4)nn3)c2F)c1Cl] |
| 64 | 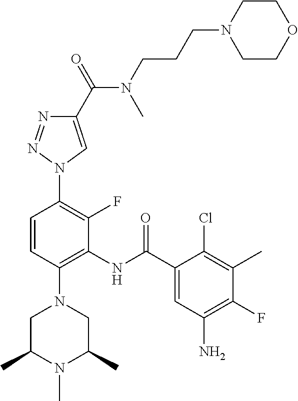 [SMILES: Cc1c(F)c(N)cc(C(=O)Nc2c(N3C[C@@H](C)N(C)[C@@H](C)C3)ccc(-n3cc(C(=O)N(C)CCCN4CCOCC4)nn3)c2F)c1Cl] |
| 65 | 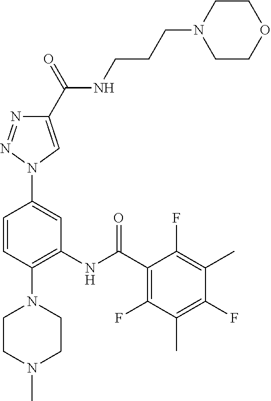 [SMILES: Cc1c(F)c(C)c(F)c(C(=O)Nc2cc(-n3cc(C(=O)NCCCN4CCOCC4)nn3)ccc2N2CCN(C)CC2)c1F] |
| 66 | 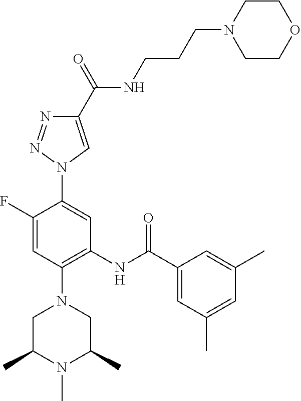 [SMILES: Cc1cc(C)cc(C(=O)Nc2cc(-n3cc(C(=O)NCCCN4CCOCC4)nn3)c(F)cc2N2C[C@@H](C)N(C)[C@@H](C)C2)c1] |
| 67 | 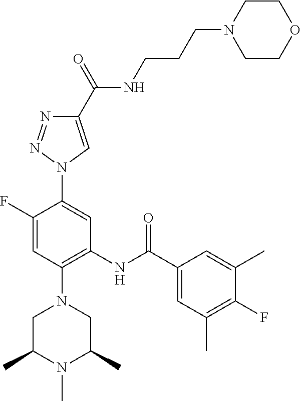 [SMILES: Cc1cc(C(=O)Nc2cc(-n3cc(C(=O)NCCCN4CCOCC4)nn3)c(F)cc2N2C[C@@H](C)N(C)[C@@H](C)C2)cc(C)c1F] |
| 68 | 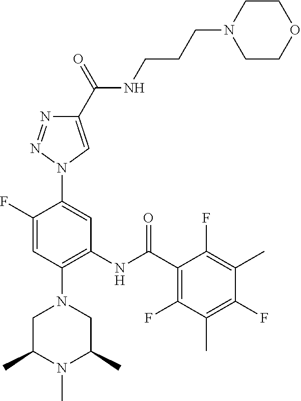 [SMILES: Cc1c(F)c(C)c(F)c(C(=O)Nc2cc(-n3cc(C(=O)NCCCN4CCOCC4)nn3)c(F)cc2N2C[C@@H](C)N(C)[C@@H](C)C2)c1F] |
| 69 | 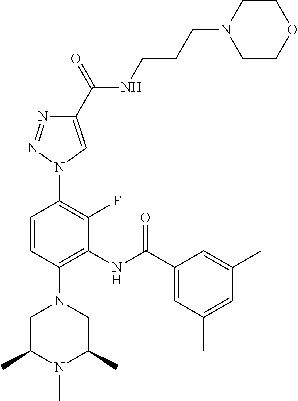 [SMILES: Cc1cc(C)cc(C(=O)Nc2c(N3C[C@@H](C)N(C)[C@@H](C)C3)ccc(-n3cc(C(=O)NCCCN4CCOCC4)nn3)c2F)c1] |
| 70 | 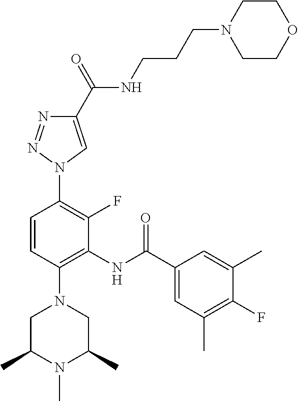 [SMILES: Cc1cc(C(=O)Nc2c(N3C[C@@H](C)N(C)[C@@H](C)C3)ccc(-n3cc(C(=O)NCCCN4CCOCC4)nn3)c2F)cc(C)c1F] |
| 71 | 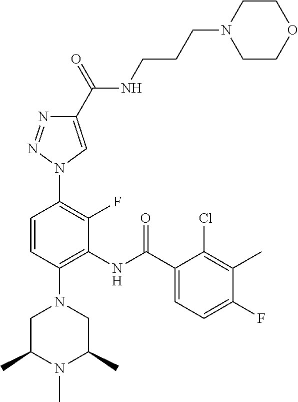 [SMILES: Cc1c(F)ccc(C(=O)Nc2c(N3C[C@@H](C)N(C)[C@@H](C)C3)ccc(-n3cc(C(=O)NCCCN4CCOCC4)nn3)c2F)c1Cl] |
| 72 | 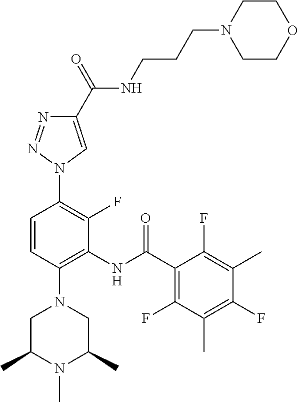 [SMILES: Cc1c(F)c(C)c(F)c(C(=O)Nc2c(N3C[C@@H](C)N(C)[C@@H](C)C3)ccc(-n3cc(C(=O)NCCCN4CCOCC4)nn3)c2F)c1F] |
| 73 | 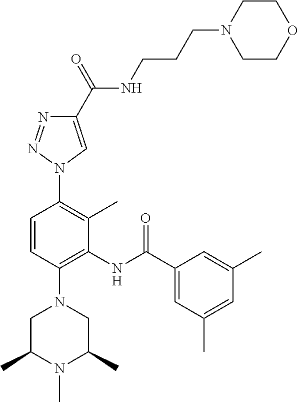 [SMILES: Cc1cc(C)cc(C(=O)Nc2c(N3C[C@@H](C)N(C)[C@@H](C)C3)ccc(-n3cc(C(=O)NCCCN4CCOCC4)nn3)c2C)c1] |
| 74 | 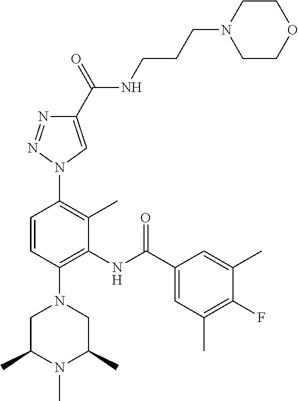 [SMILES: Cc1cc(C(=O)Nc2c(N3C[C@@H](C)N(C)[C@@H](C)C3)ccc(-n3cc(C(=O)NCCCN4CCOCC4)nn3)c2C)cc(C)c1F] |
| 75 | 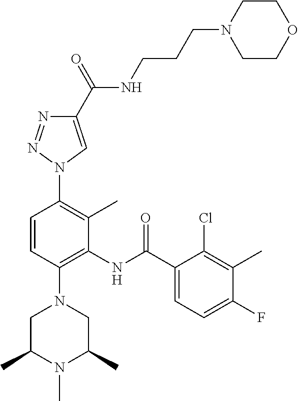 [SMILES: Cc1c(F)ccc(C(=O)Nc2c(N3C[C@@H](C)N(C)[C@@H](C)C3)ccc(-n3cc(C(=O)NCCCN4CCOCC4)nn3)c2C)c1Cl] |
| 76 | 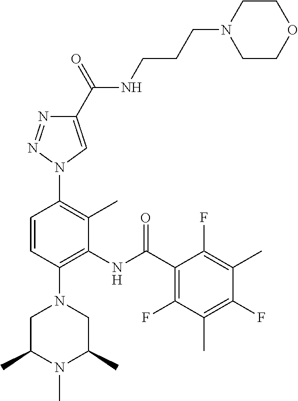 [SMILES: Cc1c(F)c(C)c(F)c(C(=O)Nc2c(N3C[C@@H](C)N(C)[C@@H](C)C3)ccc(-n3cc(C(=O)NCCCN4CCOCC4)nn3)c2C)c1F] |
| 77 | 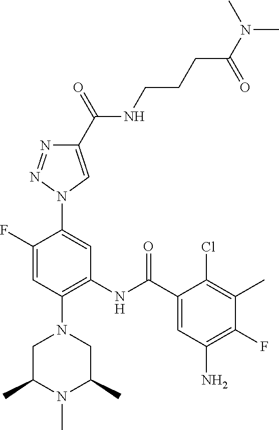 [SMILES: Cc1c(F)c(N)cc(C(=O)Nc2cc(-n3cc(C(=O)NCCCC(=O)N(C)C)nn3)c(F)cc2N2C[C@@H](C)N(C)[C@@H](C)C2)c1Cl] |
| 78 | 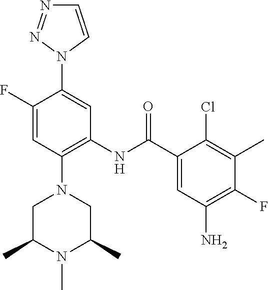 [SMILES: Cc1c(F)c(N)cc(C(=O)Nc2cc(-n3ccnn3)c(F)cc2N2C[C@@H](C)N(C)[C@@H](C)C2)c1Cl] |
| 79 | 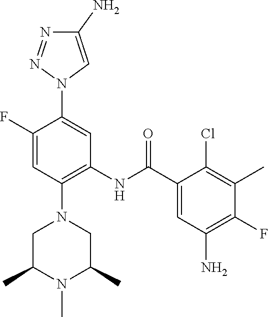 [SMILES: Cc1c(F)c(N)cc(C(=O)Nc2cc(-n3cc(N)nn3)c(F)cc2N2C[C@@H](C)N(C)[C@@H](C)C2)c1Cl] |
| 80 | 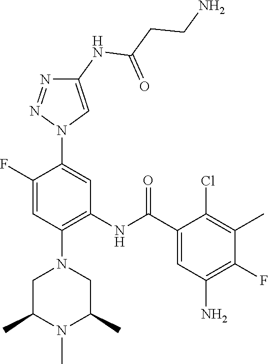 [SMILES: Cc1c(F)c(N)cc(C(=O)Nc2cc(-n3cc(NC(=O)CCN)nn3)c(F)cc2N2C[C@@H](C)N(C)[C@@H](C)C2)c1Cl] |
| 81 | 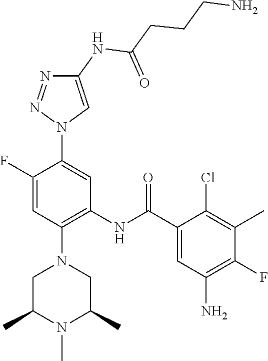 [SMILES: Cc1c(F)c(N)cc(C(=O)Nc2cc(-n3cc(NC(=O)CCCN)nn3)c(F)cc2N2C[C@@H](C)N(C)[C@@H](C)C2)c1Cl] |
| 82 | 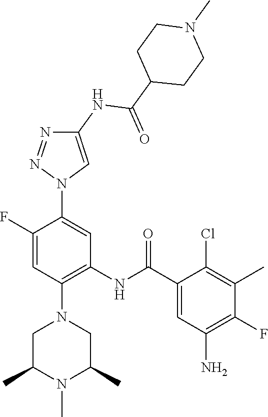 [SMILES: Cc1c(F)c(N)cc(C(=O)Nc2cc(-n3cc(NC(=O)C4CCN(C)CC4)nn3)c(F)cc2N2C[C@@H](C)N(C)[C@@H](C)C2)c1Cl] |
| 83 | 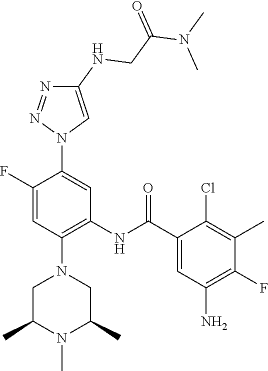 [SMILES: Cc1c(F)c(N)cc(C(=O)Nc2cc(-n3cc(NCC(=O)N(C)C)nn3)c(F)cc2N2C[C@@H](C)N(C)[C@@H](C)C2)c1Cl] |
| 84 | 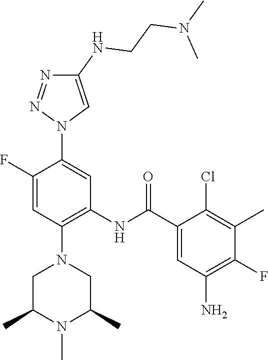 [SMILES: Cc1c(F)c(N)cc(C(=O)Nc2cc(-n3cc(NCCN(C)C)nn3)c(F)cc2N2C[C@@H](C)N(C)[C@@H](C)C2)c1Cl] |
| 85 | 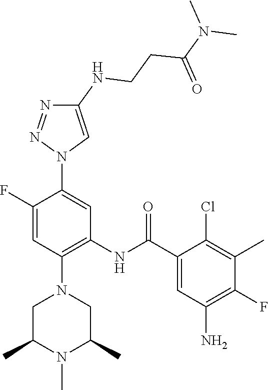 [SMILES: Cc1c(F)c(N)cc(C(=O)Nc2cc(-n3cc(NCCC(=O)N(C)C)nn3)c(F)cc2N2C[C@@H](C)N(C)[C@@H](C)C2)c1Cl] |
| 86 | 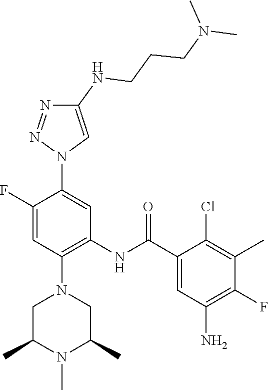 [SMILES: Cc1c(F)c(N)cc(C(=O)Nc2cc(-n3cc(NCCCN(C)C)nn3)c(F)cc2N2C[C@@H](C)N(C)[C@@H](C)C2)c1Cl] |
| 87 | 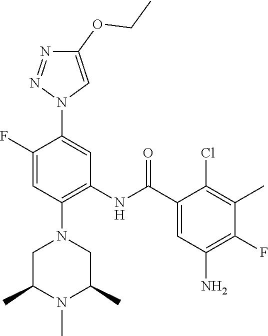 [SMILES: CCOc1cn(-c2cc(NC(=O)c3cc(N)c(F)c(C)c3Cl)c(N3C[C@@H](C)N(C)[C@@H](C)C3)cc2F)nn1] |
| 88 | 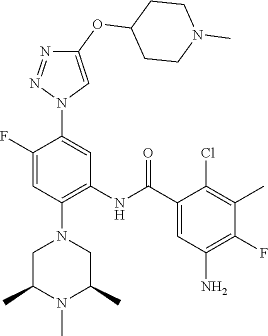 [SMILES: Cc1c(F)c(N)cc(C(=O)Nc2cc(-n3cc(OC4CCN(C)CC4)nn3)c(F)cc2N2C[C@@H](C)N(C)[C@@H](C)C2)c1Cl] |
| 89 | 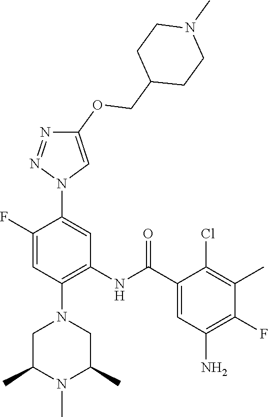 [SMILES: Cc1c(F)c(N)cc(C(=O)Nc2cc(-n3cc(OCC4CCN(C)CC4)nn3)c(F)cc2N2C[C@@H](C)N(C)[C@@H](C)C2)c1Cl] |
| 90 | 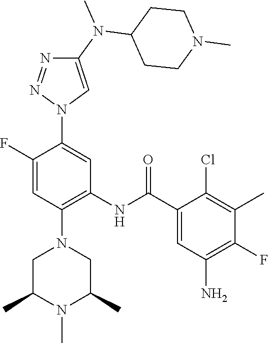 [SMILES: Cc1c(F)c(N)cc(C(=O)Nc2cc(-n3cc(N(C)C4CCN(C)CC4)nn3)c(F)cc2N2C[C@@H](C)N(C)[C@@H](C)C2)c1Cl] |
| 91 | 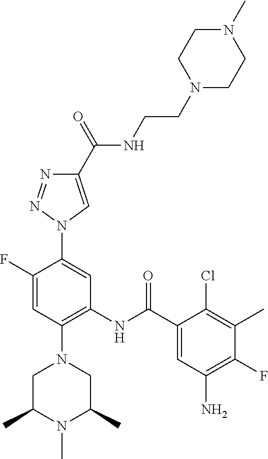 [SMILES: Cc1c(F)c(N)cc(C(=O)Nc2cc(-n3cc(C(=O)NCCN4CCN(C)CC4)nn3)c(F)cc2N2C[C@@H](C)N(C)[C@@H](C)C2)c1Cl] |
| 92 | 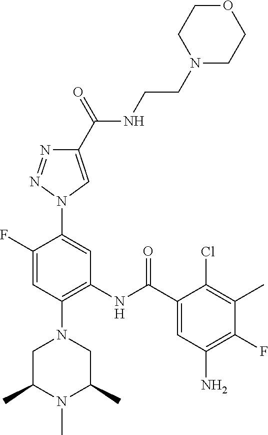 [SMILES: Cc1c(F)c(N)cc(C(=O)Nc2cc(-n3cc(C(=O)NCCN4CCOCC4)nn3)c(F)cc2N2C[C@@H](C)N(C)[C@@H](C)C2)c1Cl] |
| 93 | 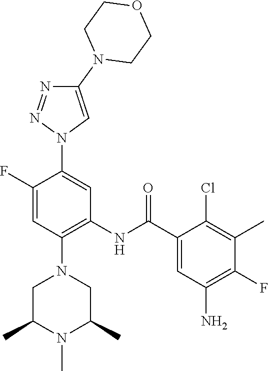 [SMILES: Cc1c(F)c(N)cc(C(=O)Nc2cc(-n3cc(N4CCOCC4)nn3)c(F)cc2N2C[C@@H](C)N(C)[C@@H](C)C2)c1Cl] |
| 94 | 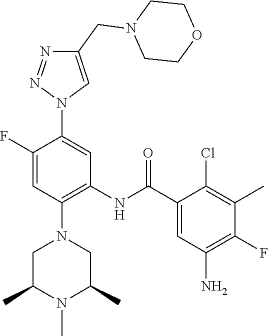 [SMILES: Cc1c(F)c(N)cc(C(=O)Nc2cc(-n3cc(CN4CCOCC4)nn3)c(F)cc2N2C[C@@H](C)N(C)[C@@H](C)C2)c1Cl] |
| 95 | 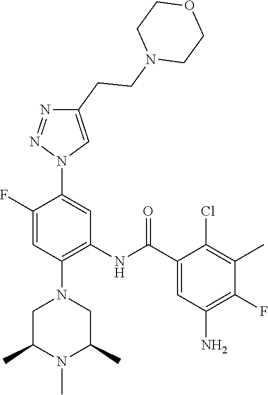 [SMILES: Cc1c(F)c(N)cc(C(=O)Nc2cc(-n3cc(CCN4CCOCC4)nn3)c(F)cc2N2C[C@@H](C)N(C)[C@@H](C)C2)c1Cl] |
| 96 | 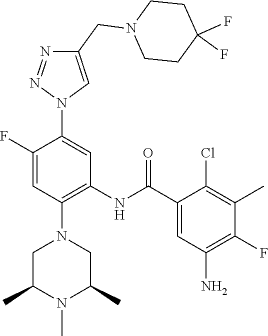 [SMILES: Cc1c(F)c(N)cc(C(=O)Nc2cc(-n3cc(CN4CCC(F)(F)CC4)nn3)c(F)cc2N2C[C@@H](C)N(C)[C@@H](C)C2)c1Cl] |
| 97 | 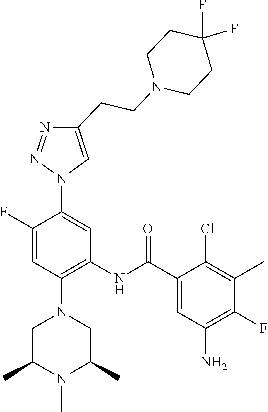 [SMILES: Cc1c(F)c(N)cc(C(=O)Nc2cc(-n3cc(CCN4CCC(F)(F)CC4)nn3)c(F)cc2N2C[C@@H](C)N(C)[C@@H](C)C2)c1Cl] |
| 98 | 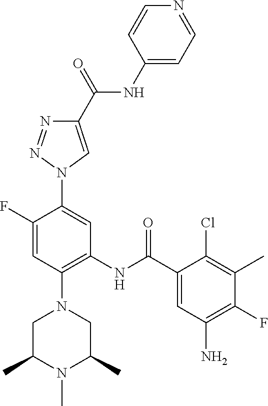 [SMILES: Cc1c(F)c(N)cc(C(=O)Nc2cc(-n3cc(C(=O)Nc4ccncc4)nn3)c(F)cc2N2C[C@@H](C)N(C)[C@@H](C)C2)c1Cl] |
| 99 | 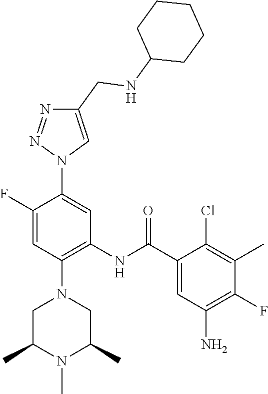 [SMILES: Cc1c(F)c(N)cc(C(=O)Nc2cc(-n3cc(CNC4CCCCC4)nn3)c(F)cc2N2C[C@@H](C)N(C)[C@@H](C)C2)c1Cl] |
| 100 | 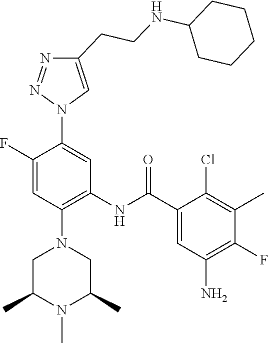 [SMILES: Cc1c(F)c(N)cc(C(=O)Nc2cc(-n3cc(CCNC4CCCCC4)nn3)c(F)cc2N2C[C@@H](C)N(C)[C@@H](C)C2)c1Cl] |
| 101 | 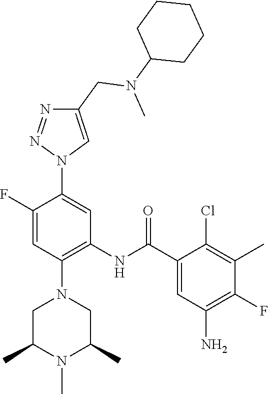 [SMILES: Cc1c(F)c(N)cc(C(=O)Nc2cc(-n3cc(CN(C)C4CCCCC4)nn3)c(F)cc2N2C[C@@H](C)N(C)[C@@H](C)C2)c1Cl] |
| 102 | 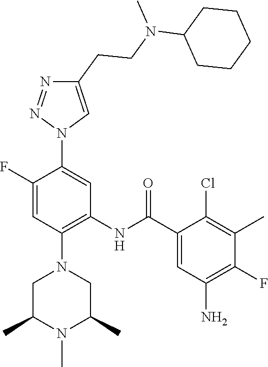 [SMILES: Cc1c(F)c(N)cc(C(=O)Nc2cc(-n3cc(CCN(C)C4CCCCC4)nn3)c(F)cc2N2C[C@@H](C)N(C)[C@@H](C)C2)c1Cl] |
| 103 | 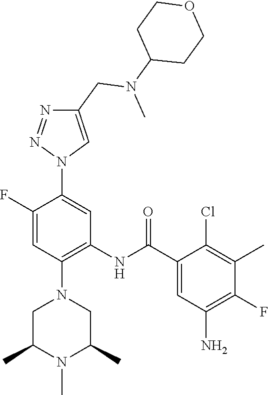 [SMILES: Cc1c(F)c(N)cc(C(=O)Nc2cc(-n3cc(CN(C)C4CCOCC4)nn3)c(F)cc2N2C[C@@H](C)N(C)[C@@H](C)C2)c1Cl] |
| 104 | 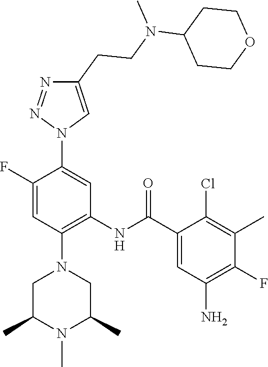 [SMILES: Cc1c(F)c(N)cc(C(=O)Nc2cc(-n3cc(CCN(C)C4CCOCC4)nn3)c(F)cc2N2C[C@@H](C)N(C)[C@@H](C)C2)c1Cl] |
| 105 | 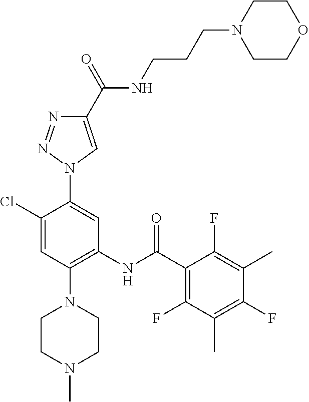 [SMILES: Cc1c(F)c(C)c(F)c(C(=O)Nc2cc(-n3cc(C(=O)NCCCN4CCOCC4)nn3)c(Cl)cc2N2CCN(C)CC2)c1F] |
| 106 | 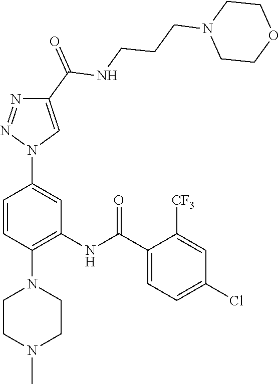 [SMILES: CN1CCN(c2ccc(-n3cc(C(=O)NCCCN4CCOCC4)nn3)cc2NC(=O)c2ccc(Cl)cc2C(F)(F)F)CC1] |
| 107 | 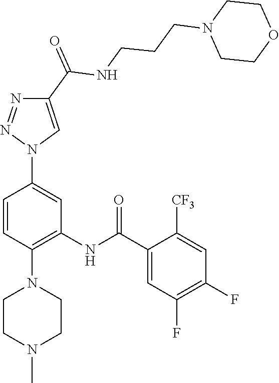 [SMILES: CN1CCN(c2ccc(-n3cc(C(=O)NCCCN4CCOCC4)nn3)cc2NC(=O)c2cc(F)c(F)cc2C(F)(F)F)CC1] |
| 108 | 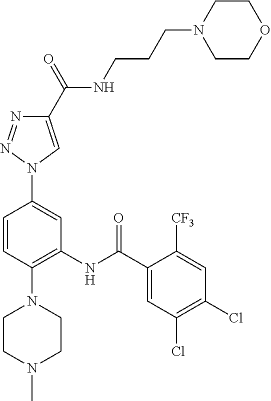 [SMILES: CN1CCN(c2ccc(-n3cc(C(=O)NCCCN4CCOCC4)nn3)cc2NC(=O)c2cc(Cl)c(Cl)cc2C(F)(F)F)CC1] |
| 109 | 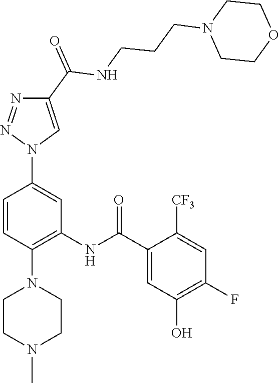 [SMILES: CN1CCN(c2ccc(-n3cc(C(=O)NCCCN4CCOCC4)nn3)cc2NC(=O)c2cc(O)c(F)cc2C(F)(F)F)CC1] |
| 110 | 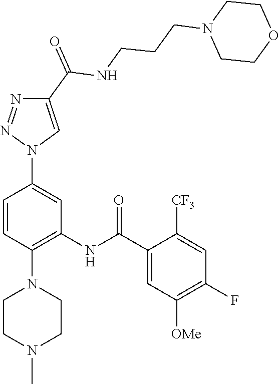 [SMILES: COc1cc(C(=O)Nc2cc(-n3cc(C(=O)NCCCN4CCOCC4)nn3)ccc2N2CCN(C)CC2)c(C(F)(F)F)cc1F] |
| 111 | 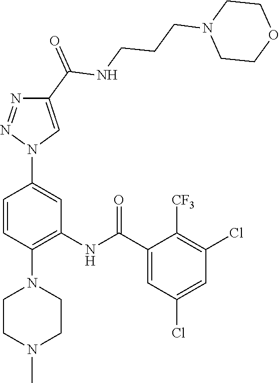 [SMILES: CN1CCN(c2ccc(-n3cc(C(=O)NCCCN4CCOCC4)nn3)cc2NC(=O)c2cc(Cl)cc(Cl)c2C(F)(F)F)CC1] |
| 112 | 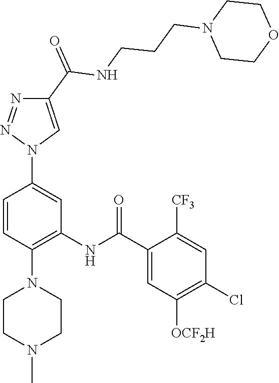 |
| 113 | 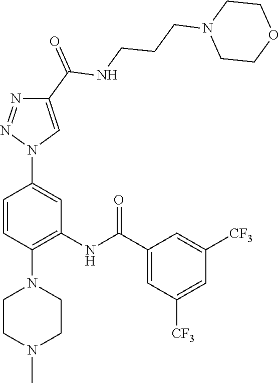 [SMILES: CN1CCN(c2ccc(-n3cc(C(=O)NCCCN4CCOCC4)nn3)cc2NC(=O)c2cc(C(F)(F)F)cc(C(F)(F)F)c2)CC1] |
| 114 | 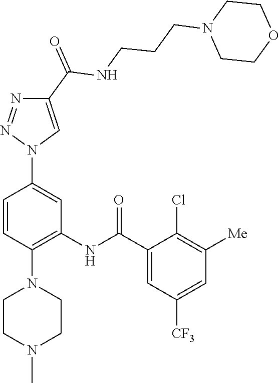 [SMILES: Cc1cc(C(F)(F)F)cc(C(=O)Nc2cc(-n3cc(C(=O)NCCCN4CCOCC4)nn3)ccc2N2CCN(C)CC2)c1Cl] |
| 115 | 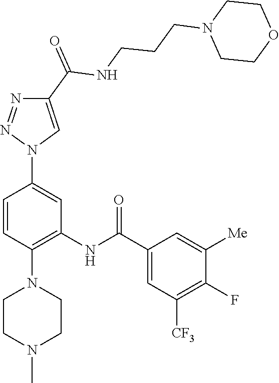 [SMILES: Cc1cc(C(=O)Nc2cc(-n3cc(C(=O)NCCCN4CCOCC4)nn3)ccc2N2CCN(C)CC2)cc(C(F)(F)F)c1F] |
| 116 | 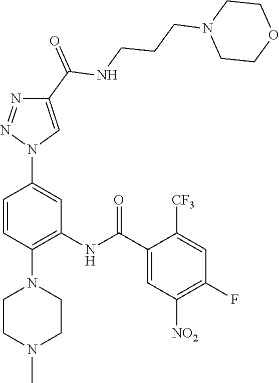 [SMILES: CN1CCN(c2ccc(-n3cc(C(=O)NCCCN4CCOCC4)nn3)cc2NC(=O)c2cc([N+](=O)[O-])c(F)cc2C(F)(F)F)CC1] |
| 117 | 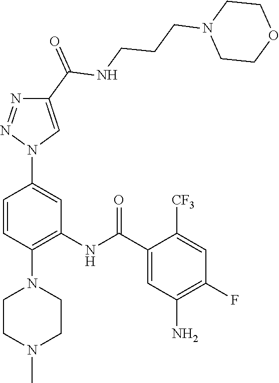 [SMILES: CN1CCN(c2ccc(-n3cc(C(=O)NCCCN4CCOCC4)nn3)cc2NC(=O)c2cc(N)c(F)cc2C(F)(F)F)CC1] |
| 118 | 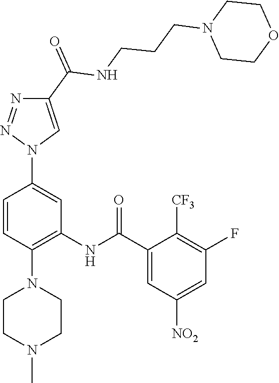 [SMILES: CN1CCN(c2ccc(-n3cc(C(=O)NCCCN4CCOCC4)nn3)cc2NC(=O)c2cc([N+](=O)[O-])cc(F)c2C(F)(F)F)CC1] |
| 119 | 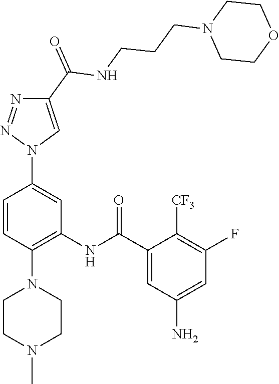 [SMILES: CN1CCN(c2ccc(-n3cc(C(=O)NCCCN4CCOCC4)nn3)cc2NC(=O)c2cc(N)cc(F)c2C(F)(F)F)CC1] |
| 120 | 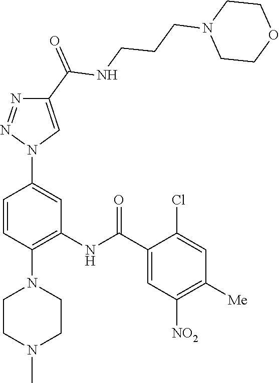 [SMILES: Cc1cc(Cl)c(C(=O)Nc2cc(-n3cc(C(=O)NCCCN4CCOCC4)nn3)ccc2N2CCN(C)CC2)cc1[N+](=O)[O-]] |
| 121 | 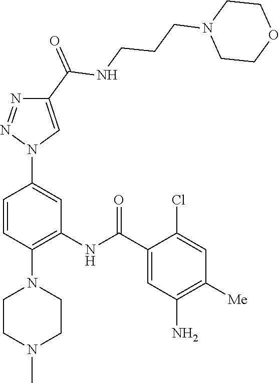 [SMILES: Cc1cc(Cl)c(C(=O)Nc2cc(-n3cc(C(=O)NCCCN4CCOCC4)nn3)ccc2N2CCN(C)CC2)cc1N] |

TABLE 3
Compounds of the disclosure.

| Cmpd. No. | Structure |
|---|---|
| 123 (DDO-001) | 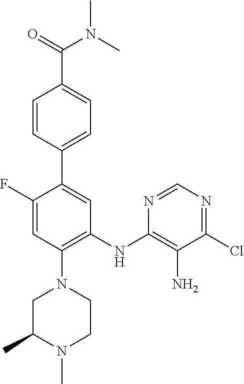 [SMILES: C[C@H]1CN(c2cc(F)c(-c3ccc(C(=O)N(C)C)cc3)cc2Nc2ncnc(Cl)c2N)CCN1C] |
| 124 (DDO-002) | 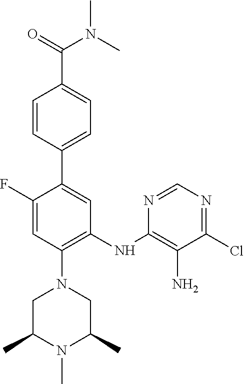 [SMILES: C[C@@H]1CN(c2cc(F)c(-c3ccc(C(=O)N(C)C)cc3)cc2Nc2ncnc(Cl)c2N)C[C@H](C)N1C] |
| 125 (DDO-003) | 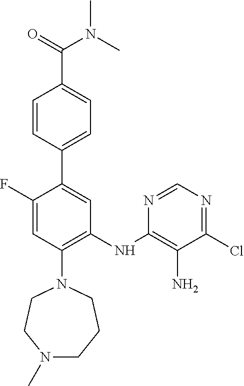 [SMILES: CN1CCCN(c2cc(F)c(-c3ccc(C(=O)N(C)C)cc3)cc2Nc2ncnc(Cl)c2N)CC1] |
| 126 (DDO-004) | 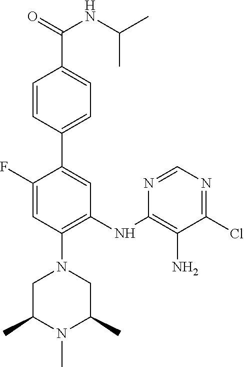 [SMILES: CC(C)NC(=O)c1ccc(-c2cc(Nc3ncnc(Cl)c3N)c(N3C[C@@H](C)N(C)[C@@H](C)C3)cc2F)cc1] |
| 127 (DDO-005) | 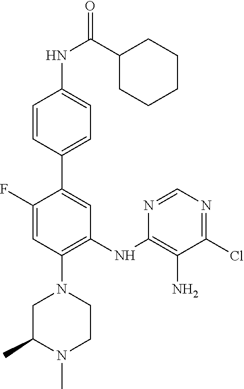 [SMILES: C[C@H]1CN(c2cc(F)c(-c3ccc(NC(=O)C4CCCCC4)cc3)cc2Nc2ncnc(Cl)c2N)CCN1C] |
| 128 (DDO-006) | 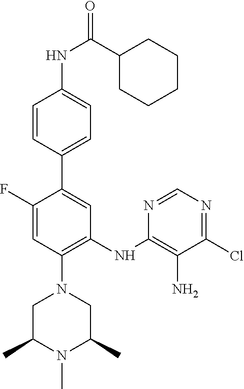 [SMILES: C[C@@H]1CN(c2cc(F)c(-c3ccc(NC(=O)C4CCCCC4)cc3)cc2Nc2ncnc(Cl)c2N)C[C@H](C)N1C] |
| 129 (DDO-007) | 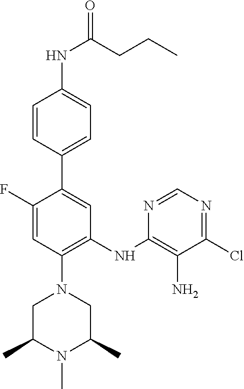 [SMILES: CCCC(=O)Nc1ccc(-c2cc(Nc3ncnc(Cl)c3N)c(N3C[C@@H](C)N(C)[C@@H](C)C3)cc2F)cc1] |
| 130 (DDO-008) | 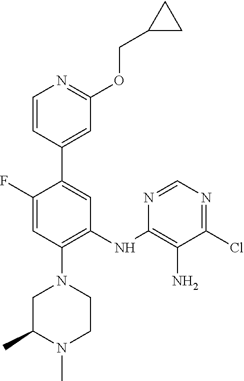 [SMILES: C[C@H]1CN(c2cc(F)c(-c3ccnc(OCC4CC4)c3)cc2Nc2ncnc(Cl)c2N)CCN1C] |
| 131 (DDO-009) | 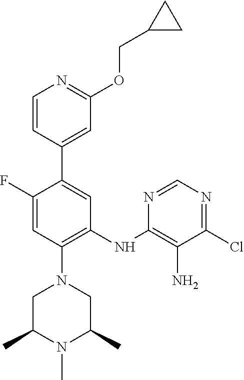 [SMILES: C[C@@H]1CN(c2cc(F)c(-c3ccnc(OCC4CC4)c3)cc2Nc2ncnc(Cl)c2N)C[C@H](C)N1C] |
| 132 (DDO-010) | 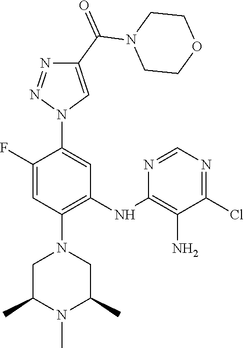 [SMILES: C[C@@H]1CN(c2cc(F)c(-n3cc(C(=O)N4CCOCC4)nn3)cc2Nc2ncnc(Cl)c2N)C[C@H](C)N1C] |
| 133 (DDO-012) | 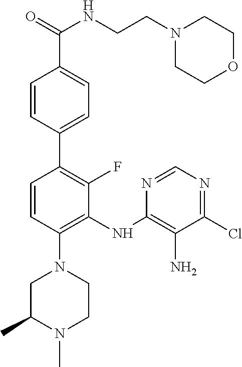 [SMILES: C[C@H]1CN(c2ccc(-c3ccc(C(=O)NCCN4CCOCC4)cc3)c(F)c2Nc2ncnc(Cl)c2N)CCN1C] |
| 134 (DDO-014) | 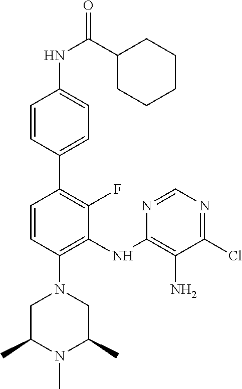 [SMILES: C[C@@H]1CN(c2ccc(-c3ccc(NC(=O)C4CCCCC4)cc3)c(F)c2Nc2ncnc(Cl)c2N)C[C@H](C)N1C] |
| 135 (DDO-015) | 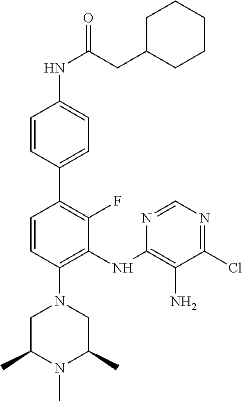 [SMILES: C[C@@H]1CN(c2ccc(-c3ccc(NC(=O)CC4CCCCC4)cc3)c(F)c2Nc2ncnc(Cl)c2N)C[C@H](C)N1C] |
| 136 (DDO-016) | 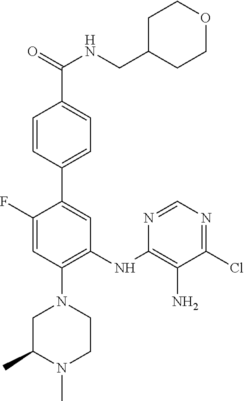 [SMILES: C[C@H]1CN(c2cc(F)c(-c3ccc(C(=O)NCC4CCOCC4)cc3)cc2Nc2ncnc(Cl)c2N)CCN1C] |
| 137 (DDO-017) | 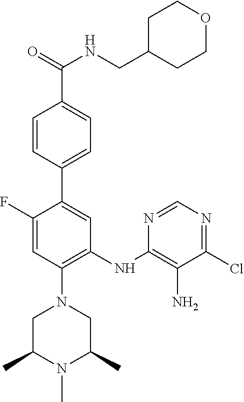 [SMILES: C[C@@H]1CN(c2cc(F)c(-c3ccc(C(=O)NCC4CCOCC4)cc3)cc2Nc2ncnc(Cl)c2N)C[C@H](C)N1C] |
| 138 DDO-018 | 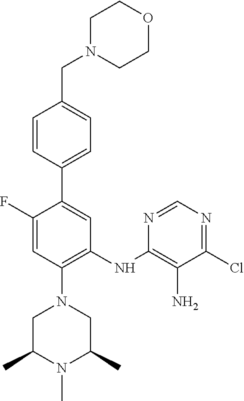 [SMILES: C[C@@H]1CN(c2cc(F)c(-c3ccc(CN4CCOCC4)cc3)cc2Nc2ncnc(Cl)c2N)C[C@H](C)N1C] |
| 139 DDO- 2213_019 | 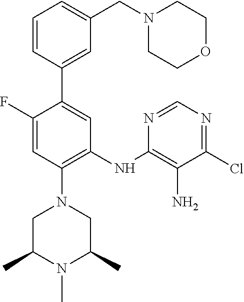 [SMILES: C[C@@H]1CN(c2cc(F)c(-c3cccc(CN4CCOCC4)c3)cc2Nc2ncnc(Cl)c2N)C[C@H](C)N1C] |
| 140 DDO- 2213_020 | 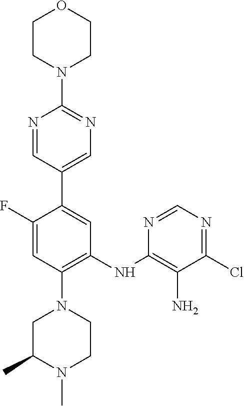 [SMILES: C[C@H]1CN(c2cc(F)c(-c3cnc(N4CCOCC4)nc3)cc2Nc2ncnc(Cl)c2N)CCN1C] |
| 141 DDO- 2213_021 | 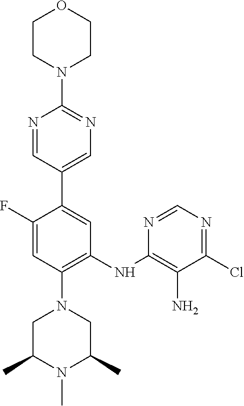 [SMILES: C[C@@H]1CN(c2cc(F)c(-c3cnc(N4CCOCC4)nc3)cc2Nc2ncnc(Cl)c2N)C[C@H](C)N1C] |
| 142 DDO- 2213_022 | 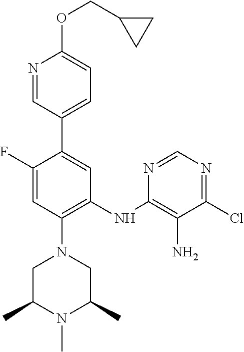 [SMILES: C[C@@H]1CN(c2cc(F)c(-c3ccc(OCC4CC4)nc3)cc2Nc2ncnc(Cl)c2N)C[C@H](C)N1C] |
| 143 DDO- 2213_023 | 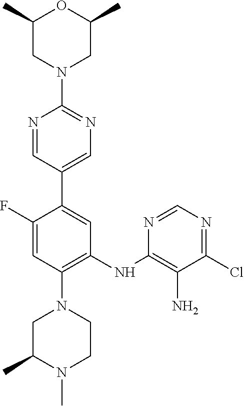 [SMILES: C[C@@H]1CN(c2ncc(-c3cc(Nc4ncnc(Cl)c4N)c(N4CCN(C)[C@@H](C)C4)cc3F)cn2)C[C@H](C)O1] |
| 144 DDO- 2213_024 | 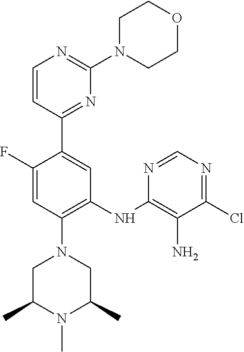 [SMILES: C[C@@H]1CN(c2cc(F)c(-c3ccnc(N4CCOCC4)n3)cc2Nc2ncnc(Cl)c2N)C[C@H](C)N1C] |
| 145 DDO- 2213_026 | 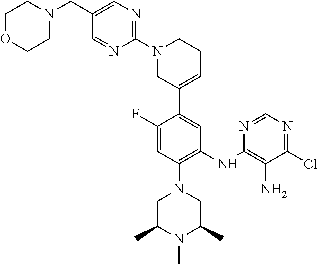 [SMILES: C[C@@H]1CN(c2cc(F)c(C3=CCCN(c4ncc(CN5CCOCC5)cn4)C3)cc2Nc2ncnc(Cl)c2N)C[C@H](C)N1C] |
| 146 DDO- 2213_027 | 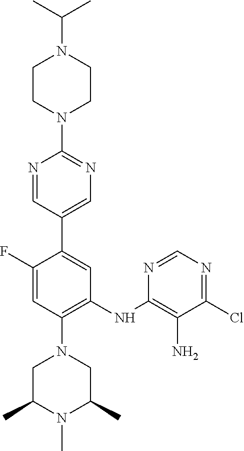 [SMILES: CC(C)N1CCN(c2ncc(-c3cc(Nc4ncnc(Cl)c4N)c(N4C[C@@H](C)N(C)[C@@H](C)C4)cc3F)cn2)CC1] |
| 147 DDO- 2213_028 | 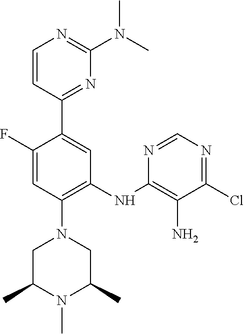 [SMILES: C[C@@H]1CN(c2cc(F)c(-c3ccnc(N(C)C)n3)cc2Nc2ncnc(Cl)c2N)C[C@H](C)N1C] |
| 148 DDO- 2213_029 | 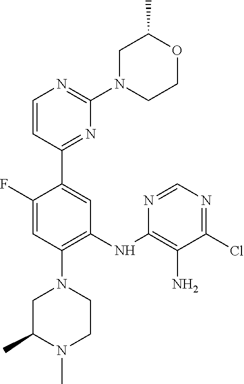 [SMILES: C[C@H]1CN(c2nccc(-c3cc(Nc4ncnc(Cl)c4N)c(N4CCN(C)[C@@H](C)C4)cc3F)n2)CCO1] |
| 149 DDO- 2213_030 | 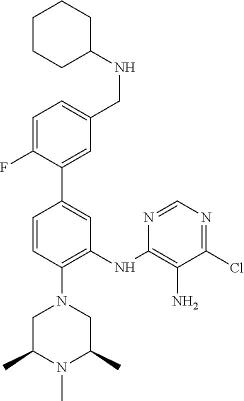 [SMILES: C[C@@H]1CN(c2ccc(-c3cc(CNC4CCCCC4)ccc3F)cc2Nc2ncnc(Cl)c2N)C[C@H](C)N1C] |

TABLE 4
Compounds of the disclosure

| 150 DDO- 2213_025 | 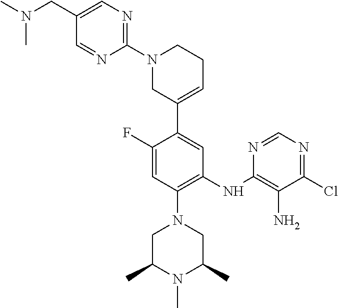 [SMILES: C[C@@H]1CN(c2cc(F)c(C3=CCCN(c4ncc(CN(C)C)cn4)C3)cc2Nc2ncnc(Cl)c2N)C[C@H](C)N1C] |
|---|---|
| 151 DDO- 2213_011 | 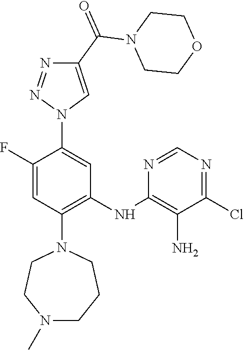 [SMILES: CN1CCCN(c2cc(F)c(-n3cc(C(=O)N4CCOCC4)nn3)cc2Nc2ncnc(Cl)c2N)CC1] |
| 152 DDO- 2213_013 | 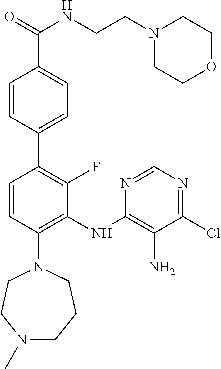 [SMILES: CN1CCCN(c2ccc(-c3ccc(C(=O)NCCN4CCOCC4)cc3)c(F)c2Nc2ncnc(Cl)c2N)CC1] |
| 153 | 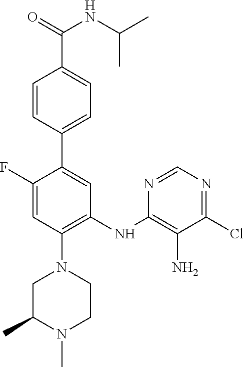 [SMILES: CC(C)NC(=O)c1ccc(-c2cc(Nc3ncnc(Cl)c3N)c(N3CCN(C)[C@@H](C)C3)cc2F)cc1] |
| 154 | 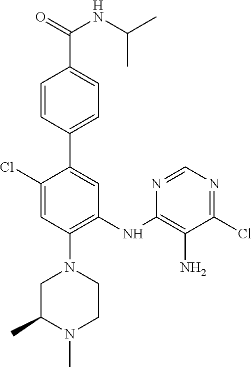 [SMILES: CC(C)NC(=O)c1ccc(-c2cc(Nc3ncnc(Cl)c3N)c(N3CCN(C)[C@@H](C)C3)cc2Cl)cc1] |
| 155 | 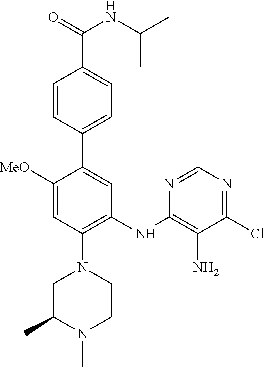 [SMILES: COc1cc(N2CCN(C)[C@@H](C)C2)c(Nc2ncnc(Cl)c2N)cc1-c1ccc(C(=O)NC(C)C)cc1] |
| 156 | 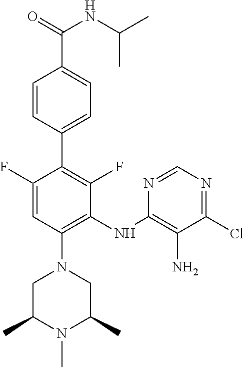 [SMILES: CC(C)NC(=O)c1ccc(-c2c(F)cc(N3C[C@@H](C)N(C)[C@@H](C)C3)c(Nc3ncnc(Cl)c3N)c2F)cc1] |
| 157 | 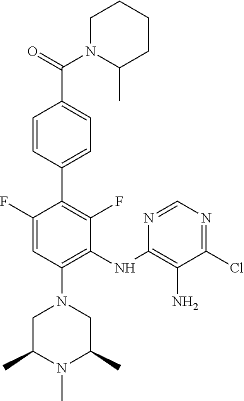 [SMILES: CC1CCCCN1C(=O)c1ccc(-c2c(F)cc(N3C[C@@H](C)N(C)[C@@H](C)C3)c(Nc3ncnc(Cl)c3N)c2F)cc1] |
| 158 | 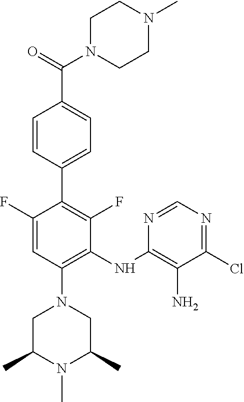 [SMILES: C[C@@H]1CN(c2cc(F)c(-c3ccc(C(=O)N4CCN(C)CC4)cc3)c(F)c2Nc2ncnc(Cl)c2N)C[C@H](C)N1C] |
| 159 | 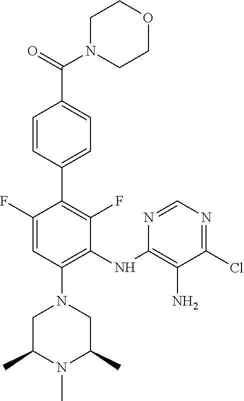 [SMILES: C[C@@H]1CN(c2cc(F)c(-c3ccc(C(=O)N4CCOCC4)cc3)c(F)c2Nc2ncnc(Cl)c2N)C[C@H](C)N1C] |
| 160 | 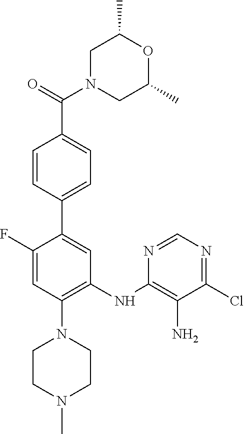 [SMILES: C[C@@H]1CN(C(=O)c2ccc(-c3cc(Nc4ncnc(Cl)c4N)c(N4CCN(C)CC4)cc3F)cc2)C[C@H](C)O1] |
| 161 | 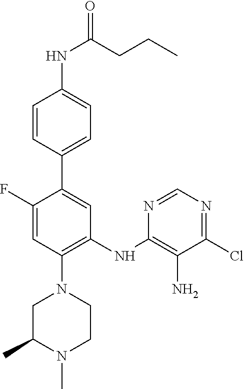 [SMILES: CCCC(=O)Nc1ccc(-c2cc(Nc3ncnc(Cl)c3N)c(N3CCN(C)[C@@H](C)C3)cc2F)cc1] |
| 162 | 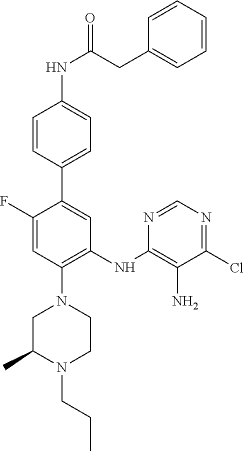 [SMILES: CCCN1CCN(c2cc(F)c(-c3ccc(NC(=O)Cc4ccccc4)cc3)cc2Nc2ncnc(Cl)c2N)C[C@@H]1C] |
| 163 | 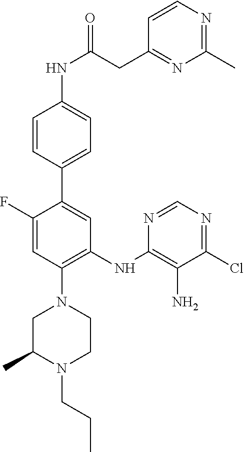 [SMILES: CCCN1CCN(c2cc(F)c(-c3ccc(NC(=O)Cc4ccnc(C)n4)cc3)cc2Nc2ncnc(Cl)c2N)C[C@@H]1C] |
| 164 | 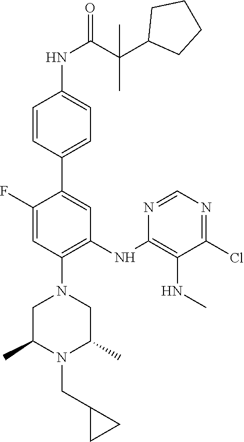 [SMILES: CNc1c(Cl)ncnc1Nc1cc(-c2ccc(NC(=O)C(C)(C)C3CCCC3)cc2)c(F)cc1N1C[C@H](C)N(CC2CC2)[C@@H](C)C1] |
| 165 | 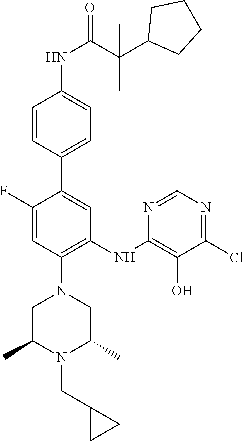 [SMILES: C[C@H]1CN(c2cc(F)c(-c3ccc(NC(=O)C(C)(C)C4CCCC4)cc3)cc2Nc2ncnc(Cl)c2O)C[C@H](C)N1CC1CC1] |
| 166 | 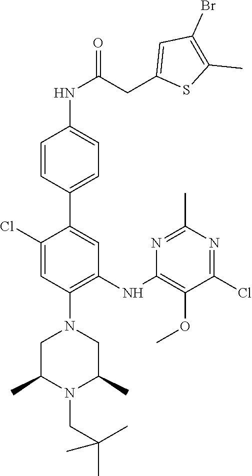 [SMILES: COc1c(Cl)nc(C)nc1Nc1cc(-c2ccc(NC(=O)Cc3cc(Br)c(C)s3)cc2)c(Cl)cc1N1C[C@@H](C)N(CC(C)(C)C)[C@@H](C)C1] |
| 167 | 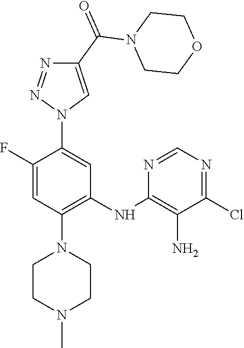 [SMILES: CN1CCN(c2cc(F)c(-n3cc(C(=O)N4CCOCC4)nn3)cc2Nc2ncnc(Cl)c2N)CC1] |
| 168 | 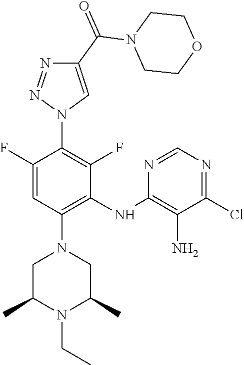 [SMILES: CCN1[C@H](C)CN(c2cc(F)c(-n3cc(C(=O)N4CCOCC4)nn3)c(F)c2Nc2ncnc(Cl)c2N)C[C@@H]1C] |
| 169 | 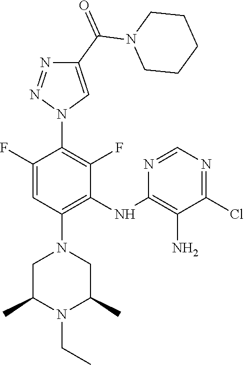 [SMILES: CCN1[C@H](C)CN(c2cc(F)c(-n3cc(C(=O)N4CCCCC4)nn3)c(F)c2Nc2ncnc(Cl)c2N)C[C@@H]1C] |
| 170 | 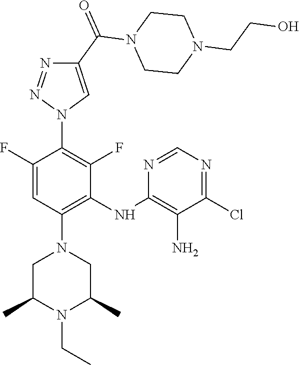 [SMILES: CCN1[C@H](C)CN(c2cc(F)c(-n3cc(C(=O)N4CCN(CCO)CC4)nn3)c(F)c2Nc2ncnc(Cl)c2N)C[C@@H]1C] |
| 171 | 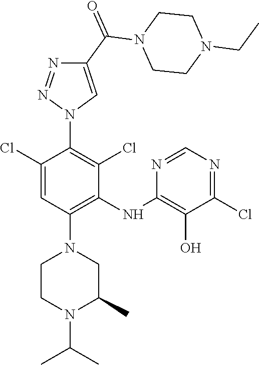 [SMILES: CCN1CCN(C(=O)c2cn(-c3c(Cl)cc(N4CCN(C(C)C)[C@H](C)C4)c(Nc4ncnc(Cl)c4O)c3Cl)nn2)CC1] |
| 172 | 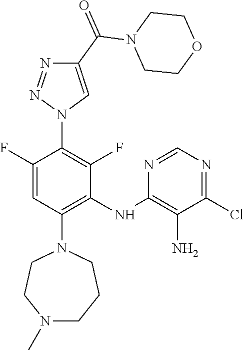 [SMILES: CN1CCCN(c2cc(F)c(-n3cc(C(=O)N4CCOCC4)nn3)c(F)c2Nc2ncnc(Cl)c2N)CC1] |
| 173 | 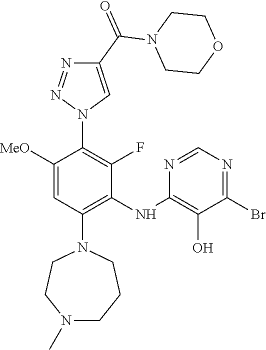 [SMILES: COc1cc(N2CCCN(C)CC2)c(Nc2ncnc(Br)c2O)c(F)c1-n1cc(C(=O)N2CCOCC2)nn1] |
| 174 | 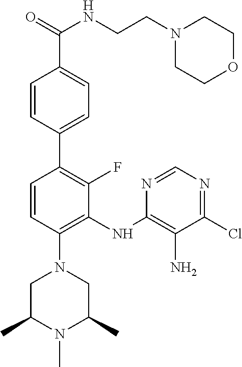 [SMILES: C[C@@H]1CN(c2ccc(-c3ccc(C(=O)NCCN4CCOCC4)cc3)c(F)c2Nc2ncnc(Cl)c2N)C[C@H](C)N1C] |
| 175 | 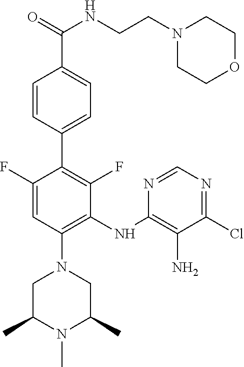 [SMILES: C[C@@H]1CN(c2cc(F)c(-c3ccc(C(=O)NCCN4CCOCC4)cc3)c(F)c2Nc2ncnc(Cl)c2N)C[C@H](C)N1C] |
| 176 | 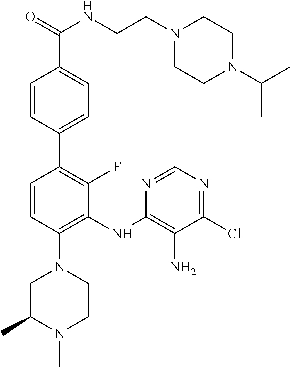 [SMILES: CC(C)N1CCN(CCNC(=O)c2ccc(-c3ccc(N4CCN(C)[C@@H](C)C4)c(Nc4ncnc(Cl)c4N)c3F)cc2)CC1] |
| 177 | 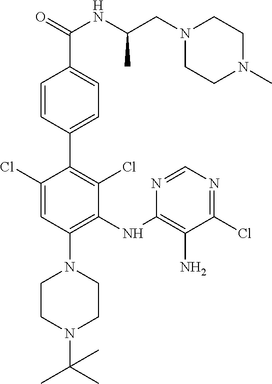 [SMILES: C[C@H](CN1CCN(C)CC1)NC(=O)c1ccc(-c2c(Cl)cc(N3CCN(C(C)(C)C)CC3)c(Nc3ncnc(Cl)c3N)c2Cl)cc1] |
| 178 | 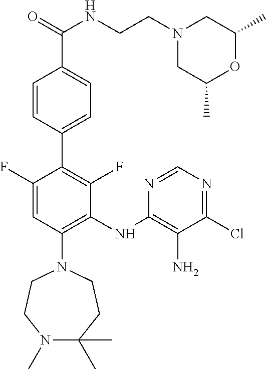 [SMILES: C[C@@H]1CN(CCNC(=O)c2ccc(-c3c(F)cc(N4CCN(C)C(C)(C)CC4)c(Nc4ncnc(Cl)c4N)c3F)cc2)C[C@H](C)O1] |
| 179 | 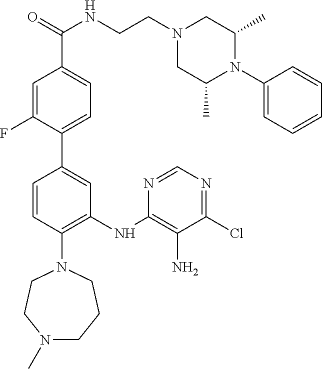 [SMILES: C[C@@H]1CN(CCNC(=O)c2ccc(-c3ccc(N4CCCN(C)CC4)c(Nc4ncnc(Cl)c4N)c3)c(F)c2)C[C@H](C)N1c1ccccc1] |
| 180 | 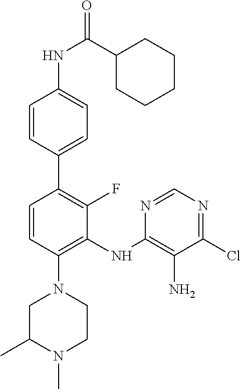 [SMILES: CC1CN(c2ccc(-c3ccc(NC(=O)C4CCCCC4)cc3)c(F)c2Nc2ncnc(Cl)c2N)CCN1C] |
| 181 | 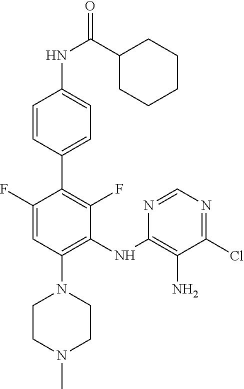 [SMILES: CN1CCN(c2cc(F)c(-c3ccc(NC(=O)C4CCCCC4)cc3)c(F)c2Nc2ncnc(Cl)c2N)CC1] |
| 182 | 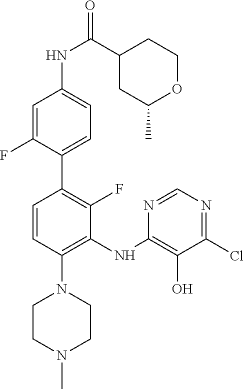 [SMILES: C[C@@H]1CC(C(=O)Nc2ccc(-c3ccc(N4CCN(C)CC4)c(Nc4ncnc(Cl)c4O)c3F)c(F)c2)CCO1] |
| 183 | 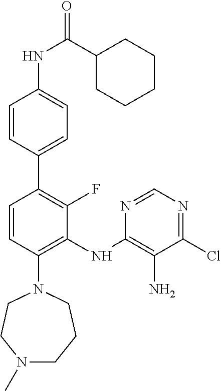 [SMILES: CN1CCCN(c2ccc(-c3ccc(NC(=O)C4CCCCC4)cc3)c(F)c2Nc2ncnc(Cl)c2N)CC1] |
| 184 | 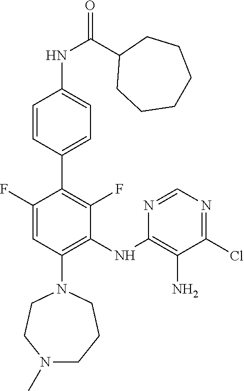 [SMILES: CN1CCCN(c2cc(F)c(-c3ccc(NC(=O)C4CCCCCC4)cc3)c(F)c2Nc2ncnc(Cl)c2N)CC1] |
| 185 | 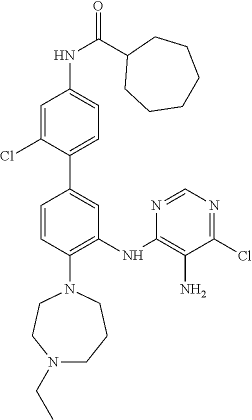 [SMILES: CCN1CCCN(c2ccc(-c3ccc(NC(=O)C4CCCCCC4)cc3Cl)cc2Nc2ncnc(Cl)c2N)CC1] |
| 186 | 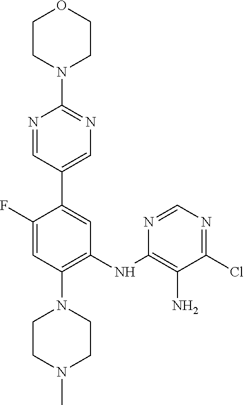 [SMILES: CN1CCN(c2cc(F)c(-c3cnc(N4CCOCC4)nc3)cc2Nc2ncnc(Cl)c2N)CC1] |
| 187 | 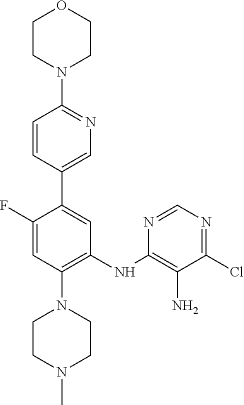 [SMILES: CN1CCN(c2cc(F)c(-c3ccc(N4CCOCC4)nc3)cc2Nc2ncnc(Cl)c2N)CC1] |
| 188 | 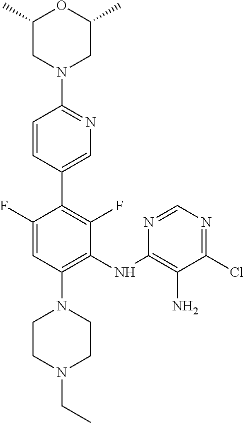 [SMILES: CCN1CCN(c2cc(F)c(-c3ccc(N4C[C@@H](C)O[C@@H](C)C4)nc3)c(F)c2Nc2ncnc(Cl)c2N)CC1] |
| 189 | 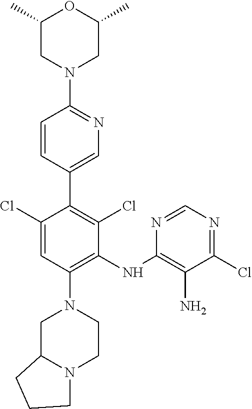 [SMILES: C[C@@H]1CN(c2ccc(-c3c(Cl)cc(N4CCN5CCCC5C4)c(Nc4ncnc(Cl)c4N)c3Cl)cn2)C[C@H](C)O1] |
| 190 | 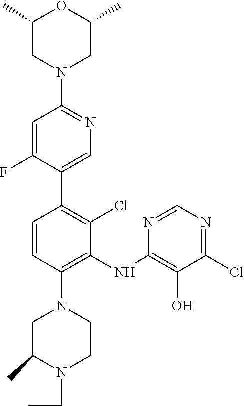 [SMILES: CCN1CCN(c2ccc(-c3cnc(N4C[C@@H](C)O[C@@H](C)C4)cc3F)c(Cl)c2Nc2ncnc(Cl)c2O)C[C@@H]1C] |
| 191 | 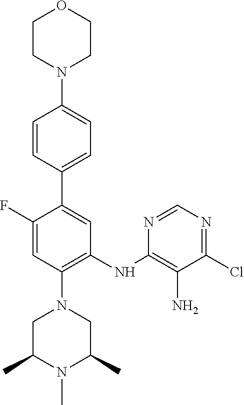 [SMILES: C[C@@H]1CN(c2cc(F)c(-c3ccc(N4CCOCC4)cc3)cc2Nc2ncnc(Cl)c2N)C[C@H](C)N1C] |
| 192 | 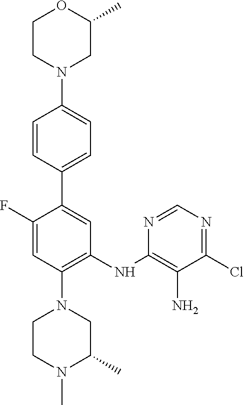 [SMILES: C[C@@H]1CN(c2ccc(-c3cc(Nc4ncnc(Cl)c4N)c(N4CCN(C)[C@@H](C)C4)cc3F)cc2)CCO1] |
| 193 | 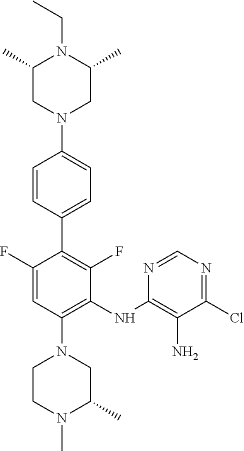 [SMILES: CCN1[C@H](C)CN(c2ccc(-c3c(F)cc(N4CCN(C)[C@@H](C)C4)c(Nc4ncnc(Cl)c4N)c3F)cc2)C[C@@H]1C] |
| 194 | 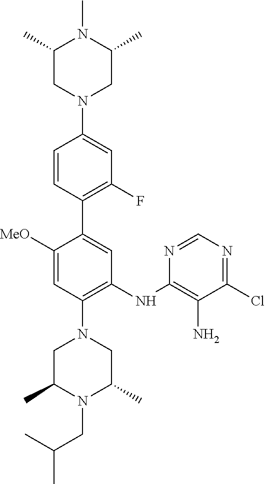 [SMILES: COc1cc(N2C[C@H](C)N(CC(C)C)[C@@H](C)C2)c(Nc2ncnc(Cl)c2N)cc1-c1ccc(N2C[C@@H](C)N(C)[C@@H](C)C2)cc1F] |
| 195 | 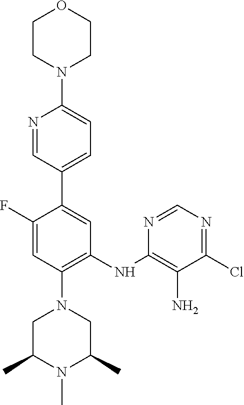 [SMILES: C[C@@H]1CN(c2cc(F)c(-c3ccc(N4CCOCC4)nc3)cc2Nc2ncnc(Cl)c2N)C[C@H](C)N1C] |
| 196 | 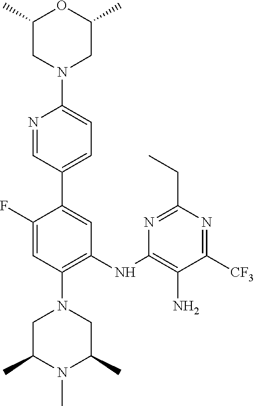 [SMILES: CCc1nc(Nc2cc(-c3ccc(N4C[C@@H](C)O[C@@H](C)C4)nc3)c(F)cc2N2C[C@@H](C)N(C)[C@@H](C)C2)c(N)c(C(F)(F)F)n1] |
| 197 | 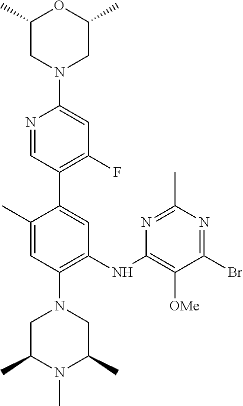 [SMILES: COc1c(Br)nc(C)nc1Nc1cc(-c2cnc(N3C[C@@H](C)O[C@@H](C)C3)cc2F)c(C)cc1N1C[C@@H](C)N(C)[C@@H](C)C1] |
| 198 | 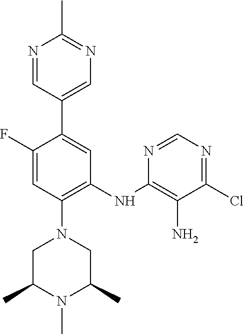 [SMILES: Cc1ncc(-c2cc(Nc3ncnc(Cl)c3N)c(N3C[C@@H](C)N(C)[C@@H](C)C3)cc2F)cn1] |
| 199 | 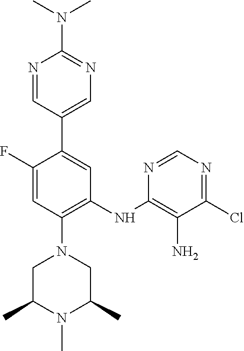 [SMILES: C[C@@H]1CN(c2cc(F)c(-c3cnc(N(C)C)nc3)cc2Nc2ncnc(Cl)c2N)C[C@H](C)N1C] |
| 200 | 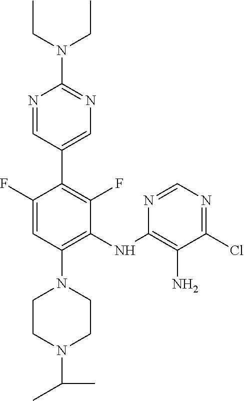 [SMILES: CCN(CC)c1ncc(-c2c(F)cc(N3CCN(C(C)C)CC3)c(Nc3ncnc(Cl)c3N)c2F)cn1] |
| 201 | 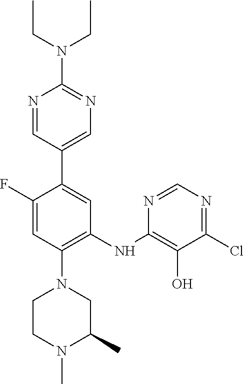 [SMILES: CCN(CC)c1ncc(-c2cc(Nc3ncnc(Cl)c3O)c(N3CCN(C)[C@H](C)C3)cc2F)cn1] |
| 202 | 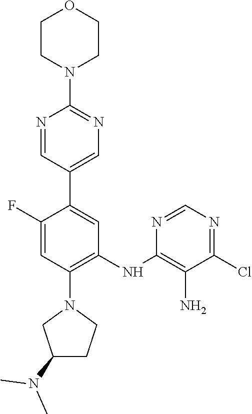 [SMILES: CN(C)[C@@H]1CCN(c2cc(F)c(-c3cnc(N4CCOCC4)nc3)cc2Nc2ncnc(Cl)c2N)C1] |
| 203 | 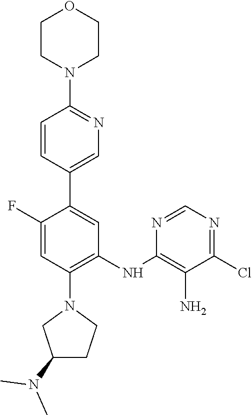 [SMILES: CN(C)[C@@H]1CCN(c2cc(F)c(-c3ccc(N4CCOCC4)nc3)cc2Nc2ncnc(Cl)c2N)C1] |
| 204 | 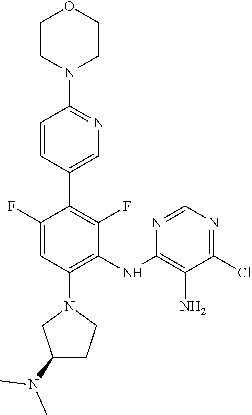 [SMILES: CN(C)[C@@H]1CCN(c2cc(F)c(-c3ccc(N4CCOCC4)nc3)c(F)c2Nc2ncnc(Cl)c2N)C1] |
| 205 | 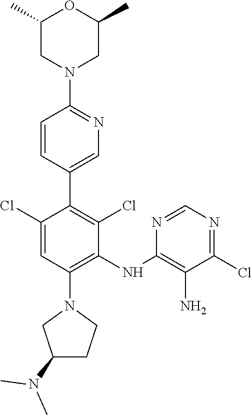 [SMILES: C[C@H]1CN(c2ccc(-c3c(Cl)cc(N4CC[C@@H](N(C)C)C4)c(Nc4ncnc(Cl)c4N)c3Cl)cn2)C[C@H](C)O1] |
| 206 | 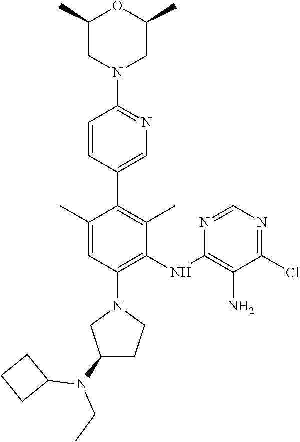 [SMILES: CCN(C1CCC1)[C@@H]1CCN(c2cc(C)c(-c3ccc(N4C[C@@H](C)O[C@@H](C)C4)nc3)c(C)c2Nc2ncnc(Cl)c2N)C1] |
| 207 | 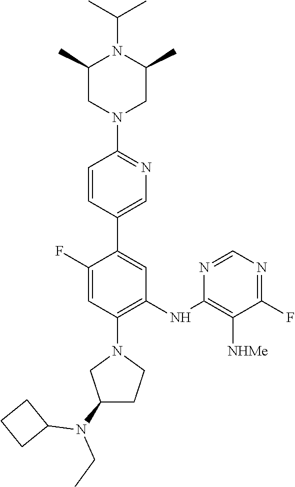 [SMILES: CCN(C1CCC1)[C@@H]1CCN(c2cc(F)c(-c3ccc(N4C[C@@H](C)N(C(C)C)[C@@H](C)C4)nc3)cc2Nc2ncnc(F)c2NC)C1] |
| 208 | 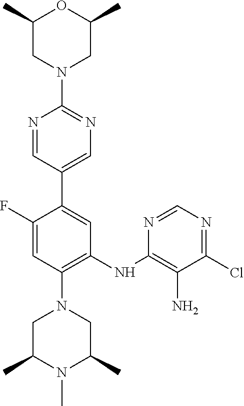 [SMILES: C[C@@H]1CN(c2ncc(-c3cc(Nc4ncnc(Cl)c4N)c(N4C[C@@H](C)N(C)[C@@H](C)C4)cc3F)cn2)C[C@H](C)O1] |
| 209 | 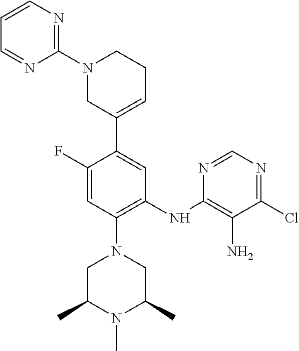 [SMILES: C[C@@H]1CN(c2cc(F)c(C3=CCCN(c4ncccn4)C3)cc2Nc2ncnc(Cl)c2N)C[C@H](C)N1C] |
| 210 | 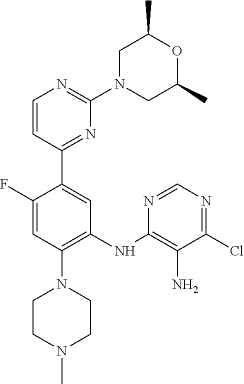 [SMILES: C[C@@H]1CN(c2nccc(-c3cc(Nc4ncnc(Cl)c4N)c(N4CCN(C)CC4)cc3F)n2)C[C@H](C)O1] |
| 211 | 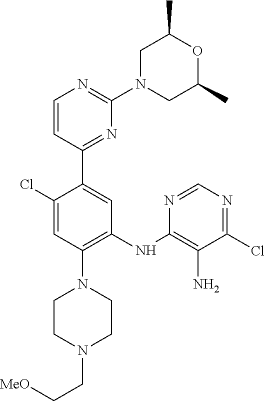 [SMILES: COCCN1CCN(c2cc(Cl)c(-c3ccnc(N4C[C@@H](C)O[C@@H](C)C4)n3)cc2Nc2ncnc(Cl)c2N)CC1] |
| 212 | 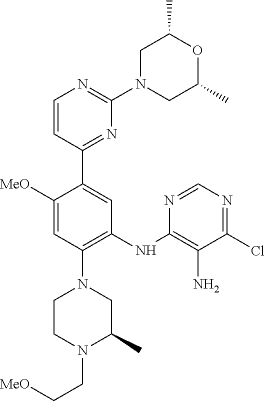 [SMILES: COCCN1CCN(c2cc(OC)c(-c3ccnc(N4C[C@@H](C)O[C@@H](C)C4)n3)cc2Nc2ncnc(Cl)c2N)C[C@H]1C] |
| 213 | 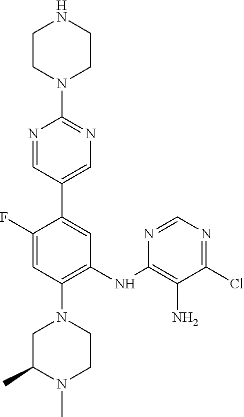 [SMILES: C[C@H]1CN(c2cc(F)c(-c3cnc(N4CCNCC4)nc3)cc2Nc2ncnc(Cl)c2N)CCN1C] |
| 214 | 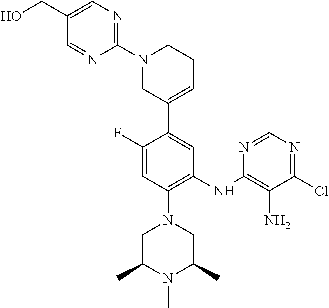 [SMILES: C[C@@H]1CN(c2cc(F)c(C3=CCCN(c4ncc(CO)cn4)C3)cc2Nc2ncnc(Cl)c2N)C[C@H](C)N1C] |
| 215 | 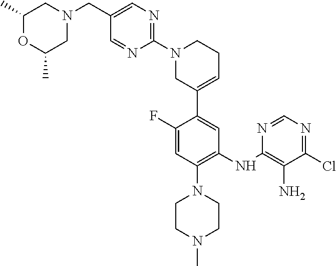 [SMILES: C[C@@H]1CN(Cc2cnc(N3CCC=C(c4cc(Nc5ncnc(Cl)c5N)c(N5CCN(C)CC5)cc4F)C3)nc2)C[C@H](C)O1] |
| 216 | 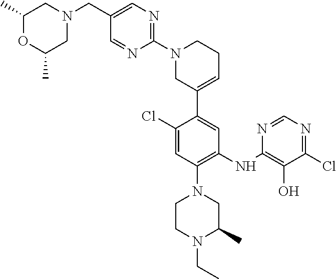 [SMILES: CCN1CCN(c2cc(Cl)c(C3=CCCN(c4ncc(CN5C[C@@H](C)O[C@@H](C)C5)cn4)C3)cc2Nc2ncnc(Cl)c2O)C[C@H]1C] |
| 217 | 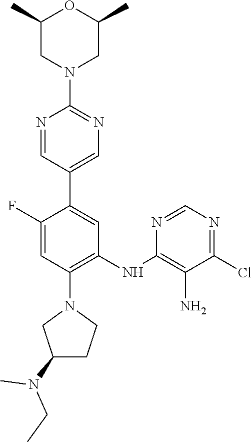 [SMILES: CCN(C)[C@@H]1CCN(c2cc(F)c(-c3cnc(N4C[C@@H](C)O[C@@H](C)C4)nc3)cc2Nc2ncnc(Cl)c2N)C1] |
| 218 | 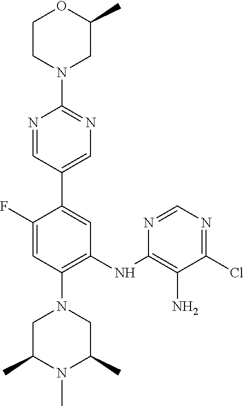 [SMILES: C[C@@H]1CN(c2cc(F)c(-c3cnc(N4CCO[C@@H](C)C4)nc3)cc2Nc2ncnc(Cl)c2N)C[C@H](C)N1C] |
| 219 | 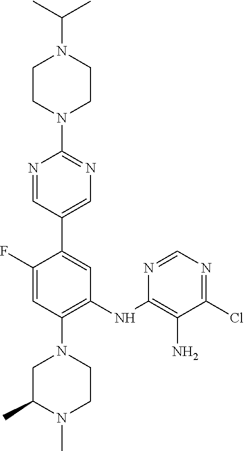 [SMILES: CC(C)N1CCN(c2ncc(-c3cc(Nc4ncnc(Cl)c4N)c(N4CCN(C)[C@@H](C)C4)cc3F)cn2)CC1] |
| 220 | 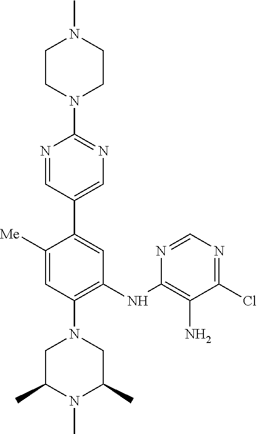 [SMILES: Cc1cc(N2C[C@@H](C)N(C)[C@@H](C)C2)c(Nc2ncnc(Cl)c2N)cc1-c1cnc(N2CCN(C)CC2)nc1] |
| 221 | 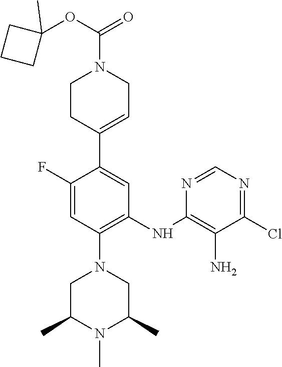 [SMILES: C[C@@H]1CN(c2cc(F)c(C3=CCN(C(=O)OC4(C)CCC4)CC3)cc2Nc2ncnc(Cl)c2N)C[C@H](C)N1C] |
| 222 | 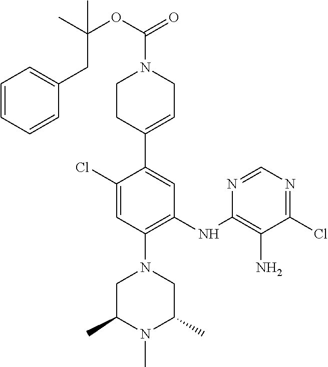 [SMILES: C[C@H]1CN(c2cc(Cl)c(C3=CCN(C(=O)OC(C)(C)Cc4ccccc4)CC3)cc2Nc2ncnc(Cl)c2N)C[C@H](C)N1C] |
| 223 | 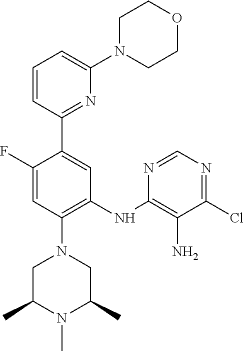 [SMILES: C[C@@H]1CN(c2cc(F)c(-c3cccc(N4CCOCC4)n3)cc2Nc2ncnc(Cl)c2N)C[C@H](C)N1C] |
| 224 | 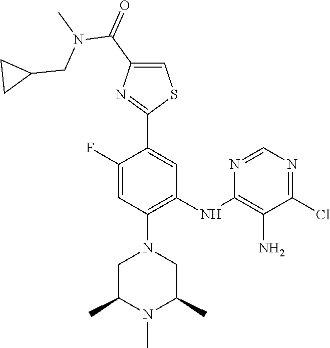 [SMILES: C[C@@H]1CN(c2cc(F)c(-c3nc(C(=O)N(C)CC4CC4)cs3)cc2Nc2ncnc(Cl)c2N)C[C@H](C)N1C] |
| 225 | 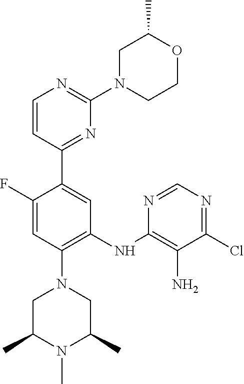 [SMILES: C[C@@H]1CN(c2cc(F)c(-c3ccnc(N4CCO[C@@H](C)C4)n3)cc2Nc2ncnc(Cl)c2N)C[C@H](C)N1C] |
| 226 | 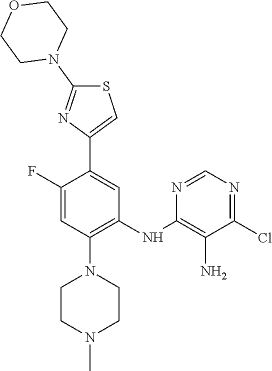 [SMILES: CN1CCN(c2cc(F)c(-c3csc(N4CCOCC4)n3)cc2Nc2ncnc(Cl)c2N)CC1] |
| 227 | 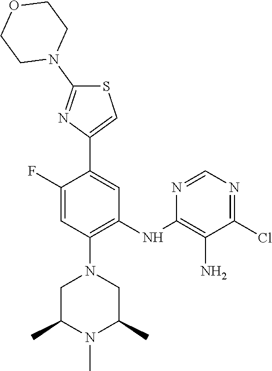 [SMILES: C[C@@H]1CN(c2cc(F)c(-c3csc(N4CCOCC4)n3)cc2Nc2ncnc(Cl)c2N)C[C@H](C)N1C] |
| 228 | 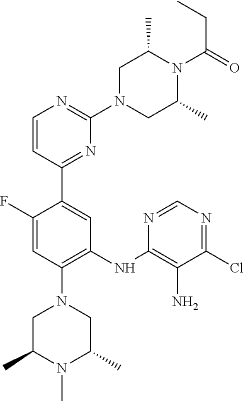 [SMILES: CCC(=O)N1[C@H](C)CN(c2nccc(-c3cc(Nc4ncnc(Cl)c4N)c(N4C[C@H](C)N(C)[C@@H](C)C4)cc3F)n2)C[C@@H]1C] |
| 229 | 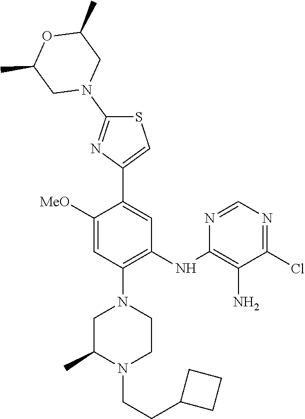 [SMILES: COc1cc(N2CCN(CCC3CCC3)[C@@H](C)C2)c(Nc2ncnc(Cl)c2N)cc1-c1csc(N2C[C@@H](C)O[C@@H](C)C2)n1] |
| 230 | 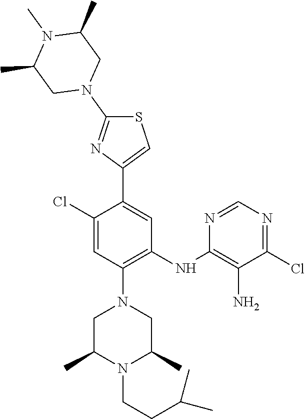 [SMILES: CC(C)CCN1[C@H](C)CN(c2cc(Cl)c(-c3csc(N4C[C@@H](C)N(C)[C@@H](C)C4)n3)cc2Nc2ncnc(Cl)c2N)C[C@@H]1C] |
| 231 | 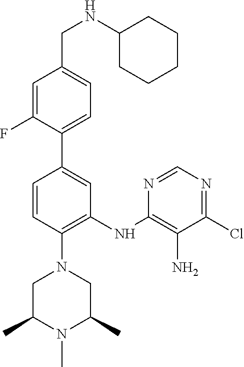 [SMILES: C[C@@H]1CN(c2ccc(-c3ccc(CNC4CCCCC4)cc3F)cc2Nc2ncnc(Cl)c2N)C[C@H](C)N1C] |
| 232 | 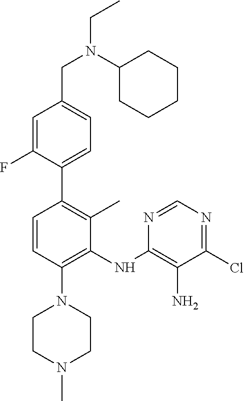 [SMILES: CCN(Cc1ccc(-c2ccc(N3CCN(C)CC3)c(Nc3ncnc(Cl)c3N)c2C)c(F)c1)C1CCCCC1] |
| 233 | 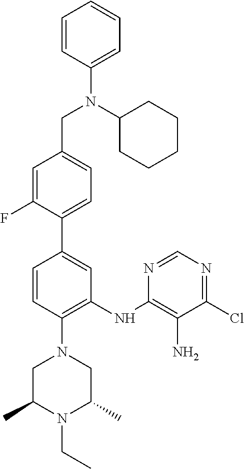 [SMILES: CCN1[C@@H](C)CN(c2ccc(-c3ccc(CN(c4ccccc4)C4CCCCC4)cc3F)cc2Nc2ncnc(Cl)c2N)C[C@@H]1C] |
| 234 | 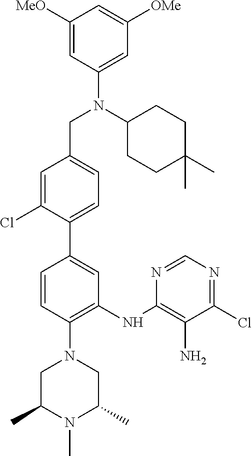 [SMILES: COc1cc(OC)cc(N(Cc2ccc(-c3ccc(N4C[C@H](C)N(C)[C@@H](C)C4)c(Nc4ncnc(Cl)c4N)c3)c(Cl)c2)C2CCC(C)(C)CC2)c1] |
| 235 | 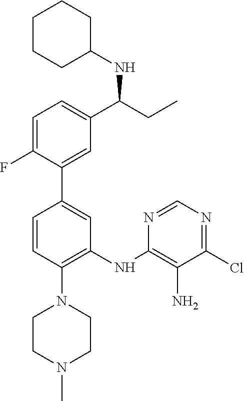 [SMILES: CC[C@H](NC1CCCCC1)c1ccc(F)c(-c2ccc(N3CCN(C)CC3)c(Nc3ncnc(Cl)c3N)c2)c1] |
| 236 | 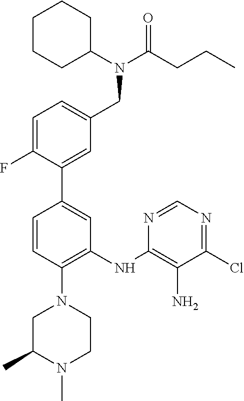 |
| 237 | 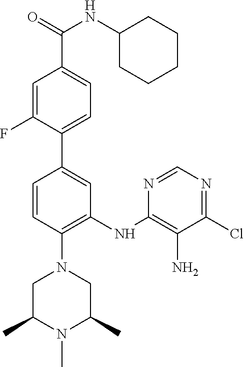 [SMILES: C[C@@H]1CN(c2ccc(-c3ccc(C(=O)NC4CCCCC4)cc3F)cc2Nc2ncnc(Cl)c2N)C[C@H](C)N1C] |
| 238 | 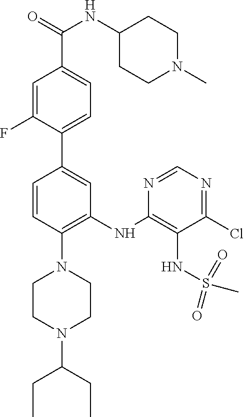 [SMILES: CCC(CC)N1CCN(c2ccc(-c3ccc(C(=O)NC4CCN(C)CC4)cc3F)cc2Nc2ncnc(Cl)c2NS(C)(=O)=O)CC1] |
| 239 | 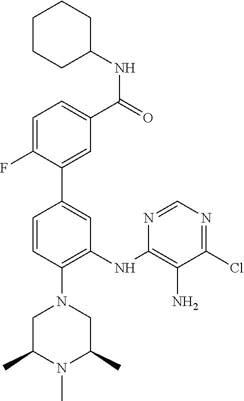 [SMILES: C[C@@H]1CN(c2ccc(-c3cc(C(=O)NC4CCCCC4)ccc3F)cc2Nc2ncnc(Cl)c2N)C[C@H](C)N1C] |
| 240 | 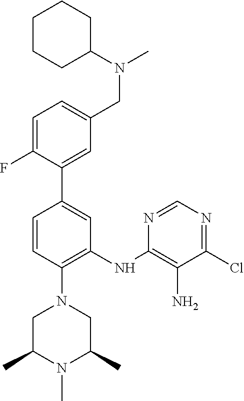 [SMILES: C[C@@H]1CN(c2ccc(-c3cc(CN(C)C4CCCCC4)ccc3F)cc2Nc2ncnc(Cl)c2N)C[C@H](C)N1C] |
| 241 | 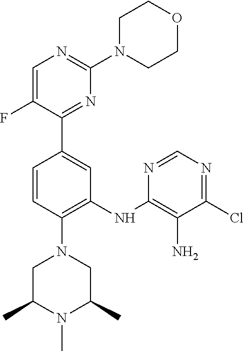 [SMILES: C[C@@H]1CN(c2ccc(-c3nc(N4CCOCC4)ncc3F)cc2Nc2ncnc(Cl)c2N)C[C@H](C)N1C] |
| 242 | 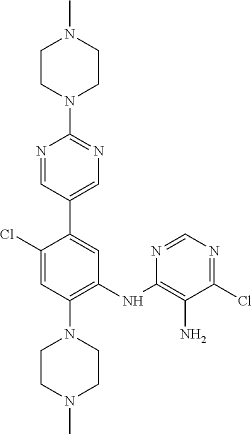 [SMILES: CN1CCN(c2ncc(-c3cc(Nc4ncnc(Cl)c4N)c(N4CCN(C)CC4)cc3Cl)cn2)CC1] |
| 243 | 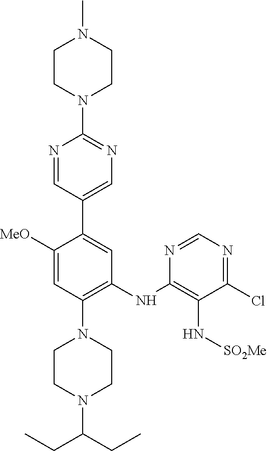 [SMILES: CCC(CC)N1CCN(c2cc(OC)c(-c3cnc(N4CCN(C)CC4)nc3)cc2Nc2ncnc(Cl)c2NS(C)(=O)=O)CC1] |
| 244 | 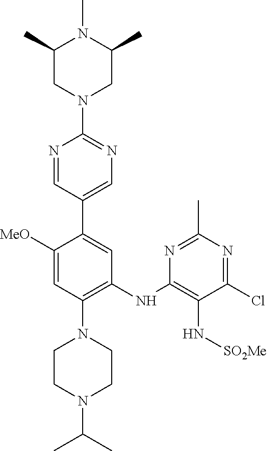 [SMILES: COc1cc(N2CCN(C(C)C)CC2)c(Nc2nc(C)nc(Cl)c2NS(C)(=O)=O)cc1-c1cnc(N2C[C@@H](C)N(C)[C@@H](C)C2)nc1] |
| 245 | 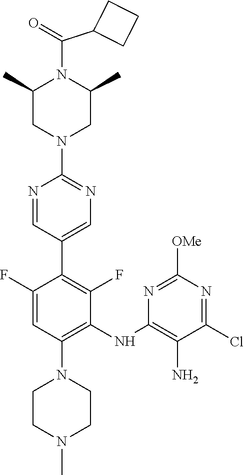 [SMILES: COc1nc(Cl)c(N)c(Nc2c(N3CCN(C)CC3)cc(F)c(-c3cnc(N4C[C@@H](C)N(C(=O)C5CCC5)[C@@H](C)C4)nc3)c2F)n1] |
| 246 | 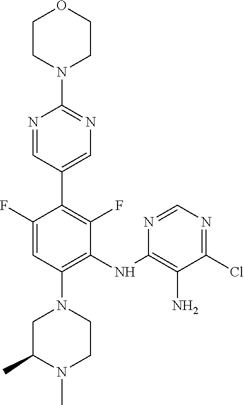 [SMILES: C[C@H]1CN(c2cc(F)c(-c3cnc(N4CCOCC4)nc3)c(F)c2Nc2ncnc(Cl)c2N)CCN1C] |
| 247 | 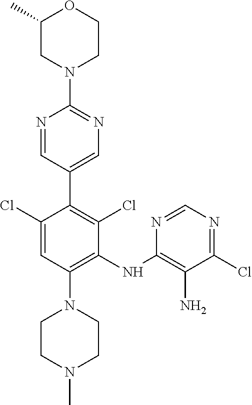 [SMILES: C[C@H]1CN(c2ncc(-c3c(Cl)cc(N4CCN(C)CC4)c(Nc4ncnc(Cl)c4N)c3Cl)cn2)CCO1] |
| 248 | 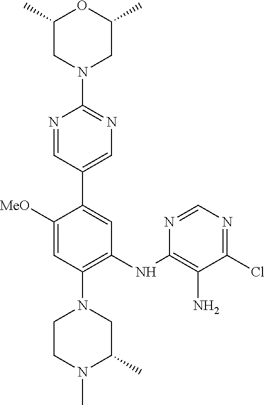 [SMILES: COc1cc(N2CCN(C)[C@@H](C)C2)c(Nc2ncnc(Cl)c2N)cc1-c1cnc(N2C[C@@H](C)O[C@@H](C)C2)nc1] |
| 249 | 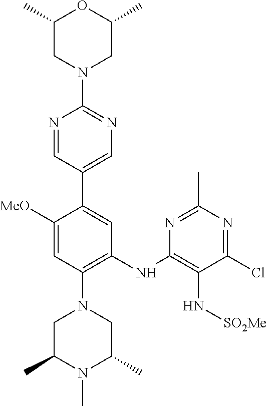 [SMILES: COc1cc(N2C[C@H](C)N(C)[C@@H](C)C2)c(Nc2nc(C)nc(Cl)c2NS(C)(=O)=O)cc1-c1cnc(N2C[C@@H](C)O[C@@H](C)C2)nc1] |
| 250 | 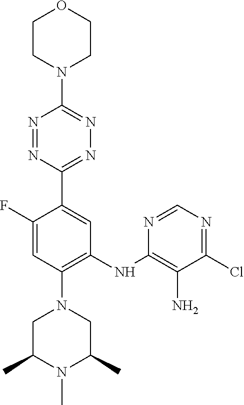 [SMILES: C[C@@H]1CN(c2cc(F)c(-c3nnc(N4CCOCC4)nn3)cc2Nc2ncnc(Cl)c2N)C[C@H](C)N1C] |
| 251 | 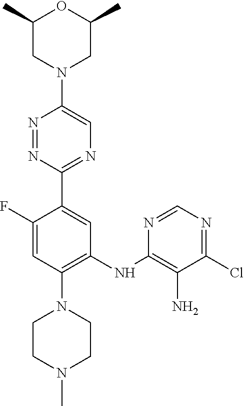 [SMILES: C[C@@H]1CN(c2cnc(-c3cc(Nc4ncnc(Cl)c4N)c(N4CCN(C)CC4)cc3F)nn2)C[C@H](C)O1] |
| 252 | 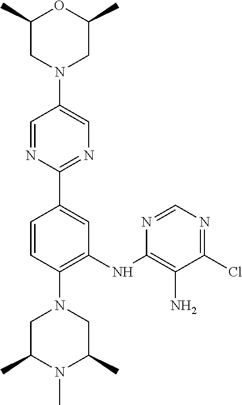 [SMILES: C[C@@H]1CN(c2cnc(-c3ccc(N4C[C@@H](C)N(C)[C@@H](C)C4)c(Nc4ncnc(Cl)c4N)c3)nc2)C[C@H](C)O1] |
| 253 | 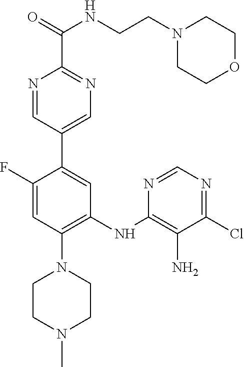 [SMILES: CN1CCN(c2cc(F)c(-c3cnc(C(=O)NCCN4CCOCC4)nc3)cc2Nc2ncnc(Cl)c2N)CC1] |
| 254 | 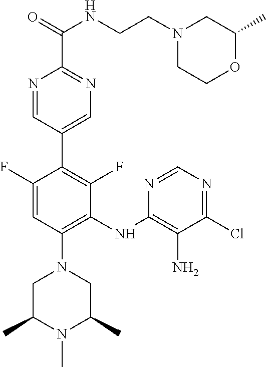 [SMILES: C[C@@H]1CN(c2cc(F)c(-c3cnc(C(=O)NCCN4CCO[C@@H](C)C4)nc3)c(F)c2Nc2ncnc(Cl)c2N)C[C@H](C)N1C] |
| 255 | 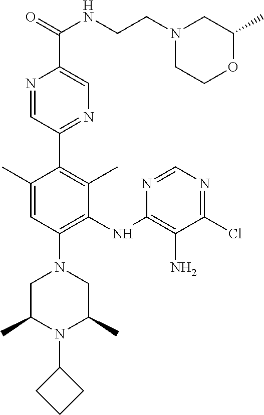 |
| 256 | 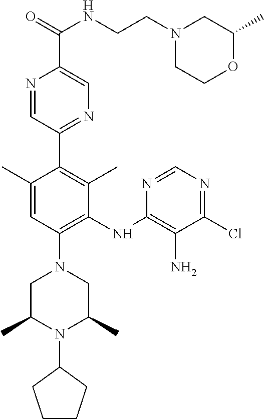 |
| 257 | 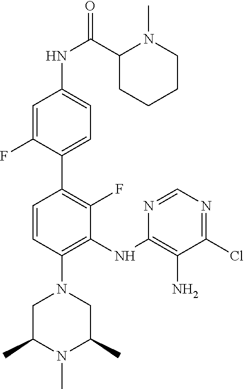 [SMILES: C[C@@H]1CN(c2ccc(-c3ccc(NC(=O)C4CCCCN4C)cc3F)c(F)c2Nc2ncnc(Cl)c2N)C[C@H](C)N1C] |
| 258 | 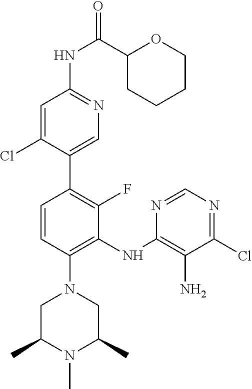 [SMILES: C[C@@H]1CN(c2ccc(-c3cnc(NC(=O)C4CCCCO4)cc3Cl)c(F)c2Nc2ncnc(Cl)c2N)C[C@H](C)N1C] |
| 259 | 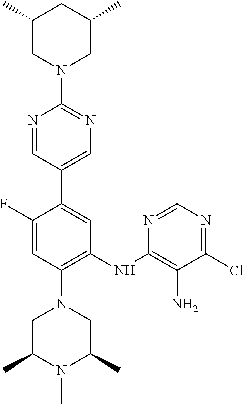 [SMILES: C[C@@H]1C[C@H](C)CN(c2ncc(-c3cc(Nc4ncnc(Cl)c4N)c(N4C[C@@H](C)N(C)[C@@H](C)C4)cc3F)cn2)C1] |

In some embodiments, the compound is a compound selected from Table 1, or a pharmaceutically acceptable salt, solvate, polymorph or prodrug thereof. In some embodiments, the compound is a compound selected from Table 2, or a pharmaceutically acceptable salt, solvate, polymorph or prodrug thereof. In some embodiments, the compound is a compound selected from Table 3, or a pharmaceutically acceptable salt, solvate, polymorph or prodrug thereof. In some embodiments, the compound is a compound selected from Table 4, or a pharmaceutically acceptable salt, solvate, polymorph or prodrug thereof.

### Further Forms of Compounds

In some embodiments, a compound disclosed herein possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In some embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In some embodiments, diastereomers are separated by separation/resolution techniques based upon differences in solubility. In some embodiments, separation of stereoisomers is performed by chromatography or by the forming of diastereomeric salts and separation is carried out by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In some embodiments, upon in vivo administration, a prodrug is chemically converted to the biologically, pharmaceutically or therapeutically active form of the compound. In some embodiments, a prodrug is enzymatically metabolized by one or more steps or processes to the biologically, pharmaceutically or therapeutically active form of the compound.

In some embodiments, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some embodiments, some of the herein-described compounds may be a prodrug for another derivative or active compound.

In some embodiments, sites on the aromatic ring portion of compounds described herein are susceptible to various metabolic reactions. Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C, ¹⁴C, ¹⁵N, ¹⁸O, ¹⁷O, ³⁵S, ¹⁸F, ³⁶Cl, and ¹²⁵I. In some embodiments, isotopically-labeled compounds described herein, for example those into which radioactive isotopes such as ³H and ¹⁴C are incorporated, are useful in drug and/or substrate tissue distribution assays. In some embodiments, substitution with isotopes such as deuterium affords certain therapeutic advantages resulting from greater metabolic stability, such as, for example, increased in vivo half-life or reduced dosage requirements.

In some embodiments, the compounds described herein are metabolized upon administration to an organism in need to produce a metabolite that is then used to produce a desired effect, including a desired therapeutic effect.

“Pharmaceutically acceptable” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound disclosed herein with acids. Pharmaceutically acceptable salts are also obtained by reacting a compound disclosed herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some embodiments, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In some embodiments, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compound disclosed herein is formulated into a variety of topically administrable compositions, such as solutions, suspensions, lotions, gels, pastes, shampoos, scrubs, rubs, smears, medicated sticks, medicated bandages, balms, creams or ointments. In some embodiments, the compounds disclosed herein are administered topically to the skin.

In some embodiments, the compounds disclosed herein are administered by inhalation.

In some embodiments, the compounds disclosed herein are formulated for intranasal administration. Such formulations include nasal sprays, nasal mists, and the like.

In some embodiments, the compounds disclosed herein are formulated as eye drops.

In any of the aforementioned embodiments are further embodiments in which the effective amount of the compound disclosed herein is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned embodiments are further embodiments comprising single administrations of the effective amount of the compound disclosed herein, including further embodiments in which (i) the compound is administered once; (ii) the compound is administered to the mammal multiple times over the span of one day; (iii) the compound is administered continually; or (iv) the compound is administered continuously.

In any of the aforementioned embodiments are further embodiments comprising multiple administrations of the effective amount of the compound disclosed herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In some embodiments, the compound disclosed herein is administered in a local rather than systemic manner.

In some embodiments, the compound disclosed herein is administered topically. In some embodiments, the compound disclosed herein is administered systemically.

In some embodiments, the pharmaceutical formulation is in the form of a tablet. In other embodiments, pharmaceutical formulations of the compounds disclosed herein are in the form of a capsule.

In some embodiments, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In some embodiments, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In some embodiments, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In some embodiments, the compositions containing the compound disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In some embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 0.01 mg to about 1000 mg per day. In one embodiment, the desired dose is conveniently presented in a single dose or in divided doses.

### Methods of Treatment

Provided herein are methods for the treatment of diseases mediated by MLL1 through inhibiting MLL1-WDR5 protein-protein interaction, wherein the diseases, such as for example MLL gene fusion type leukemia can be treated through inhibition of the enzymatic activity of MLL1. In some embodiments, described herein is a method of treating a disease or condition including administering to a subject in need thereof an effective amount of a compound disclosed herein.

In some embodiments, the disease or condition being treated is a cancer. In some embodiments, the cancer is a blood cancer.

### Leukemia

Leukemia is characterized by an abnormal increase of white blood cells in the blood or bone marrow. Among all types of cancers, the morbidity of leukemia is the highest for patients below 35 years old. Over 70% of infant leukemia patients bear a translocation involving chromosome 11, resulting in the fusion of the MLL1 gene with other genes (Nat. Rev. Cancer., 2007, 7(11):823-833). MLL1 translocations are also found in approximately 10% of adult acute myeloid leukemia (AML) patients who were previously treated with topoisomerase II inhibitors for other types of cancers.

MLL1 enzymatic activity is determined by MLL1 and WDR5 protein-protein interaction; MLL1 enzymatic activity affects the methylation level of H3K4. The H3K4 methylation level increases abnormally in MLL fusion type leukemia, and the downstream Hox and Meis-1 gene expression levels are up-regulated abnormally. When MLL1-WDR5 protein-protein interaction is inhibited, MLL1 catalytic activity decreases, H3K4 methylation level decreases, Hox and Meis-1 gene expression levels are downregulated, inhibiting leukemia cell proliferation.

In some embodiments, the cancer is leukemia. In some embodiments, the leukemia is acute leukemia. In some embodiments, the acute leukemia is acute leukemia with MLL1 gene rearrangement.

### Acute Myeloid Leukemia (AML)

The CEBPA gene is mutated in 9% of patients with acute myeloid leukemia (AML). Selective expression of a short (30-kDa) CCAAT-enhancer binding protein-a (C/EBPa) translational isoform, termed p30, represents the most common type of CEBPA mutation in AML. The molecular mechanisms underlying p30-mediated transformation remain incompletely understood. Studies have shown that C/EBPa p30, but not the normal p42 isoform, preferentially interacts with WDR5, a key component of SET/MLL (SET-domain/mixed-lineage leukemia) histone-methyltransferase complexes. Accordingly, p30-bound genomic regions are enriched for MLL-dependent H3K4me3 marks. The p30-dependent increase in self-renewal and inhibition of myeloid differentiation required WDR5, as downregulation of the latter inhibited proliferation and restored differentiation in p30-dependent AML models. Small-molecule inhibitors of WDR5-MLL binding selectively inhibited proliferation and induced differentiation in p30-expressing human AML cells revealing the mechanism of p30-dependent transformation and establish the p30 cofactor WDR5 as a therapeutic target in CEBPA-mutant AML (Nat Chem Biol. 2015; 11(8):571-8).

In some embodiments, the leukemia is AML leukemia.

### MYCN-Amplified Neuroblastoma

MYCN gene amplification in neuroblastoma drives a gene expression program that correlates strongly with aggressive disease. Mechanistically, trimethylation of histone H3 lysine 4 (H3K4) at target gene promoters is a prerequisite for the transcriptional program to be enacted. WDR5 is a histone H3K4 presenter that has been found to have an essential role in H3K4 trimethylation. The relationship between WDR5-mediated H3K4 trimethylation and N-Myc transcriptional programs in neuroblastoma cells was investigated. N-Myc upregulated WDR5 expression in neuroblastoma cells. Gene expression analysis revealed that WDR5 target genes included those with MYC-binding elements at promoters such as MDM2. WDR5 has been shown to form a protein complex at the MDM2 promoter with N-Myc, but not p53, leading to histone H3K4 trimethylation and activation of MDM2 transcription (Cancer Res 2015; 75(23); 5143-54). RNAi-mediated attenuation of WDR5 upregulated expression of wild-type but not mutant p53, an effect associated with growth inhibition and apoptosis. Similarly, a small-molecule antagonist of WDR5 reduced N-Myc/WDR5 complex formation, N— Myc target gene expression, and cell growth in neuroblastoma cells. In MYCN-transgenic mice, WDR5 was overexpressed in precancerous ganglion and neuroblastoma cells compared with normal ganglion cells. Clinically, elevated levels of WDR5 in neuroblastoma specimens have an independent predictor of poor overall survival. WDR5 has been identified as a relevant cofactor for N-Myc-regulated transcriptional activation and tumorogenesis and as a novel therapeutic target for MYCN-amplified neuroblastomas (Cancer Res 2015; 75(23); 5143-54, Mol Cell. 2015; 58(3):440-52).

In some embodiments, the cancer is a solid tumor. In some embodiments, the cancer is a neuroblastoma.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The terms below, as used herein, have the following meanings, unless indicated otherwise:

“Oxo” refers to the ═O substituent.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methythexyl, 1-ethyl-propyl, and the like. In some embodiments, the alkyl is methyl or ethyl. Unless stated otherwise specifically in the specification, an alkyl group may be optionally substituted as described below.

“Alkylene” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized n-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

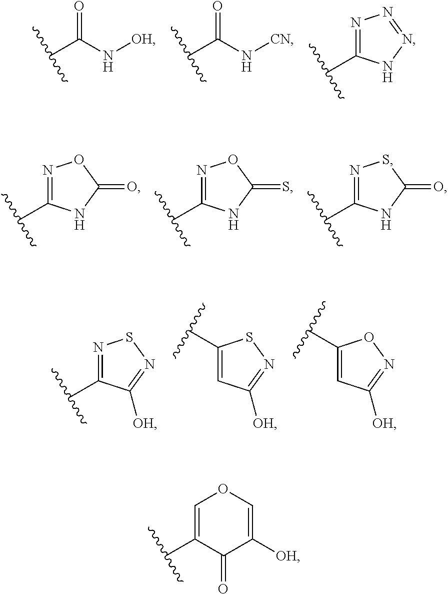

and the like.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. In some embodiments, a cycloalkyl is a C₃-C₆cycloalkyl. In some embodiments, the cycloalkyl is monocyclic, bicyclic or polycyclic. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, decalinyl and adamantyl. In some embodiments, the cycloalkyl is monocyclic. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the cycloalkyl is bicyclic. Bicyclic cycloalkyl groups include fused bicyclic cycloalkyl groups, spiro bicyclic cycloalkyl groups, and bridged bicyclic cycloalkyl groups. In some embodiments, cycloalkyl groups are selected from among spiro[2.2]pentyl, bicyclo[1.1.1]pentyl, bicyclo[3.3.0]octane, bicyclo[4.3.0]nonane, bicyclo[2.1.1]hexane, bicyclo [2.2.1]heptane, bicyclo[2.2.2]octane, bicyclo[3.2.2]nonane, bicyclo[3.3.2]decane, norbornyl, 3,4-dihydronaphthalen-1(2H)-one and decalinyl. In some embodiments, the cycloalkyl is polycyclic. Polycyclic radicals include, for example, adamantyl, and. In some embodiments, the polycyclic cycloalkyl is adamantyl. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, bicyclic ring (which may include a fused bicyclic heterocycloalkyl (when fused with an aryl or a heteroaryl ring, the heterocycloalkyl is bonded through a non-aromatic ring atom), bridged heterocycloalkyl or spiro heterocycloalkyl), or polycyclic. In some embodiments, the heterocycloalkyl is monocyclic or bicyclic. In some embodiments, the heterocycloalkyl is monocyclic. In some embodiments, the heterocycloalkyl is bicyclic. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆ alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (=O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

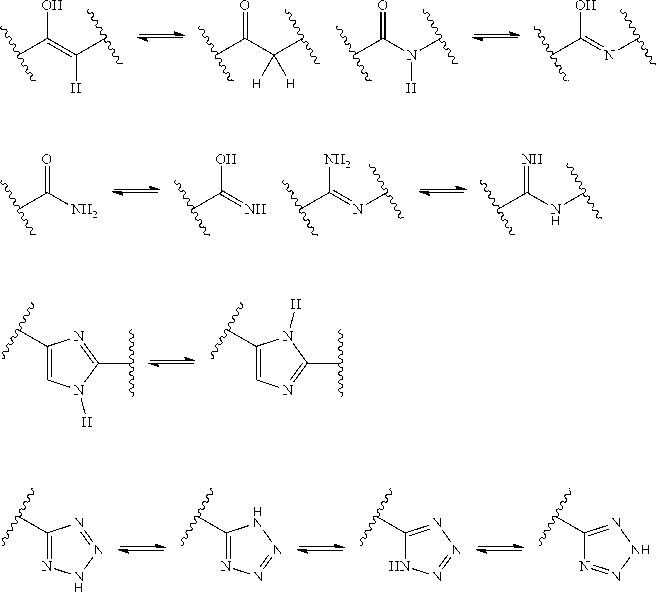

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound of Formula (I) and a co-agent, are both administered to a patient simultaneously in the form of a single entity or dosage. The term “non-fixed combination” means that the active ingredients, e.g., a compound of Formula (I) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The term “subject” or “patient” encompasses mammals. Examples of mammals include, but are not limited to, humans. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Methods of Synthesis

In some embodiments, the syntheses of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

It is understood that other analogous procedures and reagents could be used, and that these Schemes are only meant as non-limiting examples.

### EXAMPLES

### Preparation of Compounds

### Abbreviations

DCM: Dichloromethane
DIEA: Diisopropylethylamine
DMF: Dimethyl formamide
DMSO: Dimethyl sulfoxide
ESI: Electrospray ionization
HPLC: High performance liquid chromatography
HRMS: High resolution mass spectrometry
h or hr(s): Hour(s)
HATU: 1-[Bis(dimethylamino)methylene]-1H-1,2,3-triazolo[4,5-b]pyridinium 3-oxid hexafluorophosphate
min(s): Minutes
m/z: Mass-to-charge ratio
¹H NMR: Proton nuclear magnetic resonance
¹³C NMR: Carbon nuclear magnetic resonance
rt: Room temperature

### Example 1. Synthesis of 1-(3-(5-amino-2-chloro-4-fluoro-3-methylbenzamido)-4-(4-methylpiperazin-1-yl)phenyl)-N-(2-ethoxyethyl)-1H-1,2,3-triazole-4-carboxamide (Compound 1, Table 1)

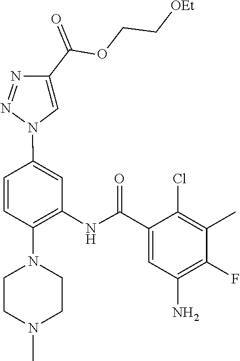 [SMILES: CCOCCOC(=O)c1cn(-c2ccc(N3CCN(C)CC3)c(NC(=O)c3cc(N)c(F)c(C)c3Cl)c2)nn1]

The title compound was synthesized according to Schemes 1-3 (Example 2) starting with 1-(3-(5-amino-2-chloro-4-fluoro-3-methylbenzamido)-4-(4-methylpiperazin-1-yl)phenyl)-1H-1,2,3-triazole-4-carboxylic acid (80 mg, 163.96 μmol, 1 eq.) and 2-ethoxyethanol (22 mg, 245.94 μmol, 23.83 μL, 1.5 eq.) in DMF (1 mL) was added DMAP (24 mg, 196.76 μmol, 1.2 eq.), 2-chloro-1-methyl-pyridin-1-ium; iodide (50 mg, 196.76 μmol, 1.2 eq.) and Et₃N (33 mg, 327.93 μmol, 45.64 μL, 2 eq.) in one portion. The mixture was stirred at 30° C. and stirred for 12 hrs. The residue was concentrated under reduce pressure to remove DMF. The residue was purified by prep. HPLC (Column: Welch Xtimate C18 150*25 mm*5 μm; mobile phase: [water (0.05% ammonia hydroxide v/v)-ACN]; 7.8 min). Example 1 (20.7 mg, 36.40 μmol, 22.20% yield, 98.471% purity) was obtained as a white solid.

¹H NMR: (DMSO-d₆, 400 MHz) δ_{H}=9.56-9.45 (m, 2H), 8.65 (s, 1H), 7.74 (dd, J=2.8, 8.8 Hz, 1H), 7.43 (d, J=8.8 Hz, 1H), 6.88 (d, J=9.2 Hz, 1H), 5.53 (s, 2H), 4.48-4.41 (m, 2H), 3.74-3.68 (m, 2H), 3.51 (q, J=6.8 Hz, 3H), 3.00 (s, 4H), 2.69 (s, 3H), 2.40-2.30 (m, 3H), 2.26 (d, J=2.4 Hz, 3H), 1.15-1.10 (m, 1H), 1.12 (t, J=7.2 Hz, 2H).

HPLC: Rₜ=3.556 min in 8 min chromatography, Xbridge C18 2.1*50 mm 5 μm, 8 um, purity 98.41%.

LCMS: Rₜ=0.693 min in 1.5 min chromatography, Agilent Pursit 5 C18 20*2.0 mm, 8 μm, purity 100%, MS ESI calcd. for 559.21 [M+H]⁺560.21, found 560.1.

### Example 2. Synthesis of isobutyl 1-(3-(5-amino-2-chloro-4-fluoro-3-methylbenzamido)-4-(4-methylpiperazin-1-yl)phenyl)-1H-1,2,3-triazole-4-carboxylate (Compound 2, Table 1)

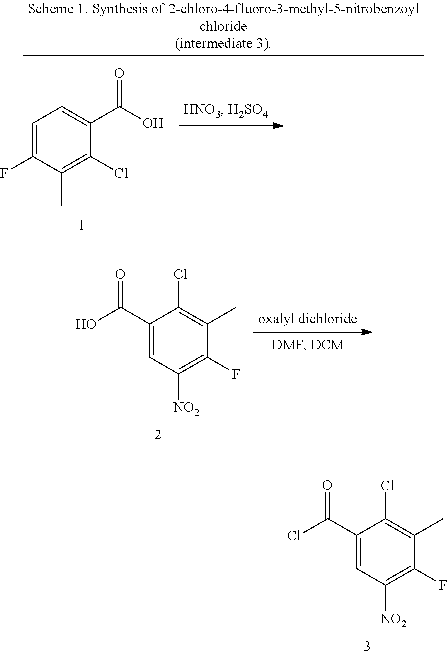

Step 1: To a mixture of 2-chloro-4-fluoro-3-methylbenzoic acid (10 g, 53.03 mmol, 1 eq.) in H₂SO₄ (100 mL) was added HNO₃ (5.65 g, 58.33 mmol, 4.04 mL, 65% purity, 1.1 eq.) dropwise at 0° C. The mixture was allowed to warm up to 25° C. and stirred for 2 hrs. The mixture was poured into ice water. The mixture was filtered via a filter paper, and the filter cake was dried under reduced pressure. The product was used directly to the next step without further purification. 2-Chloro-4-fluoro-3-methyl-5-nitrobenzoic acid (10 g, 34.79 mmol, 65.61% yield, 81.27% purity) was obtained as an off-white solid.

¹H NMR: (DMSO-d₆, 400 MHz) δ_{H}=8.36 (d, J=8.0 Hz, 1H), 2.39 (d, J=2.8 Hz, 1H).

HPLC: Rₜ=2.60 min in 8 min chromatography, Ultimate XB-C18 3.0*50 mm, 3 m, purity 81.27%.

LCMS: Rₜ=1.284 min in 4 min chromatography, Xtimate C18, 3 μm, 2.1*30 mm, purity 81.30%.

Step 2: To a solution of 2-chloro-4-fluoro-3-methyl-5-nitrobenzoic acid (500 mg, 2.14 mmol, 1 eq.) and DMF (7.82 mg, 107.03 μmol, 8.23 μL, 0.05 eq.) in DCM (5 mL) was added (COCl)₂ (408 mg, 3.21 mmol, 281.07 μL, 1.5 eq.) drop-wise at 0° C. The reaction mixture was stirred at 20° C. for 1 hr to give a yellow solution. The reaction mixture was concentrated to dryness. The crude product 2-chloro-4-fluoro-3-methyl-5-nitro-benzoyl chloride (500 mg, crude) was obtained as yellow oil and used into the next step without further purification.

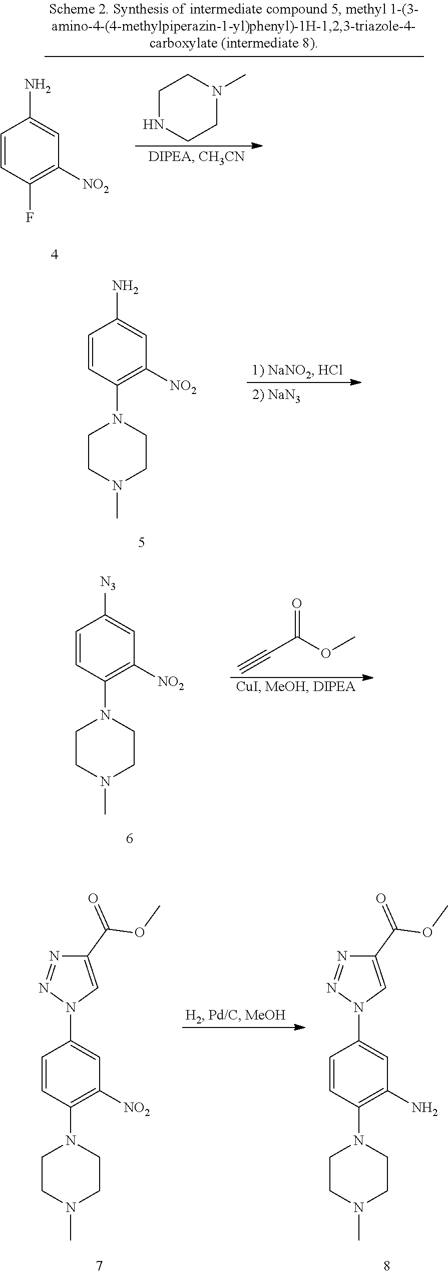

Step 1: To a mixture of 4-fluoro-3-nitroaniline (50 g, 320.28 mmol, 1 eq.) and 1-methylpiperazine (64.16 g, 640.56 mmol, 71.05 mL, 2 eq.) in CH₃CN (500 mL) was added DIEA (82.79 g, 640.56 mmol, 111.57 mL, 2 eq.). The mixture was stirred at 90° C. for 12 hrs. The mixture was diluted with DCM (200 mL), washed with brine (50 mL*3). The combined organic layer was dried over Na₂SO₄ and concentrated to give crude product. The crude product was purified by column chromatography on silica gel (DCM: MeOH=20:1). 4-(4-Methylpiperazin-1-yl)-3-nitroaniline (72 g, 302.15 mmol, 94.34% yield, 99.15% purity) was obtained as a red solid.

¹H NMR: (DMSO-d₆, 400 MHz) δ_{H}=7.16 (d, J=8.8 Hz, 1H), 6.68 (d, J=2.4 Hz, 1H), 6.78 (dd, J=2.8, 8.8 Hz, 1H), 5.43 (s, 2H), 2.82-2.79 (m, 4H), 2.50-2.34 (m, 4H), 2.19 (s, 3H).

Step 2: To a mixture of 4-(4-methylpiperazin-1-yl)-3-nitroaniline (35 g, 148.14 mmol, 1 eq.) in HCl (2 M, 875.00 mL, 11.81 eq.) was a solution of NaNO₂ (15.33 g, 222.20 mmol, 1.5 eq.) in H₂O (90 mL) dropwise at 0° C. After 0.5 hr, a solution of NaN₃ (19.26 g, 296.27 mmol, 2 eq.) in H₂O (90 mL) was added into the mixture. After 0.5 hr, the mixture was allowed to warm up to 25° C. and stirred for 2 hrs. The pH of the mixture was adjusted to around 9 with 2N NaOH. The mixture was filtered via a filter paper. The filter cake was obtained as a red solid. The product was used directly to the next step without further purification. 1-(4-Azido-2-nitrophenyl)-4-methylpiperazine (30 g, 109.33 mmol, 73.80% yield, 95.58% purity) was obtained as a red solid.

¹H NMR: (DMSO-d₆, 400 MHz) δ_{H}=7.56 (d, J=2.0 Hz, 1H), 7.39-7.32 (m, 2H), 2.95-2.92 (m, 4H), 2.41-2.38 (m, 4H), 2.20 (s, 3H).

Step 3: To a mixture of 1-(4-azido-2-nitrophenyl)-4-methylpiperazine (30 g, 114.39 mmol, 1 eq.) and methyl prop-2-ynoate (28.85 g, 343.16 mmol, 28.57 mL, 3 eq.) in MeOH (1500 mL) was added CuI (6.54 g, 34.32 mmol, 0.3 eq.) and DIEA (2.96 g, 22.88 mmol, 3.98 mL, 0.2 eq.). The mixture was stirred at 65° C. for 48 hrs. The mixture was diluted with DCM (2000 mL), washed with 2N NH₃·H₂O (1000 mL*2) and brine (500 mL*2). The combined organic layer was dried over Na₂SO₄ and concentrated to give crude product. The product was purified by column chromatography on silica gel (DCM: MeOH=10:1). Methyl 1-(4-(4-methylpiperazin-1-yl)-3-nitrophenyl)-1H-1,2,3-triazole-4-carboxylate (32 g, 84.28 mmol, 73.68% yield, 91.22% purity) was obtained as a red solid.

¹H NMR: (DMSO-d₆, 400 MHz) δ_{H}=9.54 (s, 1H), 8.45 (d, J=2.4 Hz, 1H), 8.15 (dd, J=2.8, 9.2 Hz, 1H), 7.51 (d, J=8.8 Hz, 1H), 3.90 (s, 3H), 3.12-3.09 (m, 4H), 2.50-2.45 (m, 4H), 2.24 (s, 3H).

Step 4: To a solution of methyl 1-(4-(4-methylpiperazin-1-yl)-3-nitrophenyl)-1H-1,2,3-triazole-4-carboxylate (10 g, 28.87 mmol, 1 eq.) in MeOH (100 mL) was added Pd/C (1 g, 10% purity) under N₂ atmosphere. The suspension was degassed and purged with H₂ for 3 times. The mixture was stirred under H₂ (15 Psi.) at 30° C. for 12 h. The mixture was filtered via a celite pad, and the pad was washed with MeOH (100 mL*3). The filtrate was concentrated to give the desired product. Methyl 1-(3-amino-4-(4-methylpiperazin-1-yl)phenyl)-1H-1,2,3-triazole-4-carboxylate (6.1 g, 19.09 mmol, 66.11% yield, 99% purity) was obtained as a red solid.

¹H NMR: (DMSO-d₆, 400 MHz) δ_{H}=9.28 (s, 1H), 7.26 (d, J=2.0 Hz, 1H), 7.08-7.02 (m, 2H), 5.15 (s, 2H), 3.88 (s, 3H), 2.85 (s, 4H), 2.56-2.51 (m, 4H), 2.24 (s, 3H).

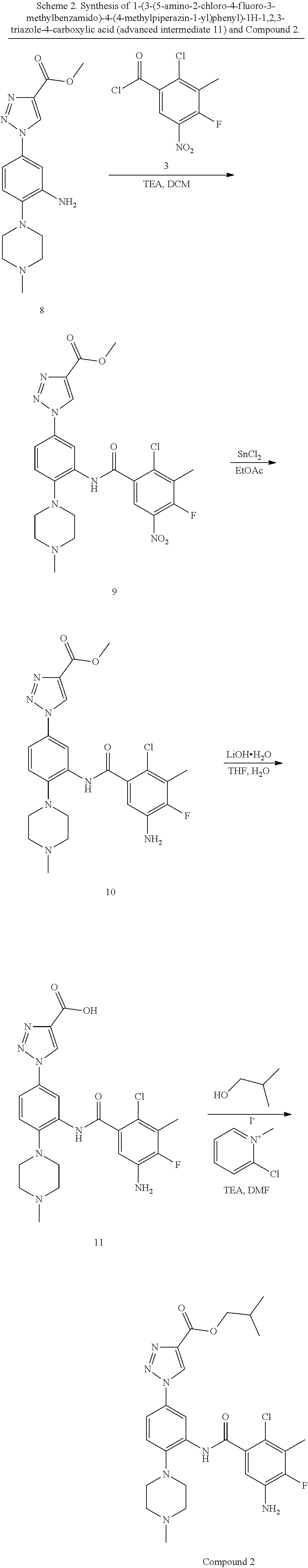

Step 5: To a solution of methyl 1-(3-amino-4-(4-methylpiperazin-1-yl)phenyl)-1H-1,2,3-triazole-4-carboxylate (3.8 g, 12.01 mmol, 1 eq.) and 2-chloro-4-fluoro-3-methyl-5-nitrobenzoyl chloride (3.33 g, 13.21 mmol, 1.1 eq.) in DCM (50 mL) was added Et₃N (6.08 g, 60.06 mmol, 8.36 mL, 5 eq.) at 0° C. The reaction mixture was stirred at 15° C. for 2 hr to give a brown mixture. The reaction mixture was concentrated. The residue was purified by flash silica gel chromatography (ISCO®; 80 g SepaFlash® Silica Flash Column, Eluent of 0-10% MeOH/DCM ether gradient at 60 m/min). The product methyl 1-(3-(2-chloro-4-fluoro-3-methyl-5-nitrobenzamido)-4-(4-methylpiperazin-1-yl)phenyl)-1H-1,2,3-triazole-4-carboxylate (3.3 g, 4.34 mmol, 36.14% yield, 70.2% purity) was obtained as a yellow solid.

¹H NMR: (CDCl₃, 400 MHz) δ_{H}=9.36 (s, 1H), 9.00-8.89 (m, 1H), 8.62-8.55 (m, 1H), 8.32 (d, J=7.6 Hz, 1H), 7.76-7.65 (m, 1H), 7.48-7.39 (m, 1H), 4.01 (s, 3H), 3.04-2.92 (m, 4H), 2.78-2.47 (m, 7H), 2.46-2.31 (m, 3H).

Step 6: To a solution of methyl 1-(3-(2-chloro-4-fluoro-3-methyl-5-nitrobenzamido)-4-(4-methylpiperazin-1-yl)phenyl)-1H-1,2,3-triazole-4-carboxylate (5 g, 9.40 mmol, 1 eq.) in EtOAc (100 mL) was added SnCl₂·2H₂O (8.48 g, 37.60 mmol, 4 eq.). The reaction mixture was stirred at 80° C. for 3 hr to give a yellow mixture. The reaction mixture was adjusted to pH-8 by aq.NaHCO₃. The resulting mixture was filtered, and the filter cake was washed with EtOAc (50 mL×3). The filtrate was concentrated. The residue was purified by flash silica gel chromatography (ISCO®; 120 g SepaFlash® Silica Flash Column, Eluent of 0-10% MeOH/DCM ether gradient at 60 mL/min). The product methyl 1-(3-(5-amino-2-chloro-4-fluoro-3-methylbenzamido)-4-(4-methylpiperazin-1-yl)phenyl)-1H-1,2,3-triazole-4-carboxylate (2.5 g, 4.27 mmol, 45.46% yield, 85.8% purity) was obtained as a yellow solid.

¹H NMR: (CDCl₃, 400 MHz) δ_{H}=9.39 (s, 1H), 8.97 (d, J=2.4 Hz, 1H), 8.58 (s, 1H), 7.66 (dd, J=2.8, 8.8 Hz, 1H), 7.39 (d, J=8.8 Hz, 1H), 7.06 (d, J=9.2 Hz, 1H), 4.00 (s, 3H), 3.88 (s, 2H), 2.97 (t, J=4.8 Hz, 4H), 2.75-2.48 (m, 4H), 2.43-2.31 (m, 6H).

Step 7: To a mixture of methyl 1-(3-(5-amino-2-chloro-4-fluoro-3-methylbenzamido)-4-(4-methylpiperazin-1-yl)phenyl)-1H-1,2,3-triazole-4-carboxylate (1.4 g, 2.79 mmol, 1 eq.) in THF (20 mL) and H₂O (2 mL) was added LiOH·H₂O (234.09 mg, 5.58 mmol, 2 eq.). The reaction mixture was stirred at 15° C. for 2 hr to give a yellow mixture. The reaction mixture was then adjusted to pH-5 by aq. HCl (1 N). The resulting mixture was concentrated to dryness. The crude product 1-(3-(5-amino-2-chloro-4-fluoro-3-methylbenzamido)-4-(4-methylpiperazin-1-yl)phenyl)-1H-1,2,3-triazole-4-carboxylic acid 1-(3-(5-amino-2-chloro-4-fluoro-3-methylbenzamido)-4-(4-methylpiperazin-1-yl)phenyl)-1H-1,2,3-triazole-4-carboxylic acid 1-(3-(5-amino-2-chloro-4-fluoro-3-methylbenzamido)-4-(4-methylpiperazin-1-yl)phenyl)-1H-1,2,3-triazole-4-carboxylic acid (1.6 g, crude) was obtained as a yellow solid and used into the next step without further purification.

¹H NMR: (DMSO-d₆, 400 MHz) δ_{H}=11.23-11.01 (m, 1H), 9.65 (s, 1H), 9.39 (s, 1H), 8.65 (s, 1H), 7.76 (dd, J=2.8, 8.8 Hz, 1H), 7.43 (d, J=8.8 Hz, 1H), 6.88 (d, J=9.2 Hz, 1H), 3.71-3.61 (m, 2H), 3.51-3.43 (m, 2H), 3.28-3.18 (m, 6H), 2.81 (d, J=3.6 Hz, 3H), 2.27 (d, J=2.4 Hz, 3H).

Step 8: To a mixture of 1-(3-(5-amino-2-chloro-4-fluoro-3-methylbenzamido)-4-(4-methylpiperazin-1-yl)phenyl)-1H-1,2,3-triazole-4-carboxylic acid 1-(3-(5-amino-2-chloro-4-fluoro-3-methylbenzamido)-4-(4-methylpiperazin-1-yl)phenyl)-1H-1,2,3-triazole-4-carboxylic acid (150 mg, 307.43 μmol, 1 eq.) and 2-methylpropan-1-ol (34.18 mg, 461.15 μmol, 42.62 μL, 1.5 eq.) in DMF (1 mL) was added DMAP (45.07 mg, 368.92 μmol, 1.2 eq), Et₃N (62.22 mg, 614.86 μmol, 85.58 μL, 2 eq.) and 2-chloro-1-methyl-pyridin-1-ium; iodide (157.09 mg, 614.86 μmol, 2 eq.). The reaction mixture was stirred at 30° C. for 12 hr to give a yellow mixture. Water (10 mL) was added to the reaction mixture. The resulting mixture was extracted with DCM (10 mL*3). The combined organic phase was washed with brine (20 mL), dried over anhydrous Na₂SO₄, filtered and concentrated. The residue was purified by prep-HPLC (Column: Phenomenex luna C18 80*40 mm*3 μm; Condition: water (0.05% ammonia hydroxide v/v)-ACN; Begin B: 40%, End B: 70%; Gradient Time(min): 20 min). The product Example 2 (20.2 mg, 36.63 μmol, 11.91% yield, 99.14% purity) was obtained as an off-white solid.

¹H NMR: (DMSO-d₆, 400 MHz) δ_{H}=9.48 (s, 1H), 9.45 (s, 1H), 8.65 (s, 1H), 7.73 (dd, J=2.8, 8.8 Hz, 1H), 7.43 (d, J=8.8 Hz, 1H), 6.90 (d, J=9.6 Hz, 1H), 5.53 (s, 2H), 4.12 (d, J=6.6 Hz, 2H), 2.93 (t, J=4.3 Hz, 4H), 2.49-2.40 (m, 4H), 2.30-2.18 (m, 6H), 2.10-1.95 (m, 1H), 0.99 (d, J=6.8 Hz, 6H).

HPLC: Rₜ=4.08 min in 8 min chromatography, Ultimate XB-C18 3.0*50 mm, 3 m, purity 99.14%.

LCMS: Rₜ=0.868 min in 4 min chromatography, Chromolith Flash RP-18, 5 μm, 3.0*25 mm, purity 100%, MS ESI calcd. for 543.22 [M+H]⁺ 544.22, found 544.2.

### Example 3. Synthesis of -methoxybenzyl 1-(3-(5-amino-2-chloro-4-fluoro-3-methylbenzamido)-4-(4-methylpiperazin-1-yl)phenyl)-1H-1,2,3-triazole-4-carboxylate (Compound 3, Table 1)

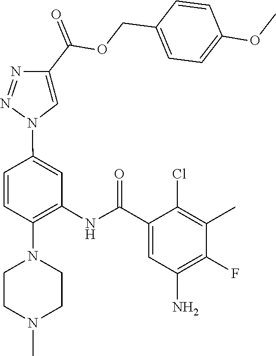 [SMILES: COc1ccc(COC(=O)c2cn(-c3ccc(N4CCN(C)CC4)c(NC(=O)c4cc(N)c(F)c(C)c4Cl)c3)nn2)cc1]

The title compound was synthesized according to Schemes 1-3 (Example 2). To a mixture of 1-(3-(5-amino-2-chloro-4-fluoro-3-methylbenzamido)-4-(4-methylpiperazin-1-yl)phenyl)-1H-1,2,3-triazole-4-carboxylic acid (51 mg, 368.92 μmol, 45.92 μL, 1.2 eq.) and 4-methoxyphenyl)methanol (150 mg, 307.43 μmol, 1 eq.) in DMF (1 mL) was added DMAP (45 mg, 368.92 μmol, 1.2 eq.), 2-chloro-1-methyl-pyridin-1-ium; iodide (157 mg, 614.86 μmol, 2 eq) and TEA (62 mg, 614.86 μmol, 85.58 μL, 2 eq) in one portion. The mixture was stirred at 30° C. and stirred for 12 hrs. The residue was poured into water (20 mL). The aqueous phase was extracted with DCM (10 mL*3). The combined organic phase was washed with brine (20 mL), dried with anhydrous Na₂SO₄, filtered and concentrated in vacuum. The residue was purified by prep. HPLC (Column: Phenomenex luna C18 80*40 mm*3 μm; mobile phase: [water (0.05% ammonia hydroxide v/v)-ACN]; B %: 42%-72%; 11 min). Example 3 (40.7 mg, 66.70 μmol, 21.70% yield, 99.647% purity) was obtained as a white solid.

¹H NMR: (DMSO-d₆, 400 MHz) δ_{H}=9.49-9.44 (m, 2H), 8.65 (s, 1H), 7.72 (dd, J=2.8, 8.8 Hz, 1H), 7.48-7.38 (m, 3H), 7.01-6.85 (m, 3H), 5.52 (s, 2H), 5.33 (s, 2H), 3.76 (s, 3H), 2.92 (t, J=4.4 Hz, 4H), 2.47 (s, 4H), 2.30-2.17 (m, 6H).

HPLC: Rₜ=3.049 min in 8 min chromatography, XBridge Shield RP18, 5 μm, 2.1*50 mm, 4 μm, purity 99.647%.

LCMS: Rₜ=2.425 min in 4 min chromatography, XBridge Shield RP18, 5 μm, 2.1*50 mm, 4 m, purity 99.821%, MS ESI calcd. for 608.06 [M+H]⁺609.06, found 608.4.

### Example 4. Synthesis of (S)-1-ethoxy-1-oxopropan-2-yl 1-(3-(5-amino-2-chloro-4-fluoro-3-methylbenzamido)-4-(4-methylpiperazin-1-yl)phenyl)-1H-1,2,3-triazole-4-carboxylate (Compound 4, Table 1)

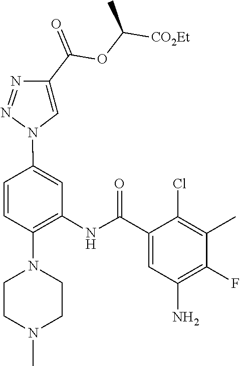 [SMILES: CCOC(=O)[C@H](C)OC(=O)c1cn(-c2ccc(N3CCN(C)CC3)c(NC(=O)c3cc(N)c(F)c(C)c3Cl)c2)nn1]

The title compound was synthesized according to Schemes 1-3 (Example 2). To a mixture of 1-(3-(5-amino-2-chloro-4-fluoro-3-methylbenzamido)-4-(4-methylpiperazin-1-yl)phenyl)-1H-1,2,3-triazole-4-carboxylic acid (150 mg, 307.43 μmol, 1 eq.) and ethyl (2S)-2-hydroxypropanoate (54 mg, 461.15 μmol, 52.89 μL, 1.5 eq.) in DMF (1 mL) was added DMAP (45 mg, 368.92 μmol, 1.2 eq.), TEA (62 mg, 614.86 mol, 85.58 μL, 2 eq.) and 2-chloro-1-methyl-pyridin-1-ium; iodide (157 mg, 614.86 μmol, 2 eq.) in one portion. The mixture was stirred at 30° C. and stirred for 12 hours. The residue was poured into water (5 mL). The aqueous phase was extracted with DCM (5 mL*3). The combined organic phase was washed with brine (10 mL), dried with anhydrous Na₂SO₄, filtered and concentrated in vacuum. The residue was purified by prep. HPLC(Column: Welch Xtimate C18 150*25 mm*5 μm; mobile phase: [water (0.05% ammonia hydroxide v/v)-ACN]; B %: 42%-75%; 11 min). Example 4 (58.14 mg, 88.49 μmol, 28.78% yield, 98.546% purity) was obtained as a white solid.

¹H NMR: (DMSO-d₆, 400 MHz) δ_{H}=9.51 (d, J=16.4 Hz, 2H), 8.66 (s, 1H), 7.74 (dd, J=2.4, 8.6 Hz, 1H), 7.44 (d, J=8.8 Hz, 1H), 6.89 (d, J=9.6 Hz, 1H), 5.53 (s, 2H), 5.29 (q, J=7.2 Hz, 1H), 4.18 (q, J=7.2 Hz, 2H), 2.93 (d, J=4.4 Hz, 4H), 2.49-2.45 (m, 4H), 2.29-2.18 (m, 6H), 1.57 (d, J=7.2 Hz, 3H), 1.21 (t, J=7.2 Hz, 3H).

HPLC: Rₜ=2.817 min in 8 min chromatography, XBridge Shield RP18, 5 μm, 2.1*50 mm, 5 μm, purity 98.546%.

LCMS: Rₜ=2.257 min in 4 min chromatography, XBridge Shield RP18, 5 μm, 2.1*50 mm, 5 um, purity 99.059%, MS ESI calcd. for 587.21 [M+H]⁺588.21, found 588.3.

### Example 5. (1-methylcyclohexyl)methyl 1-(3-(5-amino-2-chloro-4-fluoro-3-methylbenzamido)-4-(4-methylpiperazin-1-yl)phenyl)-1H-1,2,3-triazole-4-carboxylate (Compound 5, Table 1)

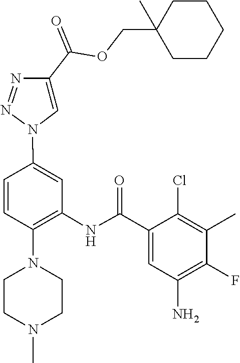 [SMILES: Cc1c(F)c(N)cc(C(=O)Nc2cc(-n3cc(C(=O)OCC4(C)CCCCC4)nn3)ccc2N2CCN(C)CC2)c1Cl]

The title compound was synthesized according to Schemes 1-3 (Example 2). To a mixture of 1-(3-(5-amino-2-chloro-4-fluoro-3-methylbenzamido)-4-(4-methylpiperazin-1-yl)phenyl)-1H-1,2,3-triazole-4-carboxylic acid (80 mg, 163.96 μmol, 1 eq.) and (1-methylcyclohexyl)methanol (32 mg, 245.94 μmol, 1.5 eq.) in DMF (1 mL) was added DMAP (24 mg, 196.76 μmol, 1.2 eq.), TEA (33 mg, 327.93 μmol, 45.64 μL, 2 eq.) and 2-chloro-1-methyl-pyridin-1-ium; iodide (50 mg, 196.76 μmol, 1.2 eq) in one portion. The mixture was stirred at 30° C. and stirred for 12 hours. The residue was poured into water (5 mL). The aqueous phase was extracted with DCM (5 mL*3). The combined organic phase was washed with brine (10 mL), dried with anhydrous Na₂SO₄, filtered and concentrated in vacuum. The residue was purified by prep. HPLC(Column: Phenomenex luna C18 100*40 mm*3 μm; mobile phase: [water (0.225% FA)-ACN]; 10 min). Example 5 (13 mg, 21.66 μmol, 13.21% yield, 99.65% purity) was obtained as a white solid.

¹H NMR: (DMSO-d₆, 400 MHz) δ_{H}=9.46 (d, J=17.6 Hz, 2H), 8.65 (s, 1H), 8.18 (s, 1H), 7.77-7.68 (m, 1H), 7.44 (d, J=8.4 Hz, 1H), 6.90 (d, J=9.2 Hz, 1H), 5.52 (s, 2H), 4.10 (s, 2H), 3.32 (s, 4H), 2.93 (s, 4H), 2.24 (d, J=13.6 Hz, 6H), 1.54-1.23 (m, 10H), 1.01 (s, 3H).

HPLC: Rₜ=3.48 min in 8 min chromatography, Ultimate XB-C18 3.0*50 mm, 3 μm, 10 m, purity 99.01%.

LCMS: Rₜ=2.076 min in 4 min chromatography, Xtimate C18, 3 μm, 2.1*30 mm, 10 um, purity 99.882%, MS ESI calcd. for 597.26 [M+H]⁺598.26, found 598.4.

### Example 6. Synthesis of (1-methylpiperidin-4-yl)methyl 1-(3-(5-amino-2-chloro-4-fluoro-3-methylbenzamido)-4-(4-methylpiperazin-1-yl)phenyl)-1H-1,2,3-triazole-4-carboxylate (Compound 6, Table 1)

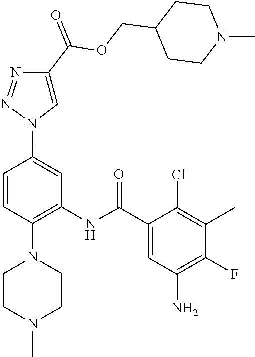 [SMILES: Cc1c(F)c(N)cc(C(=O)Nc2cc(-n3cc(C(=O)OCC4CCN(C)CC4)nn3)ccc2N2CCN(C)CC2)c1Cl]

The title compound was synthesized according to Schemes 1-3 (Example 2). To a mixture of 1-(3-(5-amino-2-chloro-4-fluoro-3-methylbenzamido)-4-(4-methylpiperazin-1-yl)phenyl)-1H-1,2,3-triazole-4-carboxylic acid (80 mg, 163.96 μmol, 1 eq.) and (1-methyl-4-piperidyl)methanol (32 mg, 245.94 μmol, 1.5 eq.) in DMF (1 mL) was added DMAP (24 mg, 196.76 μmol, 1.2 eq.), TEA (33 mg, 327.93 μmol, 45.64 μL, 2 eq.) and 2-chloro-1-methyl-pyridin-1-ium; iodide (50 mg, 196.76 μmol, 1.2 eq.) in one portion. The mixture was stirred at 30° C. and stirred for 12 hrs. The residue was poured into water (5 mL). The aqueous phase was extracted with DCM (5 mL*3). The combined organic phase was washed with brine (10 mL), dried with anhydrous Na₂SO₄, filtered and concentrated in vacuum. The residue was purified by prep. HPLC (Column: Welch Xtimate C18 150*25 mm*5 μm; mobile phase: [water (0.05% ammonia hydroxide v/v)-ACN]; 7.8 min). Example 6 (10 mg, 16.59 μmol, 10.12% yield, 99.37% purity) was obtained as a white solid.

¹H NMR: (DMSO-d₆, 400 MHz) δ_{H}=9.46 (d, J=17.6 Hz, 2H), 8.65 (s, 1H), 8.18 (s, 1H), 7.77-7.68 (m, 1H), 7.44 (d, J=8.4 Hz, 1H), 6.90 (d, J=9.2 Hz, 1H), 5.52 (s, 2H), 4.10 (s, 2H), 3.32 (s, 4H), 2.93 (s, 4H), 2.24 (d, J=13.6 Hz, 6H), 1.54-1.23 (m, 10H), 1.01 (s, 3H).

HPLC: Rₜ=4.067 min in 8 min chromatography, XBridge Shield RP18, 5 μm, 2.1*50 mm, 5 m, purity 96.881%.

LCMS: Rₜ=1.852 min in 4 min chromatography, Xbridge Shield RP-18.5 μm, 2.1*50 mm, 40 μm, purity 97.972%, MS ESI calcd. for 598.26 [(M+2H)/2]⁺300.13, found 300.2.

### Example 7. Synthesis of 1-(3-(5-amino-2-chloro-4-fluoro-3-methylbenzamido)-4-(4-methylpiperazin-1-yl)phenyl)-1H-1,2,3-triazole-4-carboxylate (Compound 7, Table 1)

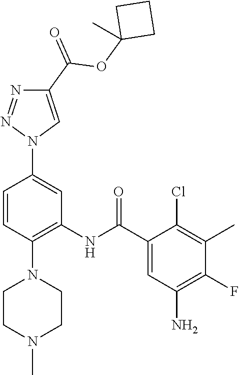 [SMILES: Cc1c(F)c(N)cc(C(=O)Nc2cc(-n3cc(C(=O)OC4(C)CCC4)nn3)ccc2N2CCN(C)CC2)c1Cl]

The title compound was synthesized according to Schemes 1-3 (Example 2). To a mixture of 1-(3-(5-amino-2-chloro-4-fluoro-3-methylbenzamido)-4-(4-methylpiperazin-1-yl)phenyl)-1H-1,2,3-triazole-4-carboxylic acid (300 mg, 614.86 μmol, 1 eq) and 1-methylcyclobutanol (79 mg, 922.29 μmol, 1.5 eq) in DMF (1 mL) was added DMAP (180 mg, 1.48 mmol, 2.4 eq), TEA (124 mg, 1.23 mmol, 171.16 μL, 2 eq) and 2-chloro-1-methyl-pyridin-1-ium iodide (314 mg, 1.23 mmol, 2 eq) in one portion. The mixture was stirred at 100° C. and stirred for 12 hrs. The residue was poured into water (20 mL). The aqueous phase was extracted with DCM (10 mL*3). The combined organic phase was washed with brine (10 mL), dried with anhydrous Na₂SO₄, filtered and concentrated in vacuum. The residue was purified by prep. HPLC (Column: Phenomenex luna C18 80*40 mm*3 μm; mobile phase: [water (0.05% ammonia hydroxide v/v)-ACN]; B %: 45%-80%; 13 min). Example 7 (10.5 mg, 18.77 μmol, 3.05% yield, 99.39% purity) was obtained as a white solid.

¹H NMR: (DMSO-d₆, 400 MHz) δ_{H}=9.52-9.33 (m, 2H), 8.65 (s, 1H), 7.71 (d, J=6.8 Hz, 1H), 7.43 (d, J=8.4 Hz, 1H), 6.89 (d, J=9.2 Hz, 1H), 5.53 (s, 2H), 4.64-3.96 (m, 1H), 2.93 (s, 4H), 2.48-2.36 (m, 5H), 2.24 (d, J=16.8 Hz, 8H), 1.90-1.69 (m, 2H), 1.64 (s, 2H), 1.48-0.81 (m, 1H).

HPLC: Rₜ=3.044 min in 8 min chromatography, XBridge Shield RP18, 5 μm, 2.1*50 mm, 10 μm, purity 99.39%.

LCMS: Rₜ=2.443 min in 4 min chromatography, XBridge Shield RP18, 5 μm, 2.1*50 mm, 6 μm, purity 99.676%, MS ESI calcd. for 555.22 [M+H]⁺556.22, found 556.4.

### Example 8. Synthesis of 1-(3-(5-amino-2-chloro-4-fluoro-3-methylbenzamido)-4-(4-methylpiperazin-1-yl)phenyl)-N-methyl-N-(3-morpholinopropyl)-1H-1,2,3-triazole-4-carboxamide (Compound 8, Table 1)

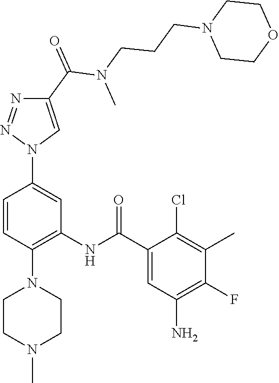 [SMILES: Cc1c(F)c(N)cc(C(=O)Nc2cc(-n3cc(C(=O)N(C)CCCN4CCOCC4)nn3)ccc2N2CCN(C)CC2)c1Cl]

The title compound was synthesized according to Schemes 1-3 (Example 2) and Scheme 4.

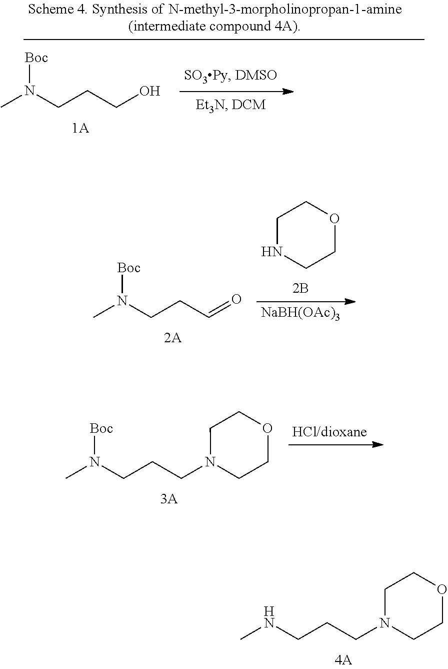

Step 1: To a mixture of tert-butyl (3-hydroxypropyl)(methyl)carbamate (200 mg, 1.06 mmol, 1 eq.) in DCM (5 mL), TEA (1.07 g, 10.57 mmol, 1.47 mL, 10 eq) and DMSO (247.71 mg, 3.17 mmol, 247.71 μL, 3 eq.) was added SO₃·Py (504.61 mg, 3.17 mmol, 3 eq.) drop-wise at 0° C. The reaction mixture was stirred at 25° C. for 4 hr. The mixture was extracted with DCM (10 mL*3). The combined organic phase was washed with NaHCO₃ (10 mL*3) and brine (10 mL*2), dried with anhydrous Na₂SO₄, filtered and concentrated in vacuum. The product was concentrated directly for the next reaction. tert-Butyl methyl(3-oxopropyl)carbamate (200 mg, crude) was obtained as yellow oil.

Step 2: To a mixture of tert-butyl methyl(3-oxopropyl)carbamate (200 mg, 1.07 mmol, 1 eq) and morpholine (93.06 mg, 1.07 mmol, 94.00 μL, 1 eq.) in DCM (10 mL) was added NaBH(OAc)₃ (679.17 mg, 3.20 mmol, 3 eq). The reaction mixture was stirred at 25° C. for 10 hr. The mixture was extracted with DCM (10 mL*3). The combined organic phase was washed with brine (10 mL*2), dried with anhydrous Na₂SO₄, filtered and concentrated in vacuum. The product was concentrated directly for the next reaction. tert-Butyl methyl(3-morpholinopropyl)carbamate (200 mg, crude) was obtained as yellow oil.

Step 3: A mixture of tert-butyl methyl(3-morpholinopropyl)carbamate (200 mg, 774.13 μmol, 1 eq.) in HCl/dioxane (5 mL) under N₂. The reaction mixture was stirred at 25° C. for 2 hr. The reaction mixture were concentrated under reduced pressure to give a residue. The product was concentrated directly for the next reaction. N-Methyl-3-morpholinopropan-1-amine (200 mg, crude, HCl) was obtained as a white solid.

Step 4: To a mixture of 1-(3-(5-amino-2-chloro-4-fluoro-3-methylbenzamido)-4-(4-methylpiperazin-1-yl)phenyl)-1H-1,2,3-triazole-4-carboxylic acid (250 mg, 512.39 μmol, 1 eq.) and N-methyl-3-morpholinopropan-1-amine (199.52 mg, 1.02 mmol, 2 eq, HCl) in DMF (2 mL) was added HATU (389.65 mg, 1.02 mmol, 2 eq.) and DIEA (198.66 mg, 1.54 mmol, 267.74 μL, 3 eq.). The reaction mixture was stirred at 25° C. for 10 hr. The mixture was extracted with DCM (10 mL*3). The combined organic phase was washed with brine (10 mL*2), dried with anhydrous Na₂SO₄, filtered and concentrated in vacuum. The reaction mixture was concentrated to dryness. The crude product was purified by reversed-phase HPLC (Column: Phenomenex Gemini-NX C18 75*30 mm*3 μm; Condition: [water (0.04% NH₃H2O+10 mM NH₄HCO₃)-ACN]; B: 19%-49%, 11 min). Example 8 (22 mg, 34.89 μmol, 6.81% yield, 99.63% purity) was obtained as a white solid.

¹H NMR: (DMSO-d₆, 400 MHz) δ_{H}=9.47 (s, 1H), 9.14 (d, J=5.3 Hz, 1H), 8.65 (s, 1H), 7.68 (dd, J=2.4, 8.8 Hz, 1H), 7.42 (d, J=8.8 Hz, 1H), 6.89 (d, J=9.2 Hz, 1H), 5.53 (s, 2H), 3.81 (t, J=7.2 Hz, 1H), 3.57 (s, 2H), 3.53-3.43 (m, 5H), 3.53-3.43 (m, 1H), 3.36 (s, 2H), 3.01 (s, 2H), 2.92 (s, 4H), 2.48-2.43 (m, 3H), 2.33 (dd, J=8.0, 15.2 Hz, 3H), 2.29-2.19 (m, 6H), 1.85-1.75 (m, 2H).

HPLC: Rₜ=2.33 min in 8 min chromatography, XBridge Shield RP18, 5 μm, 2.1*50 mm, purity 99.62%.

LCMS: Rₜ=1.877 min in 4 min chromatography, XBridge Shield RP18, 5 m, 2.1*50 mm, purity 99.52%, MS ESI calcd. For 627.28 [M+H]⁺ 628.28, found 628.7.

### Example 9. Synthesis of 1-(3-(5-amino-2-chloro-4-fluoro-3-methylbenzamido)-4-(4-methylpiperazin-1-yl)phenyl)-N-(3-hydroxypropyl)-N-methyl-1H-1,2,3-triazole-4-carboxamide (Compound 9, Table 1)

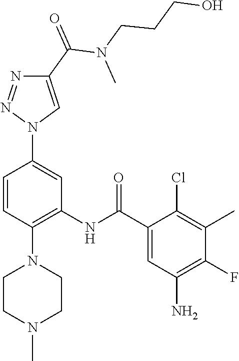 [SMILES: Cc1c(F)c(N)cc(C(=O)Nc2cc(-n3cc(C(=O)N(C)CCCO)nn3)ccc2N2CCN(C)CC2)c1Cl]

The title compound was synthesized according to Schemes 1-3 (Example 2). To a mixture of 1-(3-(5-amino-2-chloro-4-fluoro-3-methylbenzamido)-4-(4-methylpiperazin-1-yl)phenyl)-1H-1,2,3-triazole-4-carboxylic acid (100 mg, 204.95 μmol, 1 eq.) and 3-(methylamino)propan-1-ol (80 mg, 409.91 mol, 2 eq., HCl.) in DMF (1 mL) was added HATU (156 mg, 409.91 μmol, 2 eq.) and DIEA (79 mg, 614.86 μmol, 107.10 μL, 3 eq.). The reaction mixture was stirred at 25° C. for 10 hr. The mixture was extracted with DCM (10 mL*3). The combined organic phase was washed with brine (10 mL*2), dried with anhydrous Na₂SO₄, filtered and concentrated in vacuum. The reaction mixture was concentrated to dryness. The crude product was purified by prep-HPLC (Column: Phenomenex luna C18 80*40 mm*3 μm; Condition: [water(0.04% NH₃H₂O+10 mM NH₄HCO₃)-ACN]; B: 10%-50%, 12 min). Example 9 (15.8 mg, 36.77 μmol, 17.94% yield, 97.43% purity) was obtained as a white solid.

¹H NMR: (DMSO-d₆, 400 MHz) δ_{H}=9.48 (s, 1H), 9.16 (d, J=8.4 Hz, 1H), 8.66 (s, 1H), 7.70 (dd, J=2.8, 8.8 Hz, 1H), 7.43 (d, J=8.8 Hz, 1H), 6.90 (d, J=9.2 Hz, 1H), 5.54 (s, 2H), 4.53 (q, J=4.8 Hz, 1H), 3.83 (t, J=7.2 Hz, 1H), 3.58-3.40 (m, 3H), 3.36 (s, 4H), 3.03 (s, 1H), 2.93 (t, J=4.4 Hz, 4H), 2.49-2.46 (m, 2H), 2.31-2.18 (m, 6H), 1.87-1.70 (m, 2H).

HPLC: Rₜ=3.041 min in 8 min chromatography, XBridge Shield RP18, 5 m, 2.1*50 mm, purity 97.43%.

LCMS: Rₜ=2.353 min in 4 min chromatography, XBridge Shield RP18, 5 m, 2.1*50 mm, purity 97.73%, MS ESI calcd. For 558.23 [M+H]⁺ 559.23, found 559.4.

### Example 10. Synthesis of (S)-1-(3-(5-amino-2-chloro-4-fluoro-3-methylbenzamido)-4-(3,4-dimethylpiperazin-1-yl)phenyl)-N-(3-morpholinopropyl)-1H-1,2,3-triazole-4-carboxamide (Compound 10, Table 1)

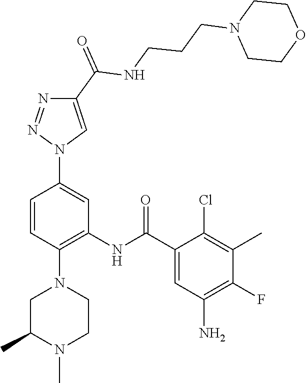 [SMILES: Cc1c(F)c(N)cc(C(=O)Nc2cc(-n3cc(C(=O)NCCCN4CCOCC4)nn3)ccc2N2CCN(C)[C@@H](C)C2)c1Cl]

The title compound was synthesized in a manner analogous to Schemes 1-3.

Step 1: To a solution of 4-fluoro-3-nitroaniline (5 g, 32.03 mmol, 1 eq.) in CH₃CN (50 mL) was added (2R)-1,2-dimethylpiperazine (3.66 g, 32.03 mmol, 1 eq.) and DIEA (8.28 g, 64.06 mmol, 11.16 mL, 2 eq.). The mixture was stirred at 80° C. for 2 hrs. The reaction mixture was partitioned between EtOAc (30 mL) and H₂O (30 mL*2). The organic phase was separated, washed with brine (20 mL*3), dried over Na₂SO₄, filtered and concentrated under reduced pressure to give a residue. The residue was purified by flash silica gel chromatography (ISCO®; 20 g SepaFlash® Silica Flash Column, Eluent of 0-8% Dichloromethane/Methanol gradient at 58 mL/min). (S)-4-(3,4-Dimethylpiperazin-1-yl)-3-nitroaniline (4.03 g, 16.10 mmol, 50.27% yield) was obtained as a red brown oil.

¹H NMR: (DMSO-d₆, 400 MHz) δ_{H}=7.14 (d, J=8.8 Hz, 1H), 6.85 (d, J=2.8 Hz, 1H), 6.80-6.75 (m, 1H), 5.43 (s, 2H), 2.80 (dd, J=2.8, 7.6 Hz, 4H), 2.46 (d, J=9.6 Hz, 1H), 2.22-2.13 (m, 4H), 2.11-2.03 (m, 1H), 0.95 (d, J=6.4 Hz, 3H).

Step 2: To a mixture of (S)-4-(3,4-dimethylpiperazin-1-yl)-3-nitroaniline (5.3 g, 21.17 mmol, 1 eq.) in HCl (2 M, 125 mL, 11.81 eq.) was added a solution of NaNO₂ (2.19 g, 31.76 mmol, 1.5 eq.) in H₂O (15 mL) dropwise at 0° C. After 0.5 hr, a solution of NaN₃ (2.75 g, 42.35 mmol, 2 eq.) in H₂O (15 mL) was added into the mixture. After 0.5 hr, the mixture was allowed to warm up to 25° C. and stirred for 2 hrs. The pH of the mixture was adjusted to around 9 with 2N NaOH. The mixture was extracted with DCM (200 mL*3). The combined organic layer was dried over Na₂SO₄, and concentrated to give crude product. (S)-4-(4-azido-2-nitrophenyl)-1,2-dimethylpiperazine (5.5 g, 19.45 mmol, 91.88% yield) was obtained as a red oil.

Step 3: To a mixture of (S)-4-(4-azido-2-nitrophenyl)-1,2-dimethylpiperazine (4.5 g, 16.29 mmol, 1 eq.) in MeOH (140 mL) was added CuI (3.10 g, 16.29 mmol, 1 eq.), DIEA (1.05 g, 8.14 mmol, 1.42 mL, 0.5 eq.) and methyl prop-2-ynoate (8.22 g, 97.72 mmol, 8.13 mL, 6 eq.). The mixture was stirred at 65° C. for 2 hrs. The mixture was diluted with DCM (50 mL), washed with 2N NH₃·H₂O (20 mL) and brine (20 mL*2). The combined organic layer was dried over Na₂SO₄, and concentrated to give crude product. The product was purified by column chromatography on silica gel (PE: EtOAc=5:1). Methyl (S)-1-(4-(3,4-dimethylpiperazin-1-yl)-3-nitrophenyl)-1H-1,2,3-triazole-4-carboxylate (4.5 g, 11.05 mmol, 67.86% yield) was obtained as a yellow solid.

¹H NMR: (DMSO-d₆, 400 MHz) δ_{H}=7.58 (d, J=2.0 Hz, 1H), 7.41-7.32 (m, 2H), 3.03-2.89 (m, 3H), 2.79-2.71 (m, 1H), 2.59-2.51 (m, 1H), 2.25-2.09 (m, 5H), 0.98 (d, J=2.4 Hz, 3H).

Step 4: To a mixture of methyl (S)-1-(4-(3,4-dimethylpiperazin-1-yl)-3-nitrophenyl)-1H-1,2,3-triazole-4-carboxylate (1 g, 2.77 mmol, 1 eq.) in MeOH (10 mL) and H₂O (2 mL) was added SnCl₂·2H₂O (1.57 g, 6.94 mmol, 2.5 eq.). The reaction mixture was stirred at 80° C. for 2 hr. The reaction mixture was quenched by addition NaHCO₃ (100 mL), and then extracted with DCM (100 mL*3). The combined organic layers were concentrated under reduced pressure to give a residue. The crude product was purified by silica gel chromatography (ISCO®; 40 g SepaFlash® Silica Flash Column, Eluent of 0-15% MeOH/DCM ether gradient at 60 mL/min). Methyl (S)-1-(3-amino-4-(3,4-dimethylpiperazin-1-yl)phenyl)-1H-1,2,3-triazole-4-carboxylate (650 mg, 1.81 mmol, 65.23% yield) was obtained as a yellow solid.

¹H NMR: (DMSO-d₆, 400 MHz) δ_{H}=9.28 (s, 1H), 7.25 (d, J=2.4 Hz, 1H), 7.07-6.99 (m, 2H), 5.14 (s, 2H), 3.87 (s, 3H), 3.01-2.91 (m, 2H), 2.82-2.68 (m, 2H), 2.42-2.27 (m, 3H), 2.23 (s, 3H), 1.01 (d, J=6.0 Hz, 3H).

Step 5: To a solution of methyl (S)-1-(3-amino-4-(3,4-dimethylpiperazin-1-yl)phenyl)-1H-1,2,3-triazole-4-carboxylate, intermediate 8 (200 mg, 605.36 μmol, 1 eq.) and 2-chloro-4-fluoro-3-methyl-5-nitrobenzoyl chloride (168 mg, 665.89 μmol, 1.1 eq.) in DCM (15 mL) was added Et₃N (306 mg, 3.03 mmol, 421.29 μL, 5 eq.) at −20° C. The reaction mixture was allowed warmed to 15° C. and stirred for 12 hr to give a brown mixture. The reaction mixture was concentrated. The residue was purified by flash silica gel chromatography (ISCO®; 12 g SepaFlash® Silica Flash Column, Eluent of 0-20% MeOH/DCM ether gradient at 20 mL/min). The product methyl (S)-1-(3-(2-chloro-4-fluoro-3-methyl-5-nitrobenzamido)-4-(3,4-dimethylpiperazin-1-yl)phenyl)-1H-1,2,3-triazole-4-carboxylate (190 mg, 271.11 μmol, 44.78% yield) was obtained as a yellow solid.

¹H NMR: (DMSO-d₆, 400 MHz) δ_{H}=9.96 (s, 1H), 9.47 (s, 1H), 8.56 (s, 1H), 8.35 (d, J=7.6 Hz, 1H), 7.77 (dd, J=2.6, 8.6 Hz, 1H), 7.37 (d, J=8.8 Hz, 1H), 3.90 (s, 3H), 3.13-3.02 (m, 2H), 2.94-2.74 (m, 2H), 2.48-2.34 (m, 5H), 2.21 (br s, 4H), 1.01 (d, J=5.9 Hz, 3H).

Step 6: To a mixture of methyl (S)-1-(3-(2-chloro-4-fluoro-3-methyl-5-nitrobenzamido)-4-(3,4-dimethylpiperazin-1-yl)phenyl)-1H-1,2,3-triazole-4-carboxylate (490 mg, 897.52 μmol, 1 eq.) in MeOH (10 mL) and H₂O (3 mL) was added SnCl₂.2H₂O (607 mg, 2.69 mmol, 3 eq.). The reaction mixture was stirred at 80° C. for 2 hr to give a brown mixture. The reaction mixture was then adjusted to pH-8 by aq. NaHCO₃. The resulting mixture was extracted with DCM (60 mL*3). The combined organic phase was washed with brine (50 mL), dried over anhydrous Na₂SO₄, filtered and concentrated. The residue was purified by flash silica gel chromatography (ISCO®; 12 g SepaFlash® Silica Flash Column, Eluent of 0-20% DCM/MeOH ether gradient at 20 mL/min). The product methyl (S)-1-(3-(5-amino-2-chloro-4-fluoro-3-methylbenzamido)-4-(3,4-dimethylpiperazin-1-yl)phenyl)-1H-1,2,3-triazole-4-carboxylate (300 mg, 473.87 μmol, 52.80% yield) was obtained as a yellow solid.

¹H NMR: (DMSO-d₆, 400 MHz) δ_{H}=9.48 (s, 2H), 8.65 (s, 1H), 7.71 (dd, J=2.8, 8.8 Hz, 1H), 7.42 (d, J=8.8 Hz, 1H), 6.88 (d, J=9.6 Hz, 1H), 5.52 (s, 2H), 3.89 (s, 3H), 3.17 (d, J=5.6 Hz, 3H), 3.05-2.75 (m, 4H), 2.26 (d, J=2.4 Hz, 3H), 2.21 (s, 3H), 0.99 (d, J=6.4 Hz, 3H).

Step 7: To a solution of methyl (S)-1-(3-(5-amino-2-chloro-4-fluoro-3-methylbenzamido)-4-(3,4-dimethylpiperazin-1-yl)phenyl)-1H-1,2,3-triazole-4-carboxylate (300 mg, 581.43 μmol, 1 eq.) in THF (3 mL) and H₂O (1 mL) was added LiOH·H₂O (48.79 mg, 1.16 mmol, 2 eq.). The mixture was stirred at 25° C. for 2 hr. The reaction mixture was adjusted to pH=5 by 1N aq. HCl, concentrated under reduced pressure to give a residue. The product was used directly to the next step without further purification. Advanced intermediate 9 (S)-1-(3-(5-amino-2-chloro-4-fluoro-3-methylbenzamido)-4-(3,4-dimethylpiperazin-1-yl)phenyl)-1H-1,2,3-triazole-4-carboxylic acid (291 mg, crude) was obtained as a brown solid.

¹H NMR: (DMSO-d₆, 400 MHz) δ_{H}=9.57 (s, 1H), 9.19 (s, 1H), 8.66-8.56 (m, 1H), 7.71 (dd, J=2.4, 8.8 Hz, 1H), 7.40 (d, J=8.8 Hz, 1H), 6.90 (d, J=9.2 Hz, 1H), 5.54 (s, 2H), 3.12 (s, 5H), 2.95 (s, 1H), 2.91-2.82 (m, 2H), 2.53 (s, 3H), 2.24 (d, J=2.0 Hz, 3H), 1.19 (d, J=6.4 Hz, 3H).

Step 8: To a solution of advanced intermediate 9 (140 mg, 278.92 μmol, 1 eq) and 3-morpholinopropan-1-amine (60.34 mg, 418.38 μmol, 61.13 μL, 1.5 eq.) in DMF (3 mL) was added HATU (212.11 mg, 557.84 μmol, 2 eq) and DIEA (108.14 mg, 836.75 μmol, 145.74 μL, 3 eq). The mixture was stirred at 25° C. for 3 hr. The mixture was concentrated under reduced pressure to give a residue. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 15-55% B in 11 min.). Example 10 (98 mg, 154.44 μmol, 55.37% yield, 98.99% purity) was obtained as a white solid.

¹H NMR: (DMSO-d₆, 400 MHz) δ_{H}=9.49-9.40 (m, 1H), 9.20 (s, 1H), 8.88-8.79 (m, 1H), 8.71-8.62 (m, 1H), 7.70 (dd, J=2.4, 8.4 Hz, 1H), 7.40 (d, J=8.8 Hz, 1H), 6.90 (d, J=9.2 Hz, 1H), 5.52 (s, 2H), 3.61 (s, 4H), 3.39-3.35 (m, 2H), 3.01-2.73 (m, 4H), 2.55-2.50 (m, 2H), 2.41-2.32 (m, 6H), 2.32-2.28 (m, 1H), 2.26 (d, J=1.6 Hz, 3H), 2.20 (s, 3H), 1.71 (d, J=6.8 Hz, 2H), 0.98 (d, J=6.0 Hz, 3H).

HPLC: Rₜ=2.460 min in 8 min chromatography, Ultimate XB-C18 (3×50 mm, 3 μm), purity 98.99%.

LCMS: Rₜ=1.999 min in 4 min chromatography, XBridge Shield RP18, 5 μm, 2.1*50 mm, purity 99.06%, MS ESI calcd. for 627.28 [M+H]⁺ 628.28, found 628.5.

### Example 11. Synthesis of 1-(3-(5-amino-2-chloro-4-fluoro-3-methylbenzamido)-4-((3S,5R)-3,4,5-trimethylpiperazin-1-yl)phenyl)-N-(3-morpholinopropyl)-1H-1,2,3-triazole-4-carboxamide (Compound 11, Table 1)

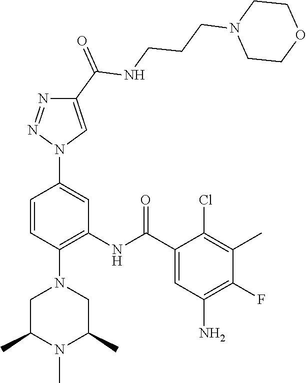 [SMILES: Cc1c(F)c(N)cc(C(=O)Nc2cc(-n3cc(C(=O)NCCCN4CCOCC4)nn3)ccc2N2C[C@@H](C)N(C)[C@@H](C)C2)c1Cl]

The title compound was synthesized in a manner analogous to Schemes 1-3.

Step 1: To a mixture of 4-fluoro-3-nitroaniline (5 g, 32.03 mmol, 1 eq) and (2S,6R)-1,2,6-trimethylpiperazine (4.93 g, 38.43 mmol, 1.2 eq) in CH₃CN (10 mL) was added DIEA (4.2 g, 32.03 mmol, 5.58 mL, 1 eq) in one portion at 25° C. under N₂. The mixture was heated to 80° C. and stirred for 10 hours. The residue was poured into water (40 mL). The aqueous phase was extracted with ethyl acetate (20 mL*3). The combined organic phase was washed with brine (20 mL*2), dried with anhydrous Na₂SO₄, filtered and concentrated in vacuum. The residue was purified by silica gel chromatography (column weight: 20 g, diameter: 100 mm, 100-200 mesh silica gel, Dichloromethane: Methanol=7:3). 3-Nitro-4-((3S,5R)-3,4,5-trimethylpiperazin-1-yl)aniline (6.8 g, 25.62 mmol, 79.98% yield, 99.57% purity) was obtained as a yellow oil.

¹H NMR: (CDCl₃-d₆, 400 MHz) δ_{H}=7.07-7.01 (m, 2H), 6.81 (dd, J=2.8, 8.6 Hz, 1H), 3.73 (br s, 2H), 2.99-2.92 (m, 2H), 2.62 (t, J=10.8 Hz, 2H), 2.47-2.36 (m, 2H), 2.31 (s, 3H), 1.10 (d, J=6.4 Hz, 6H).

Step 2: To a mixture of 3-nitro-4-((3S,5R)-3,4,5-trimethylpiperazin-1-yl)aniline (6.8 g, 25.73 mmol, 1 eq) in HCl (2 M, 155.43 mL, 12.08 eq) was added a solution of NaNO₂ (2.66 g, 38.59 mmol, 1.5 eq) in H₂O (21 mL) dropwise at 0° C. After 0.5 hr, a solution of NaN3 (3.34 g, 51.45 mmol, 2 eq) in H₂O (21 mL) was added into the mixture. After 0.5 hr, the mixture was allowed to warm up to 25° C. and stirred for 2 hrs. The pH of the mixture was adjusted to around 9 with 2N NaOH. The mixture was extracted with DCM (200 mL*3). The combined organic layer was dried over Na2SO4, and concentrated to give crude product. The crude product was used directly to the next step without further purification. (2S,6R)-4-(4-Azido-2-nitrophenyl)-1,2,6-trimethylpiperazine (7.3 g, 24.53 mmol, 95.34% yield, 97.54% purity) was obtained as a red solid.

LCMS: Rₜ=0.640 min in 2 min chromatography, Ultimate XB-C18, 3 μm, 3.0*50 mm, purity 97.54%, MS ESI calcd. for 290.15 [M+H]⁺ 291.15, found 290.9.

Step 3: To a mixture of (2S,6R)-4-(4-azido-2-nitrophenyl)-1,2,6-trimethylpiperazine (6 g, 20.67 mmol, 1 eq.) and methyl propiolate (2.09 g, 24.80 mmol, 2.06 mL, 1.2 eq.) in THF (70 mL) was added CuI (1.18 g, 6.20 mmol, 0.3 eq.) and DIEA (8.01 g, 62.00 mmol, 10.80 mL, 3 eq.) under N₂. The reaction mixture was stirred at 25° C. for 10 hr. The reaction mixture was filtered. The filter cake was washed with CH₃CN (200 mL*3). The filtrate was concentrated. The product was concentrated directly for the next reaction. Methyl 1-(3-nitro-4-((3S,5R)-3,4,5-trimethylpiperazin-1-yl)phenyl)-1H-1,2,3-triazole-4-carboxylate (9 g, 19.23 mmol, 93.05% yield) was obtained as a yellow solid.

¹H NMR: (DMSO-d₆, 400 MHz) δ_{H}=9.54 (s, 1H), 8.44 (d, J=2.4 Hz, 1H), 8.13 (dd, J=2.8, 8.8 Hz, 1H), 7.49 (d, J=9.2 Hz, 1H), 3.89 (s, 3H), 3.11 (d, J=12.0 Hz, 2H), 2.72 (t, J=11.2 Hz, 2H), 2.30-2.23 (m, 2H), 2.19 (s, 3H), 1.02 (d, J=6.4 Hz, 6H).

Step 4: To a mixture of methyl 1-(3-nitro-4-((3S,5R)-3,4,5-trimethylpiperazin-1-yl)phenyl)-1H-1,2,3-triazole-4-carboxylate (6 g, 20.67 mmol, 1 eq.) in MeOH (100 mL) and H₂O (20 mL) was added SnCl₂.2H₂O (16.27 g, 72.12 mmol, 3 eq). The mixture was stirred at 80° C. for 10 hours. The reaction mixture was quenched by addition NaHCO₃ (200 mL), and then extracted with DCM (100 mL*3). The combined organic layers were concentrated under reduced pressure to give a residue. The crude product was purified by silica gel chromatography (ISCO®; 80 g SepaFlash® Silica Flash Column, Eluent of 0-15% MeOH/DCM ether gradient at 100 mL/min). Methyl 1-(3-amino-4-((3S,5R)-3,4,5-trimethylpiperazin-1-yl)phenyl)-1H-1,2,3-triazole-4-carboxylate, intermediate 11 (4.4 g, 12.02 mmol, 50.02% yield) was obtained as a yellow solid.

¹H NMR: (DMSO-d₆, 400 MHz) δ_{H}=9.28 (s, 1H), 7.26 (d, J=2.4 Hz, 1H), 7.08-7.03 (m, 1H), 7.01-6.96 (m, 1H), 5.15 (s, 2H), 3.87 (s, 3H), 2.96 (d, J=7.6 Hz, 2H), 2.37 (d, J=5.6 Hz, 4H), 2.22 (s, 3H), 1.03 (d, J=5.2 Hz, 6H).

Step 5: To a solution of methyl 1-(3-amino-4-((3S,5R)-3,4,5-trimethylpiperazin-1-yl)phenyl)-1H-1,2,3-triazole-4-carboxylate (700 mg, 2.03 mmol, 1 eq) and TEA (1.03 g, 10.16 mmol, 1.41 mL, 5 eq) in DCM (20 mL) was added a solution of 2-chloro-4-fluoro-3-methyl-5-nitro-benzoyl chloride (461 mg, 1.83 mmol, 0.9 eq) in DCM (5 mL) drop-wise at −20° C. The reaction mixture was allowed to warm to 20° C. and stirred for 3 hrs to give a brown mixture. Water (20 mL) was added to the reaction mixture. The resulting mixture was extracted with DCM (20 mL*3). The combined organic phase was washed with brine (20 mL), dried over anhydrous Na2SO4, filtered and concentrated. The residue was purified by flash silica gel chromatography (ISCO®; 20 g SepaFlash® Silica Flash Column, Eluent of 0-15% MeOH/DCM ether gradient at 30 mL/min). The product methyl 1-(3-(2-chloro-4-fluoro-3-methyl-5-nitrobenzamido)-4-((3S,5R)-3,4,5-trimethylpiperazin-1-yl)phenyl)-1H-1,2,3-triazole-4-carboxylate (900 mg, 663.78 μmol, 32.66% yield, 41.3% purity) was obtained as a brown solid.

LCMS: Rₜ=0.796 min in 1.5 min chromatography, Chromolith Flash RP-18, 5 μm, 3.0*25 mm, purity 41.3%, MS ESI calcd. for 559.17 [M+H]⁺ 560.17, found 560.1.

Step 6: To a solution of methyl 1-(3-(2-chloro-4-fluoro-3-methyl-5-nitrobenzamido)-4-((3S,5R)-3,4,5-trimethylpiperazin-1-yl)phenyl)-1H-1,2,3-triazole-4-carboxylate (900 mg, 1.61 mmol, 1 eq.) in MeOH (20 mL) and H₂O (6 mL) was added SnCl₂·2H₂O (1.09 g, 4.82 mmol, 3 eq.). The mixture was stirred at 80° C. for 2 hr. The mixture was adjusted to pH 9 with NaHCO₃, filtered to remove the insoluble. The filter liquor was concentrated in vacuo. The mixture was diluted with DCM (50 mL*3), washed with brine (20 mL). The organic layer was dried over Na₂SO₄, filtered and concentrated under reduced pressure to give a residue. The residue was purified by flash silica gel chromatography (ISCO®; 20 g SepaFlash® Silica Flash Column, Eluent of 0-13% MeOH/DCM at 35 mL/min). 1-(3-(5-Amino-2-chloro-4-fluoro-3-methylbenzamido)-4-((3S,5R)-3,4,5-trimethylpiperazin-1-yl)phenyl)-1H-1,2,3-triazole-4-carboxylate (640 mg, 941.90 μmol, 58.60% yield) was obtained as a yellow oil.

¹H NMR: (DMSO-d₆, 400 MHz) δ_{H}=9.49-9.47 (m, 1H), 8.69-8.61 (m, 1H), 7.75-7.68 (m, 1H), 7.43-7.37 (m, 1H), 6.93-6.85 (m, 1H), 5.51 (s, 2H), 3.90 (s, 3H), 3.00-2.93 (m, 2H), 2.89 (s, 1H), 2.77-2.75 (m, 1H), 2.77-2.72 (m, 1H), 2.36-2.29 (m, 2H), 2.26 (d, J=2.4 Hz, 3H), 2.20 (s, 3H), 1.03 (d, J=6.4 Hz, 6H).

Step 7: To a solution of methyl 1-(3-(5-amino-2-chloro-4-fluoro-3-methylbenzamido)-4-((3S,5R)-3,4,5-trimethylpiperazin-1-yl)phenyl)-1H-1,2,3-triazole-4-carboxylate (300 mg, 566.05 μmol, 1 eq.) in THF (4 mL) and H₂O (2 mL) was added LiOH·H₂O (48 mg, 1.13 mmol, 2 eq.). The mixture was stirred at 25° C. for 2 hr. The reaction mixture was adjusted to pH=5 by 1N aq. HCl, concentrated under reduced pressure to give a residue. The product was used directly to the next step without further purification. 1-(3-(5-Amino-2-chloro-4-fluoro-3-methylbenzamido)-4-((3S,5R)-3,4,5-trimethylpiperazin-1-yl)phenyl)-1H-1,2,3-triazole-4-carboxylic acid advanced intermediate 12 (300 mg, crude) was obtained as a brown solid.

¹H NMR: (DMSO-d₆, 400 MHz) δ_{H}=11.45-11.29 (m, 1H), 9.78 (s, 1H), 9.39 (s, 1H), 8.64-8.57 (m, 1H), 7.82-7.74 (m, 1H), 7.43-7.37 (m, 1H), 7.12-7.04 (m, 1H), 3.57-3.43 (m, 2H), 3.31-3.16 (m, 4H), 2.96 (s, 1H), 2.79 (d, J=4.8 Hz, 3H), 2.65-2.59 (m, 1H), 2.28 (d, J=2.0 Hz, 3H), 1.40 (d, J=6.4 Hz, 6H).

Step 8: To a solution of advanced intermediate 8 (300 mg, 581.43 μmol, 1 eq.) and 3-morpholinopropan-1-amine (126 mg, 872.15 μmol, 127.43 μL, 1.5 eq.) in DMF (4 mL) was added HATU (442 mg, 1.16 mmol, 2 eq.) and DIEA (225.44 mg, 1.74 mmol, 303.83 μL, 3 eq.). The mixture was stirred at 25° C. for 12 hr. The reaction mixture was concentrated directly. The residue was purified by prep-HPLC (Column: Phenomenex Gemini-NX C18 75*30 mm*5 μm; Mobile Phase A: purified water (0.04% NH₃H2O+10 mM NH₄HCO₃); Mobile Phase B: acetonitrile; Gradient: 0-70% B in 12 min). Example 11 (33.6 mg, 51.91 μmol, 41.67% yield, 99.21% purity) was obtained as a white solid.

¹H NMR: (DMSO-d₆, 400 MHz) δ_{H}=9.47 (s, 1H), 9.20 (s, 1H), 8.82 (t, J=5.6 Hz, 1H), 8.65 (s, 1H), 7.69 (dd, J=2.8, 8.8 Hz, 1H), 7.38 (d, J=8.8 Hz, 1H), 6.89 (d, J=9.2 Hz, 1H), 5.50 (s, 2H), 3.61 (t, J=4.8 Hz, 4H), 3.36 (s, 2H), 2.94 (d, J=10.8 Hz, 2H), 2.58-2.52 (m, 2H), 2.41-2.27 (m, 8H), 2.25 (d, J=2.6 Hz, 3H), 2.18 (s, 3H), 1.70 (s, 2H), 1.02 (d, J=6.2 Hz, 6H).

HPLC: Rₜ=3.540 min in 8 min chromatography, XBridge Shield RP18, 5 μm, 2.1*50 mm, purity 99.21%.

LCMS: Rₜ=2.712 min in 4 min chromatography, XBridge Shield RP18, 5 μm, 2.1*50 mm, purity 99.72%, MS ESI calcd. for 641.30 [M+H]⁺ 642.30, found 642.5.

### Example 12. Synthesis of 1-(3-(5-amino-2-chloro-4-fluoro-3-methylbenzamido)-4-(4-methyl-1,4-diazepan-1-yl)phenyl)-N-(3-morpholinopropyl)-1H-1,2,3-triazole-4-carboxamide (Compound 12, Table 1)

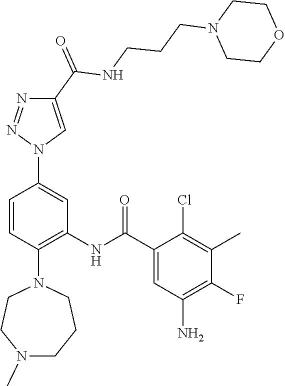 [SMILES: Cc1c(F)c(N)cc(C(=O)Nc2cc(-n3cc(C(=O)NCCCN4CCOCC4)nn3)ccc2N2CCCN(C)CC2)c1Cl]

The title compound was synthesized in a manner similar to Schemes 1-3.

Step 1: To a solution of 4-fluoro-3-nitro-aniline (10 g, 64.06 mmol, 1 eq.) in CH₃CN (100 mL) was added 1-methyl-1,4-diazepane (7.31 g, 64.06 mmol, 7.97 mL, 1 eq.) and DIEA (16.56 g, 128.11 mmol, 22.31 mL, 2 eq.). The mixture was stirred at 80° C. for 12 hrs. The mixture was diluted with H₂O (200 mL), extracted with EtOAc (500 mL), and washed with brine (300 mL). The organic layer was dried over Na₂SO₄, filtered and concentrated under reduced pressure to give a residue. The residue was purified by flash silica gel chromatography (ISCO®; 40 g SepaFlash® Silica Flash Column, Eluent of 0-14% MeOH/DCM at 80 mL/min). 4-(4-Methyl-1,4-diazepan-1-yl)-3-nitroaniline (12.6 g, 49.33 mmol, 77.02% yield) was obtained as a red brown oil.

¹H NMR: (DMSO-d₆, 400 MHz) δ_{H}=7.08 (d, J=8.8 Hz, 1H), 6.84 (d, J=2.8 Hz, 1H), 6.78 (dd, J=2.8, 8.8 Hz, 1H), 5.24 (s, 2H), 3.12-3.04 (m, 4H), 2.57-2.52 (m, 4H), 2.26 (s, 3H), 1.77-1.67 (m, 2H).

Step 2: To a mixture of 4-(4-methyl-1,4-diazepan-1-yl)-3-nitroaniline (13 g, 51.94 mmol, 1 eq.) in HCl (2 M, 259.69 mL, 10 eq.) was added a solution of NaNO₂ (5.38 g, 77.91 mmol, 1.5 eq.) in H₂O (40 mL) drop-wise at 0° C. After 0.5 hr, a solution of NaN₃ (6.75 g, 103.88 mmol, 2 eq.) in H₂O (40 mL) was added into the mixture. The mixture was allowed to warm up to 20° C. and stirred for 1 hr to give a brown mixture. The pH of the mixture was adjusted to around 9 with 2N NaOH. The resulting mixture was extracted with DCM (200 mL*3). The combined organic phase was washed with brine (200 mL), dried over anhydrous Na₂SO₄, filtered and concentrated. The product 1-(4-azido-2-nitrophenyl)-4-methyl-1,4-diazepane (12.3 g, 42.34 mmol, 81.51% yield) was obtained as brown oil and used into the next step without further purification.

¹H NMR:(CDCl₃, 400 MHz) δ_{H}=7.42 (s, 1H), 7.06 (d, J=1.6 Hz, 2H), 3.47-3.39 (m, 2H), 3.33-3.22 (m, 2H), 2.75-2.67 (m, 2H), 2.65-2.56 (m, 2H), 2.38 (s, 3H), 1.95 (td, J=5.6, 10.8 Hz, 2H).

Step 3: To a mixture of 1-(4-azido-2-nitrophenyl)-4-methyl-1,4-diazepane (9 g, 32.57 mmol, 1 eq.) and methyl prop-2-ynoate (8.22 g, 97.72 mmol, 8.13 mL, 3 eq.) in MeOH (450 mL) was added CuI (1.86 g, 9.77 mmol, 0.3 eq.) and DIEA (842 mg, 6.51 mmol, 1.13 mL, 0.2 eq). The mixture was stirred at 65° C. for 10 hrs. The mixture was diluted with DCM (500 mL), washed with 2N NH₃·H₂O (200 mL) and brine (200 mL*2). The combined organic layer was dried over Na₂SO₄, and concentrated to give crude product. The crude product was purified by column chromatography on silica gel (DCM: MeOH=3:1). Methyl 1-(4-(4-methyl-1,4-diazepan-1-yl)-3-nitrophenyl)-1H-1,2,3-triazole-4-carboxylate (7 g, 16.87 mmol, 51.78% yield) was obtained as a yellow solid.

LCMS: Rₜ=0.995 min in 2 min chromatography, Xtimate C18, 3 μm, 2.1*30 mm, purity 86.55%, MS ESI calcd. for 360.15 [M+H]⁺ 361.15, found 361.0.

Step 4: To a mixture of methyl 1-(4-(4-methyl-1,4-diazepan-1-yl)-3-nitrophenyl)-1H-1,2,3-triazole-4-carboxylate (6 g, 16.65 mmol, 1 eq.) in EtOH (60 mL) and H₂O (20 mL) was added Fe (2.79 g, 49.95 mmol, 3 eq.) and NH₄Cl (4.45 g, 83.25 mmol, 5 eq.). The reaction mixture was stirred at 80° C. for 10 hr to give a brown mixture. The reaction mixture was filtered and washed with DCM (200 mL*3). The combined organic phase was washed with brine (200 mL*2), dried with anhydrous Na₂SO₄, filtered and concentrated in vacuum. The crude product was purified by silica gel chromatography (ISCO®; 80 g SepaFlash® Silica Flash Column, Eluent of 0-20% MeOH/DCM ether gradient at 100 mL/min). The crude product ethyl 1-(3-amino-4-(4-methyl-1,4-diazepan-1-yl)phenyl)-1H-1,2,3-triazole-4-carboxylate (2.1 g, crude) was obtained as a brown solid.

LCMS: Rₜ=1.185 min in 2 min chromatography, Xtimate C18, 3 um, 2.1*30 mm, purity 62.16%, MS ESI calcd. for 344.20 [M+H]⁺ 345.20, found 345.3.

Step 5: To a mixture of ethyl 1-(3-amino-4-(4-methyl-1,4-diazepan-1-yl)phenyl)-1H-1,2,3-triazole-4-carboxylate (1.8 g, 5.23 mmol, 1 eq.) and 2-chloro-4-fluoro-3-methyl-5-nitrobenzoyl chloride (1.45 g, 5.75 mmol, 1.1 eq.) in DCM (100 mL) was added TEA (2.64 g, 26.13 mmol, 3.64 mL, 5 eq.) in one portion at 0° C. The mixture was stirred at 25° C. for 12 hours. The residue was poured into water (100 mL). The aqueous phase was extracted with DCM (70 mL*3). The combined organic phase was washed with brine (100 mL*2), dried with anhydrous Na₂SO₄, filtered and concentrated in vacuum. The residue was purified by silica gel chromatography (column weight: 20 g, diameter: 100 mm, 100-200 mesh silica gel, Dichloromethane: Methanol=5:1). 1-(3-(2-Chloro-4-fluoro-3-methyl-5-nitrobenzamido)-4-(4-methyl-1,4-diazepan-1-yl)phenyl)-1H-1,2,3-triazole-4-carboxylate (1.2 g, 1.29 mmol, 24.60% yield) was obtained as a yellow solid.

LCM:S Rₜ=0.753 min in 2 min chromatography, Xtimate C18, 3 μm, 2.1*30 mm, purity 49.76%, MS ESI calcd. for 559.17 [M+H]⁺ 560.17, found 560.0.

Step 6: To a mixture of 1-(3-(2-chloro-4-fluoro-3-methyl-5-nitrobenzamido)-4-(4-methyl-1,4-diazepan-1-yl)phenyl)-1H-1,2,3-triazole-4-carboxylate (1.2 g, 2.14 mmol, 1 eq.) in EtOH (30 mL) and H₂O (10 mL) was added SnCl₂.2H₂O (1.45 g, 6.43 mmol, 3 eq.). The reaction mixture was stirred at 80° C. for 2 hr to give a brown mixture. After cooled, the reaction mixture was adjust to pH-8 with aq.NaHCO₃. Then DCM was added until solid formed. The mixture was filtered and the cake was washed with EtOH (50 mL×2). The filtrate was concentrated. The residue was purified by flash silica gel chromatography (ISCO®; 20 g SepaFlash® Silica Flash Column, Eluent of 0-20% MeOH/DCM ether gradient at 30 mL/min). The impure product 1-(3-(5-amino-2-chloro-4-fluoro-3-methylbenzamido)-4-(4-methyl-1,4-diazepan-1-yl)phenyl)-1H-1,2,3-triazole-4-carboxylate (670 mg, 816.65 μmol, 38.11% yield) was obtained as a yellow solid.

¹H NMR: (DMSO-d₆, 400 MHz) δ_{H}=9.85 (s, 1H), 9.49-9.35 (m, 1H), 8.55-8.44 (m, 1H), 7.74-7.61 (m, 1H), 7.41-7.33 (m, 1H), 6.86 (d, J=9.2 Hz, 1H), 5.53 (s, 2H), 4.37 (q, J=7.2 Hz, 2H), 4.21-4.02 (m, 2H), 3.31-3.19 (m, 7H), 2.37-2.16 (m, 7H), 1.35 (t, J=7.2 Hz, 3H).

Step 7: To a mixture of 1-(3-(5-amino-2-chloro-4-fluoro-3-methylbenzamido)-4-(4-methyl-1,4-diazepan-1-yl)phenyl)-1H-1,2,3-triazole-4-carboxylate (650 mg, 1.23 mmol, 1 eq.) in THF (15 mL) and H₂O (5 mL) was added LiOH·H₂O (103 mg, 2.45 mmol, 2 eq.). The reaction mixture was stirred at 15° C. for 2 hr to give a brown mixture. The pH of the reaction mixture was adjust to 5 by 1N HCl. The reaction mixture was then concentrated. The crude product 1-(3-(5-amino-2-chloro-4-fluoro-3-methylbenzamido)-4-(4-methyl-1,4-diazepan-1-yl)phenyl)-1H-1,2,3-triazole-4-carboxylic acid (660 mg, crude) was obtained as a brown solid and used into the next step without further purification.

¹H NMR: (DMSO-d₆, 400 MHz) δ_{H}=11.23 (s, 1H), 9.84 (s, 1H), 9.36 (s, 1H), 8.40 (s, 1H), 7.73 (dd, J=2.8, 8.8 Hz, 1H), 7.40 (d, J=8.8 Hz, 1H), 6.88 (d, J=9.2 Hz, 1H), 5.54 (s, 2H), 3.26-3.08 (m, 4H), 2.84-2.71 (m, 6H), 2.34-2.23 (m, 6H).

Step 8: To a solution of 1-(3-(5-amino-2-chloro-4-fluoro-3-methylbenzamido)-4-(4-methyl-1,4-diazepan-1-yl)phenyl)-1H-1,2,3-triazole-4-carboxylic acid (150 mg, 298.84 μmol, 1 eq.) and 3-morpholinopropan-1-amine (65 mg, 448.26 μmol, 65.50 μL, 1.5 eq.) in DMF (1.5 mL) was added HATU (228 mg, 597.68 μmol, 2 eq.) and DIEA (116 mg, 896.52 μmol, 156.16 μL, 3 eq.). The mixture was stirred at 25° C. for 12 hr. The mixture was concentrated under reduced pressure to give a residue. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 14-54% B in 11 min.). Example 12 (16.1 mg, 25.42 μmol, 8.50% yield, 99.16% purity) was obtained as a light yellow solid.

¹H NMR: (DMSO-d₆, 400 MHz) δ_{H}=9.86 (s, 1H), 9.16 (s, 1H), 8.85-8.78 (m, 1H), 8.55-8.48 (m, 1H), 7.70-7.59 (m, 1H), 7.41-7.31 (m, 1H), 6.92-6.82 (m, 1H), 5.51 (s, 2H), 3.64-3.57 (m, 4H), 3.33- 3.32 (m, 1H), 3.27-3.18 (m, 5H), 2.65-2.59 (m, 2H), 2.58-2.53 (m, 2H), 2.40-2.33 (m, 6H), 2.25 (d, J=2.4 Hz, 3H), 2.14 (s, 3H), 1.84-1.75 (m, 2H), 1.75-1.66 (m, 2H).

HPLC: Rₜ=2.934 min in 8 min chromatography, Ultimate XB-C18 (3×50 mm, 3 μm), purity 99.16%.

LCMS: Rₜ=1.636 min in 4 min chromatography, Xtimate C18, 3 μm, 2.1*30 mm, purity 99.65%, MS ESI calcd. for 627.28 [M+H]⁺ 628.28, found 628.4.

### Example 13. Synthesis of 1-(5-(5-amino-2-chloro-4-fluoro-3-methylbenzamido)-2-fluoro-4-(4-methylpiperazin-1-yl)phenyl)-N-(3-morpholinopropyl)-1H-1,2,3-triazole-4-carboxamide (Compound 13, Table 1)

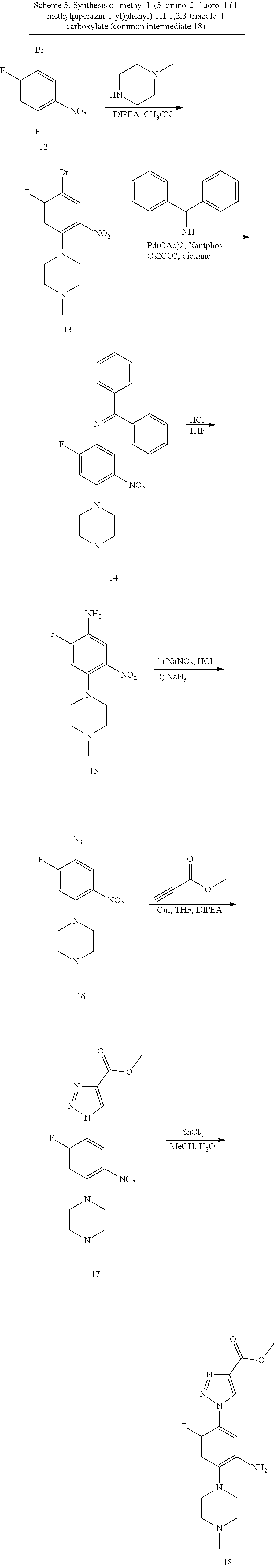

Step 1: To a mixture of 1-bromo-2,4-difluoro-5-nitrobenzene (10 g, 42.02 mmol, 1 eq) and 1-methylpiperazine (4 g, 42.02 mmol, 4.66 mL, 1 eq) in CH₃CN (200 mL) was added DIEA (11 g, 84.04 mmol, 14.64 mL, 2 eq) in one portion at 25° C. The mixture was stirred at 25° C. for 12 hours. The residue was concentrated under reduce pressure. The residue was purified by silica gel chromatography (column weight: 80 g, diameter: 100 mm, 100-200 mesh silica gel, Dichloromethane: Methanol=10:1). 1-(4-Bromo-5-fluoro-2-nitrophenyl)-4-methylpiperazine (13 g, 40.80 mmol, 97.09% yield) was obtained as a yellow oil.

¹H NMR: (DMSO-d₆, 400 MHz) δ_{H}=8.21 (d, J=7.2 Hz, 1H), 7.32 (d, J=11.6 Hz, 1H), 3.07-2.98 (m, 4H), 2.44-2.36 (m, 4H), 2.20 (s, 3H).

Step 2: To a mixture of 1-(4-bromo-5-fluoro-2-nitrophenyl)-4-methylpiperazine (13 g, 40.86 mmol, 1 eq) and diphenylmethanimine (11 g, 61.29 mmol, 10.29 mL, 1.5 eq) in dioxane (200 mL) was added Pd(OAc)₂ (917 mg, 4.09 mmol, 0.1 eq), Xantphos (4 g, 6.13 mmol, 0.15 eq) and Cs₂CO₃ (27 g, 81.72 mmol, 2 eq) in one portion at 25° C. under N₂. The mixture was stirred at 100° C. for 12 hours. The mixture was filtered via a filter paper and kieselguhr. The residue was purified by silica gel chromatography (column weight: 80 g, diameter: 100 mm, 100-200 mesh silica gel, Dichloromethane: Methanol=10:1). N-(diphenylmethylene)-2-fluoro-4-(4-methylpiperazin-1-yl)-5-nitroaniline (18.3 g, 34.96 mmol, 85.56% yield) was obtained as yellow oil.

¹H NMR: (DMSO-d₆, 400 MHz) δ_{H}=7.67 (d, J=7.6 Hz, 1H), 7.61-7.55 (m, 1H), 7.51-7.46 (m, 3H), 7.41-7.38 (m, 3H), 7.36 (d, J=8.6 Hz, 1H), 7.22-7.18 (m, 2H), 7.06 (d, J=12.6 Hz, 1H), 2.91-2.84 (m, 4H), 2.37 (d, J=4.2 Hz, 4H), 2.18 (s, 3H).

Step 3: To a mixture of N-(diphenylmethylene)-2-fluoro-4-(4-methylpiperazin-1-yl)-5-nitroaniline (18 g, 34.96 mmol, 79.95% purity, 1 eq) in THF (200 mL) was added HCl (12 M, 29.13 mL, 10 eq) in one portion. The mixture was stirred at 25° C. for 12 hours. The residue was poured into water (200 mL). The aqueous phase was washed with ethyl acetate (100 mL×3). The pH of the aqueous phase was adjusted to around 8 by progressively adding solid NaHCO₃. Then the aqueous phase was extracted with DCM (100 mL*3). The combined organic phase was washed with brine (100 mL*2), dried with anhydrous Na₂SO₄, filtered and concentrated in vacuum. The crude product was used in the next step without further purification. 2-Fluoro-4-(4-methylpiperazin-1-yl)-5-analine (8.9 g, 27.06 mmol, 77.40% yield) was obtained as yellow oil.

¹H NMR: (DMSO-d₆, 400 MHz) δ_{H}=7.22 (d, J=9.2 Hz, 1H), 7.15 (d, J=12.8 Hz, 1H), 5.46 (s, 2H), 2.83 (t, J=4.8 Hz, 4H), 2.38 (s, 4H), 2.19 (s, 3H).

Step 4: To a mixture of 2-fluoro-4-(4-methylpiperazin-1-yl)-5-nitroanaline (8.9 g, 35.00 mmol, 1 eq) in HCl (2 M, 175.02 mL, 10 eq) was added a solution of NaNO₂ (3.62 g, 52.51 mmol, 1.5 eq) in H₂O (20 mL) dropwise at 0° C. After 0.5 hr, a solution of NaN₃ (3.47 g, 53.38 mmol, 1.52 eq) in H₂O (20 mL) was added into the mixture. The mixture was allowed to warm up to 15° C. and stirred for 1 hr to give a brown mixture. The pH of the mixture was adjusted to around 9 with 2N NaOH. The resulting mixture was extracted with DCM (150 mL*3). The combined organic phase was washed with brine (150 mL), dried over anhydrous Na₂SO₄, filtered and concentrated. The product 1-(4-azido-5-fluoro-2-nitrophenyl)-4-methylpiperazine (8.7 g, 31.04 mmol, 88.68% yield) was obtained as a brown solid and used into the next step without further purification.

¹H NMR: (DMSO-d₆, 400 MHz) δ_{H}=7.82 (d, J=8.8 Hz, 1H), 7.32 (d, J=13.6 Hz, 1H), 3.03-2.94 (m, 4H), 2.45-2.35 (m, 4H), 2.20 (s, 3H).

Step 5: To a mixture of 1-(4-azido-5-fluoro-2-nitrophenyl)-4-methylpiperazine (8.2 g, 29.26 mmol, 1 eq) and methyl prop-2-ynoate (3 g, 35.11 mmol, 2.92 mL, 1.2 eq) in THF (250 mL) was added CuI (2 g, 8.78 mmol, 0.3 eq) and DIEA (11 g, 87.78 mmol, 15.29 mL, 3 eq) in one portion at 25° C. for 2 hours. The mixture was filtered via a filter paper and kieselguhr and concentrated under reduce pressure. Then the mixture was poured into water (100 mL), the aqueous phase was extracted with DCM (70 mL*3). The combined organic phase was washed with brine (20 mL), dried with anhydrous Na₂SO₄, filtered and concentrated in vacuum. The crude product was used in the next step without further purification. Methyl 1-(2-fluoro-4-(4-methylpiperazin-1-yl)-5-nitrophenyl)-1H-1,2,3-triazole-4-carboxylate (12.3 g, crude) was obtained as a yellow solid.

¹H NMR: (DMSO-d₆, 400 MHz) δ_{H}=9.24 (s, 1H), 8.41 (d, J=7.6 Hz, 1H), 7.49 (d, J=13.2 Hz, 1H), 3.88 (s, 3H), 3.16 (t, J=4.6 Hz, 4H), 2.44 (s, 4H), 2.23 (s, 3H).

Step 6: To a mixture of SnCl₂.2H₂O (11 g, 49.41 mmol, 3 eq) in MeOH (100 mL) and H₂O (25 mL) was added methyl 1-(2-fluoro-4-(4-methylpiperazin-1-yl)-5-nitrophenyl)-1H-1,2,3-triazole-4-carboxylate (6 g, 16.47 mmol, 1 eq) in one portion, then the mixture was heated to 80° C. for 4 hours. The pH was adjusted to around 8 by progressively adding NaHCO₃ (40 mL). Then the mixture was added DCM (200 mL) and stirred for 5 mins. The mixture was filtered via a filter paper. The combined organic phase was washed with brine (100 mL*2), dried with anhydrous Na₂SO₄, filtered and concentrated in vacuum. The residue was purified by silica gel chromatography (column weight: 40 g, diameter: 100 mm, 100-200 mesh silica gel, Dichloromethane: Methanol=10:1). 1-(5-Amino-2-fluoro-4-(4-methylpiperazin-1-yl)phenyl)-1H-1,2,3-triazole-4-carboxylate (2.7 g, 7.29 mmol, 44.29% yield) was obtained as a yellow solid.

¹H NMR: (DMSO-d₆, 400 MHz) δ_{H}=8.55 (d, J=2.4 Hz, 1H), 7.31 (d, J=7.2 Hz, 1H), 6.90 (d, J=12.4 Hz, 1H), 4.06 (s, 2H), 3.98 (s, 3H), 2.98 (s, 4H), 2.76-2.51 (m, 4H), 2.38 (s, 3H).

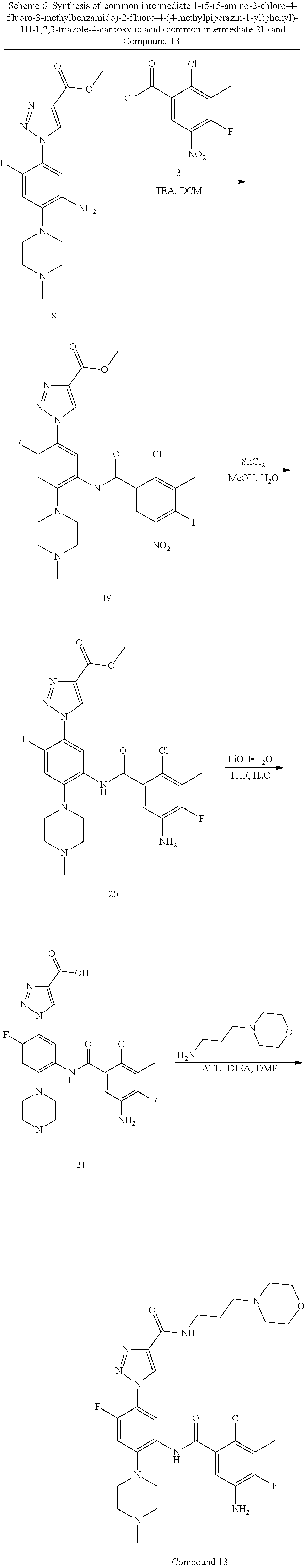

Step 7: A mixture of 1-(5-amino-2-fluoro-4-(4-methylpiperazin-1-yl)phenyl)-1H-1,2,3-triazole-4-carboxylate (200 mg, 598.18 μmol, 1 eq) and TEA (303 mg, 2.99 mmol, 416.29 μL, 5 eq) in DCM (5 mL) at 0° C. was stirred for 10 mins, then 2-chloro-4-fluoro-3-methyl-5-nitrobenzoyl chloride (136 mg, 538.36 μmol, 0.9 eq) was added in one portion at 0° C. The mixture was stirred for 12 hours. The residue was poured into water (20 mL). The aqueous phase was extracted with DCM (15 mL*3). The combined organic phase was washed with brine (20 mL), dried with anhydrous Na₂SO₄, filtered and concentrated in vacuum. The residue was purified by silica gel chromatography (column weight: 12 g, diameter: 100 mm, 100-200 mesh silica gel, Dichloromethane: Methanol=10:1). Methyl 1-(5-(2-chloro-4-fluoro-3-methyl-5-nitrobenzamido)-2-fluoro-4-(4-methylpiperazin-1-yl)phenyl)-1H-1,2,3-triazole-4-carboxylate (220 mg, 256.04 μmol, 42.80% yield) was obtained as a yellow solid.

¹H NMR: (DMSO-d₆, 400 MHz) δ_{H}=9.10 (d, J=8.0 Hz, 1H), 8.99 (s, 1H), 8.30 (d, J=7.6 Hz, 1H), 8.02 (s, 1H), 7.23-7.18 (m, 1H), 4.02 (s, 3H), 3.00 (d, J=4.8 Hz, 4H), 2.55 (d, J=2.8 Hz, 2H), 2.53 (d, J=2.8 Hz, 3H), 2.46-2.42 (m, 2H), 2.37 (s, 3H).

Step 8: To a mixture of methyl 1-(5-(2-chloro-4-fluoro-3-methyl-5-nitrobenzamido)-2-fluoro-4-(4-methylpiperazin-1-yl)phenyl)-1H-1,2,3-triazole-4-carboxylate (220 mg, 400.06 μmol, 1 eq) in MeOH (10 mL) and H₂O (3 mL) was added SnCl₂.2H₂O (271 mg, 1.20 mmol, 3 eq) in one portion. The mixture was heated to 80° C. and stirred for 4 hours. The pH was adjusted to around 8 by progressively adding NaHCO₃ (40 mL). Then the mixture was added DCM (200 mL) and stirred for 5 mins. The mixture was filtered via a filter paper. The combined organic phase was washed with brine (100 mL*2), dried with anhydrous Na₂SO₄, filtered and concentrated in vacuum. The residue was purified by silica gel chromatography (column weight: 12 g, diameter: 100 mm, 100-200 mesh silica gel, Dichloromethane: Methanol=10:1). Methyl 1-(5-(5-amino-2-chloro-4-fluoro-3-methylbenzamido)-2-fluoro-4-(4-methylpiperazin-1-yl)phenyl)-1H-1,2,3-triazole-4-carboxylate (130 mg, 240.03 μmol, 60.00% yield) was obtained as a yellow solid.

¹H NMR: (DMSO-d₆, 400 MHz) δ_{H}=9.13 (d, J=8.0 Hz, 1H), 9.05 (s, 1H), 8.57-8.51 (m, 1H), 7.16 (d, J=11.6 Hz, 1H), 7.08 (d, J=9.2 Hz, 1H), 4.01 (s, 3H), 3.89 (s, 2H), 3.01-2.98 (m, 3H), 2.97 (s, 3H), 2.61 (s, 3H), 2.44-2.40 (m, 2H), 2.38 (s, 3H).

Step 9: To a mixture of methyl 1-(5-(5-amino-2-chloro-4-fluoro-3-methylbenzamido)-2-fluoro-4-(4-methylpiperazin-1-yl)phenyl)-1H-1,2,3-triazole-4-carboxylate (130 mg, 250.03 μmol, 1 eq) in THF (5 mL) and H₂O (2 mL) was added LiOH·H₂O (21 mg, 500.07 μmol, 2 eq) in one portion at 2 5° C. and stirred for 12 hours. The pH of the mixture was adjusted to around 4 with 2N HCl. The mixture was concentrated to remove THF. The crude product was used in the next step without further purification. 1-(5-(5-Amino-2-chloro-4-fluoro-3-methylbenzamido)-2-fluoro-4-(4-methylpiperazin-1-yl)phenyl)-1H-1,2,3-triazole-4-carboxylic acid (190 mg, crude) was obtained as a yellow solid.

¹H NMR: (DMSO-d₆, 400 MHz) δ_{H}=9.76 (s, 1H), 9.14 (d, J=1.2 Hz, 1H), 8.30 (d, J=8.0 Hz, 1H), 7.45 (d, J=12.0 Hz, 1H), 6.85 (d, J=9.2 Hz, 1H), 4.10 (br s, 2H), 3.45-3.33 (m, 3H), 3.22 (br d, J=11.6 Hz, 4H), 2.80 (br d, J=4.4 Hz, 3H), 2.34-2.29 (m, 2H), 2.25 (d, J=2.0 Hz, 3H).

Step 10: To a mixture of 1-(5-(5-amino-2-chloro-4-fluoro-3-methylbenzamido)-2-fluoro-4-(4-methylpiperazin-1-yl)phenyl)-1H-1,2,3-triazole-4-carboxylic acid (190 mg, 375.57 μmol, 1 eq) and 3-morpholinopropan-1-amine (54 mg, 375.57 μmol, 54.88 μL, 1 eq) in DMF (3 mL) was added DIEA (146 mg, 1.13 mmol, 196.25 μL, 3 eq) and HATU (171 mg, 450.68 μmol, 1.2 eq) in one portion. The mixture was stirred for 12 hrs at 25° C. The residue was concentrated under reduce pressure. The residue was purified by prep. HPLC (Column: Phenomenex Gemini-NX C18 75*30 mm*3 μm; mobile phase: [water (0.05% NH₃H2O+10 mM NH₄HCO₃)-ACN]; B %: 21%-51%; 11 min). Example 13 (42.2 mg, 65.68 μmol, 17.49% yield, 98.38% purity) was obtained as a light yellow solid.

¹H NMR: (DMSO-d₆, 400 MHz) δ_{H}=9.58 (s, 1H), 8.96 (s, 1H), 8.84 (t, J=5.6 Hz, 1H), 8.25 (d, J=8.0 Hz, 1H), 7.38 (d, J=12.4 Hz, 1H), 6.83 (d, J=9.2 Hz, 1H), 5.50 (s, 2H), 3.60 (t, J=4.4 Hz, 4H), 3.38-3.34 (m, 2H), 2.99 (s, 4H), 2.49-2.44 (m, 4H), 2.41-2.32 (m, 6H), 2.25 (s, 3H), 2.23 (s, 3H), 1.70 (quin, J=6.8 Hz, 2H).

HPLC: Rₜ=2.340 min in 8 min chromatography, XBridge Shield RP18, 5 μm, 2.1*50 mm, 5 μm, purity 98.380%.

LCMS: Rₜ=1.891 min in 4 min chromatography, XBridge Shield RP18, 5 μm, 2.1*50 mm, 5 m, purity 96.336%, MS ESI calcd. for 631.26 [M+H]⁺631.26, found 632.4.

### Example 14. Synthesis of 1-(5-(5-amino-2-chloro-4-fluoro-3-methylbenzamido)-4-(3,4-dimethylpiperazin-1-yl)-2-fluorophenyl)-N-(3-morpholinopropyl)-1H-1,2,3-triazole-4-carboxamide (Compound 14, Table 1)

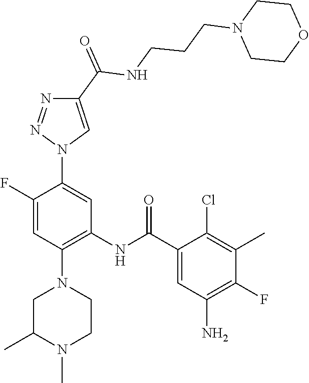 [SMILES: Cc1c(F)c(N)cc(C(=O)Nc2cc(-n3cc(C(=O)NCCCN4CCOCC4)nn3)c(F)cc2N2CCN(C)C(C)C2)c1Cl]

The title compound was synthesized according the schemes 5 and 6, (Example 13).

Step 1: To a mixture of 1-bromo-2,4-difluoro-5-nitrobenzene (5 g, 21.01 mmol, 1 eq) and 1,2-dimethylpiperazine (2 g, 21.01 mmol, 1 eq) in CH₃CN (150 mL) was added DIEA (5 g, 42.02 mmol, 7.32 mL, 2 eq) in one portion. The mixture was stirred at 25° C. for 12 hours. The residue was concentrated under reduce pressure to remove THF. The residue was purified by silica gel chromatography (column Weight: 40 g, diameter: 100 mm, 100-200 mesh silica gel, Dichloromethane: Methanol=10:1). 4-(4-bromo-5-fluoro-2-nitrophenyl)-1,2-dimethylpiperazine (6.5 g, 19.54 mmol, 93.00% yield) was obtained as a yellow solid.

¹H NMR: (DMSO-d₆, 400 MHz) δ_{H}=8.21 (d, J=7.6 Hz, 1H), 7.31 (d, J=11.2 Hz, 1H), 3.10-3.03 (m, 2H), 3.01-2.92 (m, 1H), 2.73 (td, J=2.6, 11.6 Hz, 1H), 2.62 (dd, J=9.6, 12.3 Hz, 1H), 2.25-2.20 (m, 1H), 2.19 (s, 3H), 2.16-2.08 (m, 1H), 0.97 (d, J=6.4 Hz, 3H).

Step 2: To a mixture of 4-(4-bromo-5-fluoro-2-nitrophenyl)-1,2-dimethylpiperazine (6.5 g, 19.57 mmol, 1 eq) and diphenylmethanimine (5.3 g, 29.35 mmol, 4.93 mL, 1.5 eq) in dioxane (100 mL) was added Pd(OAc)₂ (439.33 mg, 1.96 mmol, 0.1 eq), Xantphos (1.70 g, 2.94 mmol, 0.15 eq) and Cs₂CO₃ (12.75 g, 39.14 mmol, 2 eq) in one portion at 25° C. under N₂. The mixture was heated to 100° C. and stirred for 12 hrs. The residue was poured into water (200 mL). The aqueous phase was extracted with DCM (100 mL*3). The combined organic phase was washed with brine (100 mL*2), dried with anhydrous Na₂SO₄, filtered and concentrated in vacuum. The residue was purified by silica gel chromatography (column weight: 80 g, diameter: 100 mm, 100-200 mesh silica gel, Dichloromethane: Methanol=10:1). 4-(3,4-dimethylpiperazin-1-yl)-N-(diphenylmethylene)-2-fluoro-5-nitroaniline (6.66 g, 9.58 mmol, 48.95% yield) was obtained as a yellow oil.

¹H NMR: (DMSO-d₆, 400 MHz) δ_{H}=7.67 (d, J=7.2 Hz, 2H), 7.52-7.46 (m, 3H), 7.41-7.38 (m, 3H), 7.36 (d, J=8.4 Hz, 1H), 7.20 (dd, J=2.8, 6.4 Hz, 2H), 7.05 (d, J=12.4 Hz, 1H), 2.97-2.79 (m, 3H), 2.77-2.58 (m, 2H), 2.17 (s, 3H), 2.15-2.05 (m, 2H), 0.94 (d, J=6.4 Hz, 3H).

Step 3: To a mixture of 4-(3,4-dimethylpiperazin-1-yl)-N-(diphenylmethylene)-2-fluoro-5-nitroaniline (6.66 g, 15.40 mmol, 1 eq) in THF (100 mL) was added HCl (12 M, 12.83 mL, 10 eq) in one portion. The reaction mixture was stirred for 12 hours at 25° C. The residue was poured into water (200 mL). The aqueous phase was washed with ethyl acetate (100 mL×3). The pH was adjusted to around 8 by progressively adding solid NaHCO₃. Then the aqueous phase was extracted with DCM (100 mL*3). The combined organic phase was washed with brine (100 mL*2), dried with anhydrous Na₂SO₄, filtered and concentrated in vacuum. The crude product was used in the next step without further purification. 4-(3,4-dimethylpiperazin-1-yl)-2-fluoro-5-nitroaniline (4.6 g, 11.20 mmol, 72.71% yield) was obtained as a yellow gum.

¹H NMR: (DMSO-d₆, 400 MHz) δ_{H}=7.22 (d, J=9.2 Hz, 1H), 7.13 (d, J=12.8 Hz, 1H), 5.45 (s, 2H), 3.11-3.02 (m, 1H), 2.89-2.79 (m, 3H), 2.72 (d, J=10.8 Hz, 1H), 2.48-2.42 (m, 1H), 2.19 (s, 3H), 2.13 (s, 1H), 0.96 (d, J=6.4 Hz, 3H).

Step 4: To a mixture of 4-(3,4-dimethylpiperazin-1-yl)-2-fluoro-5-nitroaniline (4.6 g, 17.15 mmol, 1 eq) in HCl (2 M, 85.73 mL, 10 eq) was added a solution of NaNO₂ (1.77 g, 25.72 mmol, 1.5 eq) in H₂O (20 mL) dropwise at 0° C. After 0.5 hr, a solution of NaN₃ (1.86 g, 28.61 mmol, 1.67 eq) in H₂O (20 mL) was added into the mixture. The mixture was allowed to warm up to 15° C. and stirred for 1 hr to give a brown mixture. The pH of the mixture was adjusted to around 9 with 2N NaOH. The resulting mixture was extracted with DCM (100 mL*3). The combined organic phase was washed with brine (150 mL), dried over anhydrous Na₂SO₄, filtered and concentrated. The product 1-(4-azido-5-fluoro-2-nitrophenyl)-4-methylpiperazine (3 g, 7.89 mmol, 46.02% yield) was obtained as a brown solid.

¹H NMR: (DMSO-d₆, 400 MHz) δ_{H}=7.82 (d, J=8.8 Hz, 1H), 7.31 (d, J=13.6 Hz, 1H), 3.09-2.88 (m, 3H), 2.74 (td, J=2.8, 11.6 Hz, 1H), 2.59 (dd, J=9.6, 12.0 Hz, 1H), 2.27-2.11 (m, 4H), 1.03-0.92 (m, 3H).

Step 5: To a mixture of 1-(4-azido-5-fluoro-2-nitrophenyl)-4-methylpiperazine (2.8 g, 9.51 mmol, 1 eq) and methyl prop-2-ynoate (960 mg, 11.42 mmol, 950.40 μL, 1.2 eq) in THF (150 mL) was added CuI (543.62 mg, 2.85 mmol, 0.3 eq) and DIEA (3.7 g, 28.54 mmol, 4.97 mL, 3 eq) in one portion at 25° C. and stirred for 12 hours. The mixture was filtered via a filter paper and kieselguhr and concentrated under reduce pressure. Then the mixture was poured into water (50 mL), the aqueous phase was extracted with DCM (30 mL*3). The combined organic phase was washed with brine (50 mL), dried with anhydrous Na₂SO₄, filtered and concentrated in vacuum. The crude product was used in the next step without further purification. 1-(4-(3,4-dimethylpiperazin-1-yl)-2-fluoro-5-nitrophenyl)-1H-1,2,3-triazole-4-carboxylate (5.7 g, crude) was obtained as a yellow solid.

¹H NMR: (DMSO-d₆, 400 MHz) δ_{H}=9.24 (d, J=0.8 Hz, 1H), 8.41 (d, J=8.0 Hz, 1H), 7.50 (d, J=13.2 Hz, 1H), 3.89 (s, 3H), 3.14-3.05 (m, 3H), 3.00-2.92 (m, 1H), 2.79-2.73 (m, 3H), 2.22 (s, 3H), 1.01 (d, J=6.0 Hz, 3H).

Step 6: To a mixture of SnCl₂.2H₂O (10.33 g, 45.80 mmol, 4 eq) in MeOH (100 mL) and H₂O (30 mL) was added 1-(4-(3,4-dimethylpiperazin-1-yl)-2-fluoro-5-nitrophenyl)-1H-1,2,3-triazole-4-carboxylate (5.7 g, 11.45 mmol, 76% purity, 1 eq) in one portion, then the mixture was heated to 80° C. and stirred for 4 hours. The pH was adjusted to around 8 by progressively adding NaHCO₃ (40 mL). Then the mixture was added DCM (200 mL) and stirred for 5 mins. The mixture was filtered via a filter paper. The combined organic phase was washed with brine (100 mL*2), dried with anhydrous Na₂SO₄, filtered and concentrated in vacuum. The residue was purified by silica gel chromatography (column weight: 40 g, diameter: 100 mm, 100-200 mesh silica gel, Dichloromethane: Methanol=10:1). 1-(5-Amino-4-(3,4-dimethylpiperazin-1-yl)-2-fluorophenyl)-1H-1,2,3-triazole-4-carboxylate (2.5 g, 6.64 mmol, 58.01% yield) was obtained as a yellow solid.

¹H NMR: (DMSO-d₆, 400 MHz) δ_{H}=9.10 (d, J=2.0 Hz, 1H), 7.06-6.99 (m, 2H), 4.99 (s, 2H), 3.87 (s, 3H), 3.16 (d, J=4.8 Hz, 1H), 3.08-2.99 (m, 2H), 2.98-2.89 (m, 1H), 2.82-2.66 (m, 3H), 2.23 (s, 3H), 1.01 (d, J=6.0 Hz, 3H).

Step 7: To a solution of 2-chloro-4-fluoro-3-methyl-5-nitrobenzoyl chloride (506.40 mg, 2.01 mmol, 1 eq) and 1-(5-amino-4-(3,4-dimethylpiperazin-1-yl)-2-fluorophenyl)-1H-1,2,3-triazole-4-carboxylate (700 mg, 2.01 mmol, 1 eq) in DCM (20 mL) was added TEA (1.02 g, 10.05 mmol, 1.40 mL, 5 eq) at −20° C. The reaction mixture was allowed to warm to 20° C. and stirred for 3 hrs to give a brown mixture. Water (20 mL) was added to the reaction mixture. The resulting mixture was extracted with DCM (20 mL*3). The combined organic phase was washed with brine (20 mL), dried over anhydrous Na₂SO₄, filtered and concentrated. The residue was purified by flash silica gel chromatography (ISCO®; 12 g SepaFlash® Silica Flash Column, Eluent of 0-15% DCM/MeOH ether gradient at 25 mL/min). The impure product methyl 1-(5-(2-chloro-4-fluoro-3-methyl-5-nitrobenzamido)-4-(3,4-dimethylpiperazin-1-yl)-2-fluorophenyl)-1H-1,2,3-triazole-4-carboxylate (460 mg, 415.19 μmol, 20.66% yield) was obtained as a brown solid.

LCMS: Rₜ=0.807 min in 1.5 min chromatography, XBridge Shield RP18, 5 μm, 2.1*50 mm, purity 50.91%, MS ESI calcd. for 563.15 [M+H]⁺564.15, found 564.0.

Step 8: To a mixture of methyl 1-(5-(2-chloro-4-fluoro-3-methyl-5-nitrobenzamido)-4-(3,4-dimethylpiperazin-1-yl)-2-fluorophenyl)-1H-1,2,3-triazole-4-carboxylate (460 mg, 815.69 μmol, 1 eq) in MeOH (20 mL) and H₂O (6 mL) was added SnCl₂.2H₂O (552 mg, 2.45 mmol, 3 eq) in one portion. The mixture was heated to 80° C. for 4 hours. The pH was adjusted to around 8 by progressively adding NaHCO₃ (40 mL). Then the mixture was added DCM (100 mL) and stirred for 5 mins. The mixture was filtered via a filter paper. The combined organic phase was washed with brine (70 mL*2), dried with anhydrous Na₂SO₄, filtered and concentrated in vacuum. The residue was purified by silica gel chromatography (column weight: 12 g, diameter: 100 mm, 100-200 mesh silica gel, Dichloromethane: Methanol=10:1). 1-(5-(5-Amino-2-chloro-4-fluoro-3-methylbenzamido)-4-(3,4-dimethylpiperazin-1-yl)-2-fluorophenyl)-1H-1,2,3-triazole-4-carboxylate (180 mg, 286.84 μmol, 35.17% yield) was obtained as a yellow solid.

¹H NMR: (DMSO-d₆, 400 MHz) δ_{H}=9.25 (br s, 1H), 7.85 (dd, J=6.4, 8.8 Hz, 1H), 7.01 (dd, J=2.4, 10.6 Hz, 1H), 6.96-6.89 (m, 1H), 6.83-6.79 (m, 1H), 3.89 (s, 2H), 3.32-3.28 (m, 3H), 3.19-3.04 (m, 1H), 3.02-2.87 (m, 3H), 2.84-2.72 (m, 3H), 2.24 (s, 6H), 0.98 (d, J=6.0 Hz, 3H).

Step 9: To a mixture of 1-(5-(5-amino-2-chloro-4-fluoro-3-methylbenzamido)-4-(3,4-dimethylpiperazin-1-yl)-2-fluorophenyl)-1H-1,2,3-triazole-4-carboxylate (180 mg, 337.11 μmol, 1 eq) in THF (3 mL) and H₂O (1 mL) was added LiOH·H₂O (28.29 mg, 674.21 μmol, 2 eq) in one portion. The mixture was stirred at 25° C. for 2 hours. The pH of the mixture was adjusted to around 4 with 2N HCl. The mixture was concentrated to remove THF. The crude product was used in the next step without further purification. 1-(5-(5-Amino-2-chloro-4-fluoro-3-methylbenzamido)-4-(3,4-dimethylpiperazin-1-yl)-2-fluorophenyl)-1H-1,2,3-triazole-4-carboxylic acid (240 mg, 148.77 μmol, 44.13% yield) was obtained as a yellow solid.

LCM:S Rₜ=1.891 min in 4 min chromatography, Chromolith Flash RP-18, 5 um, 3.0*25 mm, purity 35.469%, MS ESI calcd. for 519.16 [M+H]⁺520.16, found 520.1.

Step 10: To a mixture of 1-(5-(5-amino-2-chloro-4-fluoro-3-methylbenzamido)-4-(3,4-dimethylpiperazin-1-yl)-2-fluorophenyl)-1H-1,2,3-triazole-4-carboxylic acid (240 mg, 461.60 μmol, 1 eq) and 3-morpholinopropan-1-amine (67 mg, 461.60 μmol, 67.45 μL, 1 eq) in DMF (3 mL) was added DIEA (179 mg, 1.38 mmol, 241.21 μL, 3 eq) and HATU (263 mg, 692.40 μmol, 1.5 eq) in one portion. The mixture was stirred at 25° C. for 12 hours. The residue was concentrated under reduce pressure. The residue was purified by prep. HPLC (Column: Phenomenex Gemini-NX C18 75*30 mm*3 μm; mobile phase:I[water(0.05% NH3H2O+10 mM NH₄HCO₃)-ACN]; B %: 20%-60%; 11 min). Example 14 (28.7 mg, 44.13 μmol, 9.56% yield, 99.34% purity) was obtained as a yellow solid.

¹H NMR: (DMSO-d₆, 400 MHz) δ_{H}=9.56 (s, 1H), 8.96 (s, 1H), 8.84 (s, 1H), 8.25 (d, J=8.0 Hz, 1H), 7.36 (d, J=12.0 Hz, 1H), 6.83 (d, J=9.2 Hz, 1H), 5.48 (s, 2H), 3.60 (s, 4H), 3.37-3.34 (m, 3H), 3.15-3.01 (m, 2H), 2.88 (t, J=10.4 Hz, 1H), 2.78 (d, J=10.8 Hz, 1H), 2.36 (s, 8H), 2.23 (d, J=13.6 Hz, 6H), 1.76-1.65 (m, 2H), 1.00 (d, J=5.6 Hz, 3H).

HPLC: Rₜ=1.79 min in 8 min chromatography, Ultimate XB-C18 3.0*50 mm, 3 μm, purity 99.34%.

LCMS: Rₜ=1.891 min in 4 min chromatography, XBridge Shield RP18, 5 μm, 2.1*50 mm, purity 98.051%, MS ESI calcd. for 645.28 [M+H]⁺646.28, found 646.4.

### Example 15. Synthesis of 1-(5-(5-amino-2-chloro-4-fluoro-3-methylbenzamido)-2-fluoro-4-((3S,5R)-3,4,5-trimethylpiperazin-1-yl)phenyl)-N-(3-morpholinopropyl)-1H-1,2,3-triazole-4-carboxamide (Compound 15, Table 1)

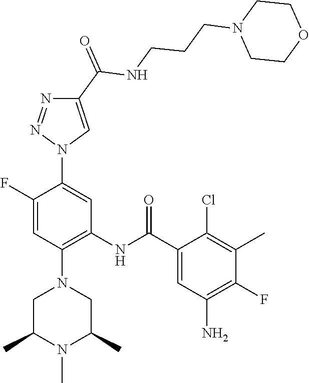 [SMILES: Cc1c(F)c(N)cc(C(=O)Nc2cc(-n3cc(C(=O)NCCCN4CCOCC4)nn3)c(F)cc2N2C[C@@H](C)N(C)[C@@H](C)C2)c1Cl]

To a mixture of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (120 mg, 224.74 μmol, 1 eq), 3-morpholinopropan-1-amine (48.62 mg, 337.11 μmol, 49.26 μL, 1.5 eq) and DIEA (87 mg, 674.21 μmol, 117.44 μL, 3 eq) in DMF (4 mL) was added HATU (128 mg, 337.11 μmol, 1.5 eq). The reaction mixture was stirred at 20° C. for 2 hr to give a brown mixture. The reaction mixture was concentrated. The residue was purified by prep-HPLC (Column: Phenomenex Gemini-NX C18 75*30 mm*3 μm; Condition: water(0.04% NH₃H2O+10 mM NH₄HCO₃)-ACN; Begin B: 20%; End B: 60%; Gradient Time(min): 12 min). The product Example 15 (17.2 mg, 25.82 μmol, 11.49% yield, 99.11% purity) was obtained as a white solid.

¹H NMR: (DMSO-d₆, 400 MHz) δ_{H}=9.59 (s, 1H), 8.95 (s, 1H), 8.84 (t, J=5.6 Hz, 1H), 8.24 (d, J=8.0 Hz, 1H), 7.33 (d, J=12.4 Hz, 1H), 6.82 (d, J=9.2 Hz, 1H), 5.47 (s, 2H), 3.60 (s, 4H), 3.08 (d, J=10.8 Hz, 2H), 2.61-2.51 (m, 4H), 2.36 (s, 8H), 2.24 (s, 3H), 2.19 (s, 3H), 1.70 (t, J=6.8 Hz, 2H), 1.02 (d, J=6.0 Hz, 6H).

HPLC: Rₜ=3.524 min in 8 min chromatography, Ultimate XB-C18 3.0*50 mm, 3 μm, purity 99.12%.

LCMS: Rₜ=2.699 min in 4 min chromatography, XBridge Shield RP18, 5 μm, 2.1*50 mm, purity 99.47%, MS ESI calcd. for 659.29 [M+H]⁺660.29, found 660.5.

### Example 16. Synthesis of 1-(3-(4-fluoro-3,5-dimethylbenzamido)-4-(4-methylpiperazin-1-yl)phenyl)-N-(3-morpholinopropyl)-1H-1,2,3-triazole-4-carboxamide (Compound 16, Table 1)

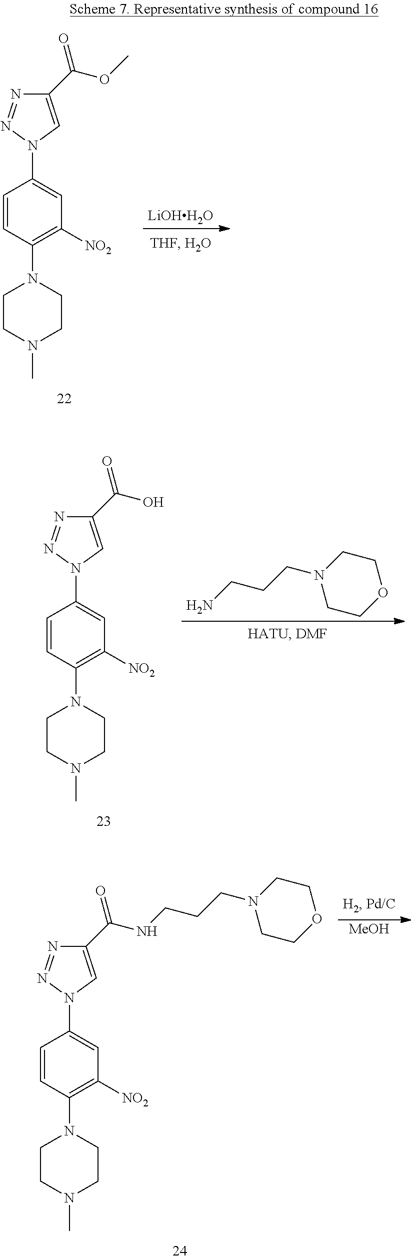

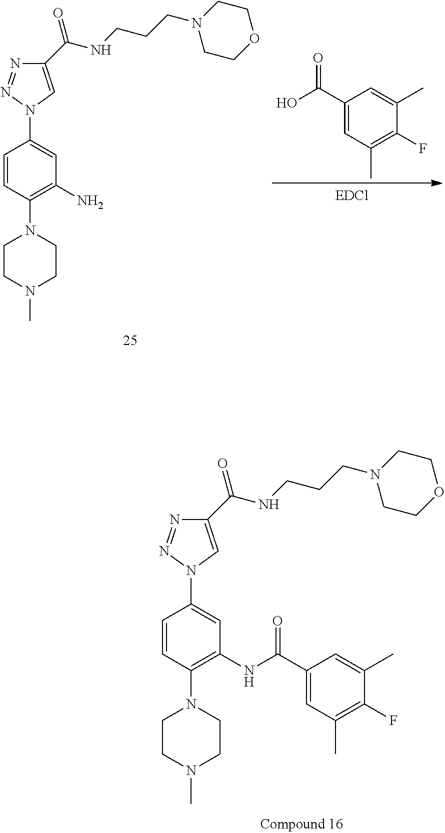 [SMILES: CN1CCN(c2ccc(-n3cc(C(=O)NCCCN4CCOCC4)nn3)cc2N)CC1 ; Cc1cc(C(=O)O)cc(C)c1F ; Cc1cc(C(=O)Nc2cc(-n3cc(C(=O)NCCCN4CCOCC4)nn3)ccc2N2CCN(C)CC2)cc(C)c1F]

Step 1: To a mixture of methyl 1-(4-(4-methylpiperazin-1-yl)-3-nitrophenyl)-1H-1,2,3-triazole-4-carboxylate (10 g, 28.87 mmol, 1 eq) in THF (60 mL) and H₂O (30 mL) was added LiOH·H₂O (6 g, 144.37 mmol, 5 eq). The mixture was stirred at 25° C. for 10 hrs. The mixture was concentrated to remove THF. The pH of the mixture was adjusted to around 4 with 2N HCl. The mixture was filtered via a filter paper. The filter cake was dried under reduced pressure. The product was used directly to the next step without further purification. 1-(4-(4-methylpiperazin-1-yl)-3-nitrophenyl)-1H-1,2,3-triazole-4-carboxylic acid (7 g, 19.41 mmol, 67.23% yield) was obtained as a yellow solid.

¹H NMR: (DMSO-d₆, 400 MHz) δ_{H}=9.45 (s, 1H), 8.51 (d, J=2.4 Hz, 1H), 8.21 (dd, J=2.4, 9.0 Hz, 1H), 7.59 (d, J=9.2 Hz, 1H), 3.37-3.30 (m, 4H), 3.03 (s, 4H), 2.63 (s, 3H).

Step 2: To a mixture of 1-(4-(4-methylpiperazin-1-yl)-3-nitrophenyl)-1H-1,2,3-triazole-4-carboxylic acid (5 g, 15.05 mmol, 1 eq.) and 3-morpholinopropan-1-amine (2 g, 15.05 mmol, 2.20 mL, 1 eq.) in DMF (70 mL) was added DIEA (6 g, 45.14 mmol, 7.86 mL, 3 eq.) in one portion at 25° C., then HATU (7 g, 18.06 mmol, 1.2 eq) was added in one portion. The mixture was stirred at 25° C. for 10 hrs. The mixture was concentrated to remove DMF. The residue was triturated with CH₃CN (50 mL) to give a yellow solid (3 g). The residue was purified by silica gel chromatography (column weight: 40 g, 100-200 mesh silica gel, Dichloromethane: Methanol=7:3). 1-(4-(4-Methylpiperazin-1-yl)-3-nitrophenyl)-N-(3-morpholinopropyl)-1H-1,2,3-triazole-4-carboxamid (3 g., crude) was obtained as a yellow solid.

¹H NMR: MeOD-d₄, 400 MHz) δ_{H}=9.00 (s, 1H), 8.47 (d, J=2.4 Hz, 1H), 8.17 (dd, J=2.4, 8.8 Hz, 1H), 7.62 (d, J=9.2 Hz, 1H), 3.89 (s, 4H), 3.55 (t, J=6.4 Hz, 2H), 3.40 (d, J=4.4 Hz, 4H), 3.30-3.25 (m, 4H), 3.14-2.99 (m, 6H), 2.87 (s, 3H), 2.08-2.00 (m, 2H).

Step 3: To a mixture of 1-(4-(4-methylpiperazin-1-yl)-3-nitrophenyl)-N-(3-morpholinopropyl)-1H-1,2,3-triazole-4-carboxamid (2 g, 4.36 mmol, 1 eq.) in MeOH (80 mL) was added wet Pd/C (590 mg, 4.36 mmol, 10% purity, 1 eq.). The reaction mixture was degassed and refilled with H₂. The reaction mixture was stirred under H₂ (15 psi) for 12 hr at 30° C. to give a black mixture. The reaction mixture was filtered to remove Pd/C. And the filter cake was washed with MeOH (30 mL). Then the filtrate was concentrated. The crude product 1-(3-amino-4-(4-methylpiperazin-1-yl)phenyl)-N-(3-morpholinopropyl)-1H-1,2,3-triazole-4-carboxamide (1.9 g, crude) was obtained as a yellow solid and used into the next step without further purification.

¹H NMR: (MeOD, 400 MHz) δ_{H}=8.71 (s, 1H), 8.52 (d, J=3.6 Hz, 1H), 8.18 (d, J=7.6 Hz, 1H), 7.24 (d, J=2.8 Hz, 1H), 3.81-3.70 (m, 6H), 3.51 (t, J=6.8 Hz, 2H), 3.22-3.11 (m, 7H), 2.75 (s, 3H), 2.70-2.61 (m, 6H), 1.93-1.86 (m, 2H).

Step 4: To a mixture of 1-(3-amino-4-(4-methylpiperazin-1-yl)phenyl)-N-(3-morpholinopropyl)-1H-1,2,3-triazole-4-carboxamide (180 mg, 420.04 μmol, 1 eq) and 4-fluoro-3,5-dimethyl-benzoic acid (71 mg, 420.04 μmol, 1 eq) in pyridine (1 mL) was added EDCI (121 mg, 630.06 μmol, 1.5 eq) in one portion at 25° C. under N₂. The mixture was stirred at 100° C. for 12 hrs. The residue was concentrated under reduce pressure to remove pyridine. The residue was purified by prep. HPLC (Column: Phenomenex Gemini-NX C18 75*30 mm*3 μm; mobile phase: [water (0.04% NH₃H₂O+10 mM NH₄HCO₃)-ACN]; B %:15%-65%; 15 min). Example 16 (14.7 mg, 25.05 μmol, 5.96% yield, 98.61% purity) was obtained as a light yellow solid.

¹H NMR: (DMSO-d₆, 400 MHz) δ_{H}=9.66 (s, 1H), 9.21 (s, 1H), 8.85 (t, J=5.6 Hz, 1H), 8.71 (d, J=2.4 Hz, 1H), 7.75-7.68 (m, 3H), 7.46 (d, J=8.8 Hz, 1H), 3.61 (t, J=4.4 Hz, 4H), 2.94 (t, J=4.4 Hz, 4H), 2.55-2.51 (m, 6H), 2.36 (t, J=6.8 Hz, 6H), 2.33 (s, 6H), 2.25 (s, 3H), 1.70 (quin, J=6.8 Hz, 2H).

HPLC: Rₜ=2.739 min in 8 min chromatography, XBridge Shield RP18, 5 μm, 2.1*50 mm, 8 m, purity 98.61%.

LCMS: Rₜ=2.199 min in 4 min chromatography, XBridge Shield RP18, 5 μm, 2.1*50 mm, 8 m, purity 98.58%, MS ESI calcd. for 578.31 [M+H]⁺579.31, found 579.5.

### Example 17. Synthesis of 1-[3-[(2-chloro-4-fluoro-3-methyl-benzoyl)amino]-4-(4-methylpiperazin-1-yl)phenyl]-N-(3-morpholinopropyl)triazole-4-carboxamide (Compound 17, Table 1)

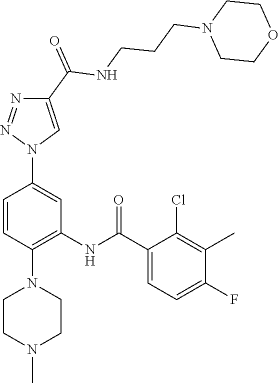 [SMILES: Cc1c(F)ccc(C(=O)Nc2cc(-n3cc(C(=O)NCCCN4CCOCC4)nn3)ccc2N2CCN(C)CC2)c1Cl]

The title compound was synthesized according to Scheme 7 (Example 16) and Scheme 8.

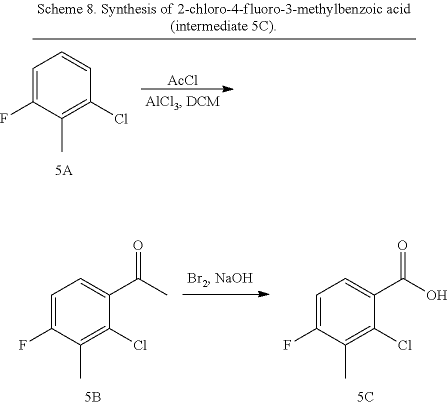

Step 1: To a mixture of 1-chloro-3-fluoro-2-methylbenzene (10 g, 69.17 mmol, 8.40 mL, 1 eq.), AlCl₃ (18.45 g, 138.34 mmol, 7.56 mL, 2 eq.) in DCM (100 mL) was added acetyl chloride (5.43 g, 69.17 mmol, 4.94 mL, 1 eq.) dropwise at 0° C. The mixture was allowed to warm up to 25° C. and stirred for 2 hrs. The mixture was poured into ice-water and acidified with 2N HCl to pH=4. The mixture was extracted with DCM (100 mL*3), and washed with brine (100 mL*3). The combined organic layer was dried over Na₂SO₄, and concentrated to give crude product. The crude product was purified by column chromatography on silica gel (PE: EtOAc=50:1). 1-(2-Chloro-4-fluoro-3-methyl-phenyl)ethanone (8 g, 42.87 mmol, 61.98% yield) was obtained as a yellow oil.

¹H NMR: (DMSO-d₆, 400 MHz) δ_{H}=7.42-7.35 (m, 1H), 7.03 (t, J=8.8 Hz, 1H), 2.63 (s, 3H), 2.35 (d, J=2.4 Hz, 3H).

Step 2: To a solution of NaOH (8.57 g, 214.35 mmol, 10 eq.) in H₂O (40 mL) was added Br₂ (10.28 g, 64.30 mmol, 3.31 mL, 3 eq.) dropwise at 10° C. A solution of (4 g, 21.43 mmol, 1 eq.) in dioxane (40 mL) was added into the mixture at 0° C. dropwise. The mixture was allowed to warm up to 25° C. and stirred for 2 hrs. The mixture was extracted with DCM (50 mL*2). The pH of the aqueous phase was adjusted to around 4 with 2N HCl. The mixture was filtered via a filter paper, and the filter cake was dried under reduced pressure. The product was used directly to the next step without further purification. 2-Chloro-4-fluoro-3-methyl-benzoic acid 5C (4 g, 21.21 mmol, 98.95% yield) was obtained as a white solid.

¹H NMR: (DMSO-d₆, 400 MHz) δ_{H}=7.70-7.64 (m, 1H), 7.28 (m, J=8.8 Hz, 1H), 2.30 (d, J=2.4 Hz, 3H).

Step 3: To a solution of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) and 2-chloro-4-fluoro-3-methyl-benzoic acid (66 mg, 350.03 μmol, 1 eq.) in pyridine (1 mL) was added EDCI (101 mg, 525.05 μmol, 1.5 eq.). The reaction mixture was stirred at 100° C. for 12 hr to give a yellow mixture. The reaction mixture was concentrated to dryness. Water (15 mL) was added to the reaction mixture. The resulting mixture was extracted with DCM (15 mL*3). The combined organic phase was washed with brine (30 mL), dried over anhydrous Na₂SO₄, filtered and concentrated to dryness. The residue was purified by prep-HPLC (Column: Gemini NX C18 30×150 mm, 5 μm; Condition: water (0.05% ammonia, v/v)-ACN; Begin B: 30%; End B: 70%; Gradient Time(min): 11 min). Example 17 (9.5 mg, 15.68 μmol, 4.48% yield, 98.9% purity) was obtained as a white solid.

¹H NMR: (DMSO-d₆, 400 MHz) δ_{H}=9.66 (s, 1H), 9.20 (s, 1H), 8.90-8.78 (m, 1H), 8.65-8.54 (m, 1H), 7.72 (d, J=8.8 Hz, 1H), 7.56 (t, J=7.2 Hz, 1H), 7.45-7.31 (m, 2H), 4.04-3.45 (m, 4H), 3.00-2.90 (m, 4H), 2.46-2.32 (m, 13H), 2.24-2.13 (m, 5H), 1.80-1.63 (m, 2H).

HPLC: Rₜ=2.85 min in 8 min chromatography, Ultimate XB-C18 3.0*50 mm, 3 μm, purity 98.95%.

LCMS: Rₜ=1.688 min in 4 min chromatography, Xtimate C18, 3 μm, 2.1*30 mm, purity 99.76%, MS ESI calcd. for 598.26 [M+H]⁺ 599.26, found 599.

### Example 18. 1-[3-[(2-chloro-4-fluoro-3-methyl-5-nitro-benzoyl)amino]-4-(4-methylpiperazin-1-yl)phenyl]-N-(3-morpholinopropyl)triazole-4-carboxamide (Compound 18, Table 1)

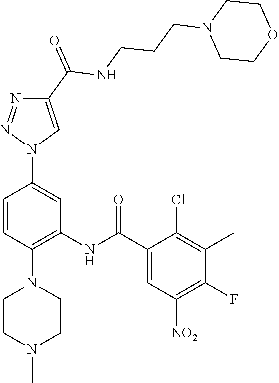 [SMILES: Cc1c(F)c([N+](=O)[O-])cc(C(=O)Nc2cc(-n3cc(C(=O)NCCCN4CCOCC4)nn3)ccc2N2CCN(C)CC2)c1Cl]

The title compound was synthesized by Scheme 7 (Example 16). To a solution of 1-(3-amino-4-(4-methylpiperazin-1-yl)phenyl)-N-(3-morpholinopropyl)-1H-1,2,3-triazole-4-carboxamide (150 mg, 350.03 mol, 1 eq.) and pyridine (55.4 mg, 700.07 μmol, 56.51 μL, 2 eq.) in DCM (5 mL) was added 2-chloro-4-fluoro-3-methyl-5-nitro-benzoyl chloride (133 mg, 525.05 μmol, 1.5 eq.) in DCM (5 mL). The reaction mixture was stirred at 0° C. for 0.5 hr to give a brown mixture. The reaction mixture was concentrated to dryness. The residue was purified by prep-HPLC (Column: Xtimate C18 30×100 mm; 3 μm; Condition: water (0.225% formic acid, v/v)-ACN; Begin B: 2%, End B: 32%; Gradient Time(min): 8 min). Example 18 (36.3 mg, 53.98 μmol, 15.42% yield, 99.2% purity, 0.5FA) was obtained as a white solid.

¹H NMR: (DMSO-d₆, 400 MHz) δ_{H}=9.97 (s, 1H), 9.20 (s, 1H), 8.84 (t, J=5.6 Hz, 1H), 8.55 (d, J=2.0 Hz, 1H), 8.38 (d, J=7.6 Hz, 1H), 8.14 (s, 1H), 7.76 (dd, J=2.8, 8.8 Hz, 1H), 7.38 (d, J=8.8 Hz, 1H), 3.65-3.57 (m, 3H), 3.42-3.37 (m, 3H), 3.02-2.95 (m, 4H), 2.62-2.56 (m, 4H), 2.46-2.37 (m, 9H), 2.28 (s, 3H), 1.78-1.66 (m, 2H).

HPLC: Rₜ=2.92 min in 8 min chromatography, Ultimate XB-C18 3.0*50 mm, 3 m, purity 99.21%.

LCMS: Rₜ=1.715 min in 4 min chromatography, Xtimate C18, 3 m, 2.1*30 mm, purity 99.31%, MS ESI calcd. for 643.24 [M+H]⁺644.24, found 644.3.

### Example 19. Synthesis of 1-(3-(4-fluoro-3-nitrobenzamido)-4-(4-methylpiperazin-1-yl)phenyl)-N-(3-morpholinopropyl)-1H-1,2,3-triazole-4-carboxamide (Compound 19, Table 1)

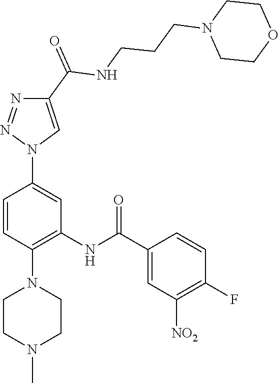 [SMILES: CN1CCN(c2ccc(-n3cc(C(=O)NCCCN4CCOCC4)nn3)cc2NC(=O)c2ccc(F)c([N+](=O)[O-])c2)CC1]

The title compound was synthesized according the Scheme 7 (Example 16). To a solution of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) in DCM (1.5 mL) was added 4-fluoro-3-nitrobenzoyl chloride (107 mg, 525.04 μmol, 1.5 eq.), then N,N-diethylethanamine (106 mg, 1.05 mmol, 146.16 μL, 3 eq.). The mixture was stirred at 25° C. for 14 hr. The mixture was diluted with DCM (100 mL), and washed with brine (50 mL×3). The organic layer was dried over Na₂SO₄, filtered and concentrated under reduced pressure to give a residue. The residue was purified by prep-HPLC (Column: Gemini NX C18 80×40 mm, 3 μm; Mobile Phase A: purified water (0.05% ammonia, v/v, 10 mM NH₄HCO₃); Mobile Phase B: acetonitrile; Gradient: 29-53% B in 8 min). Example 19 (43 mg, 69.90 μmol, 19.97% yield, 96.83% purity) was obtained as a white solid.

¹H NMR: (DMSO-d₆, 400 MHz) δ_{H}=10.05 (s, 1H), 9.21 (s, 1H), 8.84 (t, J=5.6 Hz, 1H), 8.72 (dd, J=2.4, 7.2 Hz, 1H), 8.56 (d, J=2.8 Hz, 1H), 8.43-8.36 (m, 1H), 7.89-7.74 (m, 2H), 7.44 (d, J=8.8 Hz, 1H), 3.61 (t, J=4.8 Hz, 4H), 2.96 (t, J=4.8 Hz, 4H), 2.58-2.51 (m, 6H), 2.41-2.34 (m, 6H), 2.25 (s, 3H), 1.77-1.65 (m, 2H).

HPLC: Rₜ=2.65 min in 8 min chromatography, Ultimate XB-C18 3.0*50 mm, 3 μm, purity 96.83%.

LCMS: Rₜ=1.571 min in 4 min chromatography, Xtimate C18, 3 μm, 2.1*30 mm, purity 98.96%, MS ESI calcd. for 595.27 [M+H]⁺ 596.27, found 596.3.

### Example 20. Synthesis of 1-(3-(4-fluoro-3-methylbenzamido)-4-(4-methylpiperazin-1-yl)phenyl)-N-(3-morpholinopropyl)-1H-1,2,3-triazole-4-carboxamide (Compound 20, Table 1)

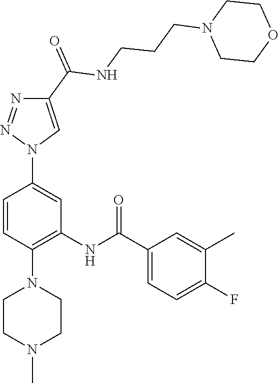 [SMILES: Cc1cc(C(=O)Nc2cc(-n3cc(C(=O)NCCCN4CCOCC4)nn3)ccc2N2CCN(C)CC2)ccc1F]

The title compound was synthesized according to Scheme 7 (Example 16). To a solution of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) in pyridine (1.5 mL) was added 4-fluoro-3-methyl-benzoic acid (54 mg, 350.03 mol, 1 eq.) and EDCI (101 mg, 525.04 μmol, 1.5 eq.). The mixture was stirred at 100° C. for 14 hr. The mixture was diluted with DCM (100 mL), and washed with brine (50 mL×3). The organic layer was dried over Na₂SO₄, filtered and concentrated under reduced pressure to give a residue. The residue was purified by prep-HPLC (Column: Gemini NX C18 80×40 mm, 3 m; Mobile Phase A: water (0.05% ammonia, v/v, 10 mM NH₄HCO₃); Mobile Phase B: acetonitrile; Gradient: 33-55% B in 8 min.). Example 20 (32.6 mg, 56.16 μmol, 16.05% yield, 97.28% purity) was obtained as a white solid.

¹H NMR: (DMSO-d₆, 400 MHz) δ_{H}=9.68 (s, 1H), 9.21 (s, 1H), 8.84 (t, J=5.6 Hz, 1H), 8.69 (d, J=2.4 Hz, 1H), 7.95-7.84 (m, 2H), 7.72 (dd, J=2.8, 8.8 Hz, 1H), 7.49-7.35 (m, 2H), 3.62 (t, J=4.8 Hz, 4H), 2.95 (t, J=4.8 Hz, 4H), 2.61-2.52 (m, 6H), 2.41-2.34 (m, 9H), 2.26 (s, 3H), 1.77-1.65 (m, 2H).

HPLC: Rₜ=2.80 min in 8 min chromatography, Ultimate XB-C18 3.0*50 mm, 3 m, purity 97.28%.

LCMS: Rₜ=1.677 min in 4 min chromatography, Xtimate C18, 3 m, 2.1*30 mm, purity 96.272%, MS ESI calcd. for 564.30 [M+H]⁺ 565.30, found 565.4.

### Example 21. Synthesis of 1-(3-(4-fluoro-3-(trifluoromethyl)benzamido)-4-(4-methylpiperazin-1-yl)phenyl)-N-(3-morpholinopropyl)-1H-1,2,3-triazole-4-carboxamide (Compound 21, Table 1)

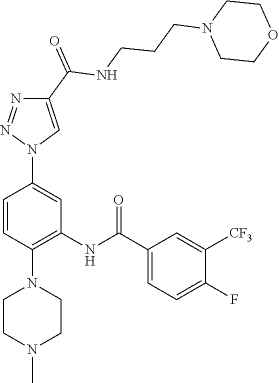 [SMILES: CN1CCN(c2ccc(-n3cc(C(=O)NCCCN4CCOCC4)nn3)cc2NC(=O)c2ccc(F)c(C(F)(F)F)c2)CC1]

The title compound was synthesized according to Scheme 7 (Example 16). To a solution of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) in pyridine (1.5 mL) was added 4-fluoro-3-(trifluoromethyl)benzoic acid (73 mg, 350.03 μmol, 1 eq.) and EDCI (101 mg, 525.04 μmol, 1.5 eq.). The mixture was stirred at 100° C. for 14 hr. The mixture was diluted with DCM (100 mL), and washed with brine (50 mL×3). The organic layer was dried over Na₂SO₄, filtered and concentrated under reduced pressure to give a residue. The residue was purified by prep-HPLC (Column: Gemini NX C18 30×150 mm, 5 μm; Mobile Phase A: purified water (0.05% ammonia, v/v); Mobile Phase B: acetonitrile; Gradient: 40-60% B in 11 min.). Example 21 (30.9 mg, 48.84 μmol, 13.95% yield, 97.78% purity) was obtained as a white solid.

¹H NMR: (DMSO-d₆, 400 MHz) δ_{H}=10.03-9.95 (m, 1H), 9.21 (s, 1H), 8.85 (t, J=5.6 Hz, 1H), 8.59 (d, J=2.4 Hz, 1H), 8.40-8.27 (m, 2H), 7.83-7.72 (m, 2H), 7.44 (d, J=8.8 Hz, 1H), 3.62 (t, J=4.8 Hz, 4H), 2.96 (t, J=4.4 Hz, 4H), 2.56-2.52 (m, 6H), 2.41-2.33 (m, 6H), 2.24 (s, 3H), 1.81-1.63 (m, 2H).

HPLC: Rₜ=3.01 min in 8 min chromatography, Ultimate XB-C18 3.0*50 mm, 3 μm, purity 97.78%.

LCMS: Rₜ=1.799 min in 4 min chromatography, Xtimate C18, 3 μm, 2.1*30 mm, purity 98.318%, MS ESI calcd. for 618.27 [M+H]⁺ 619.27, found 619.

### Example 22. Synthesis of 1-(3-(4-hydroxy-2-(trifluoromethyl)benzamido)-4-(4-methylpiperazin-1-yl)phenyl)-N-(3-morpholinopropyl)-1H-1,2,3-triazole-4-carboxamide (Compound 22, Table 1)

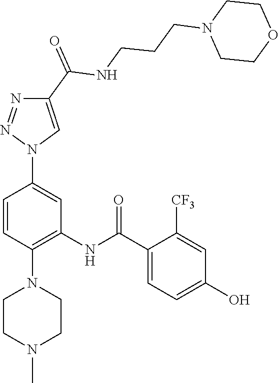 [SMILES: CN1CCN(c2ccc(-n3cc(C(=O)NCCCN4CCOCC4)nn3)cc2NC(=O)c2ccc(O)cc2C(F)(F)F)CC1]

The title compound was synthesized according to Scheme 7 (Example 16). To a solution of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) in DCM (2 mL) was added 4-hydroxy-2-(trifluoromethyl)benzoyl chloride (117.91 mg, 525.05 μmol, 1.5 eq.) and pyridine (138 mg, 1.75 mmol, 141.26 μL, 5 eq.). The mixture was stirred at 25° C. for 14 hr. The mixture was diluted with DCM (100 mL), and washed with brine (50 mL×3). The organic layer was dried over Na₂SO₄, filtered and concentrated under reduced pressure to give a residue. The residue was purified by prep-HPLC (Column: Waters Xbridge 150*25 mm*5 μm; Mobile Phase A: purified water (0.05% NH₃H2O+10 mM NH₄HCO₃); Mobile Phase B: acetonitrile; Gradient: 18-48% B in 10 min). Example 22 (3 mg, 4.66 μmol, 1.33% yield, 95.78% purity) was obtained as a white solid.

¹H NMR: (DMSO-d₆, 400 MHz) δ_{H}=9.40 (s, 1H), 9.19 (s, 1H), 8.85-8.80 (m, 1H), 8.60-8.57 (m, 1H), 7.73-7.68 (m, 1H), 7.62 (s, 1H), 7.42 (d, J=8.8 Hz, 1H), 7.18 (d, J=2.4 Hz, 2H), 3.61 (t, J=4.8 Hz, 4H), 2.92 (t, J=4.8 Hz, 4H), 2.50-2.42 (m, 6H), 2.40-2.34 (m, 6H), 2.40-2.33 (m, 1H), 2.21 (s, 3H), 1.71 (s, 2H).

HPLC: Rₜ=2.54 min in 8 min chromatography, Ultimate XB-C18 3.0*50 mm, 3 μm, purity 95.78%.

LCMS: Rₜ=1.542 min in 4 min chromatography, Xtimate C18, 3 μm, 2.1*30 mm, purity 97.77%, MS ESI calcd. for 616.63 [M+H]⁺ 617.63, found 617.4.

### Example 23. Synthesis of 1-(3-(3,5-dimethylbenzamido)-4-(4-methylpiperazin-1-yl)phenyl)-N-(3-morpholinopropyl)-1H-1,2,3-triazole-4-carboxamide (Compound 23, Table 1)

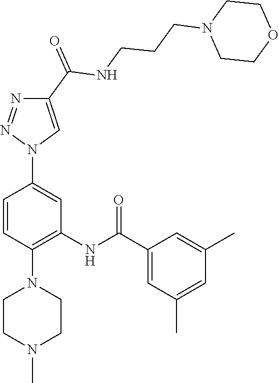 [SMILES: Cc1cc(C)cc(C(=O)Nc2cc(-n3cc(C(=O)NCCCN4CCOCC4)nn3)ccc2N2CCN(C)CC2)c1]

The title compound was synthesized according to Scheme 7 (Example 16). To a mixture of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) and 3,5-dimethylbenzoic acid (53 mg, 350.03 μmol, 1 eq.) in pyridine (1 mL) was added EDCI (101 mg, 525.05 μmol, 1.5 eq.) in one portion at 25° C. The mixture was stirred at 25° C. for 5 min, then heated to 80° C. and stirred for 10 hours. The mixture was concentrated to remove pyridine. The residue was purified by prep. HPLC (Column: Gemini NX C18 30×150 mm, 5 μm; mobile phase A: [purified water (0.05% ammonia, v/v)]; mobile phase B: ACN; B %: 33-63%; 11 min). Example 23 (21.1 mg, 36.42 mol, 10.41% yield, 96.79% purity) was obtained as yellow solid.

¹H NMR: (DMSO-d₆, 400 MHz) δ_{H}=9.68 (s, 1H), 9.20 (s, 1H), 8.87-8.78 (m, 2H), 7.68 (dd, J=2.8, 8.8 Hz, 1H), 7.57 (s, 2H), 7.48 (d, J=8.8 Hz, 1H), 7.30 (s, 1H), 3.60 (t, J=4.8 Hz, 4H), 3.37 (s, 2H), 2.94 (t, J=4.8 Hz, 4H), 2.53 (d, J=10.4 Hz, 4H), 2.42-2.31 (m, 12H), 2.25 (s, 3H), 1.70 (quin, J=6.8 Hz, 2H).

HPLC: Rₜ=2.89 min in 8 min chromatography, Ultimate XB-C18 3.0*50 mm, 3 m, purity 96.79%.

LCMS: Rₜ=2.73 min in 4 min chromatography, XBridge Shield RP18, 5 m, 2.1*50 mm, purity 99.431%, MS ESI calcd. for 560.32 [M+H]⁺561.32, found 561.4.

### Example 24. Synthesis of 1-(3-(3,5-dichlorobenzamido)-4-(4-methylpiperazin-1-yl)phenyl)-N-(3-morpholinopropyl)-1H-1,2,3-triazole-4-carboxamide (Compound 24, Table 1)

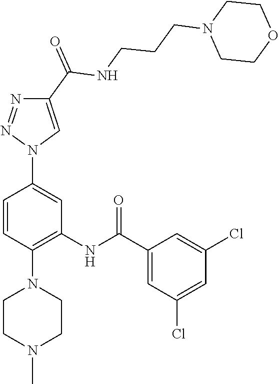 [SMILES: CN1CCN(c2ccc(-n3cc(C(=O)NCCCN4CCOCC4)nn3)cc2NC(=O)c2cc(Cl)cc(Cl)c2)CC1]

The title compound was synthesized according to Scheme 7 (Example 16). To a solution of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) in pyridine (2 mL) was added 3,5-dichlorobenzoic acid (74 mg, 385.04 μmol, 1.1 eq.) and EDCI (101 mg, 525.05 μmol, 1.5 eq.). The mixture was stirred at 60° C. for 2 hr. The reaction mixture was concentrated under reduced pressure to remove pyridine to give a residue. The residue was purified by prep-HPLC (Column: Gemini NX C18 30×150 mm, 5 μm; Mobile Phase A: purified water (0.05% ammonia, v/v); Mobile Phase B: acetonitrile; Gradient: 25-80% B in 11 min.) Example 24 (22.3 mg, 36.76 μmol, 10.50% yield, 99.17% purity) was obtained as a white solid.

¹H NMR: (DMSO-d₆, 400 MHz) δ_{H}=9.94 (s, 1H), 9.20 (s, 1H), 8.84 (t, J=5.6 Hz, 1H), 8.53 (d, J=2.4 Hz, 1H), 8.02-7.89 (m, 3H), 7.76 (dd, J=2.4, 8.8 Hz, 1H), 7.43 (d, J=8.8 Hz, 1H), 3.62 (t, J=4.4 Hz, 5H), 3.44-3.39 (m, 2H), 3.05-2.85 (m, 5H), 2.47-2.30 (m, 8H), 2.24 (s, 3H), 1.77-1.65 (m, 2H).

HPLC: Rₜ=2.99 min in 8 min chromatography, Ultimate XB-C18 3.0*50 mm, 3 m, purity 99.17%.

LCMS: Rₜ=1.865 min in 4 min chromatography, Xtimate C18, 3 m, 2.1*30 mm, purity 99.157%, MS ESI calcd. for 600.21 [M+H]⁺ 601.21, found 601.3.

### Example 25. Synthesis of 1-(3-(3-hydroxy-5-(trifluoromethyl)benzamido)-4-(4-methylpiperazin-1-yl)phenyl)-N-(3-morpholinopropyl)-1H-1,2,3-triazole-4-carboxamide (Compound 25, Table 1)

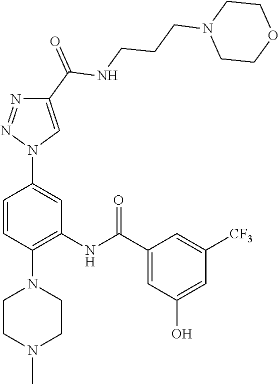 [SMILES: CN1CCN(c2ccc(-n3cc(C(=O)NCCCN4CCOCC4)nn3)cc2NC(=O)c2cc(O)cc(C(F)(F)F)c2)CC1]

The title compound was synthesized according to Scheme 7 (Example). To a mixture of 1-(3-amino-4-(4-methylpiperazin-1-yl)phenyl)-N-(3-morpholinopropyl)-1H-1,2,3-triazole-4-carboxamide (150 mg, 350.03 μmol, 1 eq) in DCM (5 mL) and 3-hydroxy-5-(trifluoromethyl)benzoyl chloride (118 mg, 525.04 mol, 1.5 eq) was added pyridine (55 mg, 700.06 μmol, 56.50 μL, 2 eq) in one portion at 0° C. The mixture was stirred at 25° C. and stirred for 1 hour. The mixture was concentrated to remove DCM and pyridine. The residue was purified by prep. HPLC (Column: Xtimate C18 30×100 mm, 3 μm; mobile phase A: [purified water (0.225% formic acid, v/v)]; mobile phase B: ACN; B %: 2-32%; 8 min). Example 25 (51.6 mg, 82.94 μmol, 23.70% yield, 99.12% purity) was obtained as a yellow solid.

¹H NMR: (DMSO-d₆, 400 MHz) δ_{H}=9.88 (s, 1H), 9.21 (s, 1H), 8.68 (d, J=2.5 Hz, 1H), 8.14 (s, 1H), 7.73 (dd, J=2.8, 8.8 Hz, 1H), 7.69 (s, 1H), 7.62 (s, 1H), 7.47 (d, J=8.8 Hz, 1H), 7.31 (s, 1H), 3.63- 3.60 (m, 6H), 3.38-3.31 (m, 2H), 2.96 (t, J=4.4 Hz, 4H), 2.56 (s, 3H), 2.43-2.35 (m, 6H), 2.26 (s, 3H), 1.71 (quin, J=6.8 Hz, 2H).

HPLC: Rₜ=2.83 min in 8 min chromatography, Ultimate XB-C18 3.0*50 mm, 3 m, purity 99.12%.

LCMS: Rₜ=1.688 min in 4 min chromatography, Xtimate C18, 3 m, 2.1*30 mm, purity 99.215%, MS ESI calcd. for 616.27 [M+H]⁺617.27, found 617.4.

### Example 26. Synthesis of 1-(3-(2,3-difluoro-5-hydroxybenzamido)-4-(4-methylpiperazin-1-yl)phenyl)-N-(3-morpholinopropyl)-1H-1,2,3-triazole-4-carboxamide (Compound 26, Table 1)

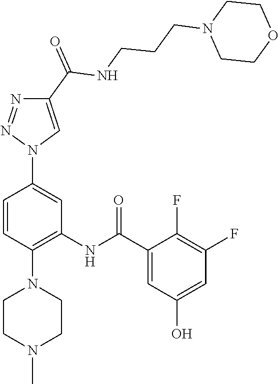 [SMILES: CN1CCN(c2ccc(-n3cc(C(=O)NCCCN4CCOCC4)nn3)cc2NC(=O)c2cc(O)cc(F)c2F)CC1]

The title compound was synthesized according to Scheme 7 (Example 16). To a mixture of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) and 2, 3-difluoro-5-hydroxy-benzoyl chloride (91.4 mg, 525.05 μmol, 1.5 eq.) in DMF (2 mL) was added pyridine (55.4 mg, 700.07 μmol, 56.51 μL, 2 eq.) in one portion at 0° C. The mixture was stirred at 25° C. for 1 hr. The mixture was concentrated in reduce pressure to remove pyridine and DMF. The residue was purified by prep. HPLC (Column: Xtimate C18 30×100 mm, 3 μm; mobile phase: [purified water (0.225% formic acid, v/v)-ACN]; B %: 0-30%; 8 min). Example 26 (35 mg, 59.46 μmol, 16.99% yield, 98.75% purity) was obtained as brown solid.

¹H NMR: (DMSO-d₆, 400 MHz) δ_{H}=9.20 (s, 1H), 8.88-8.80 (m, 2H), 7.69 (dd, J=2.8, 8.8 Hz, 1H), 7.46-7.36 (m, 2H), 6.80 (d, J=6.4 Hz, 1H), 3.62 (t, J=4.8 Hz, 3H), 3.34-3.33 (m, 5H), 2.97 (s, 3H), 2.71-2.65 (m, 3H), 2.53 (s, 2H), 2.45-2.31 (m, 9H), 1.72 (quin, J=6.8 Hz, 2H).

HPLC: Rₜ=2.69 min in 8 min chromatography, Ultimate XB-C18 3.0*50 mm, 3 m, purity 98.75%.

LCMS: Rₜ=1.638 min in 4 min chromatography, Xtimate C18, 3 m, 2.1*30 mm, purity 97.878%, MS ESI calcd. for 584.27 [M+H]⁺585.27, found 585.4.

### Example 27. Synthesis of 1-methyl-N-(2-(4-methylpiperazin-1-yl)-5-(4-((3-morpholinopropyl)carbamoyl)-1H-1,2,3-triazol-1-yl)phenyl)-1H-indazole-3-carboxamide (Compound 27, Table 1)

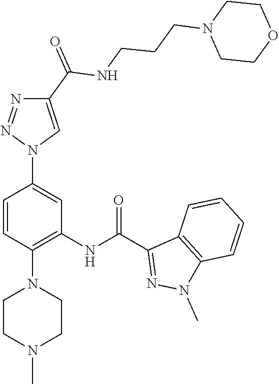 [SMILES: CN1CCN(c2ccc(-n3cc(C(=O)NCCCN4CCOCC4)nn3)cc2NC(=O)c2nn(C)c3ccccc23)CC1]

The title compound was synthesized according to Scheme 7 (Example 16). To a mixture of 1-(3-amino-4-(4-methylpiperazin-1-yl)phenyl)-N-(3-morpholinopropyl)-1H-1,2,3-triazole-4-carboxamide (150 mg, 350.03 μmol, 1 eq.) and 1-methylindazole-3-carboxylic acid (62 mg, 350.03 μmol, 1 eq.) in pyridine (1 mL) was added EDCI (101 mg, 525.05 μmol, 1.5 eq.) in one portion at 25° C. The mixture was heated to 100° C. and stirred for 10 hrs. The mixture was concentrated to remove pyridine. The residue was purified by prep. HPLC (Column: Gemini NX C18 30×150 mm, 5 μm; mobile phase: [purified water (0.05% ammonia, v/v)-ACN]; B %: 33-63%; 11 min). Example 27 (24 mg, 40.40 μmol, 11.54% yield, 98.34% purity) was obtained as a yellow solid.

¹H NMR: (DMSO-d₆, 400 MHz) δ_{H}=10.19 (s, 1H), 9.23 (s, 1H), 9.07 (d, J=2.4 Hz, 1H), 8.87 (t, J=5.6 Hz, 1H), 8.27 (d, J=8 Hz, 1H), 7.84 (d, J=8.8 Hz, 1H), 7.67 (dd, J=2.8, 8.8 Hz, 1H), 7.60-7.48 (m, 2H), 7.39 (t, J=7.6 Hz, 1H), 4.24 (s, 3H), 3.63 (t, J=4.8 Hz, 4H), 3.33-3.30 (m, 3H), 2.98 (t, J=4.8 Hz, 4H), 2.76-2.60 (m, 3H), 2.43-2.30 (m, 8H), 2.13-2.05 (m, 1H), 1.72 (quin, J=6.8 Hz, 2H).

HPLC: Rₜ=3.561 min in 8 min chromatography, XBridge Shield RP18, 5 m, 2.1*50 mm, 3 um, purity 98.397%.

LCMS: Rₜ=2.726 min in 4 min chromatography, XBridge Shield RP18, 5 m, 2.1*50 mm, purity 96.722%, MS ESI calcd. for 586.31 [M+H]⁺587.31, found 587.5.

### Example 28. Synthesis of 1-(5-(2-chloro-4-fluoro-3-methylbenzamido)-2-fluoro-4-((3S,5R)-3,4,5-trimethylpiperazin-1-yl)phenyl)-N-(3-morpholinopropyl)-1H-1,2,3-triazole-4-carboxamide (Compound 28, Table 1)

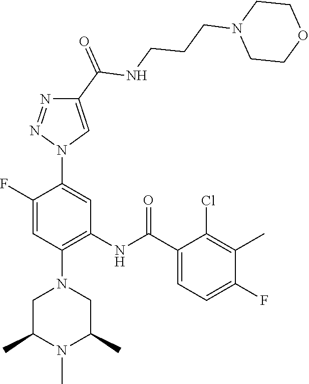 [SMILES: Cc1c(F)ccc(C(=O)Nc2cc(-n3cc(C(=O)NCCCN4CCOCC4)nn3)c(F)cc2N2C[C@@H](C)N(C)[C@@H](C)C2)c1Cl]

Steps 1: To a mixture of methyl 1-(5-amino-2-fluoro-4-((3S,5R)-3,4,5-trimethylpiperazin-1-yl)phenyl)-1H-1,2,3-triazole-4-carboxylate (100 mg, 275.94 μmol, 1 eq.) in DCM (10 mL) was added TEA (140 mg, 1.38 mmol, 192.03 μL, 5 eq.) and 2-chloro-4-fluoro-3-methylbenzoyl chloride (115 mg, 555.48 mol, 2.01 eq.) in one portion at 0° C. The mixture was stirred at 0° C. for 12 hrs. The residue was concentrated under reduce pressure. The residue was purified by silica gel chromatography (column weight: 12 g, diameter: 100 mm, 100-200 mesh silica gel, Dichloromethane: Methanol=10:1). 1-(5-(2-Chloro-4-fluoro-3-methylbenzamido)-2-fluoro-4-((3S,5R)-3,4,5-trimethylpiperazin-1-yl)phenyl)-1H-1,2,3-triazole-4-carboxylate (120 mg, 161.26 μmol) was obtained as a yellow solid.

¹H NMR: (DMSO-d₆, 400 MHz) δ_{H}=9.97 (s, 1H), 8.49 (s, 1H), 8.42 (d, J=5.2 Hz, 1H), 8.34 (d, J=8.0 Hz, 1H), 7.19 (d, J=13.2 Hz, 1H), 7.03-6.96 (m, 1H), 3.21-3.17 (m, 2H), 3.16 (s, 6H), 3.11-3.06 (m, 2H), 2.94-2.78 (m, 4H), 2.58-2.51 (m, 3H), 1.31 (s, 6H).

Step 2: To a mixture of 1-(5-(2-chloro-4-fluoro-3-methylbenzamido)-2-fluoro-4-((3S,5R)-3,4,5-trimethylpiperazin-1-yl)phenyl)-1H-1,2,3-triazole-4-carboxylate (120 mg, 225.15 μmol, 1 eq.) in THF (5 mL) and H₂O (1 mL) was added LiOH·H₂O (19 mg, 450.31 μmol, 2 eq.) in one portion. The mixture was stirred at 25° C. for 12 hrs. The residue was concentrated under reduce pressure. The crude product was used in the next step without further purification. 1-(5-(2-Chloro-4-fluoro-3-methylbenzamido)-2-fluoro-4-((3S,5R)-3,4,5-trimethylpiperazin-1-yl)phenyl)-1H-1,2,3-triazole-4-carboxylic acid (100 mg, 130.30 μmol) was obtained as a yellow solid.

Step 3: To a mixture of 1-(5-(2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (100 mg, 192.70 μmol, 1 eq.) and 3-morpholinopropan-1-amine (28 mg, 192.70 μmol, 28.16 μL, 1 eq.) in DMF (3 mL) was added DIEA (75 mg, 578.10 μmol, 100.69 μL, 3 eq.) and HATU (125 mg, 327.59 μmol, 1.7 eq.) in one portion. The mixture was stirred at 25° C. for 12 hrs. The residue was poured into water (20 mL). The aqueous phase was extracted with ethyl acetate (20 mL*3). The combined organic phase was washed with brine (20 mL), dried with anhydrous Na₂SO₄, filtered and concentrated in vacuum. The residue was purified by prep. HPLC (Column: Phenomenex Gemini-NX C18 75*30 mm*3 μm; mobile phase: [water (0.04% NH₃H2O+10 mM NH₄HCO₃)-ACN]; B %: 23%-63%; 11 min). Example 28 (17.2 mg, 26.27 μmol, 13.63% yield, 98.54% purity) was obtained as a light yellow solid.

¹H NMR: (DMSO-d₆, 400 MHz) δ_{H}=9.79 (s, 1H), 8.96 (d, J=1.6 Hz, 1H), 8.85 (t, J=5.6 Hz, 1H), 8.24 (d, J=8.0 Hz, 1H), 7.50 (dd, J=6.4, 8.0 Hz, 1H), 7.39-7.26 (m, 2H), 3.60 (t, J=4.8 Hz, 4H), 3.34-3.33 (m, 3H), 3.13 (d, J=11.2 Hz, 2H), 2.57-2.52 (m, 2H), 2.36 (t, J=6.8 Hz, 7H), 2.32 (d, J=1.6 Hz, 3H), 2.18 (s, 3H), 1.75-1.65 (m, 2H), 1.03 (d, J=6.0 Hz, 6H).

HPLC: Rₜ=2.06 min in 8 min chromatography, Ultimate XB-C18 3.0*50 mm, 3 μm, purity 98.54%.

LCMS: Rₜ=0.918 min in 4 min chromatography, Xtimate C18.3 μm, 2.1*30 mm, purity 98.477%, MS ESI calcd. for 644.28 [M+H]⁺645.28, found 645.4.

### Example 29. Synthesis of 1-(5-(5-amino-2-chloro-4-fluoro-3-methylbenzamido)-2-fluoro-4-((3S,5R)-3,4,5-trimethylpiperazin-1-yl)phenyl)-N,N-diethyl-1H-1,2,3-triazole-4-carboxamide (Compound 29, Table 1)

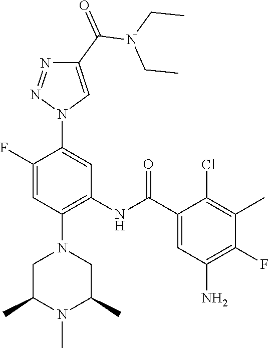 [SMILES: CCN(CC)C(=O)c1cn(-c2cc(NC(=O)c3cc(N)c(F)c(C)c3Cl)c(N3C[C@@H](C)N(C)[C@@H](C)C3)cc2F)nn1]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (100 mg, 187.28 μmol, 1 eq.) and N-ethylethanamine (27 mg, 374.56 μmol, 38.58 μL, 2 eq.) in DMF (5 mL) was added HATU (107 mg, 280.92 mol, 1.5 eq) and DIEA (73 mg, 561.84 μmol, 97.86 μL, 3 eq.). The mixture was stirred at 25° C. for 12 hr. The reaction mixture was concentrated directly. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 29-69% B in 11 min). Example 29 (10 mg, 16.82 μmol, 8.98% yield, 99.101% purity) was obtained as a red solid.

¹H NMR: (DMSO-d₆, 400 MHz) δ_{H}=9.61 (s, 1H), 8.93 (d, J=1.6 Hz, 1H), 8.23 (d, J=8.0 Hz, 1H), 7.33 (d, J=12.4 Hz, 1H), 6.81 (d, J=9.2 Hz, 1H), 5.49 (s, 2H), 3.75 (q, J=6.8 Hz, 2H), 3.47 (q, J=7.2 Hz, 2H), 3.08 (d, J=10.8 Hz, 2H), 2.58-2.51 (m, 2H), 2.39-2.28 (m, 2H), 2.24 (d, J=2.4 Hz, 3H), 2.19 (s, 3H), 1.24 (t, J=6.8 Hz, 3H), 1.16 (t, J=7.2 Hz, 3H), 1.02 (d, J=6.0 Hz, 6H).

HPLC: Rₜ=2.973 min in 8 min chromatography, XBridge Shield RP18, 5 μm, 2.1*50 mm, purity 99.10%.

LCMS: Rₜ=2.368 min in 4 min chromatography, XBridge Shield RP18, 5 μm, 2.1*50 mm, purity 98.77%, MS ESI calcd. for 588.25 [M+H]⁺589.25, found 589.3.

### Example 30. Synthesis of 5-amino-2-chloro-4-fluoro-N-(4-fluoro-5-(4-(morpholine-4-carbonyl)-1H-1,2,3-triazol-1-yl)-2-((3S,5R)-3,4,5-trimethylpiperazin-1-yl)phenyl)-3-methylbenzamide (Compound 30, Table 1)

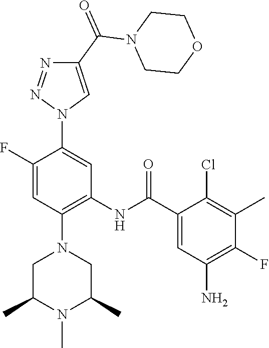 [SMILES: Cc1c(F)c(N)cc(C(=O)Nc2cc(-n3cc(C(=O)N4CCOCC4)nn3)c(F)cc2N2C[C@@H](C)N(C)[C@@H](C)C2)c1Cl]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (120 mg, 224.74 μmol, 1 eq.) and morpholine (39 mg, 449.47 μmol, 39.55 μL, 2 eq.) in DMF (5 mL) was added HATU (128 mg, 337.11 μmol, 1.5 eq.) and DIEA (87 mg, 674.21 μmol, 117.44 μL, 3 eq.). The mixture was stirred at 25° C. for 12 hr. The reaction mixture was concentrated directly. The residue was purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 20-60% B in 11 min). Example 30 (18 mg, 29.34 μmol, 13.06% yield, 98.3% purity) was obtained as a white solid.

¹H NMR: (DMSO-d₆, 400 MHz) δ_{H}=9.60 (s, 1H), 8.98 (d, J=1.2 Hz, 1H), 8.23 (d, J=8.0 Hz, 1H), 7.34 (d, J=12.4 Hz, 1H), 6.81 (d, J=9.2 Hz, 1H), 5.48 (s, 2H), 4.03 (s, 2H), 3.67 (s, 6H), 3.09 (d, J=10.8 Hz, 2H), 2.59-2.52 (m, 2H), 2.35 (s, 2H), 2.27-2.15 (m, 6H), 1.03 (d, J=6.0 Hz, 6H).

HPLC: Rₜ=3.551 min in 8 min chromatography, XBridge Shield RP18, 5 μm, 2.1*50 mm, purity 98.31%.

LCMS: Rₜ=2.719 min in 4 min chromatography, XBridge Shield RP18, 5 μm, 2.1*50 mm, purity 98.70%, MS ESI calcd. for 602.23 [M+H]⁺603.23, found 603.4.

### Example 31. Synthesis of 5-amino-2-chloro-4-fluoro-N-(4-fluoro-5-(4-(4-methylpiperazine-1-carbonyl)-1H-1,2,3-triazol-1-yl)-2-((3S,5R)-3,4,5-trimethylpiperazin-1-yl)phenyl)-3-methylbenzamide (Compound 31, Table 1)

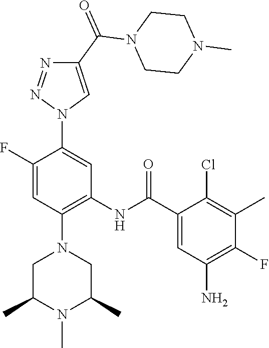 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (100 mg, 187.28 μmol, 1 eq.) and 1-methylpiperazine (38 mg, 374.56 μmol, 41.55 μL, 2 eq.) in DMF (5 mL) was added HATU (107 mg, 280.92 mol, 1.5 eq) and DIEA (73 mg, 561.84 μmol, 97.86 μL, 3 eq). The mixture was stirred at 25° C. for 12 hr. The reaction mixture was concentrated directly. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 26-66% B in 11 min.). Example 31 (13.2 mg, 20.57 mol, 10.98% yield, 96.02% purity) was obtained as a yellow solid.

¹H NMR: (DMSO-d₆, 400 MHz) δ_{H}=9.62 (s, 1H), 8.96 (d, J=1.2 Hz, 1H), 8.22 (d, J=8.0 Hz, 1H), 7.33 (d, J=12.4 Hz, 1H), 6.81 (d, J=9.2 Hz, 1H), 5.49 (s, 2H), 3.98 (s, 2H), 3.66 (s, 2H), 3.08 (d, J=10.8 Hz, 2H), 2.58-2.52 (m, 2H), 2.38 (t, J=4.8 Hz, 4H), 2.35-2.29 (m, 2H), 2.26-2.17 (m, 9H), 1.02 (d, J=6.0 Hz, 6H).

HPLC: Rₜ=3.439 min in 8 min chromatography, XBridge Shield RP18, 5 m, 2.1*50 mm, purity 96.02%.

LCMS: Rₜ=2.636 min in 4 min chromatography, XBridge Shield RP18, 5 m, 2.1*50 mm, purity 97.89%, MS ESI calcd. for 615.26 [M+H]⁺616.26, found 616.3.

### Example 32. Synthesis of 1-(5-(5-amino-2-chloro-4-fluoro-3-methylbenzamido)-2-fluoro-4-((3S,5R)-3,4,5-trimethylpiperazin-1-yl)phenyl)-N-cyclohexyl-1H-1,2,3-triazole-4-carboxamide (Compound 32, Table 1)

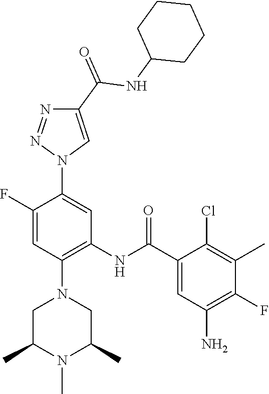 [SMILES: Cc1c(F)c(N)cc(C(=O)Nc2cc(-n3cc(C(=O)NC4CCCCC4)nn3)c(F)cc2N2C[C@@H](C)N(C)[C@@H](C)C2)c1Cl]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (100 mg, 187.28 μmol, 1 eq.) and cyclohexanamine (27.86 mg, 280.92 umol, 32.15 uL, 1.5 eq) in DMF (1 mL) was added HATU (142.42 mg, 374.56 umol, 2 eq) and DIEA (72.61 mg, 561.84 umol, 97.86 uL, 3 eq). The reaction mixture was stirred at 25° C. for 10 hr. The mixture was concentrated in vacuum. The residue was purified by prep-HPLC (Column:Phenomenex Gemini-NX C18 75*30 mm*3 um; Condition:[water (0.04% NH3H2O+10 mM NH4HCO3)ACN]; B:33%-73%, 1 1 min). Example 32 (20 mg, 31.73 umol, 16.94% yield, 97.60% purity) was obtained as a red solid.

¹H NMR (DMSO-d₆, 400 MHz) δ_{H}=9.59 (s, 1H), 8.96 (s, 1H), 8.40 (d, J=8.0 Hz, 1H), 8.23 (d, J=8.0 Hz, 1H), 7.33 (d, J=12.0 Hz, 1H), 6.82 (d, J=8.8 Hz, 1H), 5.48 (s, 2H), 3.85-3.73 (m, 1H), 3.12-3.04 (m, 2H), 2.60-2.50 (m, 2H), 2.40-2.30 (m, 2H), 2.24 (s, 3H), 2.19 (s, 3H), 1.85-1.68 (m, 4H), 1.65-1.55 (m, 4H), 1.45-1.30 (m, 5H), 1.02 (d, J=6.0 Hz, 6H)

HPLC Rₜ=3.394 min in 8 min chromatography, Xtimate C18 (2.1×30 mm, 3 μm), purity 97.599%.

LCMS Rₜ=2.230 min in 4 min chromatography, Xtimate C18 (2.1×30 mm, 3 μm), purity 97.826%, MS ESI calcd. for 614.27 [M+H]⁺615.27, found 615.4.

### Example 33. Synthesis of 1-(5-(5-amino-2-chloro-4-fluoro-3-methylbenzamido)-2-fluoro-4-((3S,5R)-3,4,5-trimethylpiperazin-1-yl)phenyl)-N-(tetrahydro-2H-pyran-4-yl)-1H-1,2,3-triazole-4-carboxamide (Compound 33, Table 1)

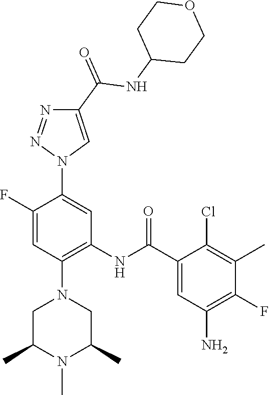 [SMILES: Cc1c(F)c(N)cc(C(=O)Nc2cc(-n3cc(C(=O)NC4CCOCC4)nn3)c(F)cc2N2C[C@@H](C)N(C)[C@@H](C)C2)c1Cl]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (150 mg, 280.92 μmol, 1 eq.) and tetrahydro-2H-pyran-4-amine (42.62 mg, 421.38 μmol, 1.5 eq.) in DMF (3 mL) was added HATU (213.63 mg, 561.84 μmol, 2 eq.) and DIEA (108.92 mg, 842.76 μmol, 146.79 μL, 3 eq.). The mixture was stirred at 25° C. for 12 hr. Concentrated under reduced pressure to give a residue. The residue was purified by prep-HPLC (Column: Phenomenex Gemini-NX C18 75*30 mm*3 μm; Mobile Phase A: purified water (0.04% NH₃H2O+10 mM NH₄HCO₃); Mobile Phase B: acetonitrile; Gradient: 23-63% B in 10 min.). Example 33 (16 mg, 25.49 μmol, 9.07% yield, 98.31% purity) was obtained as a white solid.

¹H NMR: (DMSO-d₆, 400 MHz) δ_{H}=9.59 (s, 1H), 8.98 (s, 1H), 8.61 (d, J=8.0 Hz, 1H), 8.24 (d, J=8.0 Hz, 1H), 7.33 (d, J=12.4 Hz, 1H), 6.82 (d, J=9.2 Hz, 1H), 5.47 (s, 2H), 4.11-3.98 (m, 1H), 3.88 (d, J=10.8 Hz, 2H), 3.14-2.96 (m, 3H), 2.60-2.51 (m, 3H), 2.38-2.29 (m, 2H), 2.24 (s, 3H), 2.18 (s, 3H), 1.78-1.61 (m, 4H), 1.02 (d, J=6.0 Hz, 6H).

HPLC: Rₜ=3.525 min in 8 min chromatography, XBridge Shield RP18, 5 um, 2.1*50 mm, purity 98.31%.

LCMS: Rₜ=2.709 min in 4 min chromatography, XBridge Shield RP18, 5 um, 2.1*50 mm, purity 98.77%, MS ESI calcd. for 616.25 [M+H]⁺617.25, found 316.

### Example 34. Synthesis of 1-(5-(5-amino-2-chloro-4-fluoro-3-methylbenzamido)-2-fluoro-4-((3S,5R)-3,4,5-trimethylpiperazin-1-yl)phenyl)-N-(tetrahydro-2H-pyran-4-yl)-1H-1,2,3-triazole-4-carboxamide (Compound 34, Table 1)

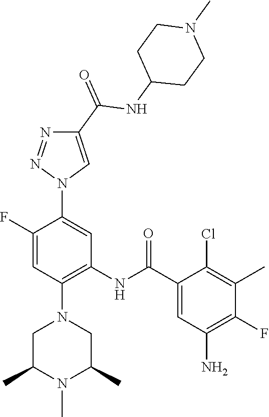 [SMILES: Cc1c(F)c(N)cc(C(=O)Nc2cc(-n3cc(C(=O)NC4CCN(C)CC4)nn3)c(F)cc2N2C[C@@H](C)N(C)[C@@H](C)C2)c1Cl]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (150 mg, 280.92 μmol, 1 eq.) and 1-methylpiperidin-4-amine (48.12 mg, 421.38 μmol, 1.5 eq.) in DMF (3 mL) was added HATU (213.63 mg, 561.84 μmol, 2 eq.) and DIEA (108.92 mg, 842.76 μmol, 146.79 μL, 3 eq.). The mixture was stirred at 25° C. for 12 hr. The reaction mixture was concentrated directly. The residue was purified by prep-HPLC (Column: Phenomenex Gemini-NX C18 75*30 mm*3 um; Mobile Phase A: purified water (0.04% NH₃H₂O+10 mM NH₄HCO₃); Mobile Phase B: acetonitrile; Gradient: 25-65% B in 10 min.). Example 34 (17.3 mg, 26.93 mol, 9.59% yield, 98.08% purity) was obtained as a white solid.

¹H NMR: (DMSO-d₆, 400 MHz) δ_{H}=9.60 (s, 1H), 8.98 (s, 1H), 8.51 (d, J=8.0 Hz, 1H), 8.25 (d, J=8.0 Hz, 1H), 7.34 (d, J=12.4 Hz, 1H), 6.83 (d, J=9.2 Hz, 1H), 5.48 (s, 2H), 3.85-3.71 (m, 1H), 3.08 (d, J=10.8 Hz, 2H), 2.77 (d, J=11.2 Hz, 2H), 2.59-2.54 (m, 2H), 2.34 (s, 2H), 2.25 (s, 3H), 2.18 (d, J=11.6 Hz, 6H), 1.95 (t, J=10.8 Hz, 2H), 1.81-1.59 (m, 4H), 1.03 (d, J=6.0 Hz, 6H).

HPLC: Rₜ=3.630 min in 8 min chromatography, XBridge Shield RP18, 5 um, 2.1*50 mm, purity 98.08%.

LCMS: Rₜ=2.804 min in 4 min chromatography, XBridge Shield RP18, 5 um, 2.1*50 mm, purity 98.535%, MS ESI calcd. for 629.28 [M+H]⁺630.28, found 630.3.

### Example 35. Synthesis of 1-(5-(5-amino-2-chloro-4-fluoro-3-methylbenzamido)-2-fluoro-4-((3S,5R)-3,4,5-trimethylpiperazin-1-yl)phenyl)-N-(3-(4-methylpiperazin-1-yl)propyl)-1H-1,2,3-triazole-4-carboxamide (Compound 35, Table 1)

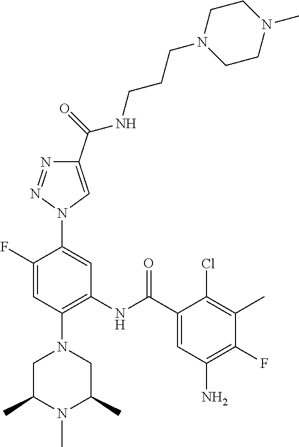 [SMILES: Cc1c(F)c(N)cc(C(=O)Nc2cc(-n3cc(C(=O)NCCCN4CCN(C)CC4)nn3)c(F)cc2N2C[C@@H](C)N(C)[C@@H](C)C2)c1Cl]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (150 mg, 280.92 μmol, 1 eq.) and 3-(4-methylpiperazin-1-yl)propan-1-amine (66.27 mg, 421.38 μmol, 1.5 eq.) in DMF (3 mL) was added HATU (213.63 mg, 561.84 μmol, 2 eq.) and DIEA (108.92 mg, 842.76 μmol, 146.79 μL, 3 eq.). The mixture was stirred at 25° C. for 12 hr. The reaction mixture was concentrated directly. The residue was purified by prep-HPLC (Column: Phenomenex Gemini-NX C18 75*30 mm*3 μm; Mobile Phase A: purified water (0.04% NH₃H2O+10 mM NH₄HCO₃); Mobile Phase B: acetonitrile; Gradient: 15-80% B in 10 min). Example 35 (14.5 mg, 21.18 μmol, 7.54% yield, 98.32% purity) was obtained as a white solid.

¹H NMR: (DMSO-d₆, 400 MHz) δ_{H}=9.60 (s, 1H), 8.95 (d, J=1.6 Hz, 1H), 8.81 (t, J=5.6 Hz, 1H), 8.23 (d, J=8.0 Hz, 1H), 7.33 (d, J=12.4 Hz, 1H), 6.82 (d, J=9.2 Hz, 1H), 5.48 (s, 2H), 3.34-3.29 (m, 4H), 3.12-3.03 (m, 2H), 2.57-2.51 (m, 3H), 2.38-2.29 (m, 8H), 2.24 (d, J=2.4 Hz, 4H), 2.18 (s, 3H), 2.14 (s, 3H), 1.73-1.64 (m, 2H), 1.02 (d, J=6.4 Hz, 6H).

HPLC: Rₜ=2.544 min in 8 min chromatography, XBridge Shield RP18, 5 um, 2.1*50 mm, purity 98.33%.

LCMS: Rₜ=2.680 min in 4 min chromatography, XBridge Shield RP18, 5 um, 2.1*50 mm, purity 97.12%, MS ESI calcd. for 672.32 [M+H]⁺673.32, found 673.

### Example 36. Synthesis of 1-(5-(5-amino-2-chloro-4-fluoro-3-methylbenzamido)-2-fluoro-4-((3S,5R)-3,4,5-trimethylpiperazin-1-yl)phenyl)-N-(cyclohexylmethyl)-1H-1,2,3-triazole-4-carboxamide (Compound 36, Table 1)

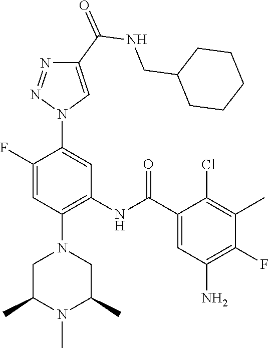 [SMILES: Cc1c(F)c(N)cc(C(=O)Nc2cc(-n3cc(C(=O)NCC4CCCCC4)nn3)c(F)cc2N2C[C@@H](C)N(C)[C@@H](C)C2)c1Cl]

To a solution of 1-(5-(5-amino-2-chloro-4-fluoro-3-methylbenzamido)-2-fluoro-4-((3S,5R)-3,4,5-trimethylpiperazin-1-yl)phenyl)-1H-1,2,3-triazole-4-carboxylic acid (150 mg, 280.92 μmol, 1 eq.) and cyclohexylmethanamine (47.70 mg, 421.38 μmol, 54.83 μL, 1.5 eq.) in DMF (3 mL) was added HATU (213.63 mg, 561.84 μmol, 2 eq.) and DIEA (108.92 mg, 842.76 μmol, 146.79 μL, 3 eq.). The mixture was stirred at 25° C. for 12 hr. The mixture was heated to 100° C. and stirred for 10 hrs. The reaction mixture was concentrated directly. The residue was purified by prep-HPLC (Column: Phenomenex Gemini-NX C18 75*30 mm*3 μm; Mobile Phase A: purified water (0.04% NH₃H2O+10 mM NH₄HCO₃); Mobile Phase B: acetonitrile; Gradient: 50-80% B in 10 min.). Example 36 (15.5 mg, 24.21 μmol, 8.62% yield, 98.29% purity) was obtained as a white solid.

¹H NMR: (DMSO-d₆, 400 MHz) δ_{H}=9.61 (s, 1H), 8.96 (d, J=1.6 Hz, 1H), 8.66 (t, J=6.0 Hz, 1H), 8.24 (d, J=8.0 Hz, 1H), 7.34 (d, J=12.4 Hz, 1H), 6.83 (d, J=9.2 Hz, 1H), 5.49 (s, 2H), 3.18-3.04 (m, 5H), 2.39-2.29 (m, 3H), 2.25 (d, J=2.4 Hz, 3H), 2.19 (s, 3H), 1.74-1.65 (m, 4H), 1.64-1.53 (m, 2H), 1.27-1.11 (m, 3H), 1.03 (d, J=6.0 Hz, 6H), 0.98-0.87 (m, 2H).

HPLC: Rₜ=3.433 min in 8 min chromatography, XBridge Shield RP18, 5 μm, 2.1*50 mm, purity 98.29%.

LCMS: Rₜ=2.715 min in 4 min chromatography, XBridge Shield RP18, 5 μm, 2.1*50 mm, purity 97.88%, MS ESI calcd. for 628.29 [M+H]⁺629.39, found 629.3.

### Example 37. Synthesis of (S)-5′-((5-amino-6-chloropyrimidin-4-yl)amino)-4′-(3,4 dimethylpiperazin-1-yl)-2′-fluoro-N,N-dimethyl-[1,1′-biphenyl]-4-carboxamide (Compound 123, Table 3)

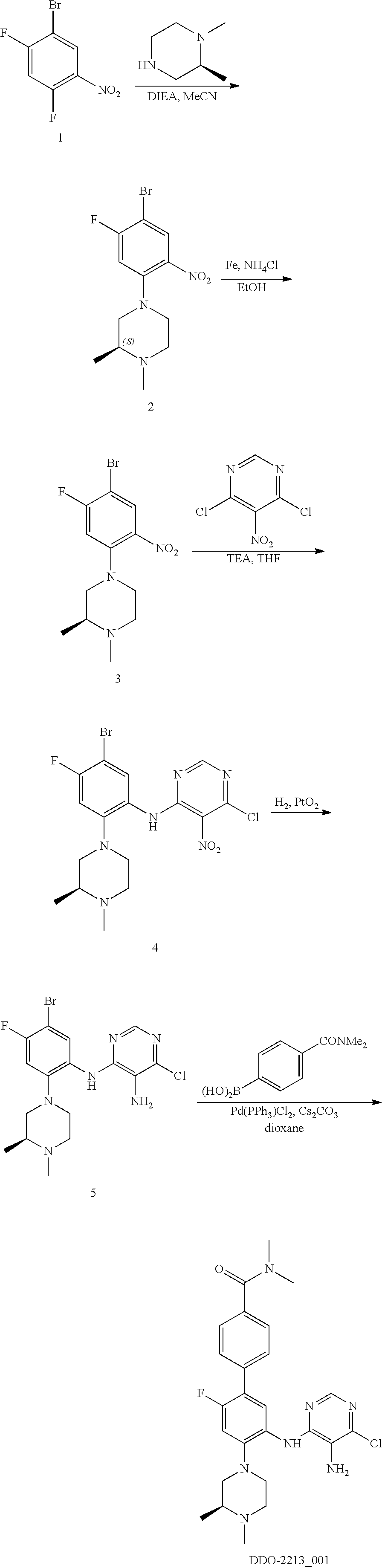

### Step 1: (S)-4-(4-bromo-5-fluoro-2-nitrophenyl)-1,2-dimethylpiperazine (Compound 2)

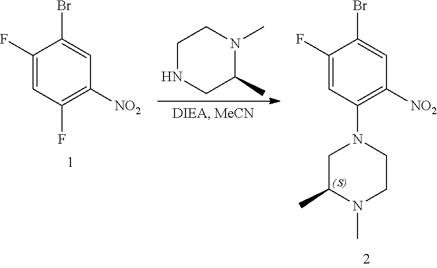

To a solution of compound 1 (2 g, 8.40 mmol, 1 eq.) in CH₃CN (20 mL) was added (2S)-1, 2-dimethylpiperazine (959 mg, 8.40 mmol, 1 eq.) and DIEA (2.17 g, 16.81 mmol, 2.93 mL, 2 eq.). The mixture was stirred at 30° C. for 2 hr. The mixture was diluted with DCM (100 mL), washed with brine (50 mL*3). The organic layer was dried over Na₂SO₄, filtered and concentrated under reduced pressure to give a residue. Compound 2 (2.43 g, 6.51 mmol, 77.47% yield) was obtained as a yellow solid. The product was used directly to the next step without further purification.

¹H NMR (CDCl₃, 400 MHz) δ_{H}=8.05 (d, J=7.2 Hz, 1H), 6.82 (d, J=10.0 Hz, 1H), 3.26-2.95 (m, 4H), 2.91-2.81 (m, 1H), 2.67-2.54 (m, 2H), 2.44-2.42 (m, 3H), 1.16 (d, J=6.4 Hz, 3H).

### Step 2: (S)-5-bromo-2-(3,4-dimethylpiperazin-1-yl)-4-fluoroaniline (Compound 3)

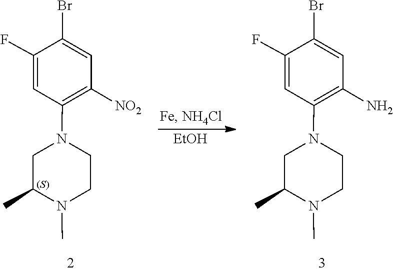

To a solution of compound 2 (2.43 g, 7.32 mmol, 1 eq.) in EtOH (22.5 mL) and H₂O (7.5 mL) was added Fe (1.23 g, 21.95 mmol, 3 eq.) and NH₄Cl (1.96 g, 36.58 mmol, 5 eq.). The mixture was stirred at 80° C. for 2 hr. The mixture was filtered to remove insolubles. The filter liquor was concentrated in vacuo. The reaction mixture was diluted with DCM (100 mL), washed with NaHCO₃ (50 mL*3). The organic layer was dried over Na₂SO₄, filtered and concentrated under reduced pressure to give a residue. The residue was purified by flash silica gel chromatography (ISCO®; 25 g SepaFlash® Silica Flash Column, Eluent of 0-10% Methanol/Dichloromethane @ 30 mL/min). Compound 3 (1.42 g, 4.14 mmol, 56.65% yield) was obtained as an orange oil.

¹H NMR (DMSO-d₆, 400 MHz) δ_{H}=6.92-6.82 (m, 2H), 4.79 (s, 2H), 3.35 (s, 1H), 2.93 (s, 2H), 2.80-2.59 (m, 2H), 2.37-2.24 (m, 2H), 2.21 (s, 3H), 0.99 (d, J=6.0 Hz, 3H).

### Step 3: (S)—N-(5-bromo-2-(3,4-dimethylpiperazin-1-yl)-4-fluorophenyl)-6-chloro-5-nitropyrimidin-4-amine (Compound 4)

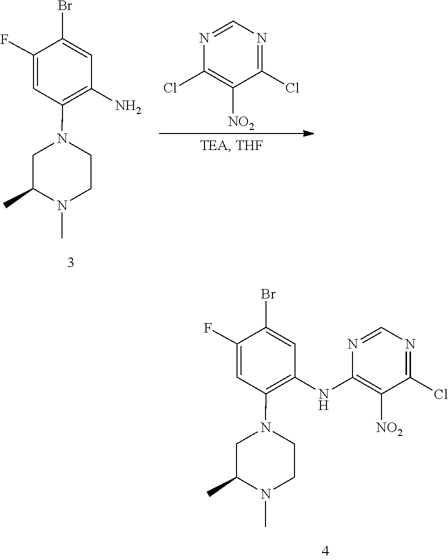 [SMILES: C[C@H]1CN(c2cc(F)c(Br)cc2N)CCN1C ; O=[N+]([O-])c1c(Cl)ncnc1Cl ; C[C@H]1CN(c2cc(F)c(Br)cc2Nc2ncnc(Cl)c2[N+](=O)[O-])CCN1C]

To a solution of compound 3 (1.42 g, 4.70 mmol, 1 eq.) and 4,6-dichloro-5-nitro-pyrimidine (1.09 g, 5.64 mmol, 1.2 eq.) in THF (15 mL) was added TEA (951 mg, 9.40 mmol, 1.31 mL, 2 eq.). The mixture was stirred at 25° C. for 3 hr. The mixture was diluted with DCM (100 mL), washed with brine (50 mL*3). The organic layer was dried over Na₂SO₄, concentrated under reduced pressure to give a residue. The residue was purified by flash silica gel chromatography (ISCO®; 12 g SepaFlash® Silica Flash Column, Eluent of 0-10% Methanol/Dichloromethane @ 30 mL/min). Compound 4 (530 mg, 1.11 mmol, 23.55% yield) was obtained as an orange oil.

¹H NMR (DMSO-d₆, 400 MHz) δ_{H}=10.27-9.91 (m, 1H), 8.59 (s, 1H), 8.09 (d, J=7.6 Hz, 1H), 7.24 (d, J=10.4 Hz, 1H), 3.42-3.24 (m, 2H), 2.96 (d, J=11.6 Hz, 2H), 2.88-2.77 (m, 2H), 2.49-2.43 (m, 1H), 2.23 (s, 3H), 0.98 (d, J=6.0 Hz, 3H).

### Step 4: (S)-N4-(5-bromo-2-(3,4-dimethylpiperazin-1-yl)-4-fluorophenyl)-6-chloropyrimidine-4,5-diamine (Compound 5)

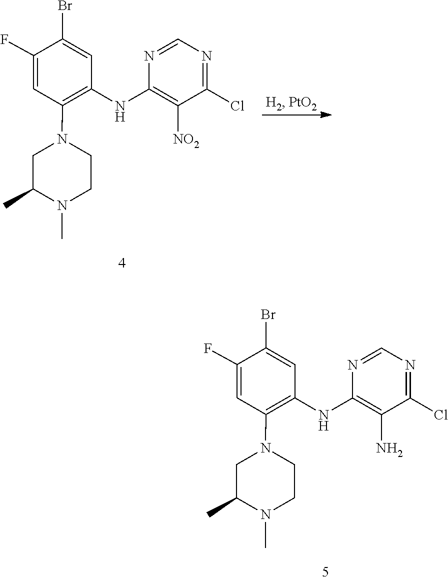 [SMILES: C[C@H]1CN(c2cc(F)c(Br)cc2Nc2ncnc(Cl)c2[N+](=O)[O-])CCN1C ; C[C@H]1CN(c2cc(F)c(Br)cc2Nc2ncnc(Cl)c2N)CCN1C]

To a solution of compound 4 (530 mg, 1.15 mmol, 1 eq.) in EtOH (50 mL) was added PtO₂ (26 mg, 115.29 μmol, 0.1 eq.). The suspension was degassed and purged with H₂ for 3 times. The mixture was stirred under H₂ (15 Psi.) at 25° C. for 5 hr. The mixture was filtered to remove the insoluble. The filter liquor was concentrated in vacuo. The reaction mixture was diluted with DCM (100 mL), washed with brine (50 mL*3). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-15% Methanol/Dichloromethane @ 30 mL/min). Compound 5 (200 mg, 331.85 μmol, 28.78% yield) was obtained as a yellow solid.

¹H NMR (DMSO-d₆, 400 MHz) δ_{H}=8.14 (s, 1H), 7.89 (s, 1H), 7.21-7.15 (m, 1H), 5.76 (s, 2H), 5.36 (s, 2H), 3.17 (s, 1H), 2.93-2.85 (m, 2H), 2.80-2.72 (m, 2H), 2.50-2.40 (m, 2H), 2.37-2.29 (m, 1H), 2.22 (s, 3H), 0.99-0.94 (m, 3H).

### Step 5: (S)-5′-((5-amino-6-chloropyrimidin-4-yl)amino)-4′-(3,4-dimethylpiperazin-1-yl)-2′-fluoro-N,N-dimethyl-[1,1′-biphenyl]-4-carboxamide (DDO-2213_001)

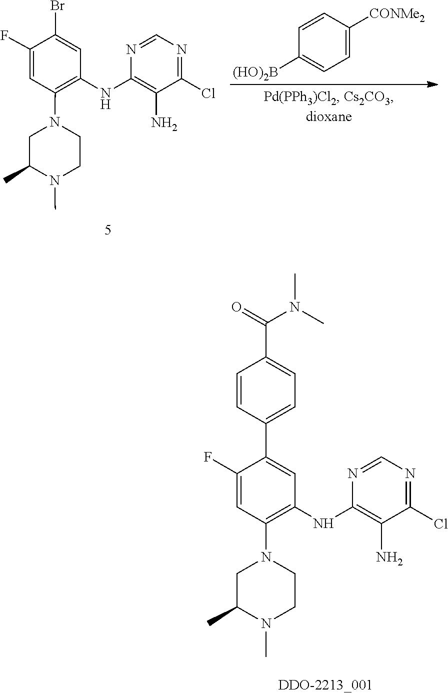

A mixture of compound 5 (200 mg, 465.42 μmol, 1 eq.), [4-(dimethylcarbamoyl)phenyl]boronic acid (90 mg, 465.42 μmol, 1 eq.), Pd(dppf)Cl₂·CH₂Cl₂ (38 mg, 46.54 μmol, 0.1 eq.) and Cs₂CO₃ (303.29 mg, 930.84 μmol, 2 eq.) in dioxane (4 mL) and H₂O (0.8 mL) was degassed and purged with N₂ for 3 times, and then the mixture was stirred at 100° C. for 12 hr under N₂ atmosphere. The mixture was filtered to remove the insoluble. The filter liquor was concentrated in vacuo. The reaction mixture was diluted with DCM (100 mL), washed with brine (20 mL). The organic layer was dried over Na₂SO₄, filtered and concentrated under reduced pressure to give a residue. The residue was purified by prep-HPLC (Column: Phenomenex Gemini-NX 80*30 mm*3 m; Mobile Phase A: purified water (0.04% NH₃H₂O+10 mM NH₄HCO₃); Mobile Phase B: MeOH; Gradient: 39-69% B in 11 min.) DDO-2213_001 (40.5 mg, 79.07 μmol, 16.99% yield, 97.23% purity) was obtained as a white solid.

¹H NMR (DMSO-d₆, 400 MHz) δ_{H}=8.10 (s, 1H), 7.95-7.89 (m, 1H), 7.81 (s, 1H), 7.61-7.55 (m, 2H), 7.49 (d, J=8.0 Hz, 2H), 7.08 (d, J=12.4 Hz, 1H), 5.36 (s, 2H), 3.04-2.90 (m, 8H), 2.76 (s, 2H), 2.47-2.41 (m, 1H), 2.34-2.25 (m, 1H), 2.19 (s, 4H), 0.96 (d, J=6.4 Hz, 3H).

HPLC Rₜ=2.90 min in 8 min chromatography, Ultimate XB-C18 3.0*50 mm, 3 μm, purity 97.23%.

LCMS Rₜ=1.983 min in 4 min chromatography, Xtimate C18, 3 μm, 3.0*50 mm, purity 95.90%, MS ESI calcd. for 497.21 [M+H]⁺ 498.21, found 498.1.

### Example 38. Synthesis of 4-[5-[(5-amino-6-chloro-pyrimidin-4-yl)amino]-2-fluoro-4-[(3R,5S)-3,4,5-trimethylpiperazin-1-yl]phenyl]-N,N-dimethyl-benzamide (Compound 124, Table 3)

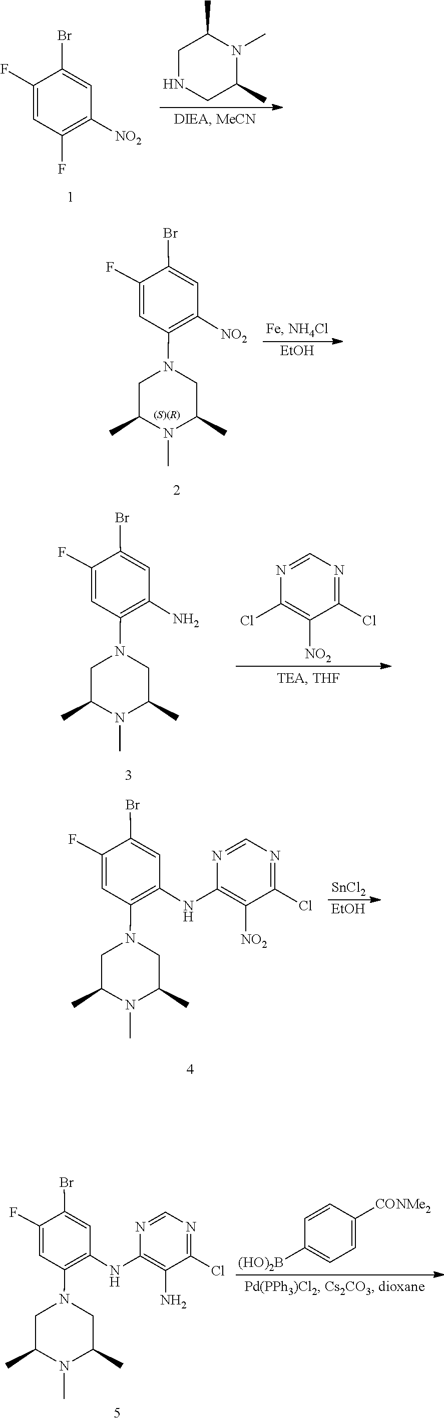

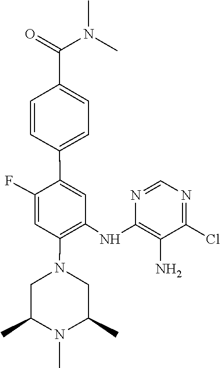 [SMILES: C[C@@H]1CN(c2cc(F)c(-c3ccc(C(=O)N(C)C)cc3)cc2Nc2ncnc(Cl)c2N)C[C@H](C)N1C]

### Step 1: (2S,6R)-4-(4-bromo-5-fluoro-2-nitrophenyl)-1,2,6-trimethylpiperazine (Compound 2)

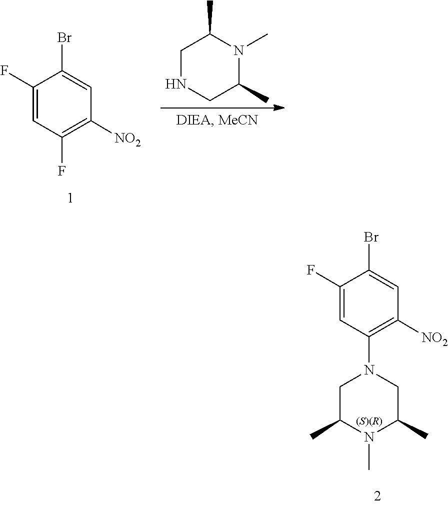

To a solution of compound 1 (10 g, 42.02 mmol, 1 eq.) in MeCN (200 mL) was added (2S,6R)-1,2,6-trimethylpiperazine (5.66 g, 44.12 mmol, 1.05 eq.) and DIEA (10.86 g, 84.04 mmol, 14.64 mL, 2 eq.). The reaction mixture was stirred at 30° C. for 2 hr to give a brown mixture. The reaction mixture was concentrated to dryness. Water (200 mL) and DCM (100 mL) were added. The resulting mixture was extracted with DCM (150 mL*2). The combined organic phase was washed with brine (200 mL), dried over anhydrous Na₂SO₄, filtered and concentrated. Compound 2 (14 g, 33.52 mmol, 79.78% yield, 82.9% purity) was obtained as a yellow solid.

¹H NMR (CDCl₃, 400 MHz) δ_{H}=8.13 (d, J=7.2 Hz, 1H), 6.92-6.77 (m, 1H), 3.17-3.00 (m, 2H), 3.00-1.98 (m, 7H), 1.33-1.05 (m, 6H).

### Step 2: 5-bromo-4-fluoro-2-((3S,5R)-3,4,5-trimethylpiperazin-1-yl)aniline (Compound 3)

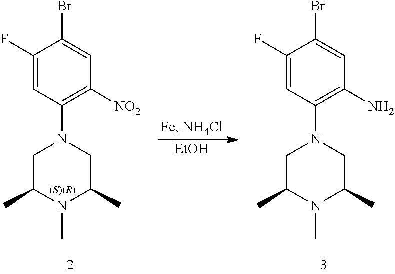 [SMILES: "EtOH" ; C[C@@H]1CN(c2cc(F)c(Br)cc2[N+](=O)[O-])C[C@H](C)N1C ; C[C@@H]1CN(c2cc(F)c(Br)cc2N)C[C@H](C)N1C]

To a mixture of compound 2 (13 g, 37.55 mmol, 1 eq.) and NH₄Cl (10.04 g, 187.76 mmol, 5 eq.) in EtOH (150 mL) and H₂O (50 mL) was added Fe (6.29 g, 112.65 mmol, 3 eq.). The reaction mixture was stirred at 80° C. for 2 hr to give a brown mixture. After cooling, the reaction mixture was filtered and the cake was washed with EtOH (100×2). The filtrate was concentrated to dryness. Aq. NaHCO₃ (200 mL) was added to the residue. The resulting mixture was extracted with DCM (200 mL*3). The combined organic phase was washed with brine (40 mL), dried over anhydrous Na₂SO₄, filtered and concentrated to dryness. The residue was purified by flash silica gel chromatography (ISCO®; 120 g SepaFlash® Silica Flash Column, Eluent of 0-15% DCM/MeOH ether gradient @ 60 mL/min). Compound 3 (8 g, 22.94 mmol, 61.09% yield, 90.68% purity) was obtained as brown oil.

¹H NMR (DMSO-d₆, 400 MHz) δ_{H}=6.88 (d, J=7.6 Hz, 1H), 6.82 (d, J=10.4 Hz, 1H), 4.79 (s, 2H), 2.93 (d, J=9.6 Hz, 2H), 2.36-2.24 (m, 4H), 2.19 (s, 3H), 1.01 (d, J=5.6 Hz, 6H).

### Step 3: N-[5-bromo-4-fluoro-2-[(3R,5S)-3,4,5-trimethylpiperazin-1-yl]phenyl]-6-chloro-5-nitro-pyrimidin-4-amine (Compound 4)

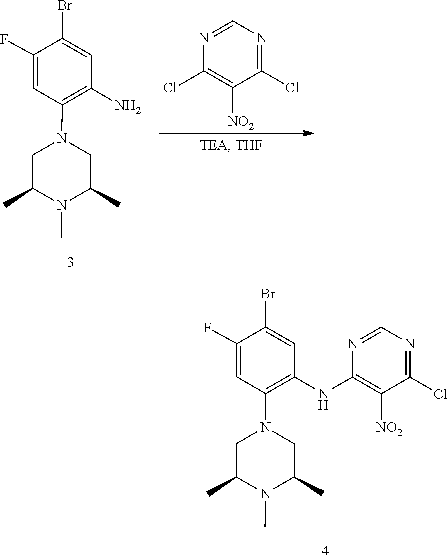 [SMILES: C[C@@H]1CN(c2cc(F)c(Br)cc2N)C[C@H](C)N1C ; O=[N+]([O-])c1c(Cl)ncnc1Cl ; C[C@@H]1CN(c2cc(F)c(Br)cc2Nc2ncnc(Cl)c2[N+](=O)[O-])C[C@H](C)N1C]

To a solution of compound 3 (8 g, 25.30 mmol, 1 eq.) and TEA (5.12 g, 50.60 mmol, 7.04 mL, 2 eq.) in THF (100 mL) was added 4,6-dichloro-5-nitro-pyrimidine (5.89 g, 30.36 mmol, 1.2 eq.). The reaction mixture was stirred at 15° C. for 12 hr to give a brown mixture. Water (200 mL) was added to the reaction mixture. The resulting mixture was extracted with DCM (200 mL*3). The combined organic phase was washed with brine (300 mL), dried over anhydrous Na₂SO₄, filtered and concentrated to dryness. The residue was purified by flash silica gel chromatography (ISCO®; 80 g SepaFlash® Silica Flash Column, Eluent of 0-15% DCM/MeOH ether gradient @ 60 mL/min). Compound 4 (5 g, 7.99 mmol, 31.58% yield, 75.7% purity) was obtained as a brown solid.

¹H NMR (DMSO-d₆, 400 MHz) δ_{H}=10.26-9.92 (m, 1H), 8.58 (s, 1H), 8.08 (d, J=7.6 Hz, 1H), 7.39-7.09 (m, 1H), 3.07-2.85 (m, 2H), 2.61-2.51 (m, 2H), 2.43-2.03 (m, 5H), 1.02 (d, J=6.0 Hz, 6H).

### Step 4: N4-[5-bromo-4-fluoro-2-[(3R,5S)-3,4,5-trimethylpiperazin-1-yl]phenyl]-6-chloro-pyrimidine-4,5-diamine (Compound 5)

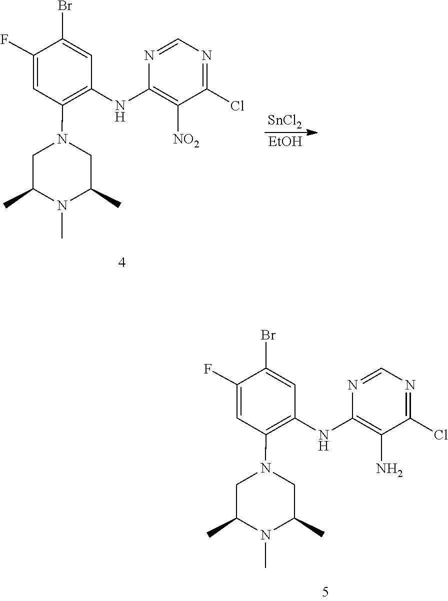

To a mixture of compound 4 (500 mg, 1.06 mmol, 1 eq.) in EtOH (20 mL) and H₂O (2 mL) was added SnCl₂·H₂O (476 mg, 2.11 mmol, 2 eq). The reaction mixture was stirred at 80° C. for 2 hr to give a brown mixture. After cooling to room temperature, the reaction mixture was then adjusted to pH=8 by sat. aq.NaHCO₃. The resulting mixture was extracted with DCM (20 mL*3). The combined organic phase was washed with brine (20 mL), dried over anhydrous Na₂SO₄, filtered and concentrated. The residue was purified by flash silica gel chromatography (ISCO®; 12 g SepaFlash® Silica Flash Column, Eluent of 0-15% DCM/MeOH ethergradient @ 25 mL/min). The product compound 5 (170 mg, 358.09 μmol, 33.93% yield, 93.47% purity) was obtained as a yellow solid.

¹H NMR (DMSO-d₆, 400 MHz) δ_{H}=8.20-8.08 (m, 2H), 7.89 (s, 1H), 7.16 (d, J=10.4 Hz, 1H), 5.35 (s, 2H), 2.90 (d, J=10.8 Hz, 2H), 2.47-2.28 (m, 4H), 2.20 (s, 3H), 0.98 (d, J=6.0 Hz, 6H).

### Step 5: 4-[5-[(5-amino-6-chloro-pyrimidin-4-yl)amino]-2-fluoro-4-[(3R,5S)-3,4,5-trimethylpiperazin-1-yl]phenyl]-N,N-dimethyl-benzamide (DDO-2213_002)

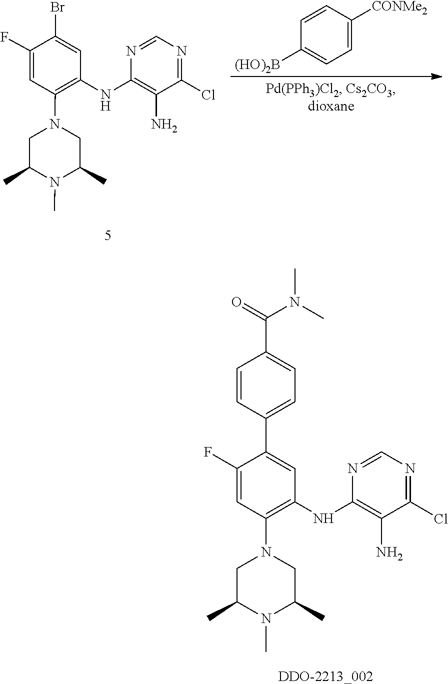

To a mixture of compound 5 (100 mg, 225.36 μmol, 1 eq.) and [4-(dimethylcarbamoyl)phenyl]boronic acid (52.19 mg, 270.43 μmol, 1.2 eq.) in dioxane (4 mL) and H₂O (0.8 mL) under N₂ was added Pd(dppf)Cl₂·CH₂Cl₂ (18 mg, 22.54 μmol, 0.1 eq.) and Cs₂CO₃ (147 mg, 450.71 μmol, 2 eq.). The reaction mixture was stirred at 100° C. for 2 hr to give a black mixture. After cooling, water (10 mL) was added to the reaction mixture. The resulting mixture was extracted with DCM (10 mL*3). The combined organic phase was washed with brine (20 mL), dried over anhydrous Na₂SO₄, filtered and concentrated to dryness. The residue was purified by prep-HPLC (Column: Phenomenex luna C18 80*40 mm*3 μm; Condition: water (0.05% ammonia hydroxide v/v)-ACN; Begin B: 27%, End B: 67%; Gradient Time(min): 13 min). DDO-2213_002 (23.2 mg, 44.81 μmol, 19.89% yield, 98.9% purity) was obtained as an off-white solid.

¹H NMR (DMSO-d₆, 400 MHz) δ_{H}=9.66 (s, 1H), 9.20 (s, 1H), 8.90-8.78 (m, 1H), 8.65-8.54 (m, 1H), 7.72 (d, J=8.8 Hz, 1H), 7.56 (t, J=7.2 Hz, 1H), 7.45-7.31 (m, 2H), 4.04-3.45 (m, 4H), 2.94 (s, 4H), 2.46-2.32 (m, 13H), 2.24-2.13 (m, 5H), 1.80-1.63 (m, 2H).

HPLC Rₜ=2.97 min in 8 min chromatography, Ultimate XB-C18 3.0*50 mm, 3 μm, purity 98.98%.

LCMS Rₜ=0.788 min in 2 min chromatography, Chromolith Flash RP-18.5 μm, 3.0*25 mm, purity 100%, MS ESI calcd. for 511.23 [M+H]⁺ 512.23, found 512.2.

### Example 39 5′-((5-amino-6-chloropyrimidin-4-yl)amino)-2′-fluoro-N,N-dimethyl-4′-(4-methyl-1,4-diazepan-1-yl)-[1,1′-biphenyl]-4-carboxamide (Compound 125, Table 3)

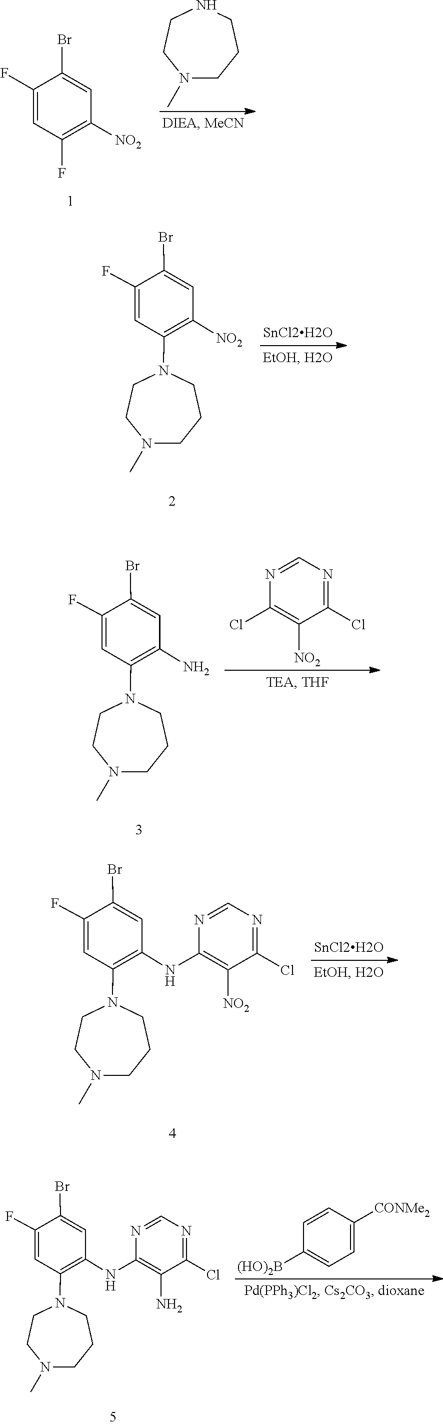

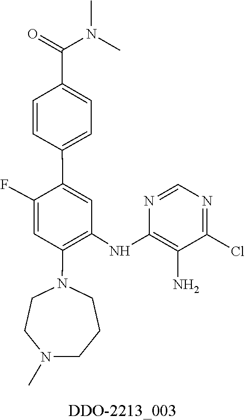 [SMILES: CN1CCCN(c2cc(F)c(-c3ccc(C(=O)N(C)C)cc3)cc2Nc2ncnc(Cl)c2N)CC1]

### Step 1: 1-(4-bromo-5-fluoro-2-nitrophenyl)-4-methyl-1,4-diazepane (Compound 2)

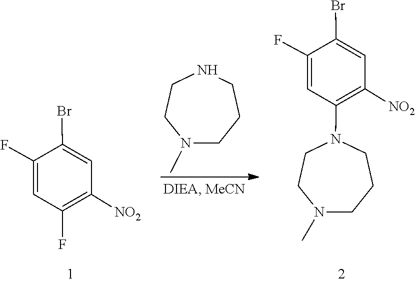

To a solution of compound 1 (6 g, 25.21 mmol, 1 eq.) in CH₃CN (30 mL) was added 1-methyl-1,4-diazepane (2.88 g, 25.21 mmol, 3.14 mL, 1 eq.) and DIEA (6.52 g, 50.42 mmol, 8.78 mL, 2 eq.). The mixture was stirred at 20° C. for 2 hr. The mixture was diluted with DCM (100 mL), washed with brine (50 mL*3). The organic layer was dried over Na₂SO₄, filtered and concentrated under reduced pressure to give a residue. The residue was purified by flash silica gel chromatography (ISCO®; 40 g SepaFlash® Silica Flash Column, Eluent of 0-15% Methanol/Dichloromethane @ 50 mL/min). Compound 2 (7.24 g, 20.00 mmol, 79.31% yield) was obtained as a yellow oil.

¹H NMR (DMSO-d₆, 400 MHz) δ_{H}=8.11 (d, J=7.6 Hz, 1H), 7.19 (d, J=12.4 Hz, 1H), 3.33 (d, J=1.6 Hz, 2H), 3.25-3.19 (m, 2H), 2.65-2.60 (m, 2H), 2.50-2.45 (m, 2H), 2.23 (s, 3H), 1.89-1.81 (m, 2H).

### Step 2: 5-bromo-4-fluoro-2-(4-methyl-1,4-diazepan-1-yl)aniline (Compound 3)

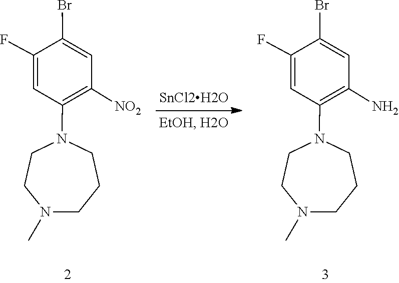 [SMILES: CN1CCCN(c2cc(F)c(Br)cc2[N+](=O)[O-])CC1 ; CN1CCCN(c2cc(F)c(Br)cc2N)CC1]

To a solution of compound 2 (4 g, 12.04 mmol, 1 eq.) in EtOH (30 mL) and H₂O (3 mL) was added SnCl₂·2H₂O (8.15 g, 36.13 mmol, 3 eq.). The mixture was stirred at 80° C. for 4 hr. The mixture was adjusted to pH 9 with aq.NaHCO₃. Filtered to remove the insolubles. The filter liquor was concentrated in vacuo. The mixture was diluted with DCM (300 mL), washed with brine (50 mL). The organic layer was dried over Na₂SO₄, filtered and concentrated under reduced pressure to give a residue. The residue was purified by flash silica gel chromatography (ISCO®; 20 g SepaFlash® Silica Flash Column, Eluent of 0-14% Methanol/Dichloromethane @ 40 mL/min). Compound 3 (3.32 g, 10.20 mmol, 84.67% yield) was obtained as a black brown solid

¹H NMR (DMSO-d₆, 400 MHz) δ_{H}=6.99-6.87 (m, 2H), 5.03-4.93 (m, 2H), 3.00 (d, J=6.0 Hz, 8H), 2.57 (s, 3H), 2.04-1.94 (m, 2H).

### Step 3: N-(5-bromo-4-fluoro-2-(4-methyl-1,4-diazepan-1-yl)phenyl)-6-chloro-5-nitropyrimidin-4-amine (Compound 4)

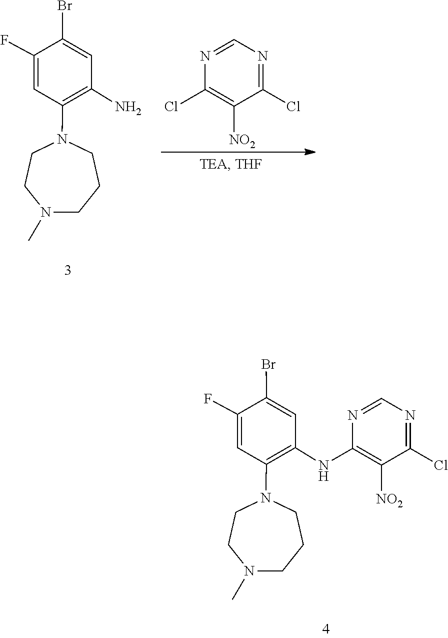 [SMILES: CN1CCCN(c2cc(F)c(Br)cc2N)CC1 ; O=[N+]([O-])c1c(Cl)ncnc1Cl ; CN1CCCN(c2cc(F)c(Br)cc2Nc2ncnc(Cl)c2[N+](=O)[O-])CC1]

To a solution of compound 3 (2 g, 6.62 mmol, 1 eq.) and 4,6-dichloro-5-nitro-pyrimidine (1.54 g, 7.94 mmol, 1.2 eq.) in THF (10 mL) was added TEA (1.34 g, 13.24 mmol, 1.84 mL, 2 eq.). The mixture was stirred at 25° C. for 3 hr. The mixture was filtered to removed the insoluble. The filter liquor was concentrated in vacuo. The reaction mixture was diluted with DCM (300), washed with brine (50 mL). The organic layer was dried over Na₂SO₄, filtered and concentrated under reduced pressure to give a residue. The residue was purified by flash silica gel chromatography (ISCO®; 40 g SepaFlash® Silica Flash Column, Eluent of 0-13% MeOH/DCM @ 40 mL/min). Compound 4 (520 mg, 932.09 μmol, 14.08% yield) was obtained as a brown solid.

¹H NMR (DMSO-d₆, 400 MHz) δ_{H}=8.54 (s, 1H), 7.84 (d, J=8.0 Hz, 1H), 7.21 (d, J=11.2 Hz, 1H), 5.77 (s, 2H), 3.24-3.02 (m, 5H), 2.77 (s, 3H), 2.10 (s, 3H), 1.19 (t, J=7.2 Hz, 1H).

### Step 4: N4-(5-bromo-4-fluoro-2-(4-methyl-1,4-diazepan-1-yl)phenyl)-6-chloropyrimidine-4,5-diamine (Compound 5)

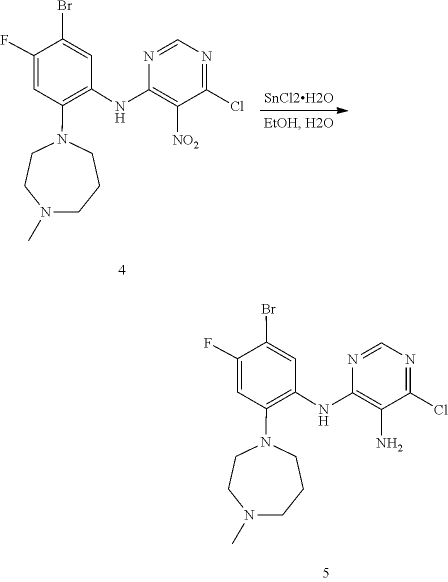 [SMILES: CN1CCCN(c2cc(F)c(Br)cc2Nc2ncnc(Cl)c2[N+](=O)[O-])CC1 ; CN1CCCN(c2cc(F)c(Br)cc2Nc2ncnc(Cl)c2N)CC1]

To a solution of compound 4 (500 mg, 1.09 mmol, 1 eq.) in EtOH (10 mL) and H₂O (3 mL) was added SnCl₂·2H₂O (736 mg, 3.26 mmol, 3 eq.). The mixture was stirred at 80° C. for 2 hr. The mixture was adjusted to pH=9 with NaHCO₃. Filtered to removed the insoluble. The filter liquor was concentrated in vacuo. The mixture was diluted with DCM (150 mL), washed with brine (30 mL). The organic layer was dried over Na₂SO₄, filtered and concentrated under reduced pressure to give a residue. The residue was purified by flash silica gel chromatography (ISCO®; 20 g SepaFlash® Silica Flash Column, Eluent of 0-16% MeOH/DCM @ 40 mL/min). Compound 5 (300 mg, 628.32 μmol, 57.77% yield) was obtained as a yellow oil.

¹H NMR (DMSO-d₆, 400 MHz) δ_{H}=8.28-8.18 (m, 1H), 7.83-7.75 (m, 2H), 7.11 (d, J=11.2 Hz, 1H), 5.41 (s, 2H), 3.20-3.09 (m, 4H), 2.75-2.64 (m, 4H), 2.34 (s, 3H), 1.87-1.79 (m, 2H).

### Step 5: 5′-((5-amino-6-chloropyrimidin-4-yl)amino)-2′-fluoro-N,N-dimethyl-4′-(4-methyl-1,4-diazepan-1-yl)-[1,1′-biphenyl]-4-carboxamide (DDO-2213_003)

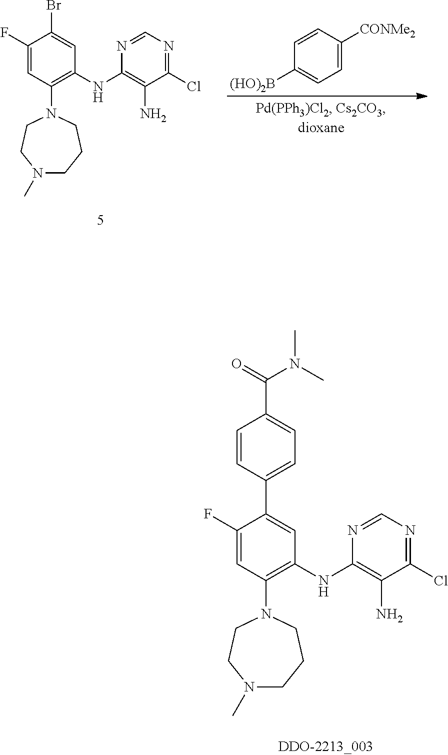

A mixture of compound 5 (150 mg, 349.07 μmol, 1 eq.), [4-(dimethylcarbamoyl)phenyl]boronic acid (81 mg, 418.88 μmol, 1.2 eq.), Pd(dppf)Cl₂·CH₂Cl₂ (29 mg, 34.91 μmol, 0.1 eq.) and Cs₂CO₃ (228 mg, 698.13 μmol, 2 eq.) in dioxane (5 mL) and H₂O (1 mL) was degassed and purged with N₂ for 3 times, and then the mixture was stirred at 100° C. for 12 hr under N₂ atmosphere. The reaction mixture was diluted with H2O (10 mL), extracted with DCM (30 mL*2), washed with brine (20 mL). The organic layer was dried over Na₂SO₄, filtered and concentrated under reduced pressure to give a residue. The residue was purified by prep-HPLC (Column: Phenomenex Gemini-NX C18 75*30 mm*3 μm; Mobile Phase A: purified water (0.04% NH₃H2O+10 mM NH₄HCO₃); Mobile Phase B: acetonitrile; Gradient: 7-47% B in 12 min.). DDO-2213_003 (18 mg, 35.56 μmol, 10.19% yield, 98.39% purity) was obtained as a white solid.

¹H NMR (DMSO-d₆, 400 MHz) δ_{H}=8.16 (s, 1H), 7.74 (s, 1H), 7.57 (s, 3H), 7.49-7.44 (m, 2H), 7.02-6.95 (m, 1H), 5.35 (s, 2H), 3.23-3.16 (m, 4H), 3.03-2.92 (m, 6H), 2.61-2.52 (m, 4H), 2.24 (s, 3H), 1.84-1.74 (m, 2H).

HPLC Rₜ=2.852 min in 8 min chromatography, X Bridge Shield RP18, 5 μm, 2.1*50 mm, purity 98.39%.

LCMS Rₜ=2.357 min in 4 min chromatography, X Bridge Shield RP18, 5 μm, 2.1*50 mm, purity 100.00%, MS ESI calcd. for 497.21 [M+H]⁺ 498.21, found 498.3.

### Example 40. 5′-((5-amino-6-chloropyrimidin-4-yl)amino)-2′-fluoro-N-isopropyl-4′-((3S,5R)-3,4,5-trimethylpiperazin-1-yl)-[1,1′-biphenyl]-4-carboxamide (Compound 126, Table 3)

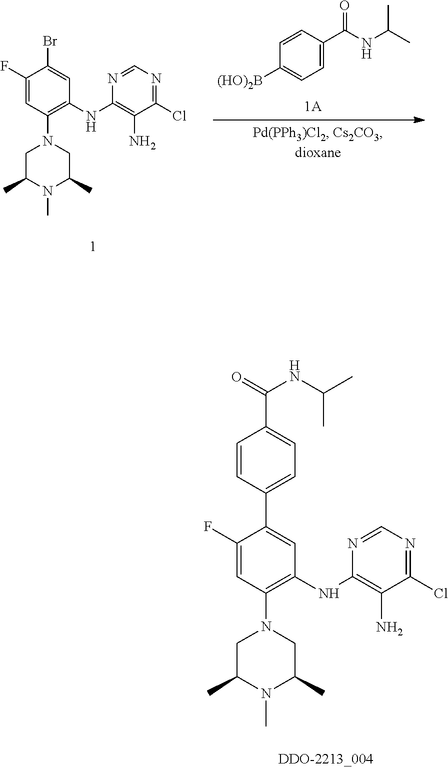 [SMILES: C[C@@H]1CN(c2cc(F)c(Br)cc2Nc2ncnc(Cl)c2N)C[C@H](C)N1C ; CC(C)NC(=O)c1ccc(B(O)O)cc1 ; CC(C)NC(=O)c1ccc(-c2cc(Nc3ncnc(Cl)c3N)c(N3C[C@@H](C)N(C)[C@@H](C)C3)cc2F)cc1]

### Step 1: 5′-((5-amino-6-chloropyrimidin-4-yl)amino)-2′-fluoro-N-isopropyl-4′-((3S,5R)-3,4,5-trimethylpiperazin-1-yl)-[1,1′-biphenyl]-4-carboxamide (DDO-2213_004)

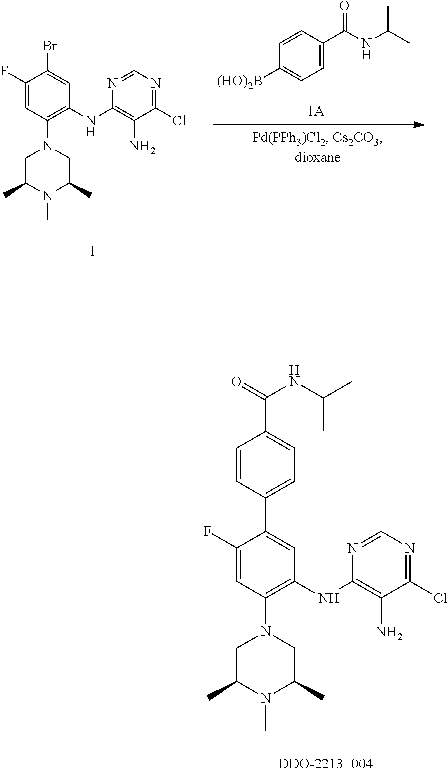 [SMILES: C[C@@H]1CN(c2cc(F)c(Br)cc2Nc2ncnc(Cl)c2N)C[C@H](C)N1C ; CC(C)NC(=O)c1ccc(B(O)O)cc1 ; CC(C)NC(=O)c1ccc(-c2cc(Nc3ncnc(Cl)c3N)c(N3C[C@@H](C)N(C)[C@@H](C)C3)cc2F)cc1]

Compound 1 is synthesized as shown in Example 38 above. To a mixture of compound 1 (200 mg, 450.71 μmol, 1 eq.) and compound 1A (112 mg, 540.85 μmol, 1.2 eq.) in dioxane (4 mL) and H₂O (0.5 mL) under N₂ was added Pd(dppf)Cl₂ (33 mg, 45.07 μmol, 0.1 eq.) and Cs₂CO₃ (294 mg, 901.42 μmol, 2 eq.). The reaction mixture was stirred at 100° C. for 2 hr. The mixture was extracted with DCM (200 mL*3). The combined organic phase was washed with brine (100 mL*2), dried with anhydrous Na₂SO₄, filtered and concentrated in vacuum. The reaction mixture was concentrated to dryness. The residue was purified by prep-HPLC (Column: Phenomenex luna C18 80*40 mm*3 μm; Condition: [water(0.05% NH₃—H₂O)-ACN)]; B:46%-76%, 11 min). DDO-2213_004 (70 mg, 127.69 μmol, 28.33% yield, 95.96% purity) was obtained as a white solid.

¹H NMR (DMSO-d₆, 400 MHz) δ_{H}=8.31-8.09 (m, 2H), 7.97-7.77 (m, 4H), 7.60 (d, J=7.2 Hz, 2H), 7.04 (d, J=12.0 Hz, 1H), 5.35 (s, 2H), 4.20-4.01 (m, 1H), 2.97 (d, J=10.4 Hz, 2H), 2.48-2.41 (m, 2H), 2.30-2.05 (m, 5H), 1.17 (d, J=6.0 Hz, 6H), 0.97 (d, J=5.2 Hz, 6H).

HPLC Rₜ=2.36 min in 8 min chromatography, Ultimate XB-C18 3.0*50 mm, 3 m, purity 95.96%.

LCMS Rₜ=1.124 min in 4 min chromatography, Xtimate C18, 3 m, 2.1*30 mm, purity 98.51%, MS ESI calcd. For 525.24 [M+H]⁺ 526.24, found 526.4.

### Example 41. (S)—N-(5′-((5-amino-6-chloropyrimidin-4-yl)amino)-4′-(3,4-dimethylpiperazin-1-yl)-2′-fluoro-[1,1′-biphenyl]-4-yl)cyclohexanecarboxamide (Compound 127, Table 3)

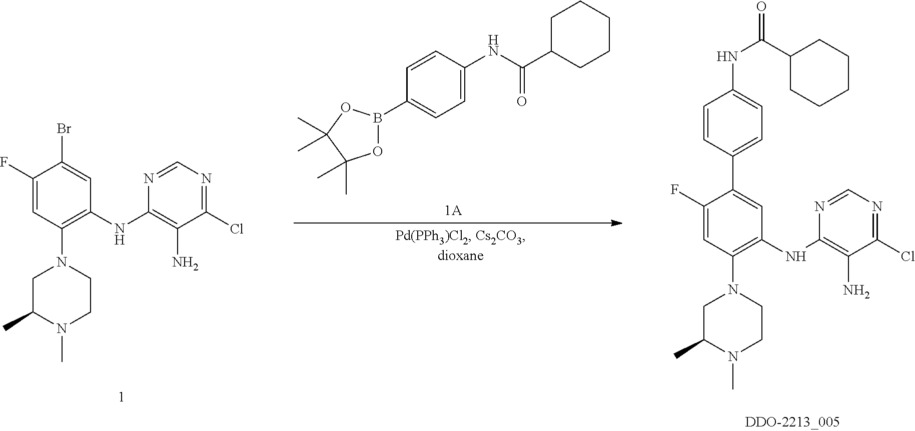 [SMILES: C[C@H]1CN(c2cc(F)c(Br)cc2Nc2ncnc(Cl)c2N)CCN1C ; CC1(C)OB(c2ccc(NC(=O)C3CCCCC3)cc2)OC1(C)C ; C[C@H]1CN(c2cc(F)c(-c3ccc(NC(=O)C4CCCCC4)cc3)cc2Nc2ncnc(Cl)c2N)CCN1C]

### Step 1: N-(4-(4,4,5,5-tetramethyl-1,3,2-dioxaborolan-2-yl)phenyl)cyclohexanecarboxamide (Compound 1A)

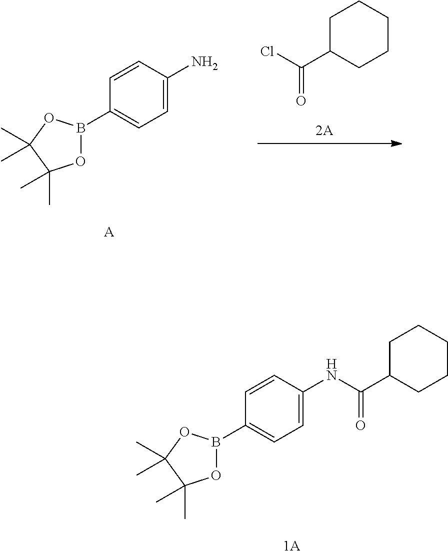

To a solution of compound A (1 g, 4.56 mmol, 1 eq.) and Et₃N (924 mg, 9.13 mmol, 1.27 mL, 2 eq.) in DCM (15 mL) at 0° C. was added cyclohexanecarbonyl chloride (736 mg, 5.02 mmol, 669 μL, 1.1 eq). The reaction mixture was stirred at 0° C. for 1 hr to give a brown mixture. Water (30 mL) was added to the reaction mixture. The reaction mixture was then adjusted to pH-8 with aq. NaHCO₃. The resulting mixture was extracted with DCM (20 mL*3). The combined organic phase was washed with brine (30 mL), dried over anhydrous Na₂SO₄, filtered and concentrated to dryness. Compound 1A (1.3 g, 2.87 mmol, 62.89% yield) was obtained as a brown solid, which was used into the next step without further purification.

¹H NMR (DMSO-d₆, 400 MHz) δ_{H}=9.92 (s, 1H), 7.60 (q, J=8.8 Hz, 4H), 3.17-2.99 (m, 1H), 2.37-2.27 (m, 1H), 1.80-1.63 (m, 5H), 1.43-1.34 (m, 2H), 1.27 (s, 12H), 1.21-1.14 (m, 2H).

### Step 2: (S)—N-(5′-((5-amino-6-chloropyrimidin-4-yl)amino)-4′-(3,4-dimethylpiperazin-1-yl)-2′-fluoro-[1,1′-biphenyl]-4-yl)cyclohexanecarboxamide (DDO-2213_005)

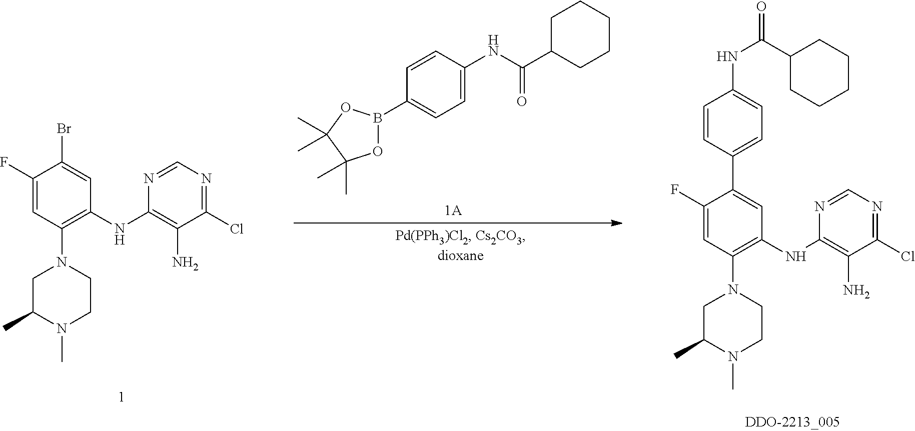 [SMILES: C[C@H]1CN(c2cc(F)c(Br)cc2Nc2ncnc(Cl)c2N)CCN1C ; CC1(C)OB(c2ccc(NC(=O)C3CCCCC3)cc2)OC1(C)C ; C[C@H]1CN(c2cc(F)c(-c3ccc(NC(=O)C4CCCCC4)cc3)cc2Nc2ncnc(Cl)c2N)CCN1C]

Compound 1 is synthesized as shown in Example 37. To a mixture of compound 1 (150 mg, 349.07 μmol, 1 eq.) and compound 1A (138 mg, 418.88 μmol, 1.2 eq.) in dioxane (4 mL) and H₂O (0.5 mL) under N₂ was added Pd(dppf)Cl₂ (26 mg, 34.91 μmol, 0.1 eq.) and Cs₂CO₃ (228 mg, 698.14 μmol, 2 eq.). The reaction mixture was stirred at 100° C. for 2 hr. The mixture was extracted with DCM (20 mL*3). The combined organic phase was washed with brine (20 mL*2), dried with anhydrous Na₂SO₄, filtered and concentrated in vacuum. The reaction mixture was concentrated to dryness. The residue was purified by prep-HPLC (Column:Phenomenex luna C18 80*40 mm*3 μm; Condition:[water(0.04% NH₃H₂O+10 mM NH₄HCO₃)-ACN)]; B:40%-70%, 11 min). DDO-2213_005 (47.9 mg, 85.78 μmol, 24.57% yield, 98.87% purity) was obtained as a white solid.

¹H NMR (DMSO-d₆, 400 MHz) δ_{H}=9.92 (s, 1H), 8.10 (s, 1H), 7.85 (d, J=8.8 Hz, 1H), 7.81 (s, 1H), 7.69 (d, J=8.8 Hz, 2H), 7.44 (d, J=7.6 Hz, 2H), 7.03 (d, J=12.4 Hz, 1H), 5.35 (s, 2H), 2.97-2.87 (m, 2H), 2.80-2.71 (m, 2H), 2.44 (t, J=10.4 Hz, 1H), 2.38-2.24 (m, 2H), 2.18 (s, 3H), 1.84-1.73 (m, 4H), 1.65 (d, J=10.8 Hz, 1H), 1.48-1.36 (m, 2H), 1.33-1.11 (m, 4H), 0.95 (d, J=6.4 Hz, 3H).

HPLC Rₜ=3.93 min in 8 min chromatography, Ultimate XB-C18 3.0*50 mm, 3 m, purity 98.87%.

LCMS Rₜ=2.162 min in 4 min chromatography, Xtimate C18, 3 m, 2.1*30 mm, purity 99.87%, MS ESI calcd. For 551.26 [M+H]⁺ 552.26, found 552.4.

### Example 42. N-(5′-((5-amino-6-chloropyrimidin-4-yl)amino)-2′-fluoro-4′-((3S,5R)-3,4,5-trimethylpiperazin-1-yl)-[1,1′-biphenyl]-4-yl)cyclohexanecarboxamide (Compound 128, Table 3)

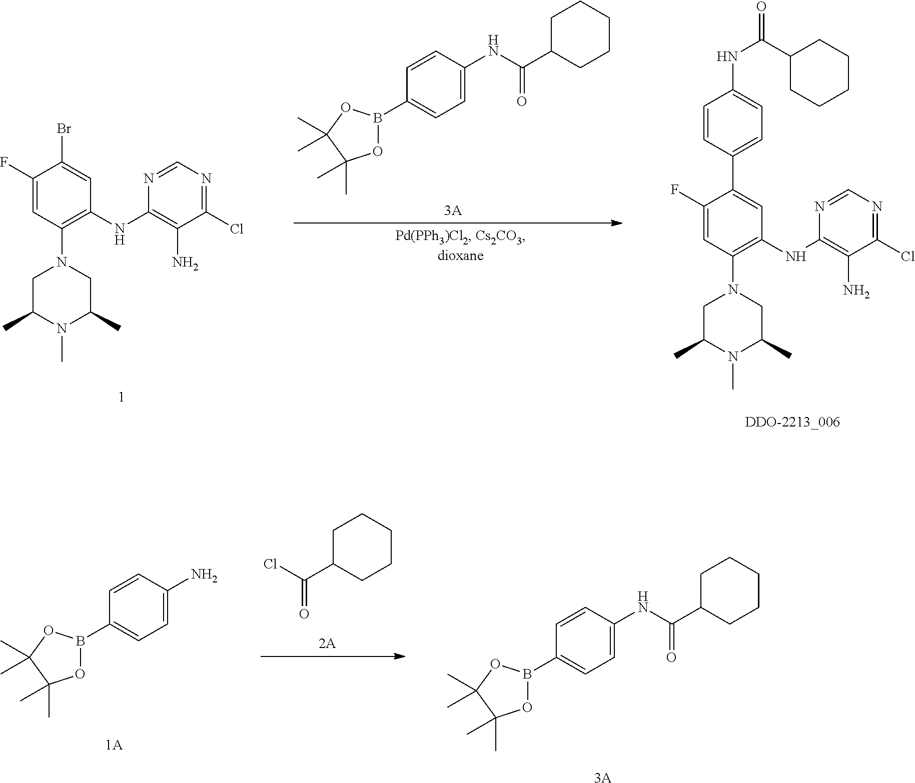

### Step 1: N-(5′-((5-amino-6-chloropyrimidin-4-yl)amino)-2′-fluoro-4′-((3S,5R)-3,4,5-trimethylpiperazin-1-yl)-[1,1′-biphenyl]-4-yl)cyclohexanecarboxamide (DDO-2213_006)

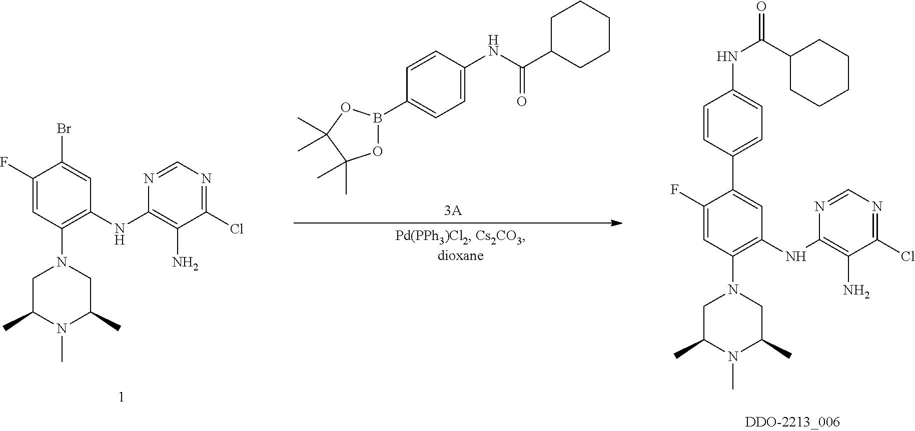 [SMILES: C[C@@H]1CN(c2cc(F)c(Br)cc2Nc2ncnc(Cl)c2N)C[C@H](C)N1C ; CC1(C)OB(c2ccc(NC(=O)C3CCCCC3)cc2)OC1(C)C ; C[C@@H]1CN(c2cc(F)c(-c3ccc(NC(=O)C4CCCCC4)cc3)cc2Nc2ncnc(Cl)c2N)C[C@H](C)N1C]

The Compound 1 is synthesized as shown in Example 38 and Compound 3A is synthesized as shown in Example 41. To a mixture of compound 1 (200 mg, 450.71 μmol, 1 eq.) and compound 3A (178 mg, 540.85 μmol, 1.2 eq.) in dioxane (4 mL) and H₂O (0.5 mL) under N₂ was added Pd(dppf)Cl₂ (33 mg, 45.07 μmol, 0.1 eq.) and Cs₂CO₃ (294 mg, 901.42 μmol, 2 eq.). The reaction mixture was stirred at 100° C. for 2 hr. The mixture was extracted with DCM (200 mL*3). The combined organic phase was washed with brine (100 mL*2), dried with anhydrous Na₂SO₄, filtered and concentrated in vacuum. The reaction mixture was concentrated to dryness. The residue was purified by prep-HPLC (Column: Phenomenex luna C18 80*40 mm*3 μm; Condition: [water(0.05% NH₃·H₂O)-ACN)]; B:50%-75%, 16 min). DDO-2213_006 (52 mg, 91.28 μmol, 20.25% yield, 99.37% purity) was obtained as a white solid.

¹H NMR (DMSO-d₆, 400 MHz) δ_{H}=9.91 (s, 1H), 8.12 (s, 1H), 7.86 (d, J=8.8 Hz, 1H), 7.81 (s, 1H), 7.69 (d, J=8.8 Hz, 2H), 7.44 (br d, J=7.6 Hz, 2H), 7.00 (d, J=12.4 Hz, 1H), 5.33 (s, 2H), 2.93 (d, J=10.8 Hz, 2H), 2.45 (t, J=10.8 Hz, 2H), 2.38-2.22 (m, 3H), 2.17 (s, 3H), 1.85-1.62 (m, 5H), 1.48-1.15 (m, 5H), 0.97 (d, J=6.0 Hz, 6H).

HPLC Rₜ=4.00 min in 8 min chromatography, Ultimate XB-C18 3.0*50 mm, 3 m, purity 99.37%.

LCMS Rₜ=2.045 min in 4 min chromatography, Xtimate C18, 3 m, 2.1*30 mm, purity 99.27%, MS ESI calcd. For 565.27 [M+H]⁺ 566.27, found 566.4.

### Example 43. N-(5′-((5-amino-6-chloropyrimidin-4-yl)amino)-2′-fluoro-4′-((3S,5R)-3,4,5-trimethylpiperazin-1-yl)-[1,1′-biphenyl]-4-yl)butyramide (Compound 129, Table 3)

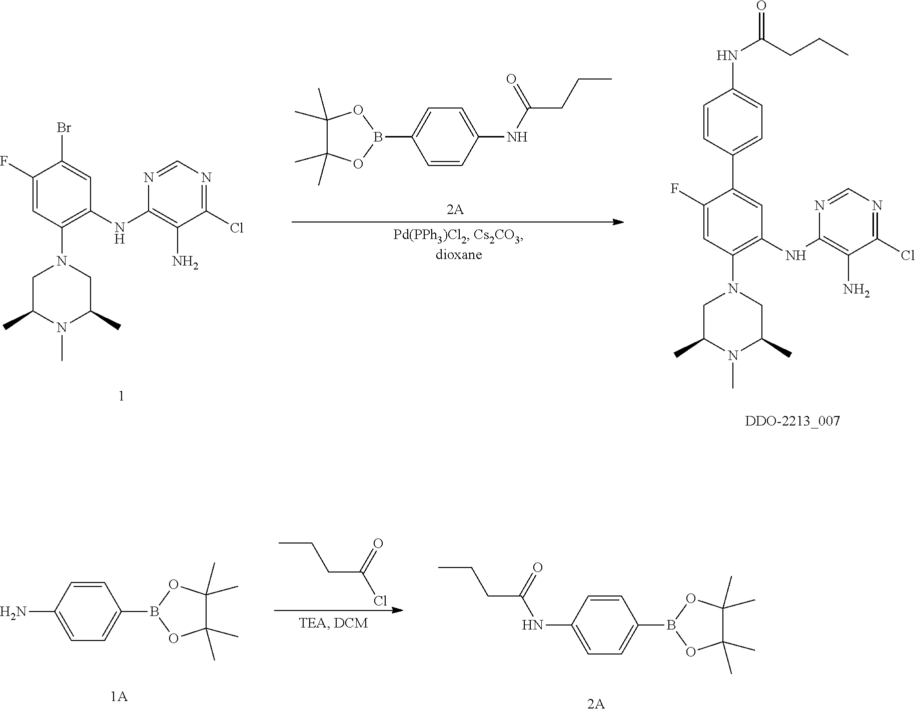

### Step 1: N-(4-(4,4,5,5-tetramethyl-1,3,2-dioxaborolan-2-yl)phenyl)butyramide (Compound 2A)

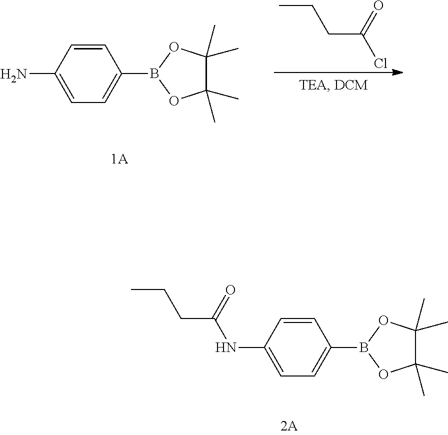

To a mixture of compound 1A (1 g, 4.56 mmol, 1 eq.) and Et₃N (924 mg, 9.13 mmol, 1.27 mL, 2 eq.) in DCM (15 mL) at 0° C. was added butanoyl chloride (535 mg, 5.02 mmol, 524.48 μL, 1.1 eq.). The reaction mixture was stirred at 0° C. for 1 hr to give a brown mixture. Water (30 mL) was added to the reaction mixture. The reaction mixture was then adjusted to pH-8 by aq. NaHCO₃. The resulting mixture was extracted with DCM (20 mL*3). The combined organic phase was washed with brine (30 mL), dried over anhydrous Na₂SO₄, filtered and concentrated. The product compound 2A (1.1 g, 3.18 mmol, 69.75% yield) was obtained as a brown solid and used into the next step without further purification.

¹H NMR (DMSO-d₆, 400 MHz) δ_{H}=10.00 (s, 1H), 7.66-7.54 (m, 4H), 3.13-3.01 (m, 2H), 1.68-1.54 (m, 2H), 1.27 (s, 12H), 0.91 (t, J=7.6 Hz, 3H).

### Step 2: N-(5′-((5-amino-6-chloropyrimidin-4-yl)amino)-2′-fluoro-4′-((3S,5R)-3,4,5-trimethylpiperazin-1-yl)-[1,1′-biphenyl]-4-yl)butyramide (DDO-2213_007)

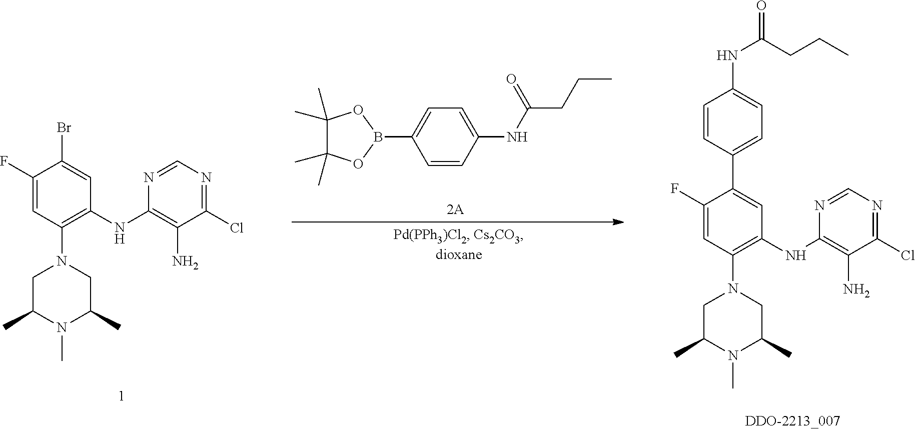 [SMILES: C[C@@H]1CN(c2cc(F)c(Br)cc2Nc2ncnc(Cl)c2N)C[C@H](C)N1C ; CCCC(=O)Nc1ccc(B2OC(C)(C)C(C)(C)O2)cc1 ; CCCC(=O)Nc1ccc(-c2cc(Nc3ncnc(Cl)c3N)c(N3C[C@@H](C)N(C)[C@@H](C)C3)cc2F)cc1]

The compound 1 is synthesized as shown in Example 38. To a mixture of compound 1 (200 mg, 450.71 μmol, 1 eq.) and compound 2A (156 mg, 540.85 μmol, 1.2 eq.) in dioxane (4 mL) and H₂O (0.5 mL) under N₂ was added Pd(dppf)Cl₂ (33 mg, 45.07 μmol, 0.1 eq.) and Cs₂CO₃ (294 mg, 901.42 μmol, 2 eq.). The reaction mixture was stirred at 100° C. for 2 hr. The mixture was extracted with DCM (200 mL*3). The combined organic phase was washed with brine (100 mL*2), dried with anhydrous Na₂SO₄, filtered and concentrated in vacuum. The reaction mixture was concentrated to dryness. The residue was purified by prep-HPLC (Column:Phenomenex luna C18 80*40 mm*3 μm; Condition:[water(0.05% NH₃·H₂O)-ACN)]; B:40%-80%, 16 min). DDO-2213_007 (33.7 mg, 63.13 μmol, 14.01% yield, 98.54% purity) was obtained as a white solid.

¹H NMR (DMSO-d₆, 400 MHz) δ_{H}=9.98 (s, 1H), 8.12 (s, 1H), 7.89-7.80 (m, 2H), 7.68 (d, J=8.0 Hz, 2H), 7.45 (d, J=8.0 Hz, 2H), 7.01 (d, J=12.4 Hz, 1H), 5.33 (s, 2H), 3.33 (s, 3H), 2.93 (d, J=10.4 Hz, 2H), 2.46-2.41 (m, 1H), 2.33-2.25 (m, 3H), 2.17 (s, 2H), 1.68-1.57 (m, 2H), 1.01-0.88 (m, 9H).

HPLC Rₜ=2.45 min in 8 min chromatography, Ultimate XB-C18 3.0*50 mm, 3 m, purity 98.54%.

LCMS Rₜ=1.221 min in 4 min chromatography, Xtimate C18, 3 m, 2.1*30 mm, purity 100%, MS ESI calcd. For 525.24 [M+H]⁺ 526.24, found 526.3.

### Example 44. (S)-6-chloro-N4-(5-(2-(cyclopropylmethoxy)pyridin-4-yl)-2-(3,4-dimethylpiperazin-1-yl)-4-fluorophenyl)pyrimidine-4,5-diamine (Compound 130, Table 3)

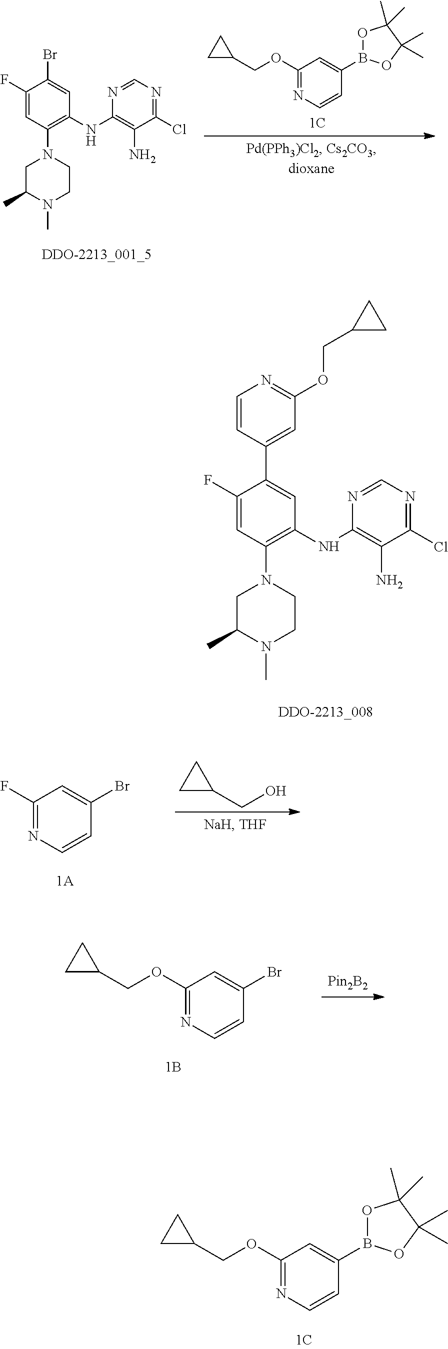

### Step 1:4-bromo-2-(cyclopropylmethoxy) Pyridine (Compound 1B)

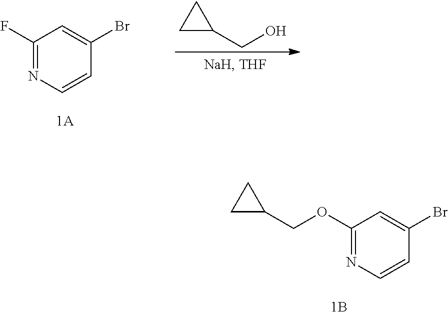 [SMILES: Fc1cc(Br)ccn1 ; OCC1CC1 ; Brc1ccnc(OCC2CC2)c1]

To a mixture of cyclopropylmethanol (492 mg, 6.82 mmol, 539.11 μL, 1.2 eq.) in THF (30 mL) was added NaH (273 mg, 6.82 mmol, 60% purity, 1.2 eq.) in one portion at 0° C. under N₂ and stirred for 10 mins. Then compound 1A (1 g, 5.68 mmol, 1 eq) was added in one portion at 0° C. under N₂. The mixture was stirred at 25° C. for 12 hours. The residue was poured into water (50 mL). The aqueous phase was extracted with DCM (30 mL*3). The combined organic phase was washed with brine (20 mL*3), dried with anhydrous Na₂SO₄, filtered and concentrated in vacuum. The residue was purified by silica gel chromatography (column weight: 4 g, diameter: 100 mm, 100-200 mesh silica gel, Petroleum ether/Ethyl acetate=5:1). Compound 1B (1 g, 4.22 mmol, 74.33% yield) was obtained as a colourless oil.

¹H NMR (DMSO-d₆, 400 MHz) δ_{H}=8.04 (d, J=5.6 Hz, 1H), 7.22-7.17 (m, 1H), 7.14-7.10 (m, 1H), 4.09 (d, J=7.2 Hz, 2H), 1.28-1.16 (m, 1H), 0.57-0.50 (m, 2H), 0.34-0.27 (m, 2H).

### Step 2: 2-(cyclopropylmethoxy)-4-(4,4,5,5-tetramethyl-1,3,2-dioxaborolan-2-yl)pyridine (Compound 1C)

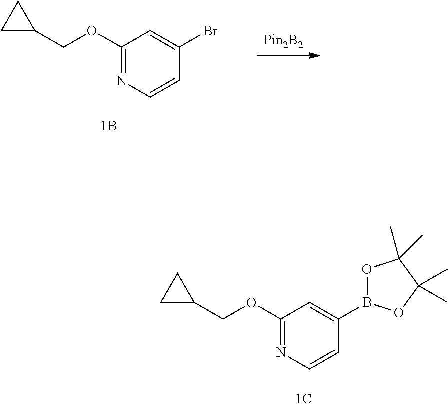 [SMILES: Brc1ccnc(OCC2CC2)c1 ; CC1(C)OB(c2ccnc(OCC3CC3)c2)OC1(C)C]

To a mixture of compound 1B (1 g, 4.38 mmol, 1 eq) in dioxane (10 mL) was added AcOK (861 mg, 8.77 mmol, 2 eq), Pin₂B₂ (1.34 g, 5.26 mmol, 1.2 eq) and Pd(dppf)Cl₂ (321 mg, 438.43 μmol, 0.1 eq) in one portion at 25° C. under N₂. The mixture was stirred at 100° C. for 12 hours. The residue was poured into water (100 mL). The aqueous phase was extracted with DCM (70 mL*3). The combined organic phase was washed with brine (100 mL), dried with anhydrous Na₂SO₄, filtered and concentrated in vacuum. The residue was purified by silica gel chromatography (column weight: 20 g, diameter: 100 mm, 100-200 mesh silica gel, Dichloromethane: Methanol=10:1). Compound 1C (1.2 g, 3.73 mmol, 85.03% yield) was obtained as a brown oil.

¹H NMR (DMSO-d₆, 400 MHz) δ_{H}=8.16 (d, J=4.8 Hz, 1H), 7.10 (d, J=5.2 Hz, 1H), 6.92 (s, 1H), 4.08 (d, J=7.2 Hz, 2H), 1.29 (s, 12H), 1.24-1.17 (m, 1H), 0.57-0.48 (m, 2H), 0.34-0.26 (m, 2H).

### Step 3: (S)-6-chloro-N4-(5-(2-(cyclopropylmethoxy)pyridin-4-yl)-2-(3,4-dimethylpiperazin-1-yl)-4-fluorophenyl)pyrimidine-4,5-diamine (DDO-2213_008)

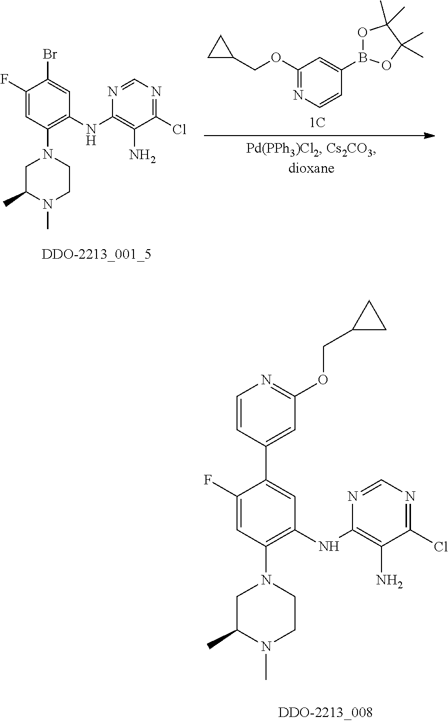 [SMILES: C[C@H]1CN(c2cc(F)c(Br)cc2Nc2ncnc(Cl)c2N)CCN1C ; CC1(C)OB(c2ccnc(OCC3CC3)c2)OC1(C)C ; C[C@H]1CN(c2cc(F)c(-c3ccnc(OCC4CC4)c3)cc2Nc2ncnc(Cl)c2N)CCN1C]

DDO-2213_001_5 is synthesized as shown in Example 37. To a mixture of DDO-2213_001_5 (150 mg, 349.07 μmol, 1 eq) and compound 1C (96 mg, 349.07 μmol, 1 eq) in dioxane (5 mL) and H₂O (0.8 mL) was added Pd(dppf)Cl₂ (26 mg, 34.91 μmol, 0.1 eq) and Cs₂CO₃ (114 mg, 349.07 μmol, 1 eq) in one portion at 25° C. under N₂. The mixture was heated to 100° C. and stirred for 12 hours. The residue was filtered via a filter paper and concentrated under reduce pressure. The residue was purified by prep. HPLC (Column: Phenomenex Gemini-NX C18 75*30 mm*3 μm; mobile phase: [water (0.04% NH₃H₂O+10 mM NH₄HCO₃)-ACN]; B %: 27%-67%; 11 min). DDO-2213_008 (19 mg, 37.83 μmol, 10.84% yield, 99.16% purity) was obtained as a white solid.

¹H NMR (DMSO-d₆, 400 MHz) δ_{H}=8.18 (d, J=5.6 Hz, 1H), 8.09 (s, 1H), 7.93 (d, J=8.8 Hz, 1H), 7.81 (s, 1H), 7.12 (d, J=5.6 Hz, 1H), 7.06 (d, J=12.8 Hz, 1H), 6.92 (s, 1H), 5.36 (s, 2H), 4.12 (d, J=7.2 Hz, 2H), 3.04-2.93 (m, 2H), 2.83-2.70 (m, 2H), 2.48-2.42 (m, 1H), 2.34-2.22 (m, 1H), 2.18 (s, 3H), 2.17-2.10 (m, 1H), 1.32-1.19 (m, 1H), 0.95 (d, J=6.4 Hz, 3H), 0.60-0.49 (m, 2H), 0.37-0.28 (m, 2H)

HPLC Rₜ=3.095 min in 8 min chromatography, XBridge Shield RP18, 5 μm, 2.1*50 mm, 8 μm, purity 99.16%.

LCMS Rₜ=2.480 min in 4 min chromatography, XBridge Shield RP18, 5 μm, 2.1*50 mm, 8 μm, purity 99.89%, MS ESI calcd. for 497.21 [M+H]⁺498.21, found 498.3.

### Example 45. 6-chloro-N4-(5-(2-(cyclopropylmethoxy)pyridin-4-yl)-4-fluoro-2-((3S,5R)-3,4,5-trimethylpiperazin-1-yl)phenyl)pyrimidine-4,5-diamine (Compound 131, Table 3)

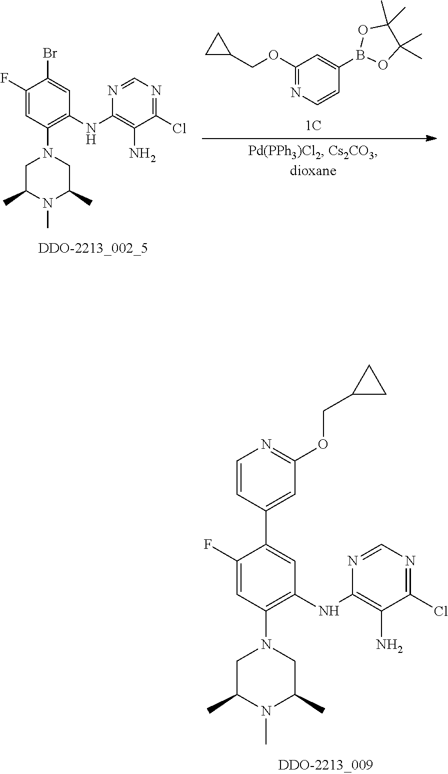 [SMILES: C[C@@H]1CN(c2cc(F)c(Br)cc2Nc2ncnc(Cl)c2N)C[C@H](C)N1C ; CC1(C)OB(c2ccnc(OCC3CC3)c2)OC1(C)C ; C[C@@H]1CN(c2cc(F)c(-c3ccnc(OCC4CC4)c3)cc2Nc2ncnc(Cl)c2N)C[C@H](C)N1C]

### Step 1:6-chloro-N4-(5-(2-(cyclopropylmethoxy)pyridin-4-yl)-4-fluoro-2-((3S,5R)-3,4,5-trimethylpiperazin-1-yl)phenyl)pyrimidine-4,5-diamine (DDO-2213_009)

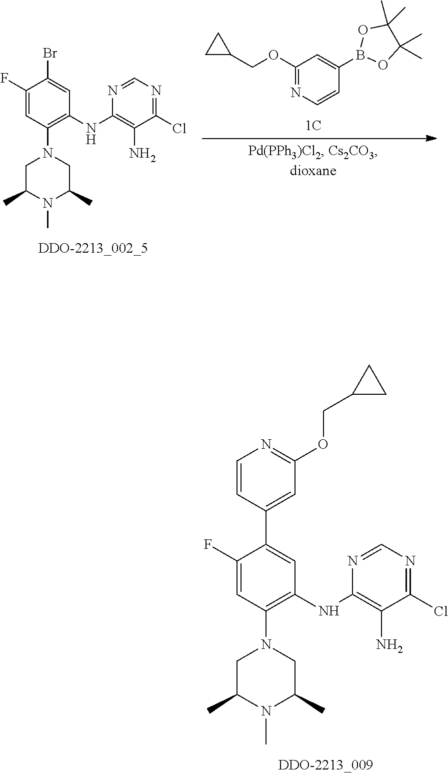 [SMILES: C[C@@H]1CN(c2cc(F)c(Br)cc2Nc2ncnc(Cl)c2N)C[C@H](C)N1C ; CC1(C)OB(c2ccnc(OCC3CC3)c2)OC1(C)C ; C[C@@H]1CN(c2cc(F)c(-c3ccnc(OCC4CC4)c3)cc2Nc2ncnc(Cl)c2N)C[C@H](C)N1C]

DDO-2213_002_5 is synthesized as shown in Example 38 and Compound 1C is synthesized as shown in Example 44. To a mixture of DDO-2213_002_5 (150 mg, 338.03 μmol, 1 eq) and compound 1C (93 mg, 338.03 μmol, 1 eq) in dioxane (5 mL) and H₂O (0.8 mL) was added Pd(dppf)Cl₂ (25 mg, 33.80 mol, 0.1 eq) and Cs₂CO₃ (110 mg, 338.03 μmol, 1 eq) in one portion at 25° C. under N₂. The mixture was heated to 100° C. and stirred for 12 hours. The residue was filtered via a filter paper and concentrated under reduce pressure. The residue was purified by prep. HPLC (Column: Phenomenex Gemini-NX C18 75*30 mm*3 μm; mobile phase: [water (0.04% NH₃H₂O+10 mM NH₄HCO₃)-ACN]; B %: 35%-74%; 11 min). DDO-2213_009 (17.7 mg, 34.45 μmol, 10.19% yield, 99.66% purity) was obtained as a white solid.

¹H NMR (DMSO-d₆, 400 MHz) δ_{H}=8.18 (d, J=5.6 Hz, 1H), 8.11 (s, 1H), 7.92 (d, J=8.6 Hz, 1H), 7.81 (s, 1H), 7.12 (d, J=5.6 Hz, 1H), 7.03 (d, J=12.8 Hz, 1H), 6.92 (s, 1H), 5.34 (s, 2H), 4.12 (d, J=7.2 Hz, 2H), 2.99 (d, J=11.2 Hz, 2H), 2.48-2.42 (m, 2H), 2.30-2.19 (m, 2H), 2.16 (s, 3H), 1.31-1.20 (m, 1H), 0.96 (d, J=6.4 Hz, 6H), 0.58-0.52 (m, 2H), 0.35-0.30 (m, 2H).

HPLC Rₜ=3.268 min in 8 min chromatography, XBridge Shield RP18, 5 μm, 2.1*50 mm, 8 μm, purity 99.66%.

LCMS Rₜ=2.611 min in 4 min chromatography, XBridge Shield RP18, 5 μm, 2.1*50 mm, 8 μm, purity 99.52%, MS ESI calcd. for 511.23 [M+H]⁺512.23, found 512.4.

### Example 46. (1-(5-((5-amino-6-chloropyrimidin-4-yl)amino)-2-fluoro-4-((3S,5R)-3,4,5-trimethylpiperazin-1-yl)phenyl)-1H-1,2,3-triazol-4-yl)(morpholino)methanone (Compound 132, Table 3)

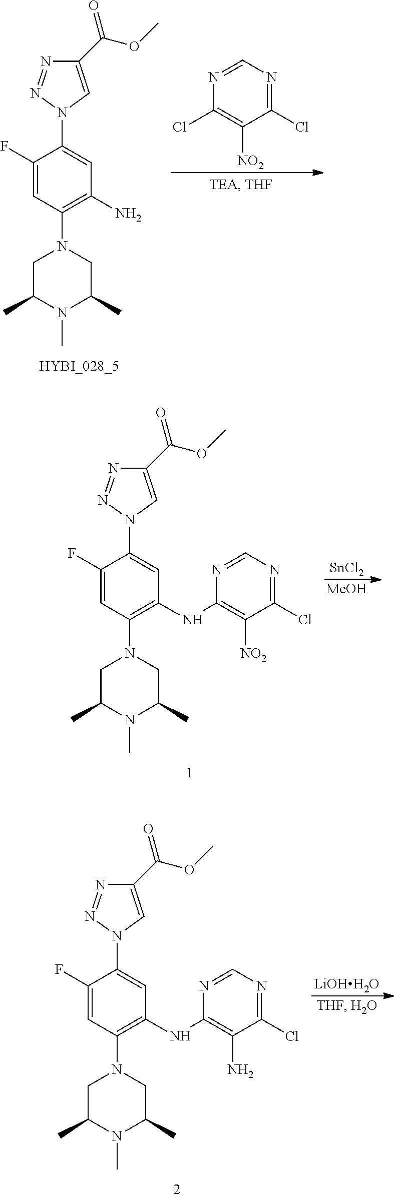

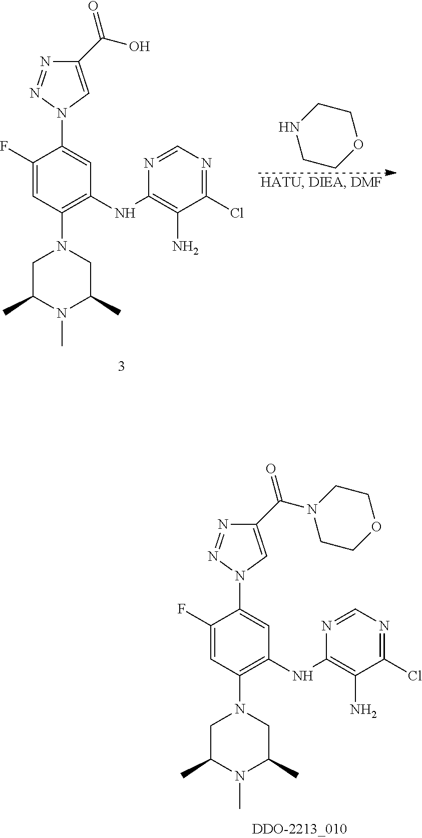

### Step 1: Synthesis of methyl 1-(5-amino-2-fluoro-4-((3S,5R)-3,4,5-trimethylpiperazin-1-yl)phenyl)-1H-1,2,3-triazole-4-carboxylate (HYBI_028_5)

Step 1A: Synthesis of Compound 2 as seen in scheme below.

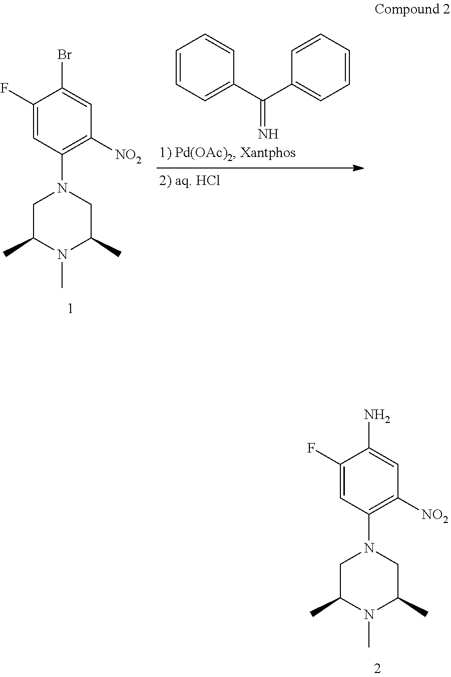 [SMILES: C[C@@H]1CN(c2cc(F)c(Br)cc2[N+](=O)[O-])C[C@H](C)N1C ; N=C(c1ccccc1)c1ccccc1 ; C[C@@H]1CN(c2cc(F)c(N)cc2[N+](=O)[O-])C[C@H](C)N1C]

To a mixture of compound 1 (8.4 g, 24.26 mmol, 1 eq) and diphenylmethanimine (6.60 g, 36.40 mmol, 6.11 mL, 1.5 eq) in dioxane (300 mL) was added Pd(OAc)₂ (545 mg, 2.43 mmol, 0.1 eq), Xantphos (2.11 g, 3.64 mmol, 0.15 eq) and Cs₂CO₃ (15.81 g, 48.53 mmol, 2 eq) in one portion at 25° C. under N₂. The mixture was heated to 100° C. and stirred for 12 hours. The mixture was filtered via a filter paper and kieselguhr. The residue was purified by silica gel chromatography (column weight: 80 g, diameter: 100 mm, 100-200 mesh silica gel, Dichloromethane: Methanol=10:1) to give the intermediate. The intermediate (13.3 g, 20.03 mmol, 1 eq) in THF (150 mL) was added HCl (12 M, 16.69 mL, 10 eq) in one portion. The mixture was stirred at 25° C. for 12 hours. The reaction mixture was poured into water (200 mL). The aqueous phase was washed with ethyl acetate (100 mL×3). The pH of the aqueous phase was adjusted to around 8 by progressively adding solid NaHCO₃. Then the aqueous phase was extracted with DCM (100 mL*3). The combined organic phase was washed with brine (100 mL*2), dried with anhydrous Na₂SO₄, filtered and concentrated in vacuum. The crude product was used in the next step without further purification. Compound 2 (4.5 g, 9.06 mmol, 45.22% yield) was obtained as a yellow oil.

¹H NMR (DMSO-d₆, 400 MHz) δ_{H}=7.23 (d, J=9.0 Hz, 1H), 7.12 (d, J=12.8 Hz, 1H), 5.46 (s, 2H), 3.31 (s, 1H), 3.17 (d, J=5.6 Hz, 3H), 2.84 (d, J=9.2 Hz, 2H), 2.17 (s, 3H), 0.99 (d, J=4.4 Hz, 6H).

### Step 1B: (2S,6R)-4-(4-azido-5-fluoro-2-nitrophenyl)-1,2,6-trimethylpiperazine

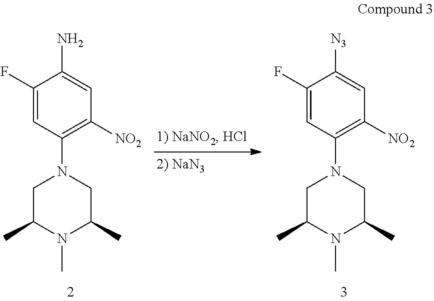 [SMILES: C[C@@H]1CN(c2cc(F)c(N)cc2[N+](=O)[O-])C[C@H](C)N1C ; C[C@@H]1CN(c2cc(F)c(N=[N+]=[N-])cc2[N+](=O)[O-])C[C@H](C)N1C]

To a mixture of compound 2 (4.5 g, 15.94 mmol, 1 eq) in HCl (2 M, 79.70 mL, 10 eq) was added a solution of NaNO₂ (1.65 g, 23.91 mmol, 1.5 eq) in H₂O (20 mL) dropwise at 0° C. After 0.5 hr, a solution of NaN₃ (1.7 g, 26.15 mmol, 1.64 eq) in H₂O (20 mL) was added into the mixture. The mixture was allowed to warm up to 15° C. and stirred for 1 hr to give a brown mixture. The pH of the mixture was adjusted to around 9 with 2N NaOH. The solid formed was filtered and the cake was washed with H₂O (50 mL×2). The cake was then dried to give the product. The product compound 3 (4 g, 10.53 mmol, 66.09% yield) was obtained as a brown solid and used into the next step without further purification.

¹H NMR (DMSO-d₆, 400 MHz) δ_{H}=7.82 (d, J=8.6 Hz, 1H), 7.30 (d, J=13.4 Hz, 1H), 3.01 (d, J=12.0 Hz, 2H), 2.63 (t, J=11.3 Hz, 2H), 2.30-2.21 (m, 2H), 2.18 (s, 3H), 1.00 (d, J=6.1 Hz, 6H).

### Step 1C: Methyl 1-(2-fluoro-5-nitro-4-((3S,5R)-3,4,5-trimethylpiperazin-1-yl)phenyl)-1H-1,2,3-triazole-4-carboxylate

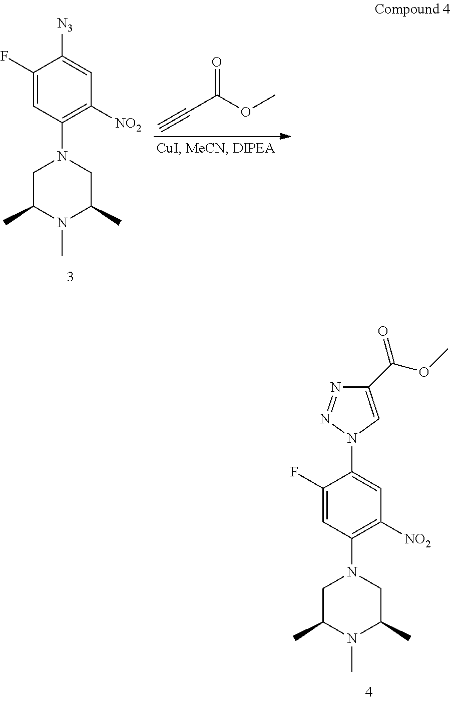

To a mixture of compound 3 (3.9 g, 12.65 mmol, 1 eq) and methyl prop-2-ynoate (1.28 g, 15.18 mmol, 1.26 mL, 1.2 eq) in CH₃CN (40 mL) was added CuI (723 mg, 3.79 mmol, 0.3 eq) and DIEA (4.90 g, 37.95 mmol, 6.61 mL, 3 eq) under N₂. The reaction mixture was stirred at 25° C. for 10 hr. The reaction mixture was filtered. The filter cake was washed with CH₃CN (100 mL×3). The filtrate was concentrated. The product was concentrated directly for the next reaction. Crude compound 4 (6 g, crude) was obtained as a Black Brown solid.

¹H NMR (DMSO-d₆, 400 MHz) δ_{H}=9.25 (s, 1H), 8.43 (d, J=8.0 Hz, 1H), 7.51 (d, J=13.2 Hz, 1H), 3.92-3.79 (m, 2H), 3.50 (s, 1H), 3.29-3.06 (m, 4H), 2.82 (t, J=11.6 Hz, 2H), 2.24 (s, 3H), 1.08-1.01 (m, 6H).

### Step 1D: Methyl 1-(5-amino-2-fluoro-4-((3S,5R)-3,4,5-trimethylpiperazin-1-yl)phenyl)-1H-1,2,3-triazole-4-carboxylate

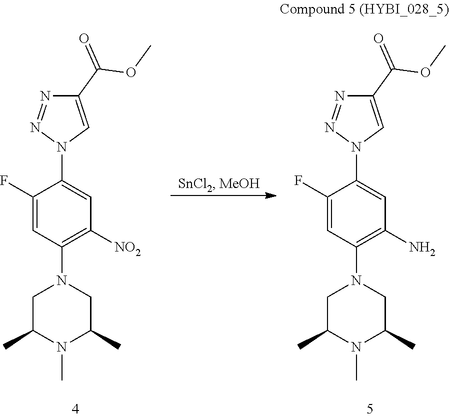

To a solution of compound 4 (6 g, 15.29 mmol, 1 eq) in MeOH (60 mL) and H₂O (12 mL) was added SnCl₂.2H₂O (10.35 g, 45.87 mmol, 3 eq). The mixture was stirred at 70° C. for 10 hours. The reaction mixture was quenched by addition NaHCO₃ (200 mL), and then extracted with DCM (100 mL*3). The combined organic layers were concentrated under reduced pressure to give a residue. The crude product was purified by silica gel chromatography (ISCO®; 40 g SepaFlash® Silica Flash Column, Eluent of 0-15% MeOH/DCM ethergradient @ 60 mL/min). Compound 5 (2 g, 4.75 mmol, 31.04% yield) was obtained as a brown solid.

¹H NMR (DMSO-d₆, 400 MHz) δ_{H}=9.10 (d, J=1.6 Hz, 1H), 7.08-6.95 (m, 2H), 5.00 (s, 2H), 3.91-3.85 (m, 3H), 3.04 (d, J=8.4 Hz, 2H), 2.43-2.32 (m, 4H), 2.22 (s, 3H), 1.03 (d, J=5.6 Hz, 6H).

### Step 2: Methyl 1-(5-((6-chloro-5-nitropyrimidin-4-yl)amino)-2-fluoro-4-((3S,5R)-3,4,5-trimethylpiperazin-1-yl)phenyl)-1H-1,2,3-triazole-4-carboxylate (Compound 1)

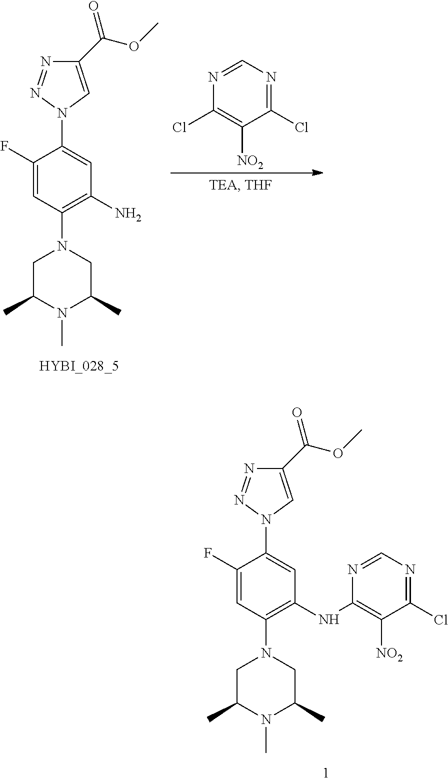 [SMILES: COC(=O)c1cn(-c2cc(N)c(N3C[C@@H](C)N(C)[C@@H](C)C3)cc2F)nn1 ; O=[N+]([O-])c1c(Cl)ncnc1Cl ; COC(=O)c1cn(-c2cc(Nc3ncnc(Cl)c3[N+](=O)[O-])c(N3C[C@@H](C)N(C)[C@@H](C)C3)cc2F)nn1]

To a solution of HYBI_028_5 (300 mg, 827.81 μmol, 1 eq.) and 4,6-dichloro-5-nitro-pyrimidine (321 mg, 1.66 mmol, 2 eq.) in THF (10 mL) was added TEA (251 mg, 2.48 mmol, 345.67 μL, 3 eq.). The mixture was stirred at 25° C. for 3 hr. The reaction mixture was diluted with DCM (100 mL*2), washed with brine (50 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-5% MeOH/DCM @ 20 mL/min). Compound 1 (240 mg, 349.44 μmol, 42.21% yield) was obtained as a brown solid.

¹H NMR (DMSO-d₆, 400 MHz) δ_{H}=4.11 (d, J=5.2 Hz, 2H), 4.08-3.99 (m, 1H), 3.91-3.88 (m, 1H), 3.57 (s, 1H), 3.32 (s, 3H), 3.17 (d, J=5.2 Hz, 6H), 2.68 (s, 1H), 2.57-2.52 (m, 4H), 1.99 (s, 1H), 1.29-1.22 (m, 1H), 1.21-1.14 (m, 1H), 1.13-0.98 (m, 1H).

### Step 3: Methyl 1-(5-((5-amino-6-chloropyrimidin-4-yl)amino)-2-fluoro-4-((3S,5R)-3,4,5-trimethylpiperazin-1-yl)phenyl)-1H-1,2,3-triazole-4-carboxylate (Compound 2)

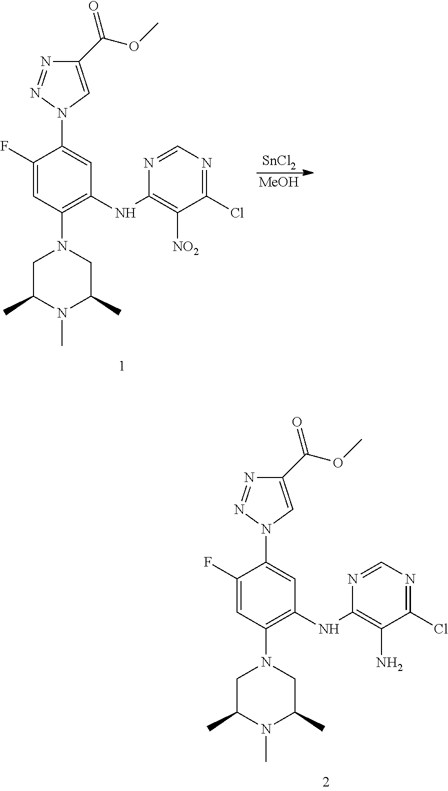

To a solution of compound 2 (240 mg, 461.61 μmol, 1 eq.) in MeOH (10 mL) and H₂O (4 mL) was added SnCl₂·2H₂O (313 mg, 1.38 mmol, 3 eq.). The mixture was stirred at 80° C. for 3 hr. The mixture was adjusted to pH 9 with NaHCO₃, filtered to remove the insoluble. The filter liquor was concentrated in vacuum. The reaction mixture was diluted with DCM (50 mL*3), washed with brine (20 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-8% MeOH/DCM @ 25 mL/min). The product compound 2 (130 mg, 202.46 μmol, 43.86% yield) was obtained as a yellow oil.

¹H NMR (DMSO-d₆, 400 MHz) δ_{H}=9.23 (d, J=1.6 Hz, 1H), 8.29-8.19 (m, 2H), 7.85 (s, 1H), 7.35-7.25 (m, 1H), 5.40 (s, 2H), 3.88 (s, 3H), 3.33 (s, 2H), 3.06-2.97 (m, 2H), 2.31-2.22 (m, 2H), 2.17 (s, 3H), 0.98 (d, J=6.0 Hz, 6H).

### Step 4:1-(5-((5-amino-6-chloropyrimidin-4-yl)amino)-2-fluoro-4-((3S,5R)-3,4,5-trimethylpiperazin-1-yl)phenyl)-1H-1,2,3-triazole-4-carboxylic Acid (Compound 3)

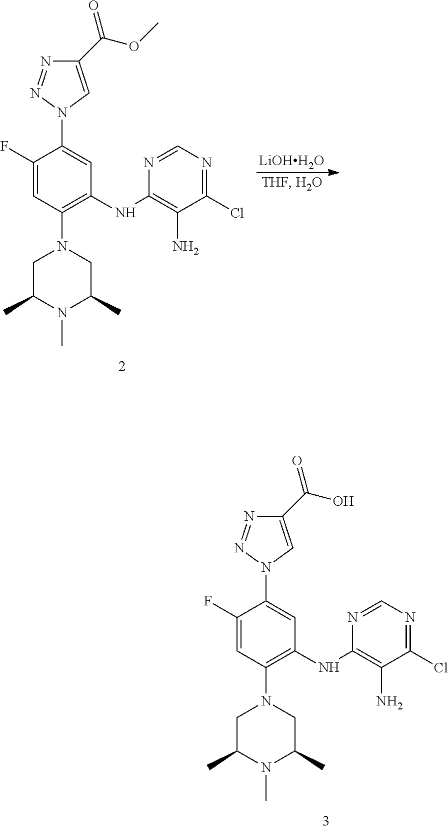

To a solution of compound 2 (130 mg, 265.34 μmol, 1 eq.) in THF (4 mL) and H₂O (1 mL) was added LiOH·H₂O (22 mg, 530.68 μmol, 2 eq.). The mixture was stirred at 25° C. for 2 hr. The reaction mixture was adjusted to pH=5 by 1N aq.HCl concentrated under reduced pressure to give a residue. The product was used directly to the next step without further purification. Compound 3 (120 mg, crude) was obtained as a yellow solid.

¹H NMR (DMSO-d₆, 400 MHz) δ_{H}=11.21 (s, 1H), 9.14-9.06 (m, 2H), 8.09 (d, J=8.0 Hz, 1H), 7.82 (s, 1H), 7.39-7.32 (m, 1H), 3.45 (s, 4H), 3.18-3.10 (m, 2H), 2.76 (d, J=4.8 Hz, 3H), 1.35 (d, J=6.4 Hz, 6H), 1.28-1.19 (m, 2H).

### Step 5: (1-(5-((5-amino-6-chloropyrimidin-4-yl)amino)-2-fluoro-4-((3S,5R)-3,4,5-trimethylpiperazin-1-yl)phenyl)-1H-1,2,3-triazol-4-yl)(morpholino)methanone (DDO-2213_010)

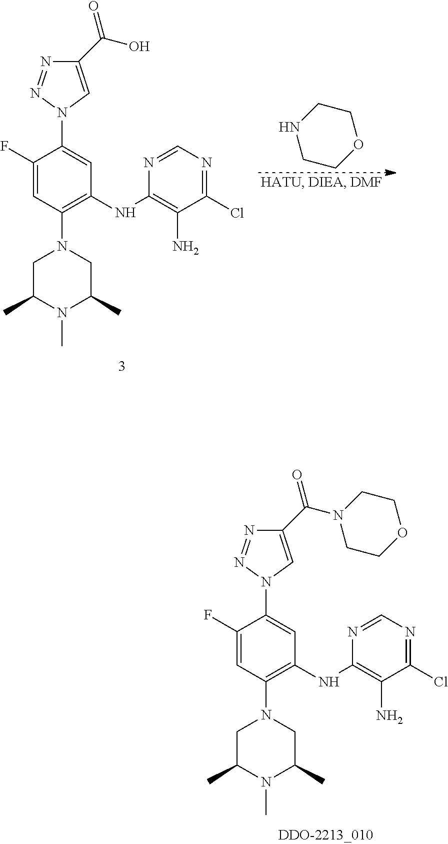

To a solution of compound 3 (120 mg, 252.15 μmol, 1 eq.) and morpholine (44 mg, 504.30 μmol, 44.38 μL, 2 eq.) in DMF (5 mL) was added HATU (144 mg, 378.23 μmol, 1.5 eq) and DIEA (98 mg, 756.45 μmol, 131.76 μL, 3 eq.). The mixture was stirred at 25° C. for 12 hr. The reaction mixture was concentrated directly. The residue was purified by prep-HPLC (Column: Phenomenex Gemini-NX C18 75*30 mm*5 μm; Mobile Phase A: purified water (0.04% NH₃H2O+10 mM NH₄HCO₃); Mobile Phase B: acetonitrile; Gradient: 10-50% B in 11 min.). DDO-2213_010 (33 mg, 60.40 μmol, 23.95% yield, 99.75% purity) was obtained as a white solid.

¹H NMR (DMSO-d₆, 400 MHz) δ_{H}=8.97 (s, 1H), 8.25 (d, J=8.0 Hz, 2H), 7.86 (s, 1H), 7.30 (d, J=12.4 Hz, 1H), 5.39 (s, 2H), 4.04 (s, 2H), 3.33 (s, 6H), 3.00 (d, J=11.2 Hz, 2H), 2.54-2.51 (m, 2H), 2.29 (d, J=6.4 Hz, 2H), 2.17 (s, 3H), 0.98 (d, J=6.0 Hz, 6H).

HPLC Rₜ=2.947 min in 8 min chromatography, XBridge Shield RP18, 5 μm, 2.1*50 mm, purity 99.75%.

LCMS Rₜ=2.268 min in 4 min chromatography, XBridge Shield RP18, 5 μm, 2.1*50 mm, purity 99.24%, MS ESI calcd. for 544.22 [M+H]⁺545.22, found 545.4.

### Example 47. (S)-3′-((5-amino-6-chloropyrimidin-4-yl)amino)-4′-(3,4-dimethylpiperazin-1-yl)-2′-fluoro-N-(2-morpholinoethyl)-[1,1′-biphenyl]-4-carboxamide (Compound 133, Table 3)

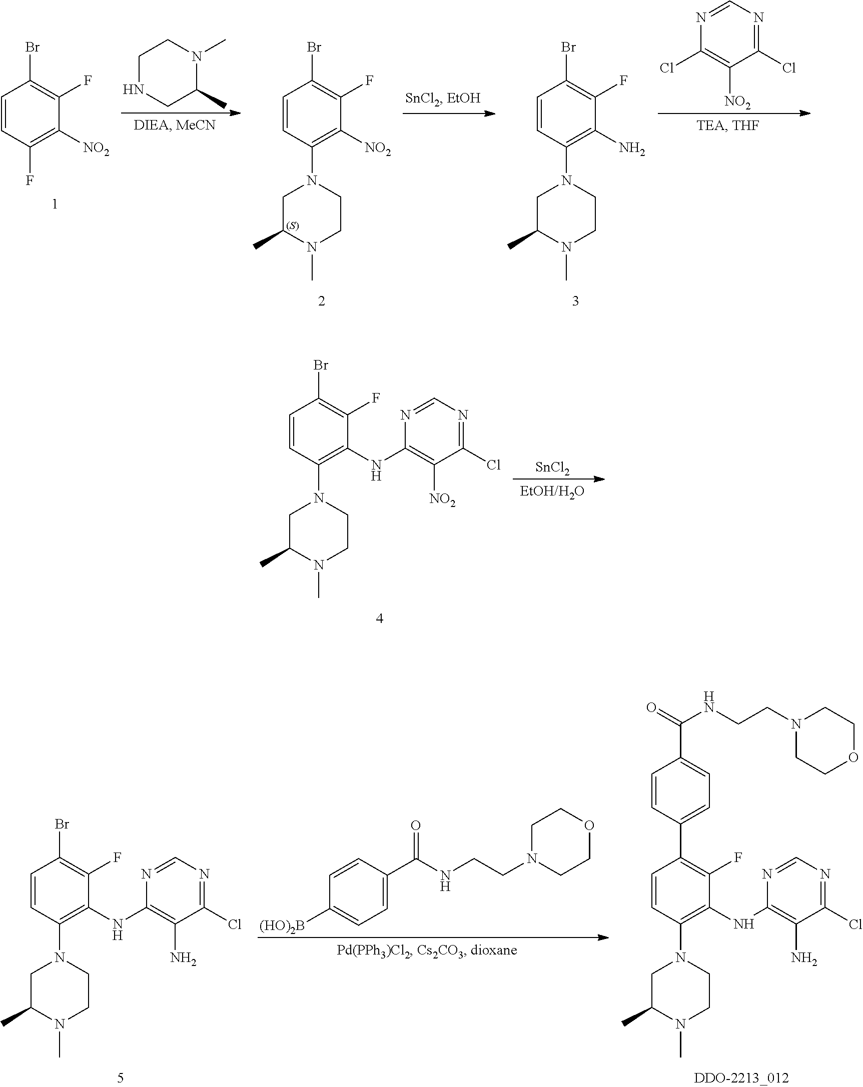

### Step 1: (S)-4-(4-bromo-3-fluoro-2-nitrophenyl)-1,2-dimethylpiperazine (Compound 2)

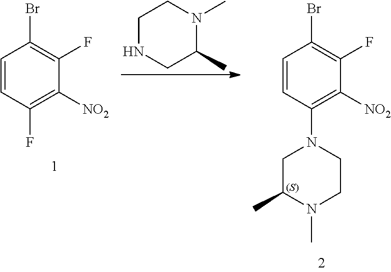

To a solution of compound 1 (2 g, 8.40 mmol, 1 eq.) in CH₃CN (20 mL) was added (2S)-1,2-dimethylpiperazine (960 mg, 8.40 mmol, 1 eq.) and DIEA (2.17 g, 16.81 mmol, 2.93 mL, 2 eq.). The mixture was diluted with DCM (200 mL), washed with brine (50 mL*3). The organic layer was dried over Na₂SO₄, filtered and concentrated under reduced pressure to give a residue. The residue was purified by flash silica gel chromatography (ISCO®; 20 g SepaFlash® Silica Flash Column, Eluent of 0-3% Methanol/Dichloromethane @ 40 mL/min). Compound 2 (2.66 g, 7.59 mmol) was obtained as a yellow solid.

¹H NMR (DMSO-d₆, 400 MHz) δ_{H}=7.84 (d, J=8.8 Hz, 1H), 7.16 (J=1.6, 9.2 Hz, 1H), 3.03 (s, 3H), 2.78-2.71 (m, 1H), 2.66-2.58 (m, 1H), 2.22-2.07 (m, 5H), 0.97 (d, J=6.4 Hz, 3H).

### Step 2: (S)-3-bromo-6-(3,4-dimethylpiperazin-1-yl)-2-fluoroaniline (Compound 3)

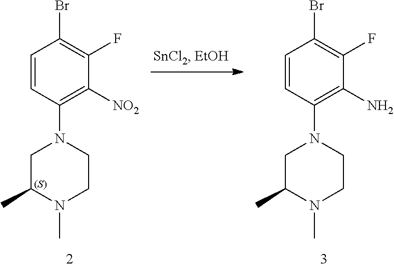

To a solution of compound 2 (2.66 g, 8.01 mmol, 1 eq.) in EtOH (40 mL) and H₂O (4 mL) was added SnCl₂·2H₂O (4.52 g, 20.02 mmol, 2.5 eq.). The mixture was stirred at 80° C. for 3 hr. The reaction mixture was adjusted to pH=9 by 1N aq.NaHCO₃. The filter liquor was diluted with DCM (100 mL), washed with brine (50 mL*2). The organic layer was dried over Na₂SO₄, filtered and concentrated under reduced pressure to give a residue. The residue was purified by flash silica gel chromatography (ISCO®; 20 g SepaFlash® Silica Flash Column, Eluent of 0-14% MeOH/DCM @ 40 mL/min). The product compound 3 (1.45 g, 4.53 mmol, 56.52% yield) was obtained as a yellow solid

¹H NMR (DMSO-d₆, 400 MHz) δ_{H}=6.82-6.75 (m, 1H), 6.73-6.67 (m, 1H), 4.89 (s, 2H), 2.89 (d, J=11.2 Hz, 2H), 2.77 (d, J=11.2 Hz, 1H), 2.68 (d, J=2.4 Hz, 1H), 2.41-2.24 (m, 3H), 2.22 (s, 3H), 1.00 (d, J=6.0 Hz, 3H).

### Step 3:(S)—N-(3-bromo-6-(3,4-dimethylpiperazin-1-yl)-2-fluorophenyl)-6-chloro-5-nitropyrimidin-4-amine (Compound 4)

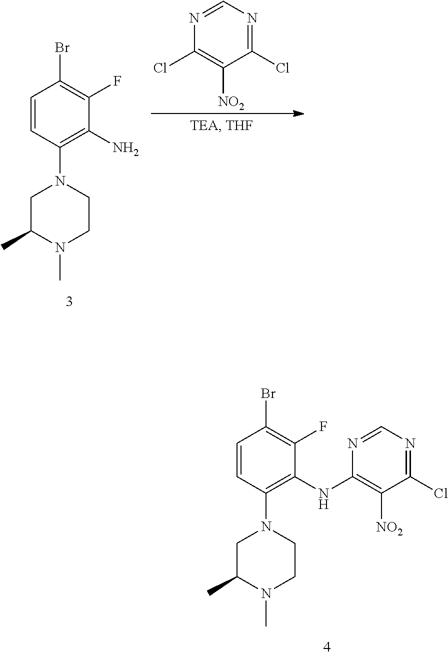 [SMILES: C[C@H]1CN(c2ccc(Br)c(F)c2N)CCN1C ; O=[N+]([O-])c1c(Cl)ncnc1Cl ; C[C@H]1CN(c2ccc(Br)c(F)c2Nc2ncnc(Cl)c2[N+](=O)[O-])CCN1C]

To a solution of compound 3 (900 mg, 2.98 mmol, 1 eq.) and 4,6-dichloro-5-nitro-pyrimidine (1.16 g, 5.96 mmol, 2 eq.) in THF (9 mL) was added TEA (905 mg, 8.93 mmol, 1.24 mL, 3 eq.). The mixture was stirred at 40° C. for 12 hr. The reaction mixture was diluted with DCM (100 mL*2), washed with brine (50 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-8% MeOH/DCM @ 30 mL/min). Compound 4 (830 mg, 1.44 mmol, 48.50% yield) was obtained as a brown solid.

¹H NMR (DMSO-d₆, 400 MHz) δ_{H}=9.89 (s, 1H), 8.46 (s, 1H), 7.69-7.56 (m, 1H), 6.98-6.87 (m, 1H), 3.27-3.18 (m, 2H), 3.01-2.86 (m, 2H), 2.83-2.67 (m, 3H), 2.66-2.54 (m, 3H), 1.15-1.08 (m, 3H).

### Step 4: (S)-N4-(3-bromo-6-(3,4-dimethylpiperazin-1-yl)-2-fluorophenyl)-6-chloropyrimidine-4,5-diamine (Compound 5)

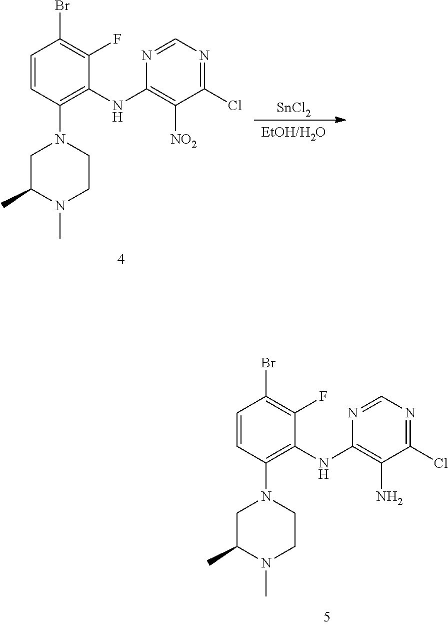

To a solution of compound 4 (830 mg, 1.81 mmol, 1 eq.) in EtOH (20 mL) and H₂O (6 mL) was added SnCl₂·2H₂O (1.22 g, 5.42 mmol, 3 eq.). The mixture was stirred at 80° C. for 3 hr. The reaction mixture was adjusted to pH=9 by 1N aq.NaHCO₃. The filter liquor was diluted with DCM (100 mL), washed with brine (50 mL*2). The organic layer was dried over Na₂SO₄, filtered and concentrated under reduced pressure to give a residue. The residue was purified by flash silica gel chromatography (ISCO®; 20 g SepaFlash® Silica Flash Column, Eluent of 0-16% MeOH/DCM @ 40 mL/min). Compound 5 (460 mg, 873.51 μmol, 48.38% yield) was obtained as a yellow solid.

¹H NMR (DMSO-d₆, 400 MHz) δ_{H}=8.06 (s, 1H), 7.73 (s, 1H), 7.49 (s, 1H), 6.86 (dd, J=1.2, 8.8 Hz, 1H), 5.48 (s, 2H), 3.44-3.26 (m, 1H), 3.20-3.15 (m, 1H), 2.99-2.90 (m, 2H), 2.73-2.66 (m, 2H), 2.45-2.37 (m, 1H), 2.17 (s, 3H), 0.90 (d, J=6.4 Hz, 3H).

### Step 5: (S)-3′-((5-amino-6-chloropyrimidin-4-yl)amino)-4′-(3,4-dimethylpiperazin-1-yl)-2′-fluoro-N-(2-morpholinoethyl)-[1,1′-biphenyl]-4-carboxamide (DDO-2213_012)

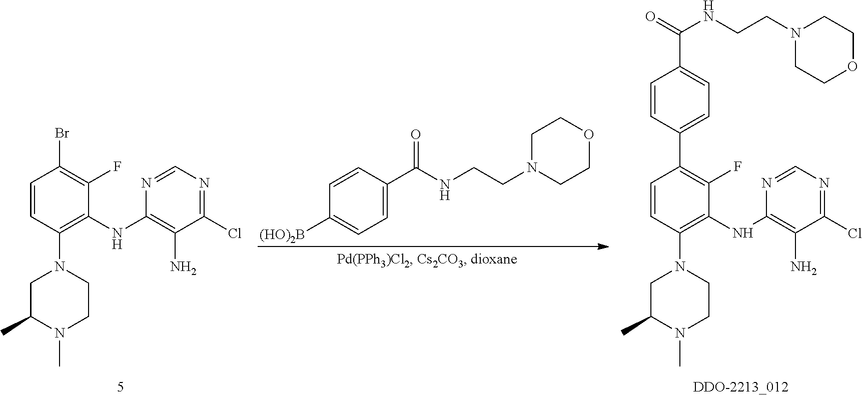 [SMILES: C[C@H]1CN(c2ccc(Br)c(F)c2Nc2ncnc(Cl)c2N)CCN1C ; O=C(NCCN1CCOCC1)c1ccc(B(O)O)cc1 ; C[C@H]1CN(c2ccc(-c3ccc(C(=O)NCCN4CCOCC4)cc3)c(F)c2Nc2ncnc(Cl)c2N)CCN1C]

A mixture of compound 5 (150 mg, 349.07 μmol, 1 eq.), [4-(2-morpholinoethylcarbamoyl)phenyl]boronic acid (68 mg, 244.35 μmol, 0.7 eq.), Pd(dppf)Cl₂·CH₂Cl₂ (29 mg, 34.91 μmol, 0.1 eq.) and Cs₂CO₃ (228 mg, 698.13 μmol, 2 eq.) in dioxane (4 mL) and H₂O (0.8 mL) was degassed and purged with N₂ for 3 times, and then the mixture was stirred at 100° C. for 12 hr under N₂ atmosphere. The reaction mixture was diluted with DCM (50 mL), washed with brine (50 mL). The organic layer was dried over Na₂SO₄, filtered and concentrated under reduced pressure to give a residue. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 10-50% B in 11 min.). The crude product DDO-2213_012 (26.1 mg, 43.16 μmol, 12.36% yield, 96.42% purity) was obtained as a white solid

¹H NMR (DMSO-d₆, 400 MHz) δ_{H}=8.46 (s, 1H), 8.03 (s, 1H), 7.90 (d, J=8.4 Hz, 2H), 7.71 (s, 1H), 7.59 (d, J=7.6 Hz, 2H), 7.40 (t, J=8.4 Hz, 1H), 6.97 (d, J=8.4 Hz, 1H), 5.45 (s, 2H), 3.57 (d, J=4.0 Hz, 4H), 3.40 (q, J=6.4 Hz, 2H), 3.36-3.30 (m, 7H), 3.00 (dd, J=12.0, 17.2 Hz, 1H), 3.07-2.93 (m, 1H), 2.82-2.64 (m, 2H), 2.52-2.48 (m, 20H), 2.48-2.44 (m, 3H), 2.42 (s, 4H), 2.22-2.17 (m, 1H), 2.15 (s, 3H), 2.11-2.02 (m, 1H), 0.90 (d, J=6.0 Hz, 3H).

HPLC Rₜ=2.029 min in 8 min chromatography, XBridge Shield RP18, 5 μm, 2.1*50 mm, purity 96.42%.

LCMS Rₜ=1.658 min in 4 min chromatography, XBridge Shield RP18, 5 μm, 2.1*50 mm, purity 97.15%, MS ESI calcd. for 582.26 [M+H]⁺ 583.26, found 583.5.

### Example 48. N-(3′-((5-amino-6-chloropyrimidin-4-yl)amino)-2′-fluoro-4′-((3S,5R)-3,4,5-trimethylpiperazin-1-yl)-[1,1′-biphenyl] yl) cyclohexanecarboxamide (Compound 134, Table 3)

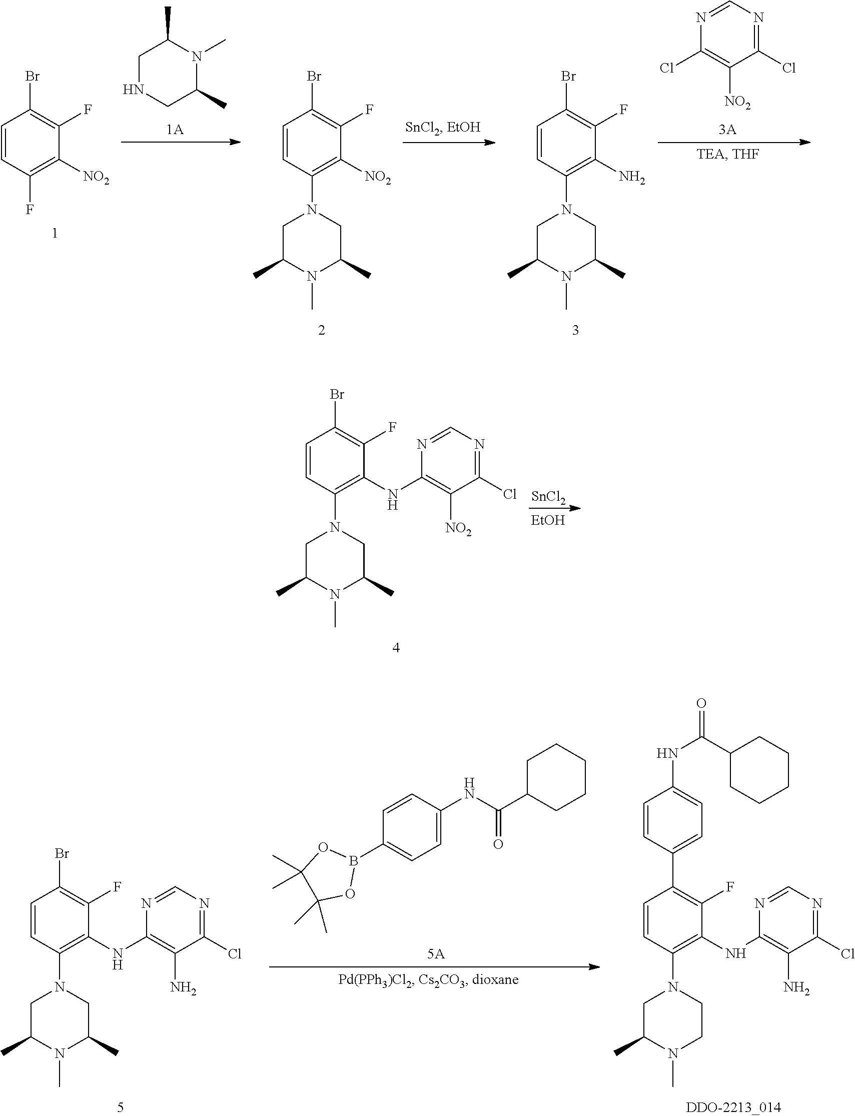

### Step 1: (2S,6R)-4-(4-bromo-3-fluoro-2-nitrophenyl)-1,2,6-trimethylpiperazine (Compound 1)

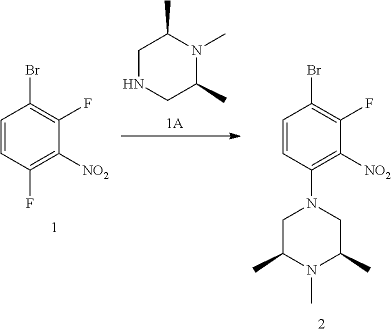

To a mixture of compound 1 (2 g, 8.40 mmol, 1 eq.) and compound 1A (1.08 g, 8.40 mmol, 1 eq.) in CH₃CN (20 mL) was added DIEA (2.17 g, 16.80 mmol, 2.93 mL, 2 eq.). The reaction mixture was stirred at 25° C. for 4 hr. The reaction mixture was extracted with DCM (100 mL*3). The combined organic phase was washed with brine (100 mL*2), dried with anhydrous Na₂SO₄, filtered and concentrated in vacuum. The reaction mixture was concentrated to dryness. The crude product was purified by silica gel chromatography (DCM: MeOH=5%). Compound 2 (2.9 g, 8.26 mmol, 98.32% yield) was obtained as yellow oil.

¹H NMR (DMSO-d₆, 400 MHz) δ_{H}=7.83 (dd, J=8.0, 9.2 Hz, 1H), 7.14 (dd, J=1.6, 9.2 Hz, 1H), 3.04-2.97 (m, 2H), 2.63 (t, J=11.2 Hz, 2H), 2.22-2.17 (m, 1H), 2.16 (s, 3H), 2.15-2.12 (m, 1H), 0.98 (d, J=6.0 Hz, 6H).

### Step 2: 3-bromo-2-fluoro-6-((3S,5R)-3,4,5-trimethylpiperazin-1-yl aniline (Compound 3)

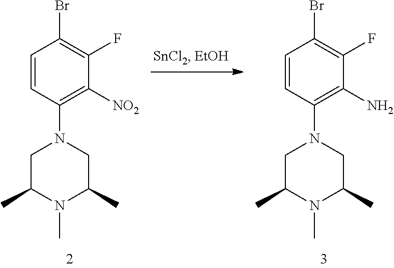

To a mixture of compound 2 (2.9 g, 8.38 mmol, 1 eq.) in EtOH (30 mL) and H₂O (5 mL) was added SnCl₂·2H₂O (5.67 g, 25.13 mmol, 3 eq.). The reaction mixture was stirred at 80° C. for 2 hr. The reaction mixture was quenched by addition NaHCO₃ (150 mL), and then extracted with DCM (100 mL*3). The combined organic layers were concentrated under reduced pressure to give a residue. The crude product was purified by silica gel chromatography (5% of MeOH in DCM). Compound 3 (1.6 g, 5.01 mmol, 59.87% yield) was obtained as brown oil.

¹H NMR (DMSO-d₆, 400 MHz) δ_{H}=6.81-6.75 (m, 1H), 6.71-6.66 (m, 1H), 4.90 (s, 2H), 2.90 (d, J=9.2 Hz, 2H), 2.42-2.30 (m, 4H), 2.21 (s, 3H), 1.01 (d, J=5.6 Hz, 6H).

### Step 3: N-(3-bromo-2-fluoro-6-((3S,5R)-3,4,5-trimethylpiperazin-1-yl)phenyl)-6-chloro-5-nitropyrimidin-4-amine (Compound 4)

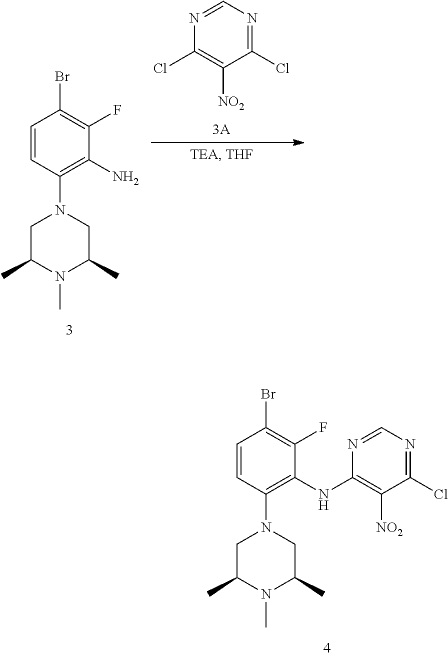

To a mixture of compound 3A (1.23 g, 6.32 mmol, 2 eq.) and TEA (960 mg, 9.49 mmol, 1.32 mL, 3 eq.) in THF (5 mL) was added the mixture of compound 3 (1 g, 3.16 mmol, 1 eq.) in THF (5 mL). The reaction mixture stirred 40° C. for 2 hr. The mixture was extracted with DCM (50 mL*3). The combined organic phase was washed with brine (50 mL*2), dried with anhydrous Na₂SO₄, filtered and concentrated in vacuum. The crude product was purified by silica gel chromatography (5% of MeOH in DCM). Compound 4 (1.3 g, 2.22 mmol, 70.18% yield) was obtained as yellow oil.

¹H NMR (DMSO-d₆, 400 MHz) δ_{H}=9.16-8.89 (m, 1H), 8.48-8.44 (m, 1H), 7.65 (t, J=8.0 Hz, 1H), 6.96 (d, J=8.8 Hz, 1H), 3.34 (s, 2H), 3.09 (q, J=7.2 Hz, 4H), 2.88-2.66 (m, 3H), 1.18 (t, J=7.2 Hz, 6H).

### Step 4: N4-(3-bromo-2-fluoro-6-((3S,5R)-3,4,5-trimethylpiperazin-1-yl)phenyl)-6-chloropyrimidine-4,5-diamine (Compound 5)

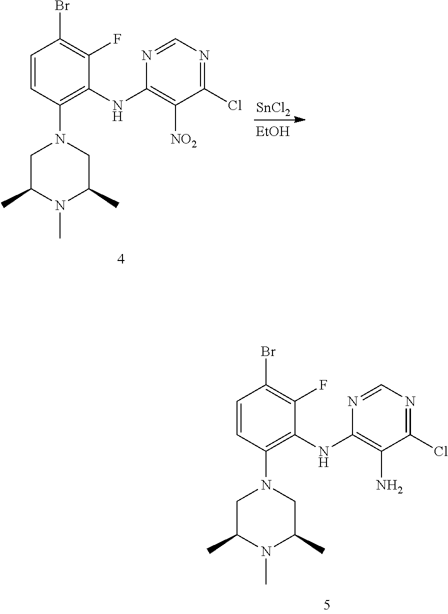

To a mixture of compound 4 (1.30 g, 2.74 mmol, 1 eq.) in EtOH (10 mL) and H₂O (2 mL) was added SnCl₂·2H₂O (1.86 g, 8.23 mmol, 3 eq.). The reaction mixture was stirred at 80° C. for 2 hr. The reaction mixture was quenched by addition NaHCO₃ (150 mL), and then extracted with DCM (100 mL*3). The combined organic layers were concentrated under reduced pressure to give a residue. The crude product was purified by silica gel chromatography (5% of MeOH in DCM). Compound 5 (640 mg, 1.34 mmol, 48.76% yield) was obtained as yellow oil.

¹H NMR (DMSO-d₆, 400 MHz) δ_{H}=8.11 (s, 1H), 7.73 (s, 1H), 7.55-7.48 (m, 1H), 6.87 (d, J=8.4 Hz, 1H), 5.47 (s, 2H), 4.11 (q, J=5.2 Hz, 2H), 3.10 (q, J=7.2 Hz, 4H), 2.38-2.11 (m, 3H), 1.09-0.91 (m, 6H).

### Step 5: N-(3′-((5-amino-6-chloropyrimidin-4-yl)amino)-2′-fluoro-4′-((3S,5R)-3,4,5-trimethylpiperazin-1-yl)-[1,1′-biphenyl]-4-yl)cyclohexanecarboxamide (DDO-2213_014)

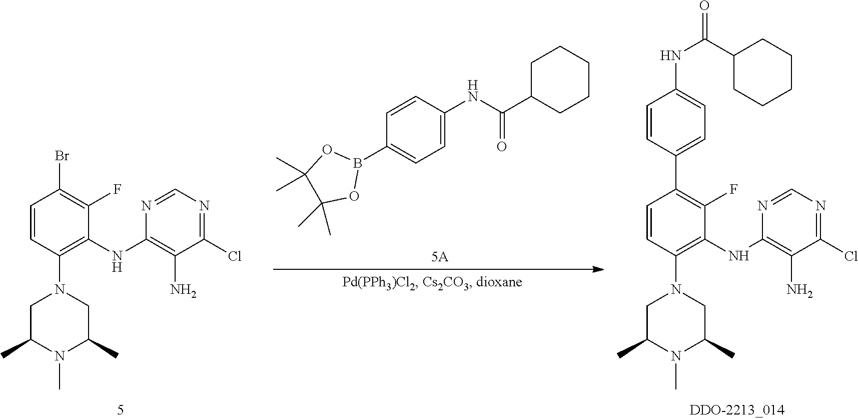 [SMILES: C[C@@H]1CN(c2ccc(Br)c(F)c2Nc2ncnc(Cl)c2N)C[C@H](C)N1C ; CC1(C)OB(c2ccc(NC(=O)C3CCCCC3)cc2)OC1(C)C ; C[C@@H]1CN(c2ccc(-c3ccc(NC(=O)C4CCCCC4)cc3)c(F)c2Nc2ncnc(Cl)c2N)C[C@H](C)N1C]

The Compound 5A is synthesized as shown in Example 42. To a mixture of compound 5 (150 mg, 338.03 μmol, 1 eq.) and compound 5A (134 mg, 405.64 μmol, 1.2 eq.) in dioxane (4 mL) and H₂O (0.5 mL) under N₂ was added Pd(dppf)Cl₂ (25 mg, 33.80 μmol, 0.1 eq.) and Cs₂CO₃ (220 mg, 676.07 μmol, 2 eq.). The reaction mixture was stirred at 100° C. for 2 hr. The mixture was extracted with DCM (20 mL*3). The combined organic phase was washed with brine (10 mL*2), dried with anhydrous Na₂SO₄, filtered and concentrated in vacuum. The crude product was purified by reversed-phase HPLC (Column:Phenomenex Gemini-NX C18 75*30 mm*3 μm; Condition: [water(0.04% NH₃H₂O+10mMNH₄HCO₃)-ACN]; B:33%-73%, 12 min). DDO-2213_014 (6.3 mg, 20.05 μmol, 5.93% yield, 96.20% purity) was obtained as a white solid.

¹H NMR (DMSO-d₆, 400 MHz) δ_{H}=9.91 (s, 1H), 8.04 (s, 1H), 7.70-7.66 (m, 3H), 7.42 (d, J=7.6 Hz, 2H), 7.32 (t, J=8.8 Hz, 1H), 6.92 (d, J=8.4 Hz, 1H), 5.45 (s, 2H), 3.04 (s, 2H), 2.39-2.13 (m, 6H), 1.78 (t, J=15.2 Hz, 5H), 1.65 (d, J=10.8 Hz, 1H), 1.48-1.12 (m, 6H), 0.96 (s, 6H).

HPLC Rₜ=2.78 min in 8 min chromatography, Ultimate XB-C18 3.0*50 mm, 3 m, purity 96.20%.

LCMS Rₜ=1.515 min in 4 min chromatography, Xtimate C18, 3 m, 2.1*30 mm, purity 95.33%, MS ESI calcd. For 565.27 [M+H]⁺ 566.27, found 566.4.

### Example 49. N-(3′-((5-amino-6-chloropyrimidin-4-yl)amino)-2′-fluoro-4′-((3S,5R)-3,4,5-trimethylpiperazin-1-yl)-[1,1′-biphenyl]-4-yl)-2-cyclohexylacetamide (Compound 135, Table 3)

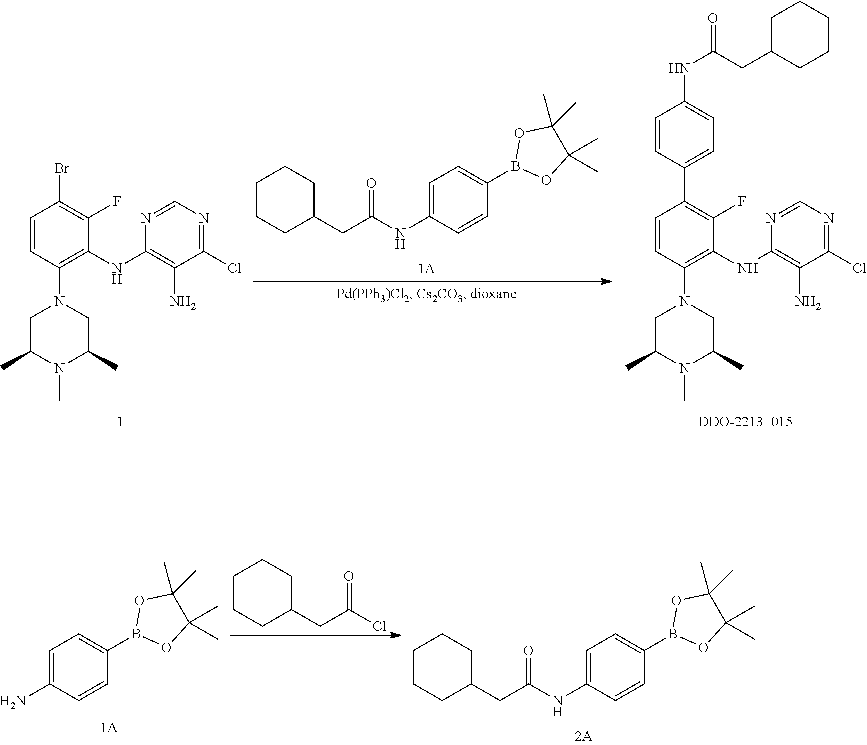

### Step 1: 2-cyclohexyl-N-(4-(4,4,5,5-tetramethyl-1,3,2-dioxaborolan-2-yl)phenyl)acetamide (DDO-2213_015)

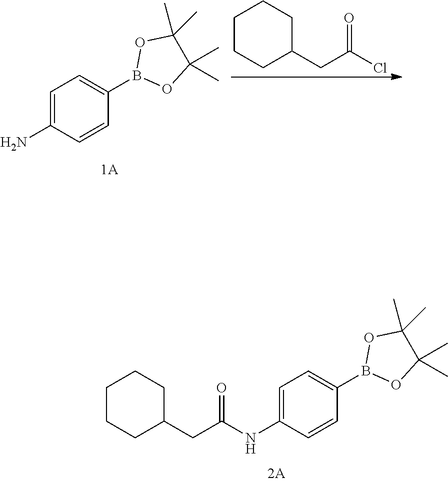

To a mixture of compound 1A (1 g, 4.56 mmol, 1 eq.) and 2-cyclohexylacetyl chloride (953 mg, 5.93 mmol, 1.3 eq.) in DCM (20 mL) was added TEA (924 mg, 9.13 mmol, 1.27 mL, 2 eq) in one portion at 0° C. The mixture was stirred at 25° C. and stirred for 4 hours. The residue was poured into water (50 mL). The aqueous phase was extracted with DCM (20 mL*3). The combined organic phase was washed with brine (50 mL), dried with anhydrous Na₂SO₄, filtered and concentrated in vacuum. The crude product was used in the next step without further purification. Compound 2A (1.7 g, 4.28 mmol, 93.75% yield, 86.4% purity) was obtained as a light yellow solid.

¹H NMR (DMSO-d₆, 400 MHz) δ_{H}=9.97 (s, 1H), 7.63-7.54 (m, 4H), 2.19 (d, J=7.0 Hz, 2H), 1.72-1.55 (m, 7H), 1.27 (s, 12H), 1.21-1.15 (m, 2H), 1.01-0.92 (m, 2H).

### Step 2: N-(3′-((5-amino-6-chloropyrimidin-4-yl)amino)-2′-fluoro-4′-((3S,5R)-3,4,5-trimethylpiperazin-1-yl)-[1,1′-biphenyl]-4-yl)-2-cyclohexylacetamide (DDO-2213_015)

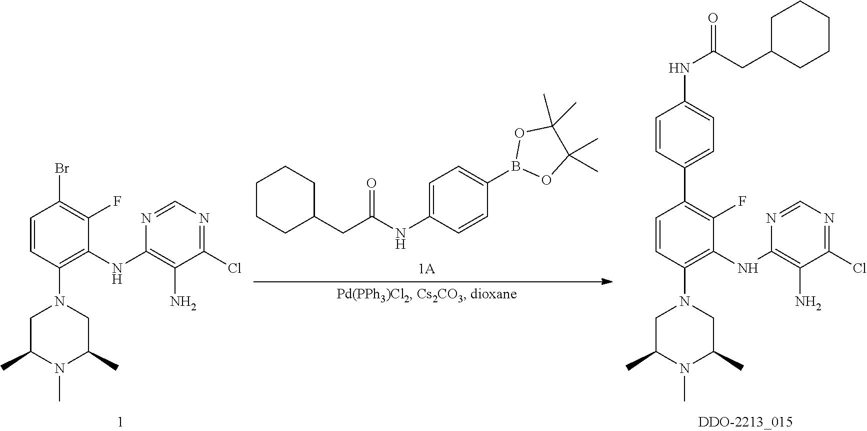 [SMILES: C[C@@H]1CN(c2ccc(Br)c(F)c2Nc2ncnc(Cl)c2N)C[C@H](C)N1C ; CC1(C)OB(c2ccc(NC(=O)CC3CCCCC3)cc2)OC1(C)C ; C[C@@H]1CN(c2ccc(-c3ccc(NC(=O)CC4CCCCC4)cc3)c(F)c2Nc2ncnc(Cl)c2N)C[C@H](C)N1C]

Compound 1 can be synthesized as shown in Example 48. To a mixture of compound 1 (155 mg, 349.07 μmol, 1 eq.) and compound 1A (144 mg, 418.88 μmol, 1.2 eq.) in dioxane (4 mL) and H₂O (0.5 mL) under N₂ was added Pd(dppf)Cl₂ (26 mg, 34.91 μmol, 0.1 eq.) and Cs₂CO₃ (227.47 mg, 698.13 μmol, 2 eq.). The reaction mixture was stirred at 100° C. for 2 hr. The mixture was extracted with DCM (20 mL*3). The combined organic phase was washed with brine (10 mL*2), dried with anhydrous Na₂SO₄, filtered and concentrated in vacuum. The reaction mixture was concentrated to dryness. The residue was purified by prep-HPLC (Column: Phenomenex Gemini-NX C18 75*30 mm*3 μm; Condition:[water(0.04% NH₃H2O+10 mM NH₄HCO₃)-ACN]; B:33%-73%, 12 min). DDO-2213_015 (48.1 mg, 92.79 μmol, 26.58% yield, 96.82% purity) was obtained as a white solid.

¹H NMR (DMSO-d₆, 400 MHz) δ_{H}=9.96 (s, 1H), 8.01 (s, 1H), 7.72-7.64 (m, 3H), 7.43 (d, J=8.0 Hz, 2H), 7.32 (t, J=8.8 Hz, 1H), 6.91 (d, J=8.8 Hz, 1H), 5.44 (s, 2H), 3.00 (d, J=10.8 Hz, 2H), 2.42 (t, J=11.2 Hz, 2H), 2.24-2.06 (m, 7H), 1.85-1.57 (m, 6H), 1.31-1.10 (m, 3H), 1.04-0.95 (m, 2H), 0.92 (d, J=6.0 Hz, 6H).

HPLC Rₜ=2.95 min in 8 min chromatography, Ultimate XB-C18 3.0*50 mm, 3 m, purity 96.82%.

LCMS Rₜ=1.616 min in 4 min chromatography, Xtimate C18, 3 m, 2.1*30 mm, purity 99.41%, MS ESI calcd. For 579.29 [M+H]⁺ 580.29, found 580.4.

### Example 50. (S)-5′-((5-amino-6-chloropyrimidin-4-yl)amino)-4′-(3,4-dimethylpiperazin-1-yl)-2′-fluoro-N-((tetrahydro-2H-pyran-4-yl)methyl)-[1,1′-biphenyl]-4-carboxamide (Compound 136, Table 3)

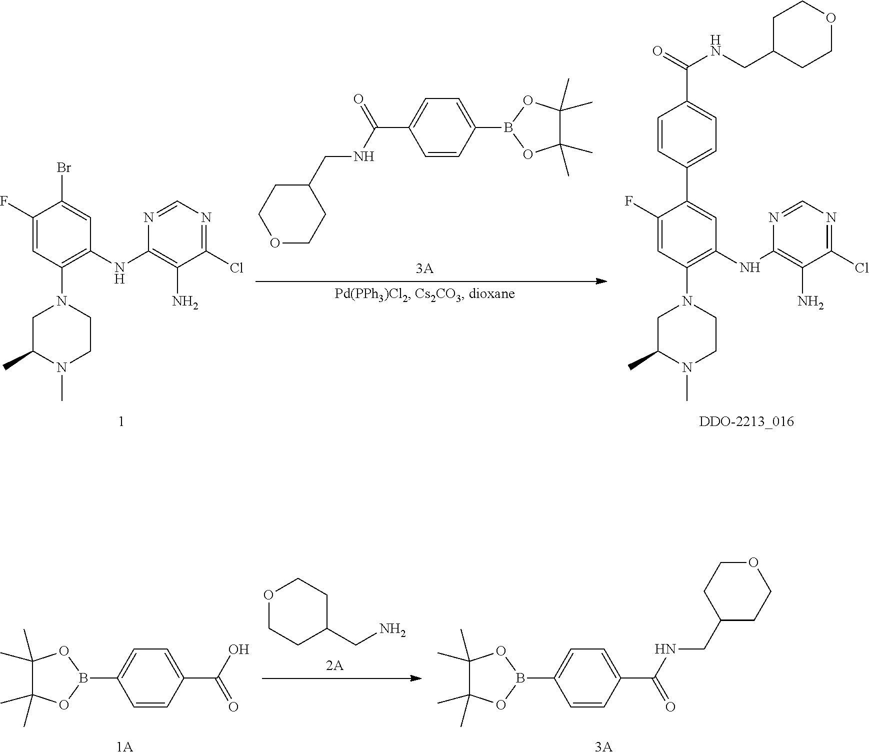

### Step 1: N-((tetrahydro-2H-pyran-4-yl)methyl)-4-(4,4,5,5-tetramethyl-1,3,2-dioxaborolan-2-yl)benzamide (Compound 3A)

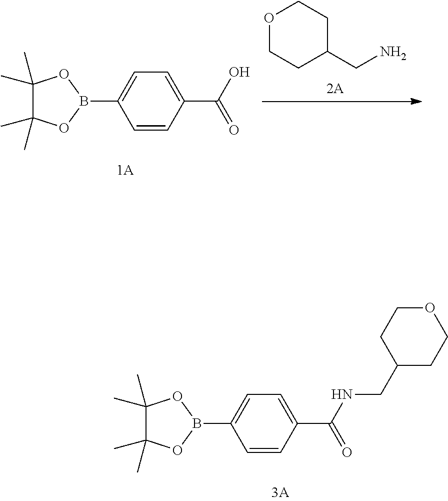

To a mixture of compound 1A (2 g, 8.06 mmol, 1 eq.) and compound 2A (1.11 g, 9.67 mmol, 1.2 eq.) in DMF (20 mL) was added HATU (4.60 g, 12.09 mmol, 1.5 eq.) and TEA (1.63 g, 16.12 mmol, 2.24 mL, 2 eq.). The mixture was extracted with DCM (100 mL*3). The combined organic phase was washed with brine (100 mL*2), dried over anhydrous Na₂SO₄, filtered and concentrated in vacuum. The crude product was purified by silica gel chromatography (ISCO®; 20 g SepaFlash® Silica Flash Column, Eluent of 0-20% MeOH/DCM ethergradient @ 40 mL/min). Compound 3A (3.5 g, 7.55 mmol, 93.63% yield) was obtained as a white solid.

¹H NMR (DMSO-d₆, 400 MHz) δ_{H}=7.85 (d, J=8.0 Hz, 2H), 7.74 (d, J=8.4 Hz, 2H), 3.87-3.81 (m, 2H), 3.35 (s, 1H), 3.29-3.21 (m, 2H), 3.17-3.12 (m, 2H), 1.30 (s, 12H), 1.19-1.16 (m, 5H).

### Step 2: (S)-5′-((5-amino-6-chloropyrimidin-4-yl)amino)-4′-(3,4-dimethylpiperazin-1-yl)-2′-fluoro-N-((tetrahydro-2H-pyran-4-yl)methyl)-[1,1′-biphenyl]-4-carboxamide (DDO-2213_016)

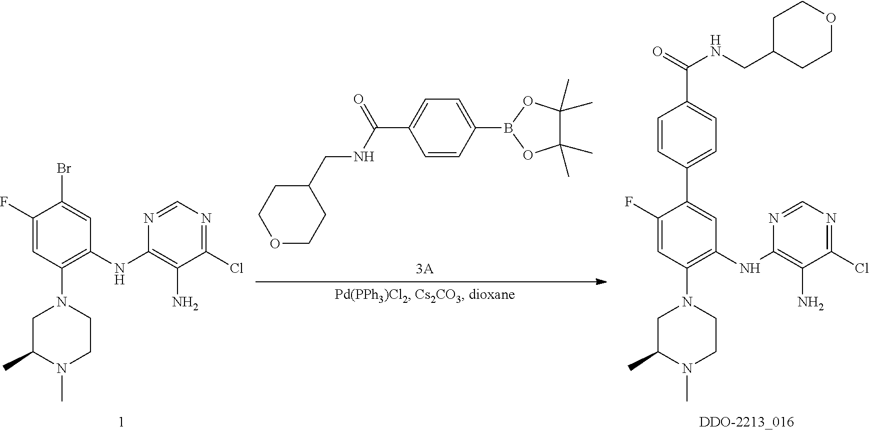 [SMILES: C[C@H]1CN(c2cc(F)c(Br)cc2Nc2ncnc(Cl)c2N)CCN1C ; CC1(C)OB(c2ccc(C(=O)NCC3CCOCC3)cc2)OC1(C)C ; C[C@H]1CN(c2cc(F)c(-c3ccc(C(=O)NCC4CCOCC4)cc3)cc2Nc2ncnc(Cl)c2N)CCN1C]

Compound 1 is synthesized as shown in Example 37. To a mixture of compound 1 (150 mg, 349.07 μmol, 1 eq.) and compound 3A (145 mg, 418.88 μmol, 1.2 eq.) in dioxane (4 mL) and H₂O (0.5 mL) under N₂ was added Pd(dppf)Cl₂ (26 mg, 34.91 μmol, 0.1 eq.) and Cs₂CO₃ (227 mg, 698.13 μmol, 2 eq.). The reaction mixture was stirred at 100° C. for 2 hr. The mixture was extracted with DCM (200 mL*3). The combined organic phase was washed with brine (100 mL*2), dried with anhydrous Na₂SO₄, filtered and concentrated in vacuum. The reaction mixture was concentrated to dryness. The residue was purified by prep-HPLC (Column:Phenomenex Gemini-NX C18 75*30 mm*3 μm; Condition: [water(0.04% NH₃H₂O+10 mM NH₄HCO₃)-ACN)]; B:23%-53%, 11 min). DDO-2213_016 (28 mg, 47.66 μmol, 13.65% yield, 96.70% purity) was obtained as a white solid.

¹H NMR (DMSO-d₆, 400 MHz) δ_{H}=8.53 (t, J=5.6 Hz, 1H), 8.11 (s, 1H), 7.92 (dd, J=2.4, 8.4 Hz, 3H), 7.81 (s, 1H), 7.61 (d, J=7.2 Hz, 2H), 7.07 (d, J=12.4 Hz, 1H), 5.36 (s, 2H), 3.92-3.81 (m, 2H), 3.31-3.22 (m, 3H), 3.17 (t, J=6.4 Hz, 2H), 3.01-2.91 (m, 2H), 2.82-2.71 (m, 2H), 2.48-2.42 (m, 1H), 2.35-2.24 (m, 1H), 2.19 (s, 3H), 1.80 (dt, J=3.6, 7.2 Hz, 1H), 1.60 (d, J=13.2 Hz, 2H), 1.27-1.14 (m, 2H), 0.96 (d, J=6.0 Hz, 3H).

HPLC Rₜ=3.371 min in 8 min chromatography, XBridge Shield RP18, 5 μm, 2.1*50 mm, purity 96.70%.

LCMS Rₜ=2.598 min in 4 min chromatography, XBridge Shield RP18, 5 μm, 2.1*50 mm, purity 97.59%, MS ESI calcd. For 567.25 [M+H]⁺ 568.25, found 568.4.

### Example 51. N-(5′-((5-amino-6-chloropyrimidin-4-yl)amino)-2′-fluoro-4′-((3S,5R)-3,4,5-trimethylpiperazin-1-yl)-[1,1′-biphenyl]-4-yl)butyramide (Compound 137, Table 3)

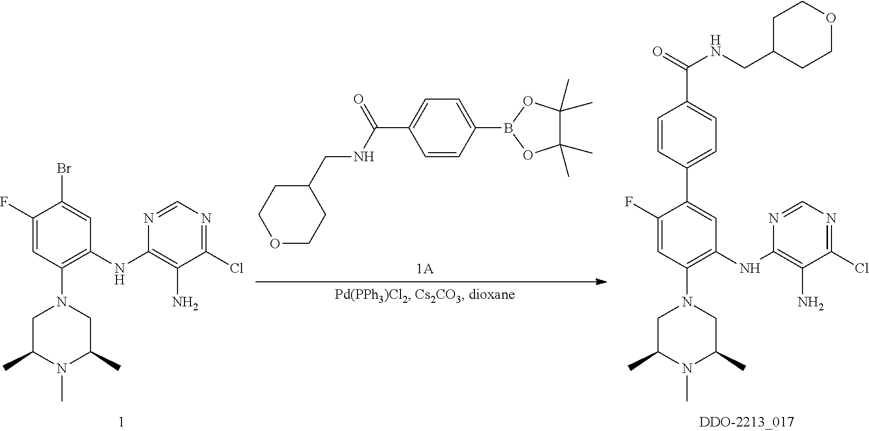 [SMILES: C[C@@H]1CN(c2cc(F)c(Br)cc2Nc2ncnc(Cl)c2N)C[C@H](C)N1C ; CC1(C)OB(c2ccc(C(=O)NCC3CCOCC3)cc2)OC1(C)C ; C[C@@H]1CN(c2cc(F)c(-c3ccc(C(=O)NCC4CCOCC4)cc3)cc2Nc2ncnc(Cl)c2N)C[C@H](C)N1C]

### Step 1: N-(5′-((5-amino-6-chloropyrimidin-4-yl)amino)-2′-fluoro-4′-((3S,5R)-3,4,5-trimethylpiperazin-1-yl)-[1,1′-biphenyl]-4-yl)butyramide (DDO-2213_017)

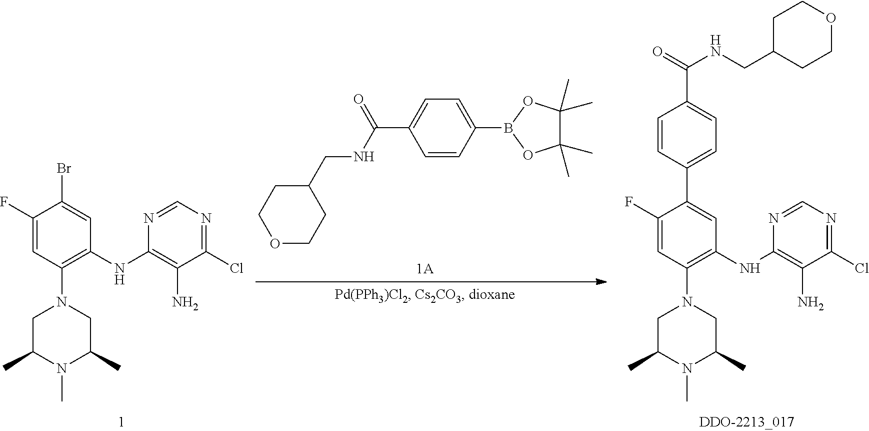 [SMILES: C[C@@H]1CN(c2cc(F)c(Br)cc2Nc2ncnc(Cl)c2N)C[C@H](C)N1C ; CC1(C)OB(c2ccc(C(=O)NCC3CCOCC3)cc2)OC1(C)C ; C[C@@H]1CN(c2cc(F)c(-c3ccc(C(=O)NCC4CCOCC4)cc3)cc2Nc2ncnc(Cl)c2N)C[C@H](C)N1C]

Compound 1 is synthesized as shown in Example 38 and Compound 1A is synthesized as shown in Example 50. To a mixture of compound 1 (200 mg, 450.71 μmol, 1 eq.) and compound 1A (187 mg, 540.85 μmol, 1.2 eq.) in dioxane (4 mL) and H₂O (0.5 mL) under N₂ was added Pd(dppf)Cl₂ (33 mg, 45.07 μmol, 0.1 eq.) and Cs₂CO₃ (294 mg, 901.42 μmol, 2 eq.). The reaction mixture was stirred at 100° C. for 2 hr. The mixture was extracted with DCM (50 mL*3). The combined organic phase was washed with brine (50 mL*2), dried with anhydrous Na₂SO₄, filtered and concentrated in vacuum. The reaction mixture was concentrated to dryness. The residue was purified by prep-HPLC (Column:Phenomenex luna C18 80*40 mm*3 μm; Condition:[water(0.05% NH₃H₂O+10 mM NH₄HCO₃)-ACN)]; B:42%-72%, 8 min). DDO-2213_017 (15.3 mg, 26.03 μmol, 5.78% yield, 99.05% purity) was obtained as a white solid.

¹H NMR (DMSO-d₆, 400 MHz) δ_{H}=8.54 (t, J=5.6 Hz, 1H), 7.96-7.88 (m, 3H), 7.80 (s, 1H), 7.61 (d, J=7.6 Hz, 2H), 7.04 (d, J=12.4 Hz, 1H), 5.36 (s, 2H), 3.90-3.79 (m, 3H), 3.17 (t, J=6.0 Hz, 2H), 2.97 (d, J=10.4 Hz, 2H), 2.48-2.41 (m, 2H), 2.27 (d, J=6.4 Hz, 3H), 2.16 (s, 3H), 1.87-1.74 (m, 1H), 1.60 (d, J=12.8 Hz, 2H), 1.26-1.14 (m, 3H), 0.97 (d, J=6.0 Hz, 6H).

HPLC Rₜ=2.16 min in 8 min chromatography, Ultimate XB-C18 3.0*50 mm, 3 m, purity 99.05%.

LCMS Rₜ=1.711 min in 4 min chromatography, Xtimate C18, 3 m, 2.1*30 mm, purity 96.71%, MS ESI calcd. For 581.27 [M+H]⁺ 582.27, found 582.4.

### Example 52. 6-chloro-N4-(6-fluoro-4′-(morpholinomethyl)-4-((3S,5R)-3,4,5-trimethylpiperazin-1-yl)-[1,1′-biphenyl]-3-yl)pyrimidine-4,5-diamine (Compound 138, Table 3)

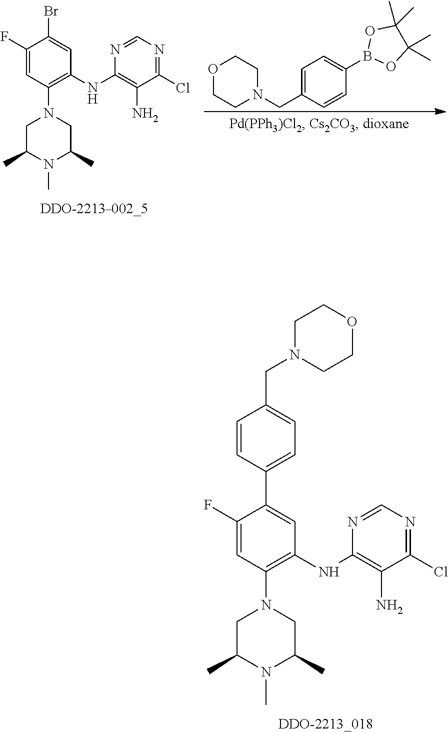

### Step 1: 6-chloro-N4-(6-fluoro-4′-(morpholinomethyl)-4-((3S,5R)-3,4,5-trimethylpiperazin-1-yl)-[1,1′-biphenyl]-3-yl)pyrimidine-4,5-diamine (DDO-2213_018)

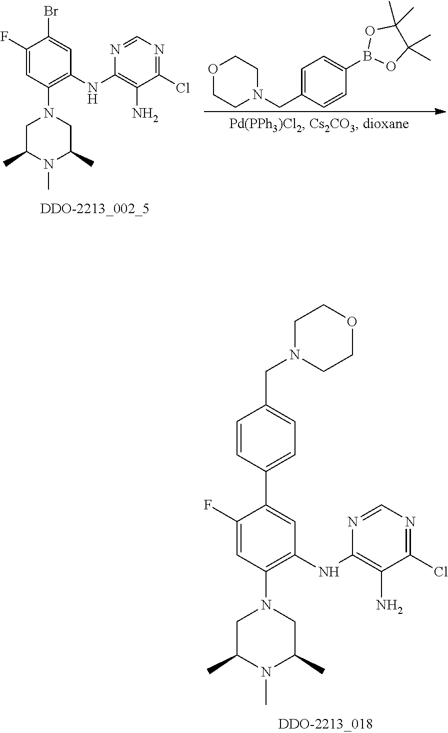

DDO-2213_002_5 is synthesized as shown in Example 38. A mixture of DDO-2213_002_5 (200 mg, 450.71 μmol, 1 eq.), 4-[[4-(4,4,5,5-tetramethyl-1,3,2-dioxaborolan-2-yl)phenyl]methyl] morpholine (164 mg, 540.85 μmol, 1.2 eq.), Pd(dppf)Cl₂·CH₂Cl₂ (37 mg, 45.07 μmol, 0.1 eq.) and Cs₂CO₃ (294 mg, 901.42 μmol, 2 eq.) in dioxane (4 mL) and H₂O (0.8 mL) was degassed and purged with N₂ for 3 times, and then the mixture was stirred at 100° C. for 12 hr under N₂ atmosphere. The mixture was diluted with DCM (50 mL), washed with brine (20 mL×3). The organic layer was dried over Na₂SO₄, filtered and concentrated under reduced pressure to give a residue. The residue was purified by prep-HPLC (Column: Phenomenex luna C18 80*40 mm*3 μm; Mobile Phase A: purified water (0.05% ammonia hydroxide v/v); Mobile Phase B: acetonitrile; Gradient: 43-73% B in 11 min.) DDO-2213_018 (50.8 mg, 91.44 μmol, 20.29% yield, 97.21% purity) was obtained as a white solid.

¹H NMR (DMSO-d₆, 400 MHz) δ_{H}=8.14 (s, 1H), 7.91 (d, J=8.4 Hz, 1H), 7.82 (s, 1H), 7.53-7.36 (m, 4H), 7.03 (d, J=12.4 Hz, 1H), 5.34 (s, 2H), 3.59 (t, J=4.4 Hz, 4H), 3.50 (s, 2H), 3.00-2.90 (m, 2H), 2.49-2.43 (m, 2H), 2.42-2.34 (m, 4H), 2.32-2.23 (m, 2H), 2.18 (s, 3H), 0.98 (d, J=6.0 Hz, 6H).

HPLC Rₜ=2.72 min in 8 min chromatography, Ultimate XB-C18 3.0*50 mm, 3 μm, purity 97.21%.

LCMS Rₜ=1.513 min in 4 min chromatography, Xtimate C18, 3 μm, 2.1*30 mm, purity 97.66%, MS ESI calcd. for 540.07 [M+H]⁺ 541.07, found 540.4.

### Example 53. 6-chloro-N4-(6-fluoro-3′-(morpholinomethyl)-4-((3S,5R)-3,4,5-trimethylpiperazin-1-yl)-[1,1′-biphenyl]-3-ylpyrimidine-4,5-diamine (Compound 139, Table 3)

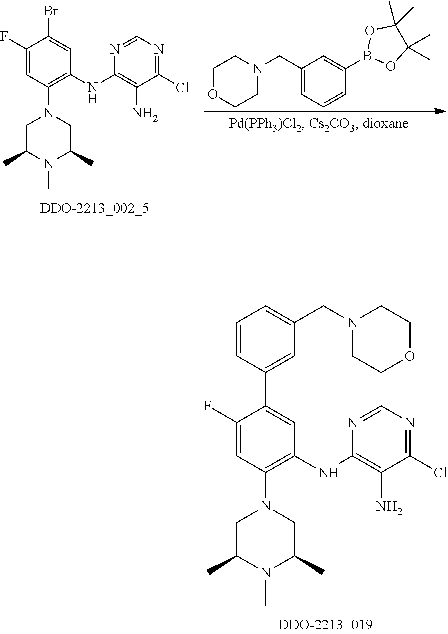

### Step 1: 6-chloro-N4-(6-fluoro-3′-(morpholinomethyl)-4-((3S,5R)-3,4,5-trimethylpiperazin-1-yl)-[1,1′-biphenyl]-3-yl)pyrimidine-4,5-diamine (DDO-2213_019)

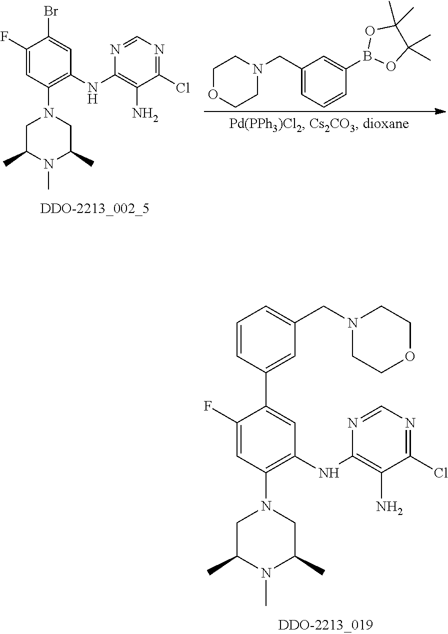

DDO-2213_002_5 is synthesized as shown in Example 38. A mixture of DDO-2213_002_5 (200 mg, 450.71 μmol, 1 eq.), 4-[[3-(4,4,5,5-tetramethyl-1,3,2-dioxaborolan-2-yl)phenyl]methyl]morpholine (164 mg, 540.85 μmol, 1.2 eq.), Pd(dppf)Cl₂·CH₂Cl₂ (37 mg, 45.07 μmol, 0.1 eq.) and Cs₂CO₃ (294 mg, 901.42 μmol, 2 eq.) in dioxane (4 mL) and H₂O (0.8 mL) was degassed and purged with N₂ for 3 times, and then the mixture was stirred at 100° C. for 12 hr under N₂ atmosphere. The reaction mixture was diluted with DCM (100 mL), washed with brine (50 mL). The organic layer was dried over Na₂SO₄, filtered and concentrated under reduced pressure to give a residue. The residue was purified by prep-HPLC (Phenomenex luna C18 80*40 mm*3 μm; water (0.05% ammonia hydroxide)-ACN). DDO-2213_019 (41.5 mg, 75.17 μmol, 16.68% yield, 97.82% purity) was obtained as a white solid.

¹H NMR (DMSO-d₆, 400 MHz) δ_{H}=8.13 (s, 1H), 7.88 (d, J=8.4 Hz, 1H), 7.81 (s, 1H), 7.46 (s, 1H), 7.43-7.38 (m, 2H), 7.34-7.28 (m, 1H), 7.02 (d, J=12.4 Hz, 1H), 5.34 (s, 2H), 3.57 (s, 4H), 3.52 (s, 2H), 3.00-2.90 (m, 2H), 2.46 (s, 2H), 2.37 (s, 4H), 2.31-2.22 (m, 2H), 2.17 (s, 3H), 0.97 (d, J=6.0 Hz, 6H).

HPLC Rₜ=2.82 min in 8 min chromatography, Ultimate XB-C18 3.0*50 mm, 3 μm, purity 97.82%.

LCMS Rₜ=1.574 min in 4 min chromatography, Xtimate C18, 3 μm, 2.1*30 mm, purity 99.81%, MS ESI calcd. for 539.26 [M+H]⁺ 540.26, found 540.4.

### Example 54. (Table 3 shows this compound as . . . ) (S)-6-chloro-N4-(2-(3,4-dimethylpiperazin-1-yl)-4-fluoro-5-(2-morpholinopyrimidin-5-yl)phenyl)pyrimidine-4,5-diamine (Compound 140, Table 3)

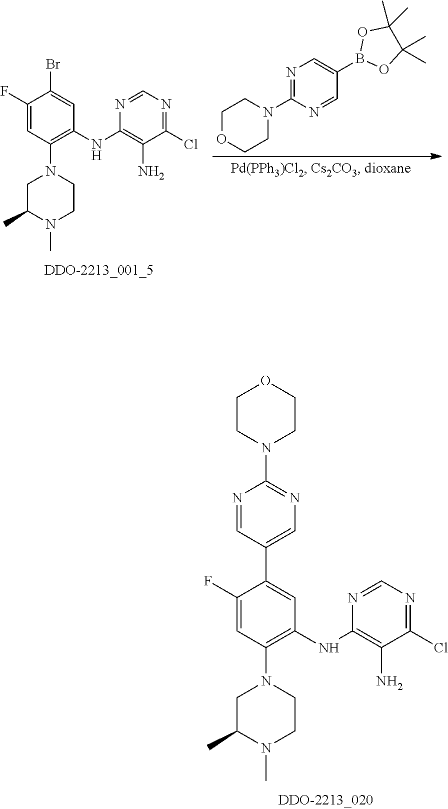

### Step 1: (S)-6-chloro-N4-(2-(3,4-dimethylpiperazin-1-yl)-4-fluoro-5-(2-morpholinopyrimidin-5-yl)phenyl)pyrimidine-4,5-diamine (DDO-2213_020)

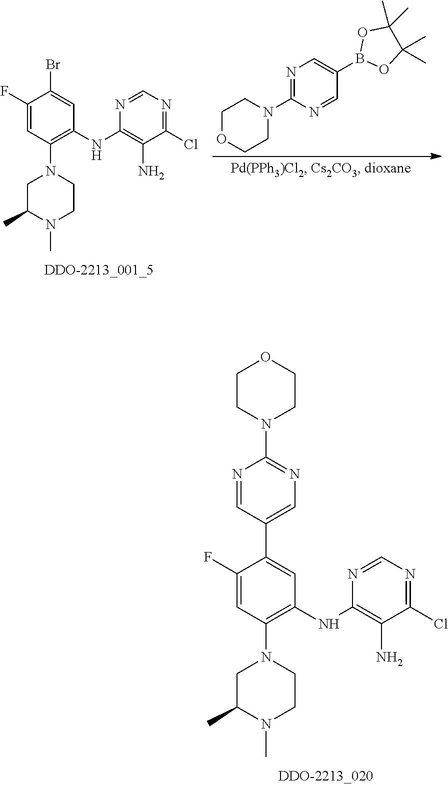

DDO-2213_001_5 is synthesized as seen in Example 37. A mixture of DDO-2213_001_5 (150 mg, 349.07 μmol, 1 eq.), 4-[5-(4,4,5,5-tetramethyl-1,3,2-dioxaborolan-2-yl)pyrimidin-2-yl]morpholine (122 mg, 418.88 μmol, 1.2 eq.), Pd(dppf)Cl₂·CH₂Cl₂ (28 mg, 34.91 μmol, 0.1 eq.) and Cs₂CO₃ (227 mg, 698.13 μmol, 2 eq.) in dioxane (4 mL) and H₂O (0.8 mL) was degassed and purged with N₂ for 3 times, and then the mixture was stirred at 100° C. for 12 hr under N₂ atmosphere. The reaction mixture was diluted with DCM (100 mL), washed with brine (50 mL). The organic layer was dried over Na₂SO₄, filtered and concentrated under reduced pressure to give a residue. The residue was purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 30-60% B in 11 min.). DDO-2213_020 (69.3 mg, 133.60 μmol, 38.27% yield, 99.09% purity) was obtained as a white solid.

¹H NMR (DMSO-d₆, 400 MHz) δ_{H}=8.55 (s, 2H), 8.11 (s, 1H), 7.90 (d, J=8.8 Hz, 1H), 7.81 (s, 1H), 7.11-7.01 (m, 1H), 5.35 (s, 2H), 3.77-3.63 (m, 8H), 2.99-2.86 (m, 2H), 2.80-2.69 (m, 2H), 2.47- 2.39 (m, 1H), 2.31-2.23 (m, 1H), 2.18 (s, 4H), 0.95 (d, J=6.0 Hz, 3H).

HPLC Rₜ=3.487 min in 8 min chromatography, XBridge Shield RP18, 5 μm, 2.1*50 mm, purity 99.09%.

LCMS Rₜ=2.674 min in 4 min chromatography, XBridge Shield RP18, 5 μm, 2.1*50 mm, purity 99.49%, MS ESI calcd. for 513.22 [M+H]⁺ 514.22, found 514.3.

### Example 55. 6-chloro-N4-(4-fluoro-5-(2-morpholinopyrimidin-5-yl)-2-((3S,5R)-3,4,5-trimethylpiperazin-1-yl)phenyl)pyrimidine-4,5-diamine (Compound 141, Table 3)

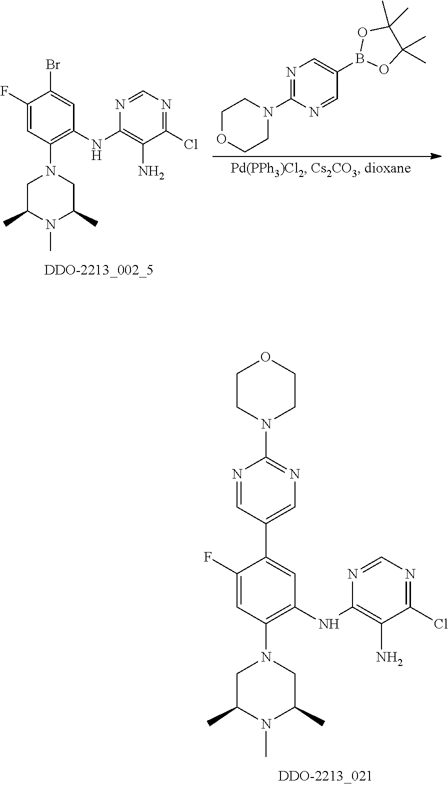

### Step 1: 6-chloro-N4-(4-fluoro-5-(2-morpholinopyrimidin-5-yl)-2-((3S,5R)-3,4,5-trimethylpiperazin-1-yl)phenyl)pyrimidine-4,5-diamine (DDO-2213_021)

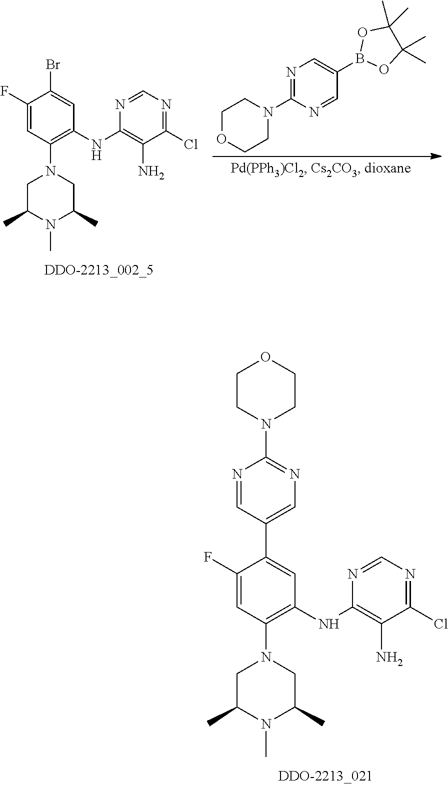

DDO-2213_002_5 is synthesized as shown in Example 38. A mixture of DDO-2213_002_5 (200 mg, 450.71 μmol, 1 eq.), 4-[5-(4,4,5,5-tetramethyl-1,3,2-dioxaborolan-2-yl)pyrimidin-2-yl]morpholine (157 mg, 540.85 μmol, 1.2 eq.), Pd(dppf)Cl₂·CH₂Cl₂ (37 mg, 45.07 μmol, 0.1 eq.) and Cs₂CO₃ (294 mg, 901.42 μmol, 2 eq.) in dioxane (4 mL) and H₂O (0.8 mL) was degassed and purged with N₂ for 3 times, and then the mixture was stirred at 100° C. for 12 hr under N₂ atmosphere. The reaction mixture was diluted with DCM (100 mL), washed with brine (50 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-10% Methanol/Dichloromethane @ 30 mL/min). The product was purified by re-crystallization from MeOH (5 mL) at 20° C. DDO-2213_021 (45 mg, 81.45 μmol, 18.07% yield, 95.57% purity) was obtained as a white solid.

¹H NMR (DMSO-d₆, 400 MHz) δ_{H}=8.55 (s, 2H), 8.18-8.07 (m, 1H), 7.93-7.86 (m, 1H), 7.84-7.78 (m, 1H), 7.10-6.99 (m, 1H), 5.35 (s, 2H), 3.81-3.59 (m, 8H), 2.97-2.87 (m, 2H), 2.48-2.38 (m, 2H), 2.31-2.20 (m, 2H), 2.16 (s, 3H), 0.96 (d, J=6.0 Hz, 6H).

HPLC Rₜ=3.06 min in 8 min chromatography, Ultimate XB-C18 3.0*50 mm, 3 μm, purity 95.57%.

LCMS Rₜ=2.000 min in 4 min chromatography, Xtimate C18, 3 μm, 2.1*30 mm, purity 97.83%, MS ESI calcd. for 527.23 [M+H]⁺ 528.23, found 528.3.

### Example 56. 6-chloro-N4-(5-(6-(cyclopropylmethoxy)pyridin-3-yl)-4-fluoro-2-((3S,5R)-3,4,5-trimethylpiperazin-1-yl)phenyl)pyrimidine-4,5-diamine (Compound 142, Table 3)

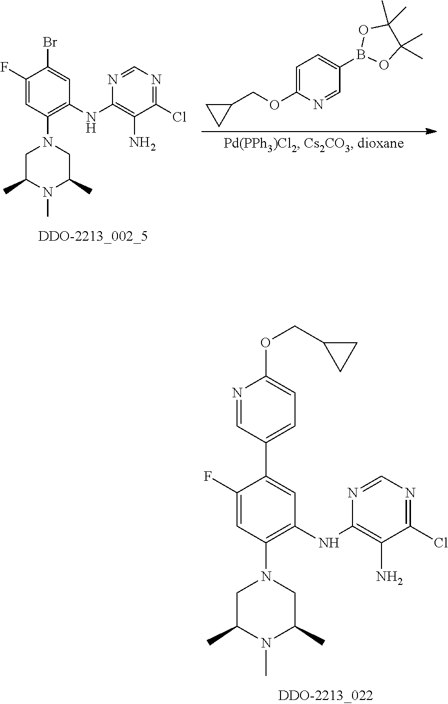

### Step 1: 6-chloro-N4-(5-(6-(cyclopropylmethoxy)pyridin-3-yl)-4-fluoro-2-((3S,5R)-3,4,5-trimethylpiperazin-1-yl)phenyl)pyrimidine-4,5-diamine (DDO-2213_022)

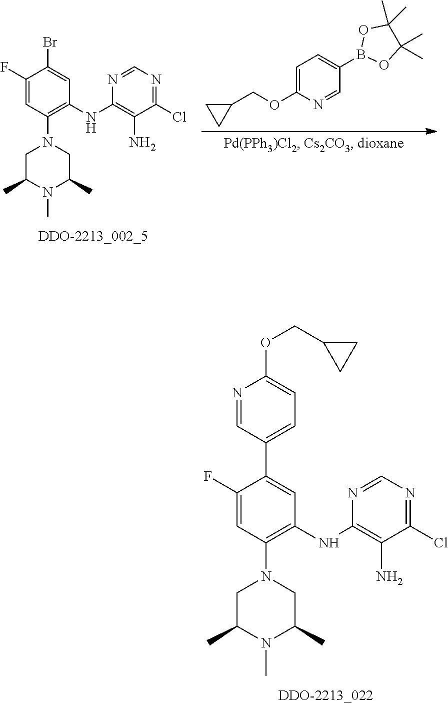

DDO-2213_002_5 is synthesized as shown in Example 38. To a mixture of DDO-2213_002_5 (200 mg, 450.71 μmol, 1 eq) and 2-(cyclopropylmethoxy)-5-(4,4,5,5-tetramethyl-1,3,2-dioxaborolan-2-yl)pyridine (124 mg, 450.71 μmol, 1 eq) in dioxane (4 mL) and H₂O (0.8 mL) was added Pd(dppf)Cl₂·CH₂Cl₂ (37 mg, 45.07 μmol, 0.1 eq) and Cs₂CO₃ (147 mg, 450.71 μmol, 1 eq) in one portion under N₂. The mixture was heated to 100° C. and stirred for 12 hours. The residue was poured into water (20 mL). The aqueous phase was extracted with ethyl acetate (10 mL*3). The combined organic phase was washed with brine (20 mL), dried with anhydrous Na₂SO₄, filtered and concentrated in vacuum. The residue was purified by prep. HPLC (Column: Phenomenex luna C18 80*40 mm*3 μm; mobile phase: [water (0.05% ammonia hydroxide v/v)-ACN]; B %:45%-95%; 20 min). DDO-2213_022 (35.9 mg, 69.70 mol, 15.46% yield, 99.41% purity) was obtained as a white solid.

¹H NMR (DMSO-d₆, 400 MHz) δ_{H}=8.27 (s, 1H), 8.13 (s, 1H), 7.89 (d, J=8.4 Hz, 1H), 7.85 (d, J=9.4 Hz, 1H), 7.81 (s, 1H), 7.04 (d, J=12.4 Hz, 1H), 6.92 (d, J=8.8 Hz, 1H), 5.35 (s, 2H), 4.12 (d, J=7.2 Hz, 2H), 2.94 (d, J=10.8 Hz, 2H), 2.45 (t, J=10.8 Hz, 2H), 2.31-2.22 (m, 2H), 2.16 (s, 3H), 1.32-1.20 (m, 1H), 0.96 (d, J=6.4 Hz, 6H), 0.60-0.51 (m, 2H), 0.37-0.29 (m, 2H)

HPLC Rₜ=2.83 min in 8 min chromatography, Ultimate XB-C18 3.0*50 mm, 3 μm, 8 μm, purity 99.41%.

LCMS Rₜ=1.701 min in 4 min chromatography, Xtimate C18, 3 μm, 2.1*30 mm, 8 m, purity 99.85%, MS ESI calcd. for 512.23 [M+H]⁺513.23, found 512.3.

### Example 57. Chloro-N4-(5-(2-((2S,6R)-2,6-dimethylmorpholino)pyrimidin-5-yl)-2-((S)-3,4-dimethylpiperazin-1-yl)-4-fluorophenyl)pyrimidine-4,5-diamine (Compound 143, Table 3)

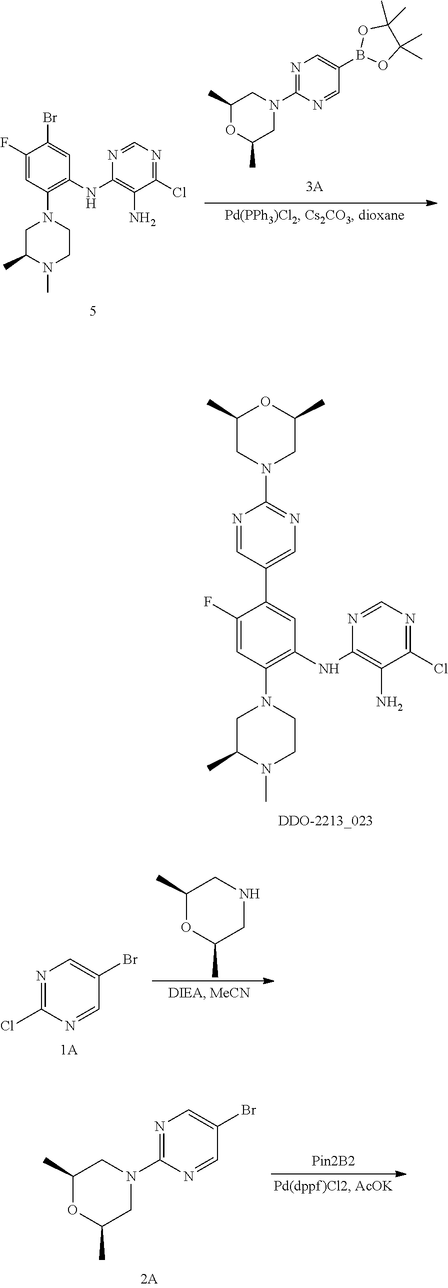

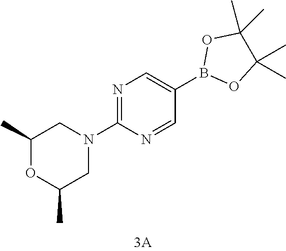

### Step 1: (2S,6R)-4-(5-bromopyrimidin-2-yl)-2,6-dimethylmorpholine (Compound 2A)

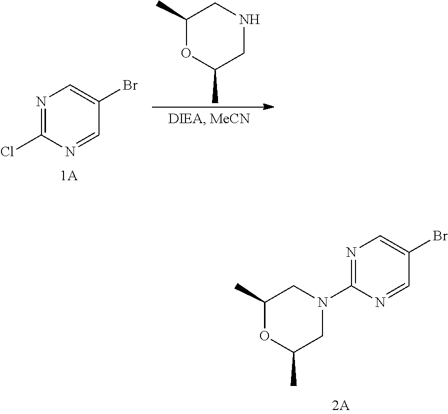

To a solution of compound 1A (1 g, 5.17 mmol, 1 eq.) and (2S,6R)-2,6-dimethylmorpholine (595 mg, 5.17 mmol, 1 eq.) in MeCN (15 mL) was added DIEA (1.34 g, 10.34 mmol, 1.80 mL, 2 eq.). The mixture was stirred at 25° C. for 12 hr. The reaction mixture was diluted with water (20 mL), extracted with DCM (100 mL) and washed with brine (20 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-2% Methanol/Dichloromethane @ 40 mL/min). Compound 2A (1.4 g, 5.02 mmol, 97.02% yield) was obtained as a white solid.

¹H NMR (DMSO-d₆, 400 MHz) δ_{H}=8.46 (s, 2H), 4.41 (dd, J=1.6, 13.2 Hz, 2H), 3.54 (dd, J=2.4, 4.4 Hz, 2H), 2.58-2.51 (m, 2H), 1.14 (d, J=6.4 Hz, 6H).

### Step 2: (2S,6R)-2,6-dimethyl-4-(5-(4,4,5,5-tetramethyl-1,3,2-dioxaborolan-2-yl)pyrimidin-2-yl)morpholine (Compound 3A)

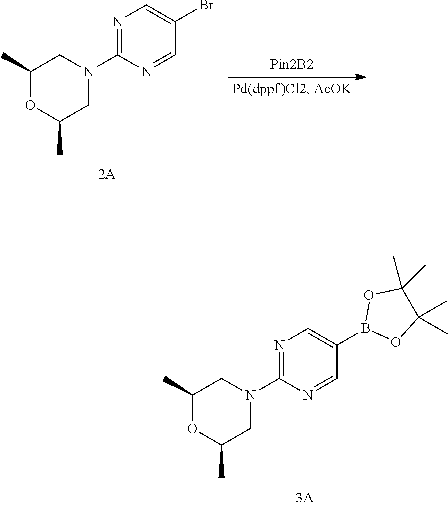

To a mixture of compound 2A (1 g, 3.67 mmol, 1 eq.) in dioxane (17 mL) was added AcOK (721 mg, 7.35 mmol, 2 eq.), Pin2B2 (1.12 g, 4.41 mmol, 1.2 eq.) and Pd(dppf)Cl₂ (269 mg, 367.46 μmol, 0.1 eq.) under N₂. The reaction mixture was stirred at 100° C. for 2 hr. The mixture was diluted with H₂O (20 mL) and extracted with DCM (100 mL*2). The combined organic phase was washed with brine (100 mL), dried over anhydrous Na₂SO₄, filtered and concentrated in vacuum. The residue was purified by flash silica gel chromatography (ISCO®; 12 g SepaFlash® Silica Flash Column, Eluent of 0-5% Ethyl acetate/DCM @ 30 mL/min). Compound 3A (1.1 g, 3.10 mmol, 84.40% yield) was obtained as a brown oil.

¹H NMR (DMSO-d₆, 400 MHz) δ_{H}=8.51-8.48 (m, 1H), 8.49 (s, 1H), 4.61-4.54 (m, 2H), 3.56-3.49 (m, 2H), 3.56-3.49 (m, 1H), 2.60-2.53 (m, 2H), 1.16 (s, 12H), 1.07 (s, 6H).

### Step 3: 6-chloro-N4-(5-(2-((2S,6R)-2,6-dimethylmorpholino)pyrimidin-5-yl)-2-((S)-3,4-dimethylpiperazin-1-yl)-4-fluorophenyl)pyrimidine-4,5-diamine (DDO-2213_023)

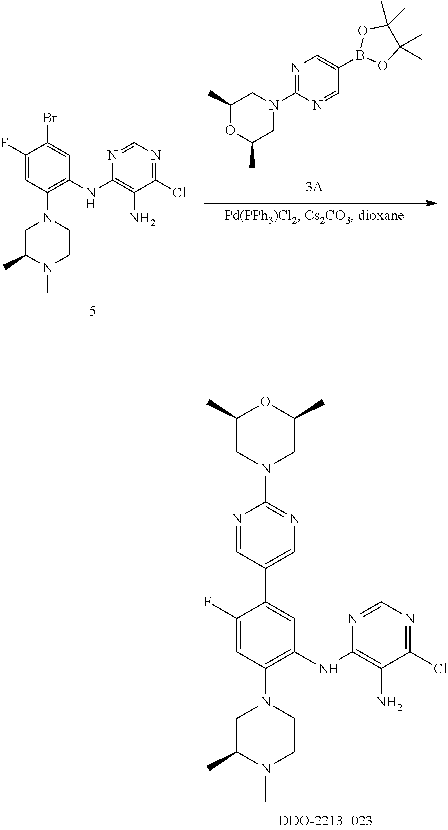

Compound 5 can be synthesized as shown in Example 37. A mixture of compound 5 (70 mg, 162.90 μmol, 1 eq.), compound 3A (63 mg, 195.48 μmol, 1.2 eq.), Pd(dppf)Cl₂·CH₂Cl₂ (13 mg, 16.29 μmol, 0.1 eq.) and Cs₂CO₃ (106 mg, 325.80 μmol, 2 eq.) in dioxane (4 mL) and H₂O (0.8 mL) was degassed and purged with N₂ for 3 times, and then the mixture was stirred at 100° C. for 12 hr under N₂ atmosphere. The reaction mixture was diluted with H₂O (10 mL), extracted with DCM (50 mL) and washed with brine (20 mL). The organic layer was dried over Na₂SO₄, filtered and concentrated under reduced pressure to give a residue. The residue was purified by prep-HPLC (Column: Phenomenex Gemini-NX C18 75*30 mm*3 μm; Mobile Phase A: purified water (0.04% NH₃H₂O+10 mM NH₄HCO₃); Mobile Phase B: acetonitrile; Gradient: 40-70% B in 11 min.). Compound DDO-2213_023 (15.1 mg, 27.77 μmol, 17.05% yield, 99.7% purity) was obtained as a white solid.

¹H NMR (DMSO-d₆, 400 MHz) δ_{H}=8.53 (s, 2H), 8.10 (s, 1H), 7.88 (d, J=8.8 Hz, 1H), 7.81 (s, 1H), 7.06 (d, J=12.4 Hz, 1H), 5.35 (s, 2H), 4.60-4.48 (m, 2H), 3.63-3.50 (m, 2H), 2.98-2.86 (m, 2H), 2.81-2.70 (m, 2H), 2.62-2.52 (m, 2H), 2.43 (s, 1H), 2.31-2.22 (m, 1H), 2.18 (s, 3H), 2.16-2.11 (m, 1H), 1.16 (d, J=6.0 Hz, 6H), 0.95 (d, J=6.0 Hz, 3H).

HPLC Rₜ=2.997 min in 8 min chromatography, X Bridge Shield RP18, 5 μm, 2.1*50 mm, purity 99.78%.

LCMS Rₜ=2.406 min in 4 min chromatography, X Bridge Shield RP18, 5 μm, 2.1*50 mm, purity 99.78%, MS ESI calcd. for 541.25 [M+H]⁺ 542.25, found 542.4.

### Example 58. 6-chloro-N4-(4-fluoro-5-(2-morpholinopyrimidin-4-yl)-2-((3S,5R)-3,4,5-trimethylpiperazin-1-yl)phenyl)pyrimidine-4,5-diamine (Compound 144, Table 3)

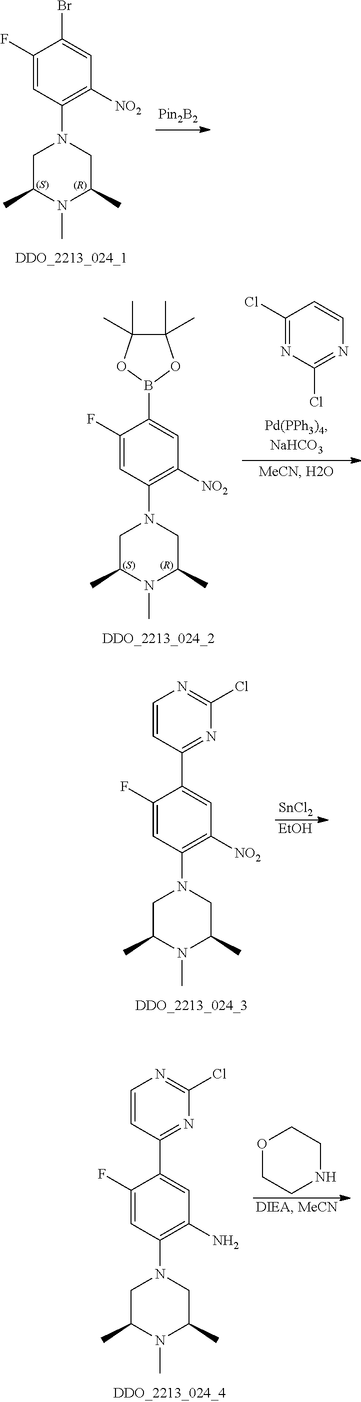

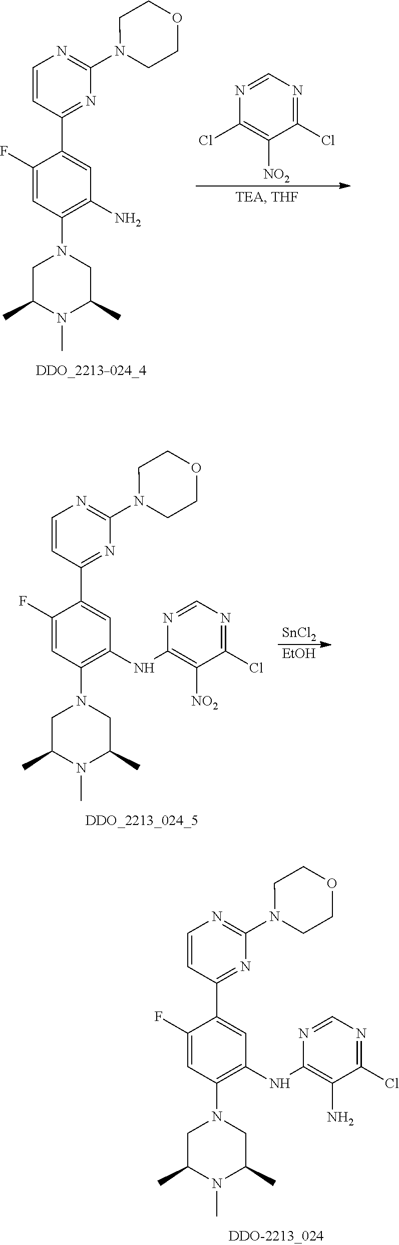

### Step 1: (2S,6R)-4-(5-fluoro-2-nitro-4-(4,4,5,5-tetramethyl-1,3,2-dioxaborolan-2-yl)phenyl)-1,2,6-trimethylpiperazine (DDO-2213_024_2)

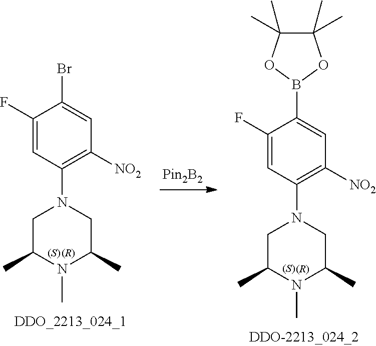

DDO-2213_024_1 can be synthesized as shown in Example 38. To a mixture of DDO-2213_024_1 (5 g, 14.44 mmol, 1 eq) in dioxane (20 mL) was added AcOK (2.83 g, 28.89 mmol, 2 eq), Pin₂B₂ (4.40 g, 17.33 mmol, 1.2 eq) and Pd(dppf)Cl₂ (1.06 g, 1.44 mmol, 0.1 eq) in one portion at 25° C. under N₂. The mixture was heated to 100° C. and stirred for 12 hours. The residue was filtered via a filter paper and concentrated under reduce pressure. DDO-2213_024_2 (8.7 g, crude) was obtained as a yellow solid. The crude product was used in the next step without further purification.

LCMS Rₜ=0.900 min in 2 min chromatography, Xtimate C18, 3 μm, 2.1*30 mm, purity 50.97%, MS ESI calcd. for 393.22 [M+H]⁺394.22, found 312.2.

### Step 2: 2-chloro-4-(2-fluoro-5-nitro-4-((3S,5R)-3,4,5-trimethylpiperazin-1-yl)phenyl)pyrimidine (DDO-2213_024_3)

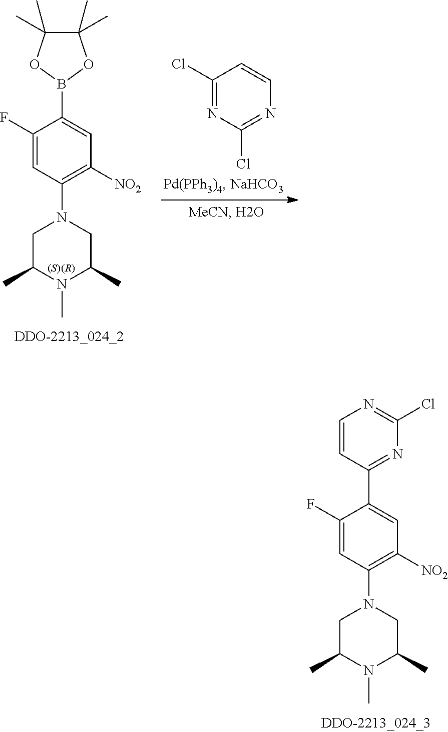

To a mixture of DDO-2213_024_2 (8.7 g, 15.49 mmol, 70% purity, 1 eq) and 2,4-dichloropyrimidine (2.31 g, 15.49 mmol, 1 eq) in CH₃CN (100 mL) and H₂O (30 mL) was added NaHCO₃ (3.90 g, 46.46 mmol, 1.81 mL, 3 eq) and Pd(PPh₃)₄ (894.75 mg, 774.30 μmol, 0.05 eq) in one portion at 25° C. under N₂. The mixture was heated to 80° C. and stirred for 16 hours. The mixture was filtered via a filter paper and concentrated under reduce pressure. The residue was purified by silica gel chromatography (column weight: 20 g, diameter: 100 mm, 100-200 mesh silica gel, Dichloromethane: Methanol=10:1). DDO-2213_024_3 (1.8 g, 4.72 mmol, 30.48% yield) was obtained as a yellow solid.

¹H NMR (CHLOROFORM-d, 400 MHz) δ_{H}=8.85 (d, J=8.4 Hz, 1H), 8.63 (d, J=5.2 Hz, 1H), 7.73 (d, J=5.6 Hz, 1H), 6.77 (d, J=14.4 Hz, 1H), 3.16 (d, J=12.4 Hz, 2H), 2.88 (t, J=11.6 Hz, 2H), 2.51-2.41 (m, 2H), 2.33 (s, 3H), 1.20-1.09 (m, 1H), 1.15 (d, J=6.4 Hz, 5H).

### Step 3: 5-(2-chloropyrimidin-4-yl)-4-fluoro-2-((3S,5R)-3,4,5-trimethylpiperazin-1-yl)aniline (DDO-2213_024_4)

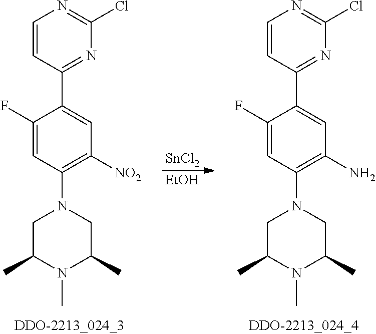

To a mixture of SnCl₂·2H₂O (3.21 g, 14.22 mmol, 3 eq.) in MeOH (20 mL) and H₂O (6 mL) was added DDO-2213_024_3 (1.8 g, 4.74 mmol, 1 eq.) in one portion, then the mixture was heated to 80° C. for 4 hours. The pH was adjusted to around 8 by progressively adding NaHCO₃ (10 mL). Then the mixture was added DCM (50 mL) and stirred for 5 mins. The mixture was filtered via a filter paper. The combined organic phase was washed with brine (20 mL*2), dried with anhydrous Na₂SO₄, filtered and concentrated in vacuum. The residue was purified by silica gel chromatography (column weight: 4 g, diameter: 100 mm, 100-200 mesh silica gel, Dichloromethane: Methanol=10:1). DDO-2213_024_3 (1.1 g, 2.81 mmol, 59.32% yield) was obtained as a yellow solid.

¹H NMR (CHLOROFORM-d, 400 MHz) δ_{H}=8.57 (d, J=5.6 Hz, 1H), 7.78 (d, J=5.2 Hz, 1H), 7.59 (d, J=7.6 Hz, 1H), 6.74 (d, J=13.2 Hz, 1H), 3.85 (s, 2H), 3.17 (d, J=11.2 Hz, 2H), 2.56-2.48 (m, 2H), 2.43 (d, J=6.0 Hz, 2H), 2.35 (s, 3H), 1.17 (d, J=6.0 Hz, 6H).

### Step 4: 5-(2-chloropyrimidin-4-yl)-4-fluoro-2-((3S,5R)-3,4,5-trimethylpiperazin-1-yl)aniline (DDO-2213_024_5)

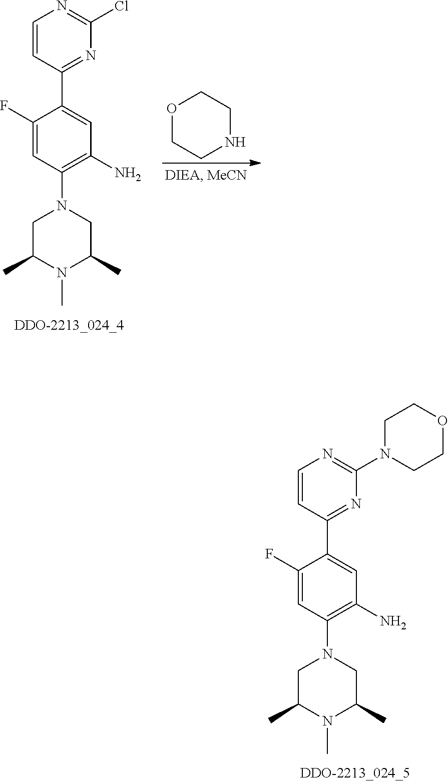 [SMILES: C[C@@H]1CN(c2cc(F)c(-c3ccnc(Cl)n3)cc2N)C[C@H](C)N1C ; C1COCCN1 ; C[C@@H]1CN(c2cc(F)c(-c3ccnc(N4CCOCC4)n3)cc2N)C[C@H](C)N1C]

To a mixture of DDO-2213_024_4 (400 mg, 1.14 mmol, 1 eq.) and morpholine (398 mg, 4.57 mmol, 402.48 μL, 4 eq) in CH₃CN (20 mL) was added DIEA (591 mg, 4.57 mmol, 796.62 μL, 4 eq) in one portion. The mixture was stirred at 80° C. for 12 hours. The residue was concentrated under reduce pressure. The residue was purified by silica gel chromatography (column weight: 12 g, diameter: 100 mm, 100-200 mesh silica gel, Dichloromethane: Methanol=10:1). DDO-2213_024_5 (430 mg, 985.91 μmol, 86.23% yield) was obtained as a yellow solid.

¹H NMR (CHLOROFORM-d, 400 MHz) δ_{H}=8.34 (d, J=5.2 Hz, 1H), 7.46 (d, J=7.6 Hz, 1H), 7.08 (dd, J=2.0, 5.2 Hz, 1H), 6.74 (d, J=12.8 Hz, 1H), 3.90-3.86 (m, 4H), 3.82-3.79 (m, 4H), 3.12 (d, J=11.2 Hz, 2H), 2.56-2.48 (m, 2H), 2.43 (s, 2H), 2.35 (s, 3H), 1.17 (d, J=6.0 Hz, 6H).

### Step 5: 6-chloro-N-(4-fluoro-5-(2-morpholinopyrimidin-4-yl)-2-((3S,5R)-3,4,5-trimethylpiperazin-1-yl)phenyl)-5-nitropyrimidin-4-amine (DDO-2213_024_6)

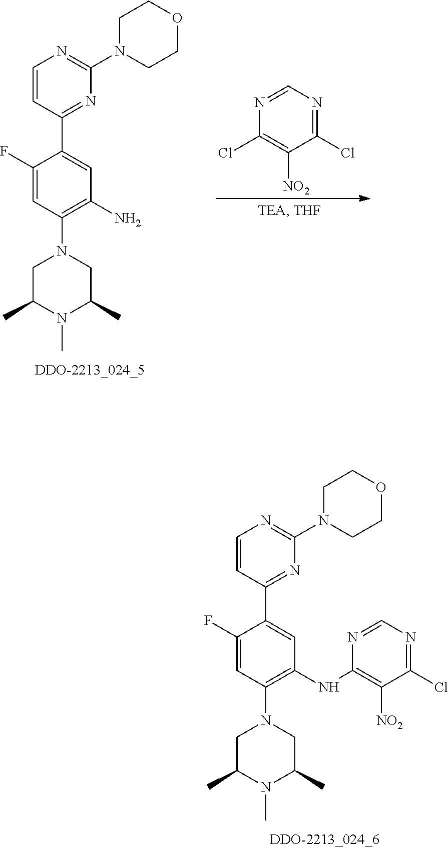 [SMILES: C[C@@H]1CN(c2cc(F)c(-c3ccnc(N4CCOCC4)n3)cc2N)C[C@H](C)N1C ; O=[N+]([O-])c1c(Cl)ncnc1Cl ; C[C@@H]1CN(c2cc(F)c(-c3ccnc(N4CCOCC4)n3)cc2Nc2ncnc(Cl)c2[N+](=O)[O-])C[C@H](C)N1C]

To a mixture of DDO-2213_024_5 (430 mg, 1.07 mmol, 1 eq) and 4,6-dichloro-5-nitro-pyrimidine (417 mg, 2.15 mmol, 2 eq) in THF (10 mL) was added TEA (325.93 mg, 3.22 mmol, 448.33 L, 3 eq) in one portion. The mixture was stirred at 25° C. for 12 hours. The residue was poured into water (20 mL). The aqueous phase was extracted with DCM (20 mL*3). The combined organic phase was washed with brine (40 mL), dried with anhydrous Na₂SO₄, filtered and concentrated in vacuum. The residue was purified by silica gel chromatography (column weight: 12 g, diameter: 100 mm, 100-200 mesh silica gel, Dichloromethane: Methanol=10:1). DDO-2213_024_6 (420 mg, 584.08 μmol, 54.40% yield) was obtained as a yellow solid.

¹H NMR (CHLOROFORM-d, 400 MHz) δ_{H}=9.98 (s, 1H), 9.23 (d, J=8.0 Hz, 1H), 8.51 (s, 1H), 8.41 (d, J=5.2 Hz, 1H), 7.11 (dd, J=2.0, 5.2 Hz, 1H), 7.02 (d, J=12.0 Hz, 1H), 3.90 (d, J=5.2 Hz, 4H), 3.82 (d, J=5.2 Hz, 4H), 2.93 (d, J=9.6 Hz, 3H), 2.80 (s, 3H), 2.51 (s, 3H), 1.23 (d, J=4.4 Hz, 6H).

### Step 6: 6-chloro-N4-(4-fluoro-5-(2-morpholinopyrimidin-4-yl)-2-((3S,5R)-3,4,5-trimethylpiperazin-1-yl)phenyl)pyrimidine-4,5-diamine (DDO-2213_024)

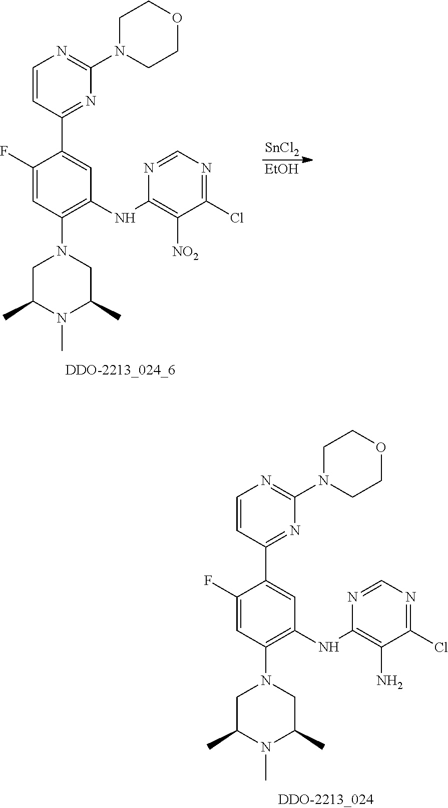

To a mixture of DDO-2213_024_6 (420 mg, 752.68 μmol, 1 eq) in MeOH (20 mL) and H₂O (6 mL) was added SnCl₂·2H₂O (509 mg, 2.26 mmol, 3 eq) in one portion. The mixture was heated to 80° C. and stirred for 4 hours. The pH was adjusted to around 8 by progressively adding NaHCO₃ (20 mL). Then the mixture was added DCM (50 mL) and stirred for 5 mins. The mixture was filtered via a filter paper. The combined organic phase was washed with brine (50 mL*2), dried with anhydrous Na₂SO₄, filtered and concentrated in vacuum. The residue was purified by prep. HPLC (Column: Phenomenex Gemini-NX C18 75*30 mm*3 μm; mobile phase: [water (0.04% NH₃H₂O+10 mM NH₄HCO₃)-ACN]; B %: 26%-66%; 13 min). DDO-2213_024 (79 mg, 149.03 μmol, 19.80% yield, 99.611% purity) was obtained as a light yellow solid.

¹H NMR (DMSO-d₆, 400 MHz) δ_{H}=8.43 (d, J=5.2 Hz, 1H), 8.40 (d, J=8.4 Hz, 1H), 8.15 (s, 1H), 7.77 (s, 1H), 7.08-7.04 (m, 1H), 6.97 (d, J=13.6 Hz, 1H), 5.33 (s, 2H), 3.74 (d, J=4.0 Hz, 4H), 3.67 (d, J=4.0 Hz, 4H), 3.07 (d, J=11.2 Hz, 2H), 2.49-2.41 (m, 3H), 2.17 (d, J=6.8 Hz, 1H), 2.14 (s, 3H), 0.95 (d, J=6.0 Hz, 6H).

HPLC Rₜ=2.802 min in 8 min chromatography, XBridge Shield RP18, 5 μm, 2.1*50 mm, purity 99.611%.

LCMS Rₜ=2.272 min in 4 min chromatography, XBridge Shield RP18, 5 μm, 2.1*50 mm, purity 96.704%, MS ESI calcd. for 527.23 [M+H]⁺528.23, found 528.3.

### Example 59. 6-chloro-N⁴-(4-fluoro-5-(1-(5-(morpholinomethyl)pyrimidin-2-yl)-1,2,5,6-tetrahydropyridin-3-yl)-2-((3S,5R)-3,4,5-trimethylpiperazin-1-yl)phenyl)pyrimidine-4,5-diamine (Compound 145, Table 3)

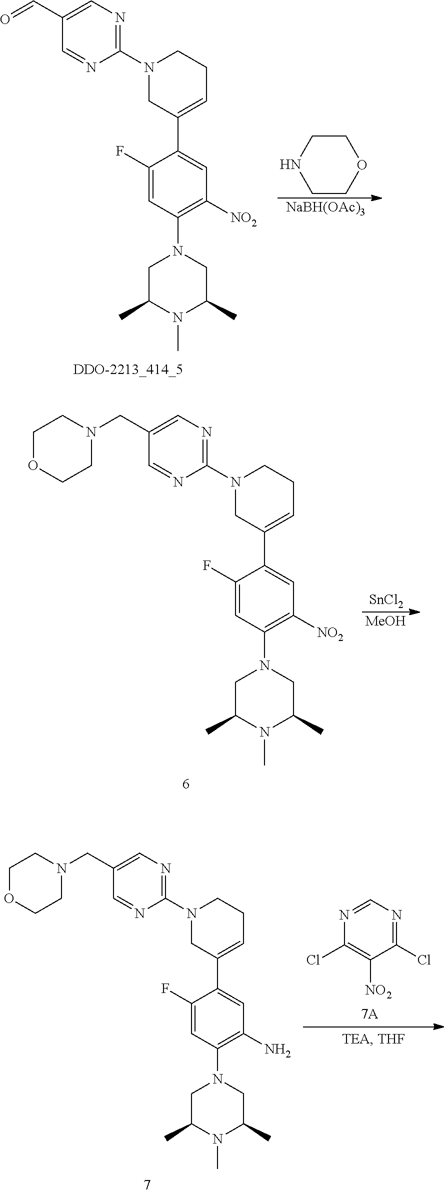

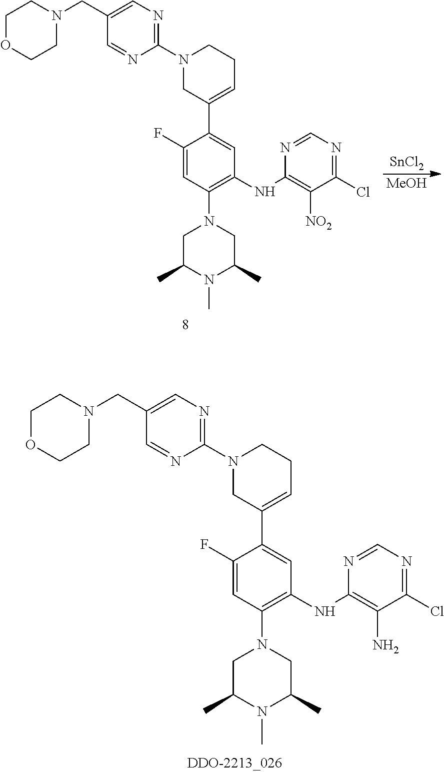

### Step 1: 4-((2-(3-(2-fluoro-5-nitro-4-((3S,5R)-3,4,5-trimethylpiperazin-1-yl)phenyl)-5,6-dihydropyridin-1(2H)-yl)pyrimidin-5-yl)methyl)morpholine (Compound 6)

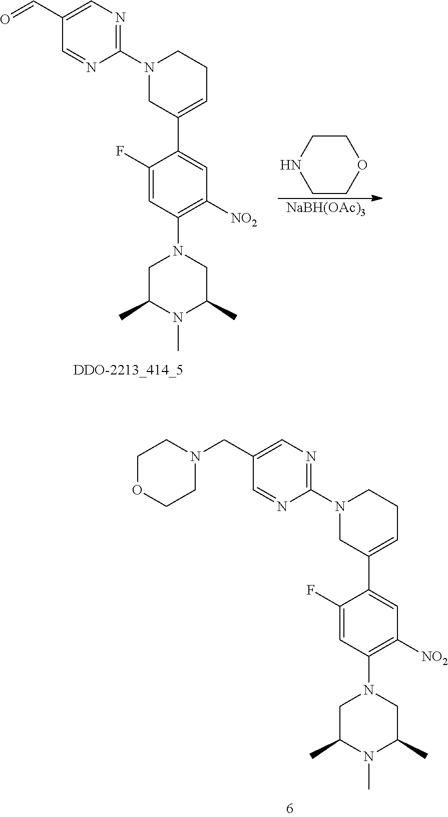 [SMILES: C[C@@H]1CN(c2cc(F)c(C3=CCCN(c4ncc(C=O)cn4)C3)cc2[N+](=O)[O-])C[C@H](C)N1C ; C1COCCN1 ; C[C@@H]1CN(c2cc(F)c(C3=CCCN(c4ncc(CN5CCOCC5)cn4)C3)cc2[N+](=O)[O-])C[C@H](C)N1C ; "NaBH(OAc)3"]

Compound DDO-2213_414_5 can be synthesized as shown in Example 64. To a solution of compound DDO-2213_414_5 (500 mg, 1.10 mmol, 1 eq.) and morpholine (288 mg, 3.30 mmol, 290.43 μL, 3 eq.) in DCM (10 mL) was added NaBH(OAc)₃ (700 mg, 3.30 mmol, 3 eq.) and TEA (557 mg, 5.50 mmol, 765.61 μL, 5 eq.). The mixture was stirred at 25° C. for 12 hr. Water (30 ml) was added to the mixture, the resulting mixture was extracted with DCM (50 mL*3). The combined organic phase was washed with brine (30 mL), dried over anhydrous Na₂SO₄, filtered and concentrated. The residue was purified by flash silica gel chromatography (ISCO®; 12 g SepaFlash® Silica Flash Column, Eluent of 0-18% MeOH/DCM @ 25 mL/min). Compound 6 (550 mg, 920.82 μmol, 83.70% yield, 88% purity) was obtained as a brown oil.

1H NMR (DMSO-d6, 400 MHz) δH=8.29 (s, 1H), 8.32-8.29 (m, 1H), 7.88 (d, J=8.0 Hz, 1H), 7.14 (d, J=13.6 Hz, 1H), 6.18 (s, 1H), 5.75 (s, 1H), 3.65 (s, 6H), 3.16 (s, 6H), 3.10 (s, 4H), 1.92-1.91 (m, 9H), 1.27-1.23 (m, 6H).

### Step 2: 4-fluoro-5-(1-(5-(morpholinomethyl)pyrimidin-2-yl)-1,2,5,6-tetrahydropyridin-3-yl)-2-((3S,5R)-3,4,5-trimethylpiperazin-1-yl)aniline (Compound 7)

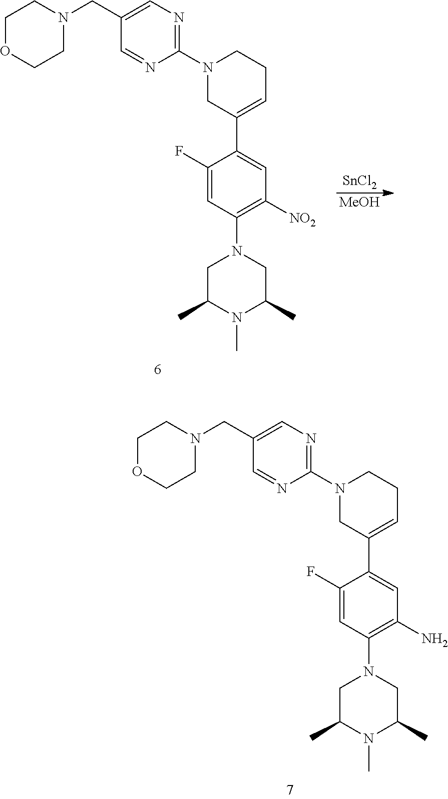 [SMILES: "MeOH" ; C[C@@H]1CN(c2cc(F)c(C3=CCCN(c4ncc(CN5CCOCC5)cn4)C3)cc2[N+](=O)[O-])C[C@H](C)N1C ; "SnCl2" ; C[C@@H]1CN(c2cc(F)c(C3=CCCN(c4ncc(CN5CCOCC5)cn4)C3)cc2N)C[C@H](C)N1C]

To a solution of compound 6 (550 mg, 1.05 mmol, 1 eq.) in MeOH (10 mL) and H₂O (5 mL) was added SnCl₂·2H₂O (708 mg, 3.14 mmol, 3 eq.). The mixture was stirred at 80° C. for 3 hr. The reaction mixture was adjusted to pH=9 by 1N aq.NaHCO₃. The filter liquor was diluted with DCM (100 mL), washed with brine (50 mL*2). The organic layer was dried over Na₂SO₄, filtered and concentrated under reduced pressure to give a residue. The product was used in the next step without further purification. Compound 7 (230 mg, crude) was obtained as a brown oil.

LCMS Rₜ=0.667 min in 1.5 min chromatography, XBridge Shield RP18, 5 μm, 3.0*25 mm, purity 64.39%, MS ESI calcd. for 495.31 [M+H]⁺496.31, found 496.3.

### Step 3: 6-chloro-N-(4-fluoro-5-(1-(5-(morpholinomethyl)pyrimidin-2-yl)-1,2,5,6-tetrahydropyridin-3-yl)-2-((3S,5R)-3,4,5-trimethylpiperazin-1-yl)phenyl)-5-nitropyrimidin-4-amine (Compound 8)

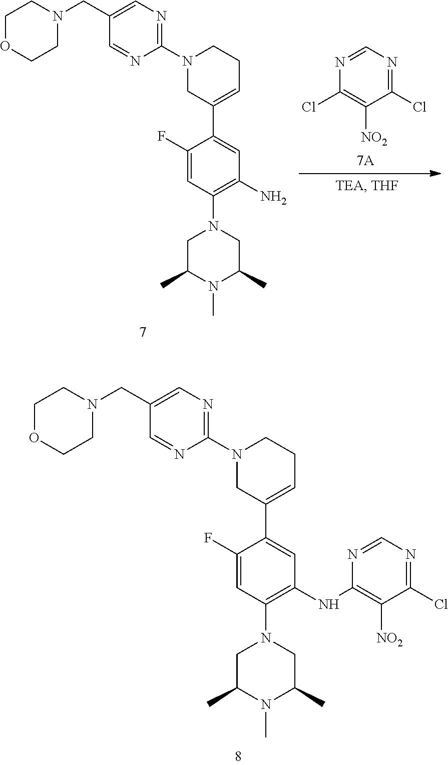 [SMILES: C[C@@H]1CN(c2cc(F)c(C3=CCCN(c4ncc(CN5CCOCC5)cn4)C3)cc2N)C[C@H](C)N1C ; O=[N+]([O-])c1c(Cl)ncnc1Cl ; C[C@@H]1CN(c2cc(F)c(C3=CCCN(c4ncc(CN5CCOCC5)cn4)C3)cc2Nc2ncnc(Cl)c2[N+](=O)[O-])C[C@H](C)N1C]

To a solution of compound 7 (110 mg, 221.94 μmol, 1 eq.) in THF (5 mL) was added TEA (67.37 mg, 665.81 μmol, 92.67 μL, 3 eq.) and compound 7A (47.36 mg, 244.13 μmol, 1.1 eq.) The mixture was stirred at 25° C. for 12 hr. The reaction mixture was concentrated directly. The product was used in the next step without further purification. Compound 8 (144 mg, 100.49 μmol, 45.28% yield) was obtained as a brown solid.

### Step 5: 6-chloro-N⁴-(4-fluoro-5-(1-(5-(morpholinomethyl)pyrimidin-2-yl)-1,2,5,6-tetrahydropyridin-3-yl)-2-((3S,5R)-3,4,5-trimethylpiperazin-1-yl)phenyl)pyrimidine-4,5-diamine (DDO-2213_026)

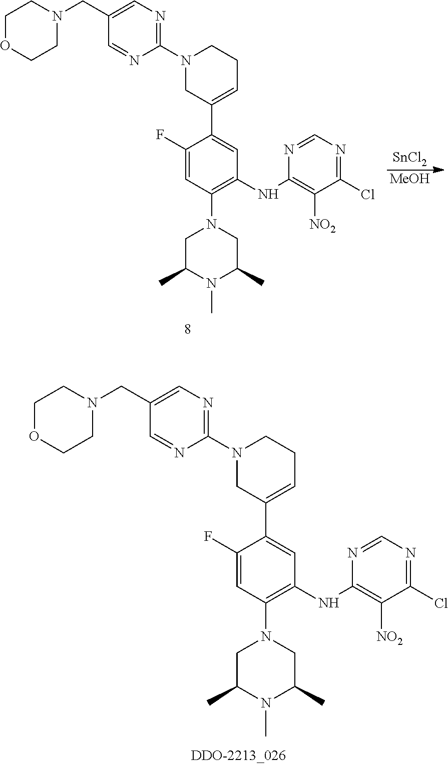

To a solution of compound 8 (144 mg, 220.47 μmol, 1 eq.) in MeOH (10 mL) and H₂O (4 mL) was added SnCl₂·2H₂O (248.74 mg, 1.10 mmol, 5 eq.). The mixture was stirred at 80° C. for 3 hr. The reaction mixture was adjusted to pH-9 by NaHCO₃. The resulting mixture was extracted with DCM (10 mL*3). The combined organic phase was washed with water (10 mL), dried over anhydrous Na₂SO₄, filtered and concentrated. The residue was purified by prep-HPLC (Column: Phenomenex Gemini-NX C18 75*30 mm*3 μm; Mobile Phase A: water(0.04% NH₃H₂O+10 mM NH₄HCO₃)-ACN; Mobile Phase B: 30%-60%; 11 min). DDO-2213_026 (15.2 mg, 23.98 μmol, 10.88% yield, 98.3% purity) was obtained as a white solid.

¹H NMR (DMSO-d₆, 400 MHz) δ_{H}=8.28 (s, 2H), 8.10 (s, 1H), 7.82 (s, 1H), 7.72 (d, J=8.0 Hz, 1H), 6.93 (d, J=12.8 Hz, 1H), 6.09 (s, 1H), 5.31 (s, 2H), 4.49 (s, 2H), 3.92 (s, 2H), 3.54 (s, 4H), 3.30 (s, 2H), 2.92 (d, J=10.4 Hz, 2H), 2.43-2.37 (m, 2H), 2.32 (s, 6H), 2.23 (s, 2H), 2.15 (s, 3H), 0.95 (d, J=5.8 Hz, 6H).

HPLC Rₜ=3.828 min in 8 min chromatography, XBridge Shield RP18, 5 μm, 2.1*50 mm, purity 98.30%.

LCMS Rₜ=2.938 min in 4 min chromatography, XBridge Shield RP18, 5 μm, 2.1*50 mm, purity 96.62%, MS ESI calcd. for 622.31 [M+H]⁺623.31, found 623.3.

### Example 60. 6-chloro-N4-(4-fluoro-5-(2-(4-isopropylpiperazin-1-yl)pyrimidin-5-yl)-2-((3S,5R)-3,4,5-trimethylpiperazin-1-yl)phenyl)pyrimidine-4,5-diamine (Compound 146, Table 3)

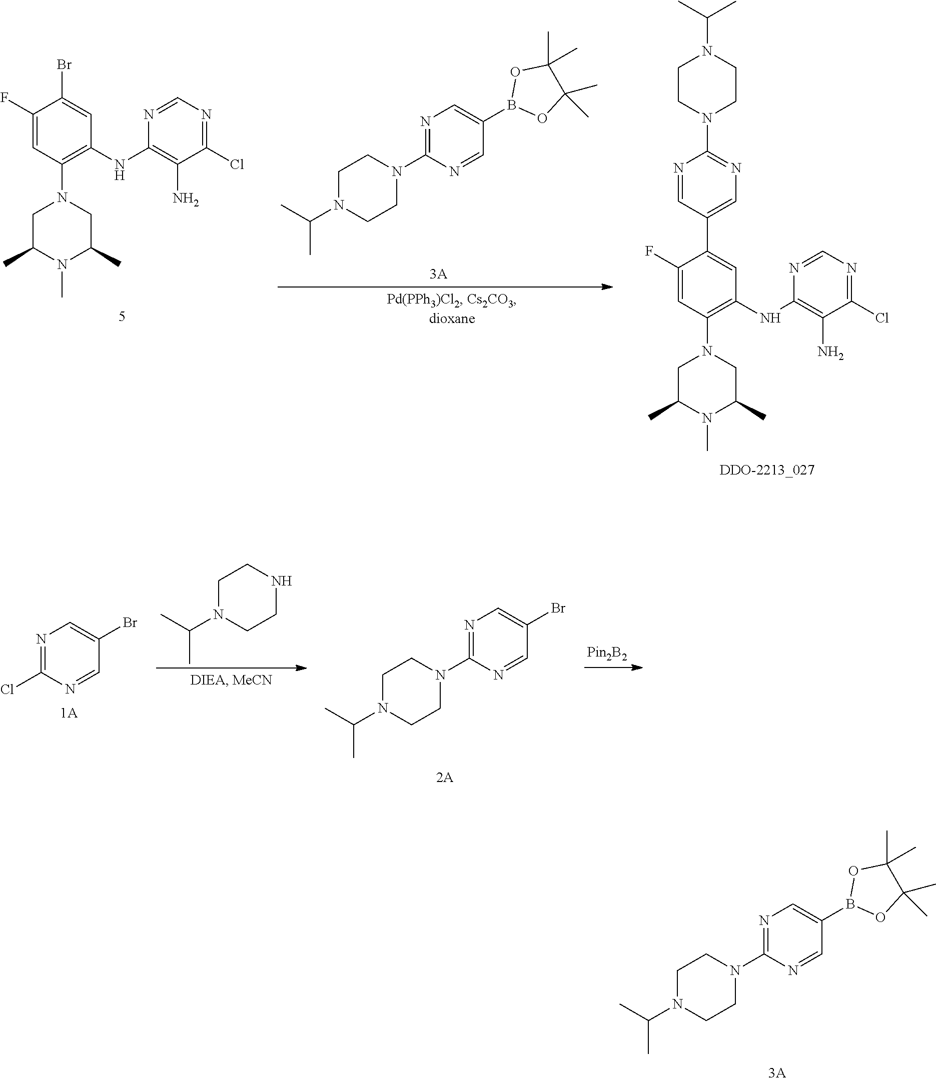

### Step 1: 5-bromo-2-(4-isopropylpiperazin-1-yl)pyrimidine (Compound 2A)

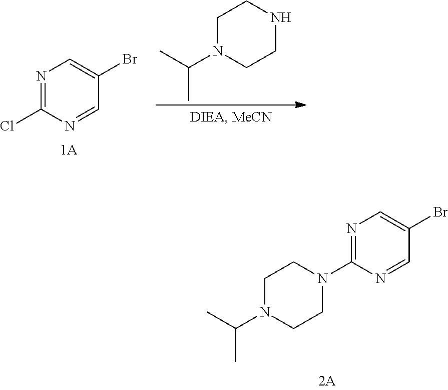

To a solution of compound 1A (1.2 g, 6.20 mmol, 1 eq.) and 1-isopropylpiperazine (795 mg, 6.20 mmol, 887.75 μL, 1 eq.) in MeCN (15 mL) was added DIEA (1.60 g, 12.41 mmol, 2.16 mL, 2 eq.). The mixture was stirred at 20° C. for 12 hr. The mixture was concentrated in vacuo. The reaction mixture was diluted with H₂O (20 mL), extracted with DCM (50 mL*3), washed with brine (50 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-4% MeOH/DCM @ 40 mL/min). Compound 2A (1.54 g, 4.86 mmol, 78.34% yield) was obtained as a yellow solid.

¹H NMR (DMSO-d₆, 400 MHz) δ_{H}=8.53 (s, 2H), 4.87-4.41 (m, 1H), 3.77-3.28 (m, 6H), 3.02 (s, 2H), 1.24 (s, 6H).

### Step 2: 2-(4-isopropylpiperazin-1-yl)-5-(4,4,5,5-tetramethyl-1,3,2-dioxaborolan-2-yl)pyrimidine (Compound 3A)

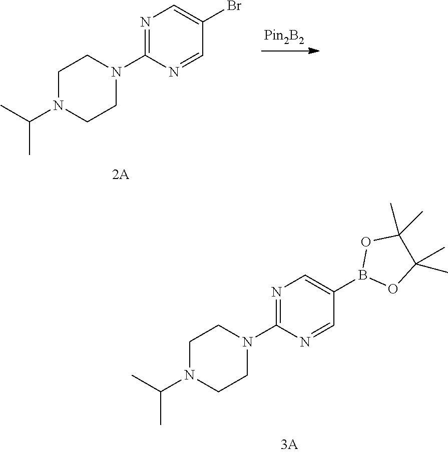 [SMILES: CC(C)N1CCN(c2ncc(Br)cn2)CC1 ; CC(C)N1CCN(c2ncc(B3OC(C)(C)C(C)(C)O3)cn2)CC1]

To a mixture of compound 2A (1.54 g, 5.40 mmol, 1 eq.) in dioxane (20 mL) was added AcOK (1.06 g, 10.80 mmol, 2 eq.), Pin₂B₂ (1.65 g, 6.48 mmol, 1.2 eq.) and Pd(dppf)Cl₂ (395 mg, 540.00 μmol, 0.1 eq.) under N₂. The reaction mixture was stirred at 80° C. for 12 hr. The mixture was extracted with DCM (100 mL*2). The combined organic phase was washed with brine (100 mL), dried with anhydrous Na₂SO₄, filtered and concentrated in vacuum. The residue was purified by flash silica gel chromatography (ISCO®; 20 g SepaFlash® Silica Flash Column, Eluent of 0-10% EtOAc/DCM @ 30 mL/min). Compound 3A (1.34 g, 2.42 mmol, 44.81% yield, 60% purity) was obtained as a brown solid.

¹H NMR (DMSO-d₆, 400 MHz) δ_{H}=8.47 (s, 1H), 8.34 (d, J=4.8 Hz, 1H), 3.81-3.65 (m, 4H), 2.76-2.63 (m, 1H), 2.50-2.44 (m, 4H), 1.91 (s, 1H), 1.28 (s, 6H), 0.99 (dd, J=3.2, 6.4 Hz, 6H).

### Step 3: 6-chloro-N4-(4-fluoro-5-(2-(4-isopropylpiperazin-1-yl)pyrimidin-5-yl)-2-((3S,5R)-3,4,5-trimethylpiperazin-1-yl)phenyl)pyrimidine-4,5-diamine (DDO-2213_027)

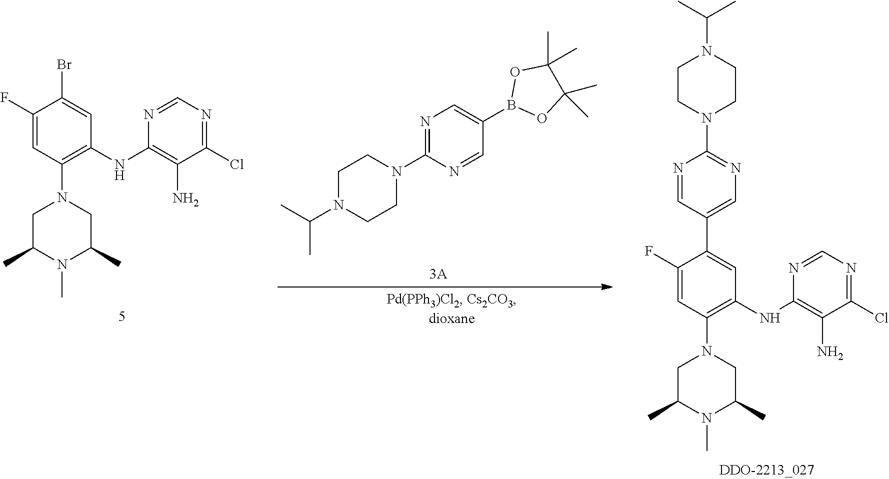 [SMILES: C[C@@H]1CN(c2cc(F)c(Br)cc2Nc2ncnc(Cl)c2N)C[C@H](C)N1C ; CC(C)N1CCN(c2ncc(B3OC(C)(C)C(C)(C)O3)cn2)CC1 ; CC(C)N1CCN(c2ncc(-c3cc(Nc4ncnc(Cl)c4N)c(N4C[C@@H](C)N(C)[C@@H](C)C4)cc3F)cn2)CC1]

Compound 5 can be synthesized as shown in Example 38. A mixture of compound 5 (200 mg, 450.71 μmol, 1 eq.), compound 3A (150 mg, 450.71 μmol, 1 eq.), Pd(dppf)Cl₂·CH₂Cl₂ (37 mg, 45.07 μmol, 0.1 eq.) and Cs₂CO₃ (294 mg, 901.42 μmol, 2 eq.) in dioxane (4 mL) and H₂O (0.8 mL) was degassed and purged with N₂ for 3 times, and then the mixture was stirred at 100° C. for 12 hr under N₂ atmosphere. The reaction mixture was diluted with H₂O (20 mL), extracted with DCM (50 mL×2), washed with brine (50 mL). The organic layer was dried over Na₂SO₄, filtered and concentrated under reduced pressure to give a residue. The residue was purified by prep-HPLC (Column: Phenomenex Gemini-NX C18 75*30 mm*3 μm; Mobile Phase A: purified water (0.05% NH₃H₂O+10 mM NH₄HCO₃); Mobile Phase B: acetonitrile; Gradient: 43-63% B in 10 min.). Compound DDO-2213_027 (35 mg, 61.25 μmol, 13.59% yield, 99.6% purity) was obtained as a white solid.

¹H NMR (DMSO-d₆, 400 MHz) δ_{H}=8.51 (d, J=0.8 Hz, 2H), 8.22-8.06 (m, 1H), 7.89 (d, J=8.4 Hz, 1H), 7.81 (s, 1H), 7.03 (d, J=12.4 Hz, 1H), 5.34 (s, 2H), 3.80-3.70 (m, 4H), 3.34-3.28 (m, 4H), 2.97-2.89 (m, 2H), 2.73-2.64 (m, 1H), 2.47-2.40 (m, 2H), 2.30-2.20 (m, 2H), 2.16 (s, 3H), 0.97 (dd, J=6.4, 12.0 Hz, 12H)

HPLC Rₜ=3.572 min in 8 min chromatography, X Bridge Shield RP18, 5 μm, 2.1*50 mm, purity 99.63%.

LCMS Rₜ=2.503 min in 4 min chromatography, X Bridge Shield RP18, 5 μm, 2.1*50 mm, purity 99.46%, MS ESI calcd. for 568.30 [M+H]⁺ 569.30, found 569.5.

### Example 61. 6-chloro-N4-(4-fluoro-5-(2-((S)-2-methylmorpholino)pyrimidin-4-yl)-2-((3S,5R)-3,4,5-trimethylpiperazin-1-yl)phenyl)pyrimidine-4,5-diamine (Compound 148, Table 3)

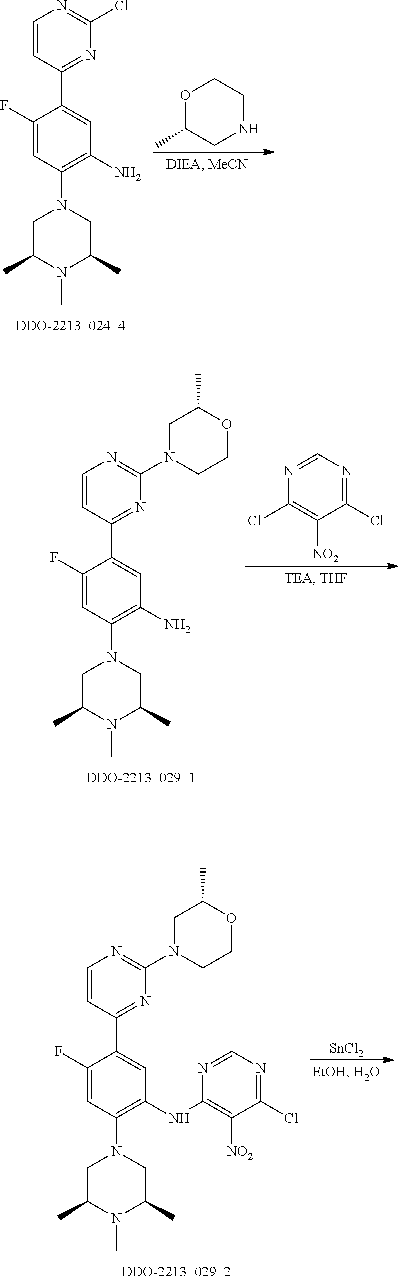

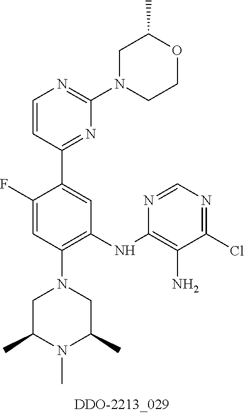 [SMILES: C[C@@H]1CN(c2cc(F)c(-c3ccnc(N4CCO[C@@H](C)C4)n3)cc2Nc2ncnc(Cl)c2N)C[C@H](C)N1C]

### Step 1: 4-fluoro-5-(2-((S)-2-methylmorpholino)pyrimidin-4-yl)-2-((3S,5R)-3,4,5-trimethylpiperazin-1-yl)aniline (DDO-2213_029_1)

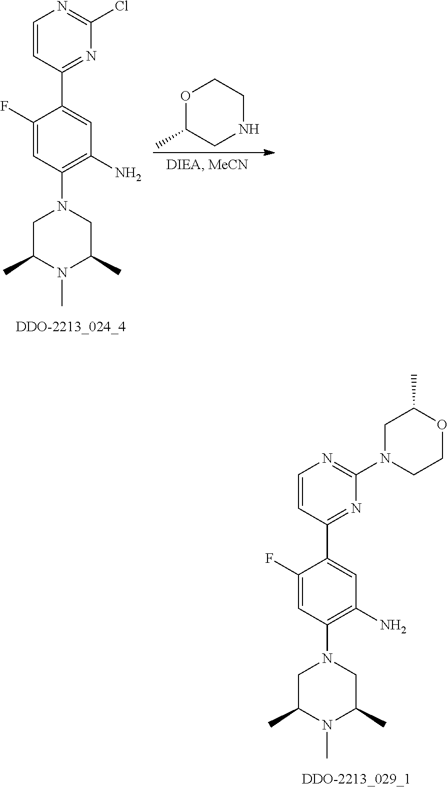

DDO-2213_024_4 can be synthesized as shown in Example 58. To a mixture of DDO-2213_024_4 (350 mg, 1.00 mmol, 1 eq.) and (2S)-2-methylmorpholine (405 mg, 4.00 mmol, 4 eq.) in CH₃CN (5 mL) was added DIEA (517 mg, 4.00 mmol, 697.06 μL, 4 eq.) in one portion. The mixture was stirred at 80° C. for 12 hours. The residue was concentrated under reduce pressure. The residue was purified by silica gel chromatography (column weight: 12 g, diameter: 100 mm, 100-200 mesh silica gel, Dichloromethane: Methanol=10:1). DDO-2213_029_1 (400 mg, 823.80 μmol, 82.34% yield) was obtained as a yellow oil.

¹H NMR (DMSO-d₆, 400 MHz) δ_{H}=8.37 (d, J=5.2 Hz, 1H), 7.41 (d, J=8.0 Hz, 1H), 6.98 (dd, J=2.0, 5.2 Hz, 1H), 6.74 (d, J=13.6 Hz, 1H), 4.71 (s, 2H), 4.60-4.46 (m, 2H), 3.91 (dd, J=2.4, 11.6 Hz, 1H), 3.56-3.50 (m, 2H), 3.08 (d, J=8.8 Hz, 2H), 2.96 (dt, J=3.2, 12.4 Hz, 1H), 2.64 (dd, J=10.4, 12.8 Hz, 1H), 2.33 (d, J=7.6 Hz, 4H), 2.21 (s, 3H), 1.17 (d, J=6.4 Hz, 3H), 1.03 (d, J=4.8 Hz, 6H).

### Step 2: 6-chloro-N-(4-fluoro-5-(2-((S)-2-methylmorpholino)pyrimidin-4-yl)-2-((3S,5R)-3,4,5-trimethylpiperazin-1-yl)phenyl)-5-nitropyrimidin-4-amine (DDO-2213_029_2)

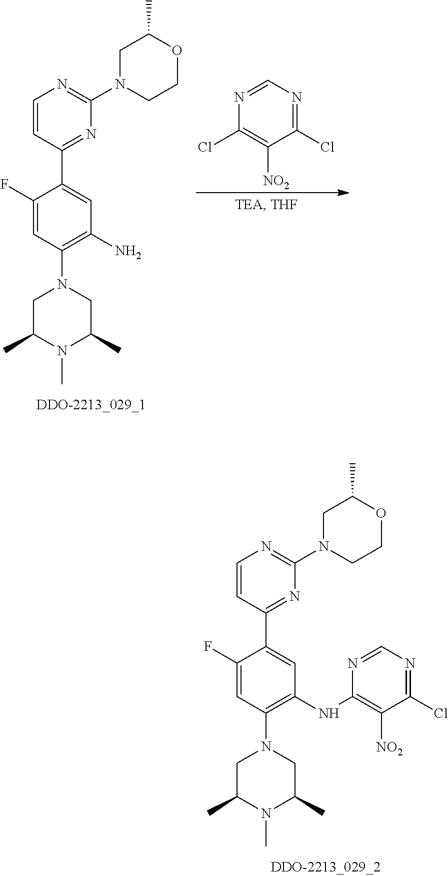 [SMILES: C[C@@H]1CN(c2cc(F)c(-c3ccnc(N4CCO[C@@H](C)C4)n3)cc2N)C[C@H](C)N1C ; O=[N+]([O-])c1c(Cl)ncnc1Cl ; C[C@@H]1CN(c2cc(F)c(-c3ccnc(N4CCO[C@@H](C)C4)n3)cc2Nc2ncnc(Cl)c2[N+](=O)[O-])C[C@H](C)N1C]

To a mixture of DDO-2213_029_1 (400 mg, 964.98 μmol, 1 eq.) and 4,6-dichloro-5-nitro-pyrimidine (374 mg, 1.93 mmol, 2 eq.) in THF (10 mL) was added TEA (293 mg, 2.89 mmol, 402.94 μL, 3 eq.) in one portion. The mixture was stirred at 25° C. for 12 hours. The residue was concentrated under reduce pressure. The residue was purified by silica gel chromatography (column weight: 40 g, diameter: 100 mm, 100-200 mesh silica gel, Dichloromethane: Methanol=10:1). DDO-2213_029_2 (390 mg, 580.33 mol, 60.14% yield) was obtained as a yellow solid.

¹H NMR (DMSO-d₆, 400 MHz) δ_{H}=8.49 (s, 1H), 8.45 (d, J=5.2 Hz, 1H), 8.32 (d, J=7.2 Hz, 1H), 7.12 (d, J=13.2 Hz, 1H), 7.07 (dd, J=2.0, 5.2 Hz, 1H), 4.57-4.43 (m, 2H), 3.92 (dd, J=2.4, 11.6 Hz, 1H), 3.58-3.46 (m, 2H), 3.31-3.19 (m, 2H), 3.03-2.91 (m, 2H), 2.71-2.61 (m, 3H), 2.53 (s, 3H), 1.21-1.11 (m, 9H), 1.10-1.02 (m, 2H).

### Step 3: 6-chloro-N4-(4-fluoro-5-(2-((S)-2-methylmorpholino)pyrimidin-4-yl)-2-((3S,5R)-3,4,5-trimethylpiperazin-1-yl)phenyl)pyrimidine-4,5-diamine (DDO-2213_029)

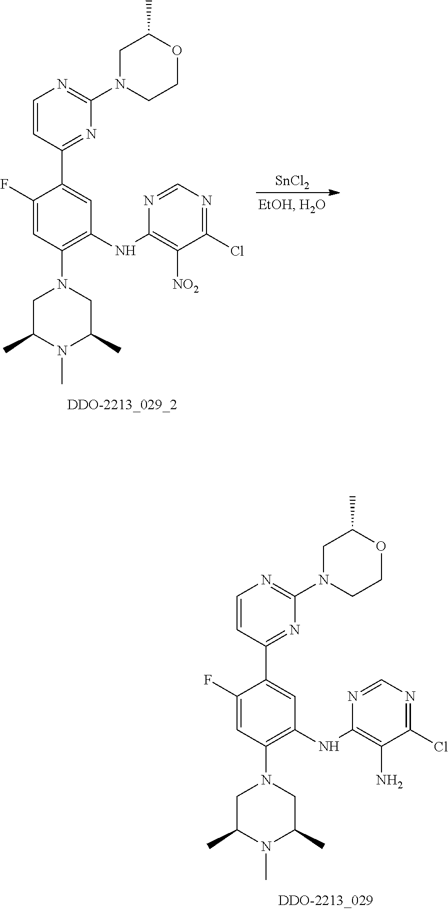 [SMILES: C[C@@H]1CN(c2cc(F)c(-c3ccnc(N4CCO[C@@H](C)C4)n3)cc2Nc2ncnc(Cl)c2[N+](=O)[O-])C[C@H](C)N1C ; "SnCl2" ; C[C@@H]1CN(c2cc(F)c(-c3ccnc(N4CCO[C@@H](C)C4)n3)cc2Nc2ncnc(Cl)c2N)C[C@H](C)N1C]

To a mixture of DDO-2213_029_2 (390 mg, 681.78 μmol, 1 eq.) in MeOH (5 mL) and H₂O (2 mL) was added SnCl₂·2H₂O (462 mg, 2.05 mmol, 3 eq.) in one portion. The mixture was stirred at 80° C. for 4 hours. The pH was adjusted to around 8 by progressively adding NaHCO₃ (5 mL). Then the mixture was added DCM (50 mL) and stirred for 5 mins. The mixture was filtered via a filter paper. The combined organic phase was washed with brine (20 mL*2), dried with anhydrous Na₂SO₄, filtered and concentrated in vacuum. The residue was purified by prep. HPLC (Column: Phenomenex Gemini-NX C18 75*30 mm*3 μm; mobile phase: [water (0.04% NH₃H₂O+10 mM NH₄HCO₃)-ACN]; B %: 28%-68%; 11 min). DDO-2213_029 (11.5 mg, 20.90 μmol, 3.06% yield, 98.49% purity) was obtained as a white solid.

¹H NMR (DMSO-d₆, 400 MHz) δ_{H}=8.46-8.42 (m, 2H), 8.15 (s, 1H), 7.78 (s, 1H), 7.06 (dd, J=2.0, 5.2 Hz, 1H), 6.98 (d, J=13.6 Hz, 1H), 5.34 (s, 2H), 4.58-4.43 (m, 2H), 3.91 (dd, J=2.4, 11.6 Hz, 1H), 3.57-3.46 (m, 2H), 3.07 (d, J=11.2 Hz, 2H), 3.02-2.92 (m, 1H), 2.70-2.60 (m, 1H), 2.48-2.41 (m, 2H), 2.23-2.17 (m, 2H), 2.15 (s, 3H), 1.15 (d, J=6.4 Hz, 3H), 0.96 (d, J=6.0 Hz, 6H).

HPLC Rₜ=3.095 min in 8 min chromatography, Ultimate XB-C18 3.0*50 mm, 3 μm, purity 98.49%.

LCMS Rₜ=2.480 min in 4 min chromatography, Xtimate C18.3 μm, 2.1*30 mm, purity 98.102%, MS ESI calcd. for 542.05 [M+H]⁺543.05, found 542.4.

### Example 62. 6-chloro-N4-(5′-((cyclohexylamino)methyl)-2′-fluoro-4-((3S,5R)-3,4,5-trimethylpiperazin-1-yl)-[1,1′-biphenyl]-3-yl)pyrimidine-4,5-diamine (Compound 149, Table 3)

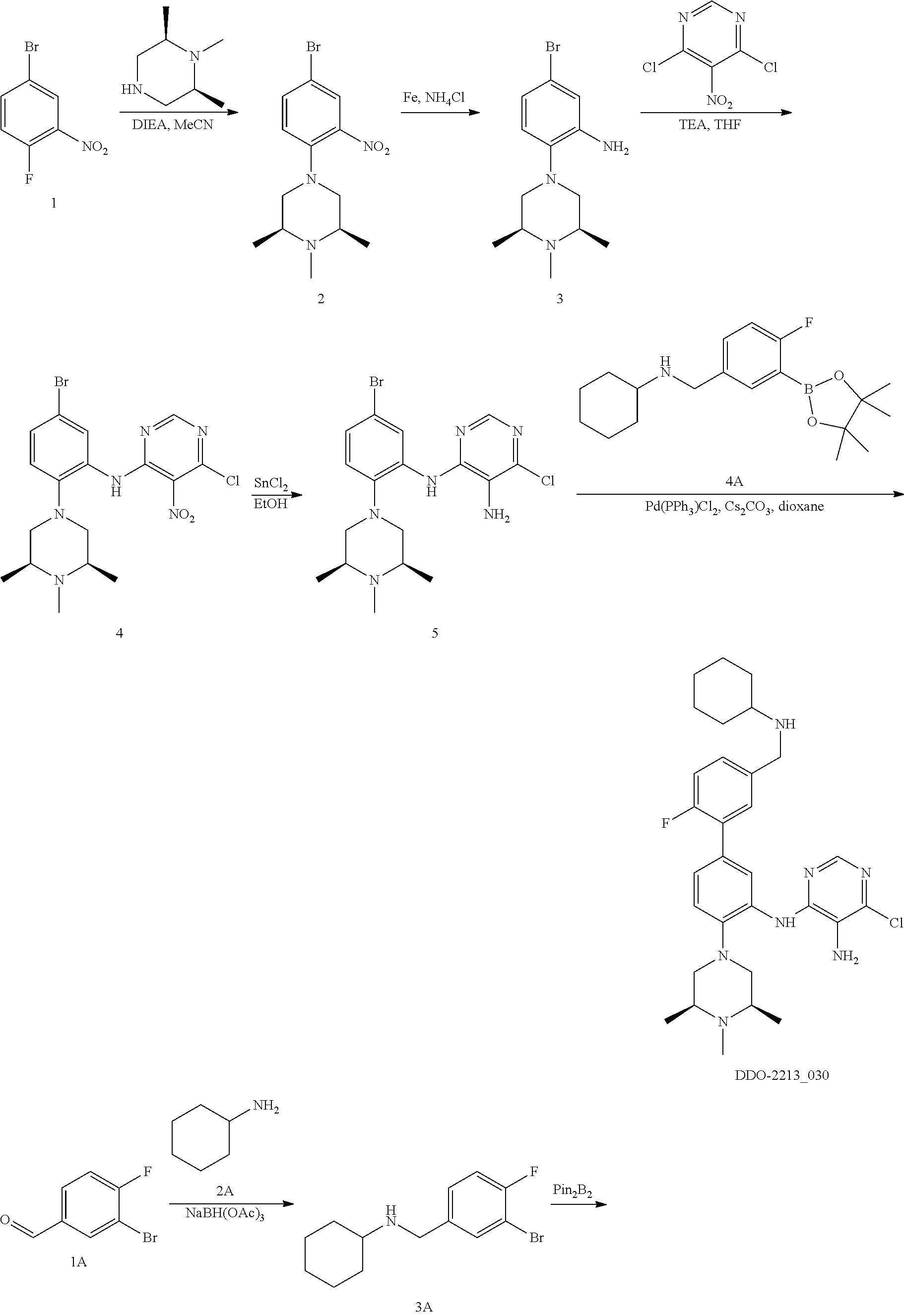

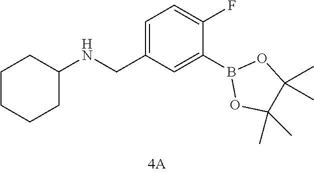 [SMILES: CC1(C)OB(c2cc(CNC3CCCCC3)ccc2F)OC1(C)C]

### Step 1: N-(3-bromo-4-fluorobenzyl)cyclohexanamine (Compound 3A)

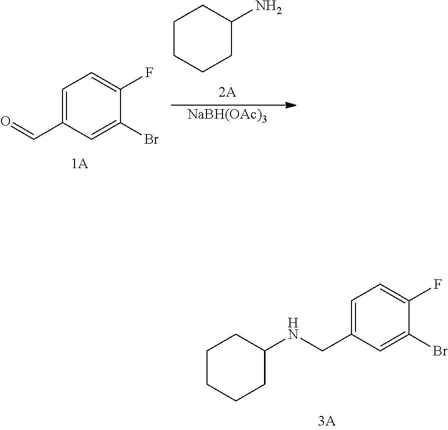

To a mixture of compound 2A (977 mg, 9.85 mmol, 1.13 mL, 1 eq.) and compound 1A (2 g, 9.85 mmol, 1 eq.) in DCM (20 mL) was added NaBH(OAc)₃ (6.26 g, 29.56 mmol, 3 eq.) at 0° C. The reaction mixture was stirred at 20° C. for 2 hr to give a brown mixture. The mixture was extracted with DCM (100 mL*3). The combined organic phase was washed with brine (100 mL*2), dried with anhydrous Na₂SO₄, filtered and concentrated in vacuum. The crude product was purified by silica gel chromatography (ISCO®; 20 g SepaFlash® Silica Flash Column, Eluent of 0-20% MeOH/DCM ethergradient @ 40 mL/min). Compound 3A (3 g, 9.49 mmol, 96.33% yield) was obtained as a white solid.

¹H NMR (DMSO-d₆, 400 MHz) δ_{H}=7.68 (dd, J=2.0, 6.8 Hz, 1H), 7.40-7.35 (m, 1H), 7.32-7.26 (m, 1H), 2.40-2.33 (m, 1H), 1.88 (s, 3H), 1.83 (d, J=12.4 Hz, 2H), 1.70-1.61 (m, 2H), 1.53 (dd, J=3.2, 8.8 Hz, 1H), 1.23-0.98 (m, 5H).

### Step 2: N-(4-fluoro-3-(4,4,5,5-tetramethyl-1,3,2-dioxaborolan-2-yl)benzyl)cyclohexanamine (Compound 4A)

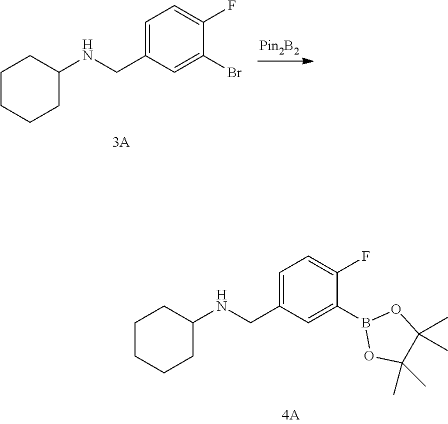 [SMILES: Fc1ccc(CNC2CCCCC2)cc1Br ; CC1(C)OB(c2cc(CNC3CCCCC3)ccc2F)OC1(C)C]

To a mixture of compound 3A (1 g, 3.49 mmol, 1 eq.) in dioxane (10 mL) was added AcOK (686 mg, 6.99 mmol, 2 eq.), Pin₂B₂ (1.06 g, 4.19 mmol, 1.2 eq.) and Pd(dppf)Cl₂ (256 mg, 349.43 μmol, 0.1 eq.) under N₂. The reaction mixture was stirred at 100° C. for 2 hr. The mixture was extracted with DCM (80 mL*3). The combined organic phase was washed with brine (100 mL), dried with anhydrous Na₂SO₄, filtered and concentrated in vacuum. The crude product was purified by silica gel chromatography (ISCO®; 12 g SepaFlash® Silica Flash Column, Eluent of 0-20% MeOH/DCM ethergradient @ 20 mL/min). Compound 4A (890 mg, 2.06 mmol, 58.85% yield) was obtained as a black oil.

¹H NMR (DMSO-d₆, 400 MHz) δ_{H}=8.72 (s, 1H), 7.83 (dd, J=2.4, 5.6 Hz, 1H), 7.75-7.69 (m, 1H), 7.24 (t, J=8.8 Hz, 1H), 4.20-4.14 (m, 1H), 3.47-3.41 (m, 1H), 2.10 (d, J=10.4 Hz, 1H), 1.81-1.74 (m, 1H), 1.61 (d, J=12.4 Hz, 1H), 1.31 (s, 6H), 1.15 (s, 2H), 1.06 (s, 12H).

### Step 3: (2S,6R)-4-(4-bromo-2-nitrophenyl)-1,2,6-trimethylpiperazine (Compound 2)

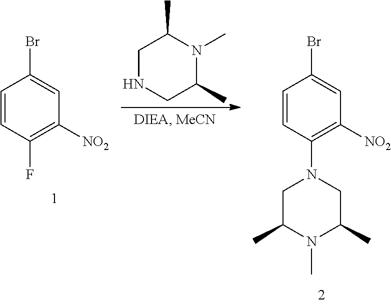

To a solution of compound 1 (2 g, 9.09 mmol, 1.12 mL, 1 eq.) in CH₃CN (20 mL) was added (2R,6S)-1,2,6-trimethylpiperazine (1.17 g, 9.09 mmol, 1 eq.) and DIEA (2.35 g, 18.18 mmol, 3.17 mL, 2 eq.). The mixture was stirred at 30° C. for 12 hr. The reaction mixture was diluted with water (100 mL), extracted with DCM (100 mL) and washed with brine (100 mL). The organic layer was dried over Na₂SO₄, filtered and concentrated under reduced pressure to give a residue. The product was used directly to the next step without further purification. Compound 2 (2.9 g, 7.72 mmol, 84.95% yield) was obtained as an orange solid.

¹H NMR (DMSO-d₆, 400 MHz) δ_{H}=7.97 (d, J=2.4 Hz, 1H), 7.64 (dd, J=2.0, 8.8 Hz, 1H), 7.13 (d, J=8.4 Hz, 1H), 3.42-3.22 (m, 2H), 3.15 (d, J=12.4 Hz, 2H), 3.06-2.89 (m, 2H), 2.66 (s, 3H), 1.42 (d, J=6.0 Hz, 6H).

### Step 4: 5-bromo-2-((3S,5R)-3,4,5-trimethylpiperazin-1-yl)aniline (Compound 3)

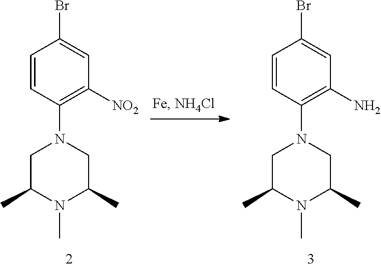

To a solution of compound 2 (2.9 g, 8.84 mmol, 1 eq.) in EtOAc (22.5 mL) and H₂O (7.5 mL) was added Fe (1.48 g, 26.51 mmol, 3 eq.) and NH₄Cl (2.36 g, 44.18 mmol, 5 eq.). The mixture was stirred at 80° C. for 4 hr. The mixture was filtered to removed the insoluble. The filter liquor was concentrated in vacuo. The reaction mixture was diluted with aq.NaHCO₃ (100 mL), extracted with DCM (100 mL) and washed with brine (100 mL). The organic layer was dried over Na₂SO₄, filtered and concentrated under reduced pressure to give a residue. The residue was purified by flash silica gel chromatography (ISCO®; 25 g SepaFlash® Silica Flash Column, Eluent of 0-10% Methanol/Dichloromethane @ 30 mL/min). Compound 3 (2.32 g, 7.53 mmol, 85.23% yield) was obtained as an orange oil.

¹H NMR (DMSO-d₆, 400 MHz) δ_{H}=6.82 (d, J=2.4 Hz, 1H), 6.76 (d, J=8.4 Hz, 1H), 6.64 (dd, J=2.4, 8.4 Hz, 1H), 4.99 (s, 2H), 2.86 (d, J=8.4 Hz, 2H), 2.37-2.27 (m, 4H), 2.20 (s, 3H), 1.01 (d, J=5.6 Hz, 6H).

### Step 5: N-(5-bromo-2-((3S,5R)-3,4,5-trimethylpiperazin-1-yl)phenyl)-6-chloro-5-nitropyrimidin-4-amine (Compound 4)

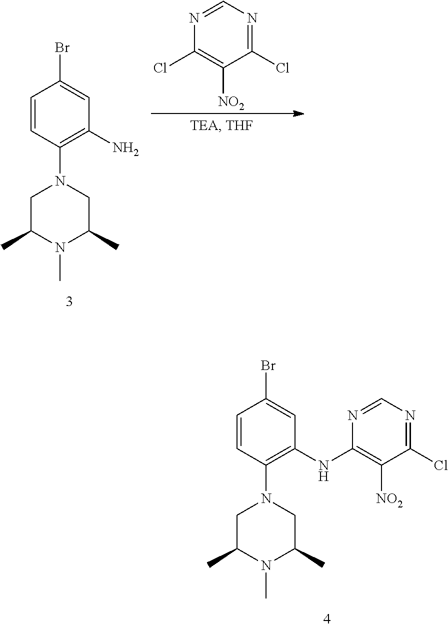

To a solution of compound 3 (2.32 g, 7.78 mmol, 1 eq.) and 4,6-dichloro-5-nitro-pyrimidine (1.81 g, 9.34 mmol, 1.2 eq.) in THF (24 mL) was added TEA (1.57 g, 15.56 mmol, 2.17 mL, 2 eq.). The mixture was stirred at 25° C. for 3 hr. The mixture was filtered to removed the insoluble. The filter liquor was concentrated in vacuo. The reaction mixture was diluted with water (100 mL), extracted with DCM (100 mL) and washed with brine (100 mL). The organic layer was dried over Na₂SO₄, filtered and concentrated under reduced pressure to give a residue. The residue was purified by flash silica gel chromatography (ISCO®; 12 g SepaFlash® Silica Flash Column, Eluent of 0-10% Methanol/Dichloromethane @ 30 mL/min). Compound 4 (1.56 g, 1.23 mmol, 15.84% yield) was obtained as a brown solid.

¹H NMR (DMSO-d₆, 400 MHz) δ_{H}=8.90 (s, 1H), 8.66 (s, 1H), 7.39 (d, J=6.0 Hz, 2H), 3.17 (d, J=4.4 Hz, 3H), 3.11-3.04 (m, 7H), 1.30-1.25 (m, 6H).

### Step 6: 6-chloro-N4-(5′-((cyclohexylamino)methyl)-2′-fluoro-4-((3S,5R)-3,4,5-trimethylpiperazin-1-yl)-[1,1′-biphenyl]-3-yl)pyrimidine-4,5-diamine (Compound 5)

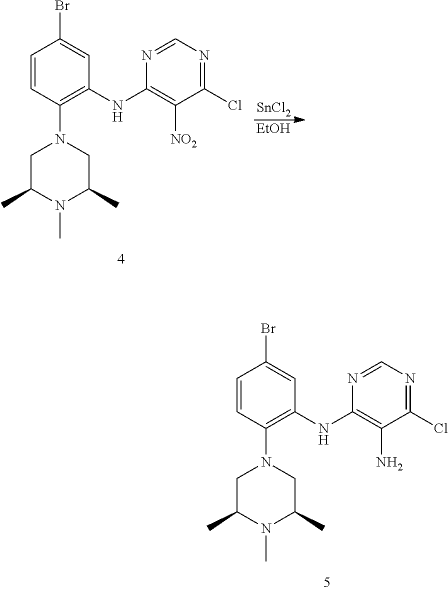

To a solution of compound 4 (1.56 g, 3.42 mmol, 1 eq.) in EtOH (10 mL) and H₂O (1 mL) was added SnCl₂·2H₂O (1.54 g, 6.85 mmol, 2 eq.). The mixture was stirred at 80° C. for 2 hr. The mixture was adjusted to pH=9 with aq.NaHCO₃. The mixture was added DCM until there's a precipitate, filtered and concentrated under reduced pressure to give a residue. The residue was purified by flash silica gel chromatography (ISCO®; 20 g SepaFlash® Silica Flash Column, Eluent of 0-13% MeOH/DCM @ 35 mL/min). Compound 5 (640 mg, 941.90 μmol, 58.60% yield) was obtained as a yellow oil.

¹H NMR (DMSO-d₆, 400 MHz) δ_{H}=8.41-8.29 (m, 2H), 7.99 (s, 1H), 7.21 (dd, J=2.4, 8.4 Hz, 1H), 7.14-7.07 (m, 1H), 5.31 (s, 2H), 3.20-3.15 (m, 1H), 3.18 (d, J=2.8 Hz, 1H), 2.80 (d, J=10.8 Hz, 2H), 2.49-2.42 (m, 2H), 2.38 (d, J=6.4 Hz, 2H), 2.20 (s, 3H), 0.99 (d, J=6.0 Hz, 6H).

### Step 7: 6-chloro-N4-(5′-((cyclohexylamino)methyl)-2′-fluoro-4-((3S,5R)-3,4,5-trimethylpiperazin-1-yl)-[1,1′-biphenyl]-3-yl)pyrimidine-4,5-diamine (DDO-2213_030)

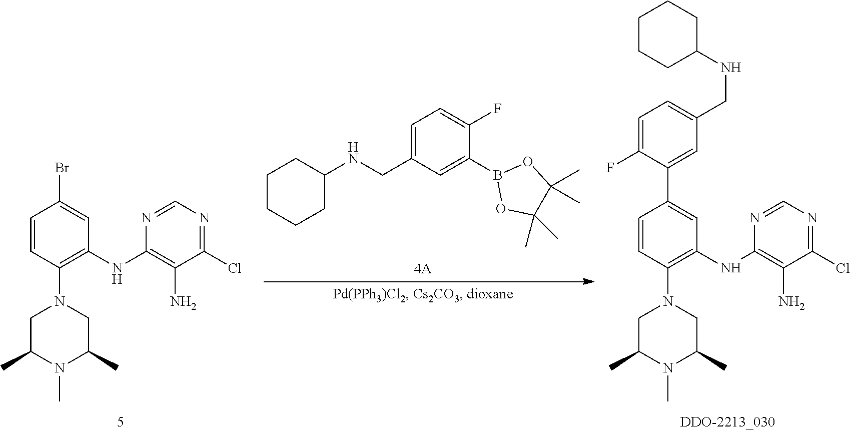

A mixture of compound 5 (200 mg, 469.76 μmol, 1 eq.), compound 4A (157 mg, 469.76 μmol, 1 eq.), Pd(dppf)Cl₂·CH₂Cl₂ (38 mg, 46.98 μmol, 0.1 eq.) and Cs₂CO₃ (306 mg, 939.51 μmol, 2 eq.) in dioxane (4 mL) and H₂O (0.8 mL) was degassed and purged with N₂ for 3 times, and then the mixture was stirred at 100° C. for 12 hr under N₂ atmosphere. The reaction mixture was diluted with water (20 mL), extracted with DCM (50 mL) and washed with brine (20 mL). The organic layer was dried over Na₂SO₄, filtered and concentrated under reduced pressure to give a residue. The residue was purified by prep-HPLC (Column: Phenomenex Gemini-NX C18 150*30 mm*5 μm; Mobile Phase A: purified water (0.05% HCl); Mobile Phase B: acetonitrile; Gradient: 10-40% B in 10 min.). DDO-2213_030 (15.7 mg, 26.38 μmol, 5.62% yield, 98.89% purity, HCl) was obtained as a yellow solid.

¹H NMR (DMSO-d₆, 400 MHz) δ_{H}=11.70-10.88 (m, 1H), 9.49-9.16 (m, 2H), 8.96-8.61 (m, 1H), 8.23-7.99 (m, 1H), 7.90-7.79 (m, 2H), 7.62 (d, J=2.4 Hz, 1H), 7.47-7.27 (m, 3H), 4.18 (s, 2H), 3.49-3.23 (m, 3H), 3.10 (s, 3H), 2.79 (d, J=4.8 Hz, 3H), 2.20-2.05 (m, 2H), 1.85-1.70 (m, 2H), 1.66-1.55 (m, 1H), 1.36 (d, J=6.4 Hz, 7H), 1.31-1.13 (m, 5H).

HPLC Rₜ=3.398 min in 8 min chromatography, Ultimate XB-C18 (3×50 mm, 3 μm), purity 98.89%.

LCMS Rₜ=1.883 min in 4 min chromatography, X Bridge Shield RP18, 5 μm, 2.1*50 mm, purity 97.55%, MS ESI calcd. for 551.29 [M+H]⁺ 552.29, found 552.4.

### Example 63. 6-chloro-N4-(5-(2-(dimethylamino)pyrimidin-4-yl)-4-fluoro-2-((3S,5R)-3,4,5-trimethylpiperazin-1-yl)phenyl)pyrimidine-4,5-diamine (Compound 147, Table 3)

### Step 1: 4-(5-amino-2-fluoro-4-((3S,5R)-3,4,5-trimethylpiperazin-1-yl)phenyl)-N,N-dimethylpyrimidin-2-amine (DDO-2213_028_1)

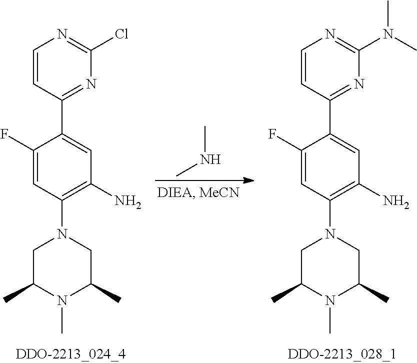

DDO-2213_024_4 is synthesized as shown in Example 58. To a mixture of DDO-2213_024_4 (350 mg, 1.00 mmol, 1 eq) and N-methylmethanamine (408 mg, 5.00 mmol, 458.33 μL, 5 eq, HCl) in CH₃CN (10 mL) was added DIEA (1.03 g, 8.00 mmol, 1.39 mL, 8 eq) in one portion. The mixture was heated to 80° C. and stirred for 12 hours. The residue was concentrated under reduce pressure. The residue was purified by silica gel chromatography (column weight: 12 g, diameter: 100 mm, 100-200 mesh silica gel, Dichloromethane: Methanol=10:1). DDO-2213_028_1 (330 mg, 865.10 μmol, 86.47% yield) was obtained as a yellow solid.

¹H NMR (DMSO-d₆, 400 MHz) δ_{H}=8.34 (d, J=5.2 Hz, 1H), 7.45 (d, J=8.0 Hz, 1H), 6.91 (dd, J=2.0, 5.2 Hz, 1H), 6.79 (d, J=12.8 Hz, 1H), 4.87 (s, 2H), 3.62-3.53 (m, 2H), 3.17 (s, 6H), 3.09 (d, J=7.2 Hz, 2H), 2.67-2.58 (m, 2H), 2.50 (s, 3H), 1.28 (s, 6H).

### Step 2: 4-(5-((6-chloro-5-nitropyrimidin-4-yl)amino)-2-fluoro-4-((3S,5R)-3,4,5-trimethylpiperazin-1-yl)phenyl)-N,N-dimethylpyrimidin-2-amine (DDO-2213_028_2)

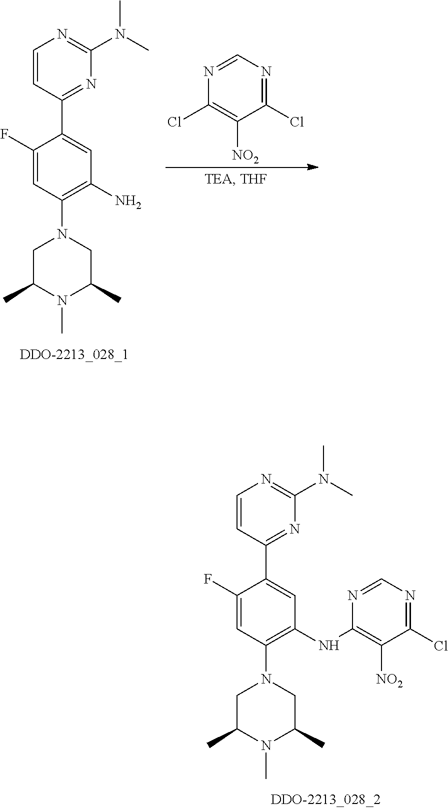

To a mixture of DDO-2213_028_1 (330 mg, 920.62 μmol, 1 eq) and 4,6-dichloro-5-nitro-pyrimidine (358 mg, 1.84 mmol, 2 eq) in THF (5 mL) was added TEA (279 mg, 2.76 mmol, 384.41 μL, 3 eq) in one portion. The mixture was stirred at 25° C. for 12 hours. The residue was concentrated under reduce pressure. The residue was purified by silica gel chromatography (column weight: 12 g, diameter: 100 mm, 100-200 mesh silica gel, Dichloromethane: Methanol=10:1). DDO-2213_028_2 (300 mg, 484.04 mol, 52.58% yield) was obtained as a yellow solid.

¹H NMR (DMSO-d₆, 400 MHz) δ_{H}=9.97 (s, 1H), 8.49 (s, 1H), 8.42 (d, J=5.2 Hz, 1H), 8.34 (d, J=8.0 Hz, 1H), 7.19 (d, J=13.2 Hz, 1H), 7.03-6.96 (m, 1H), 3.21-3.17 (m, 2H), 3.16 (s, 6H), 3.11-3.06 (m, 2H), 2.94-2.78 (m, 4H), 2.58-2.51 (m, 3H), 1.31 (s, 6H).

### Step 3: 6-chloro-N4-(5-(2-(dimethylamino)pyrimidin-4-yl)-4-fluoro-2-((3S,5R)-3,4,5-trimethylpiperazin-1-yl)phenyl)pyrimidine-4,5-diamine (DDO-2213_028)

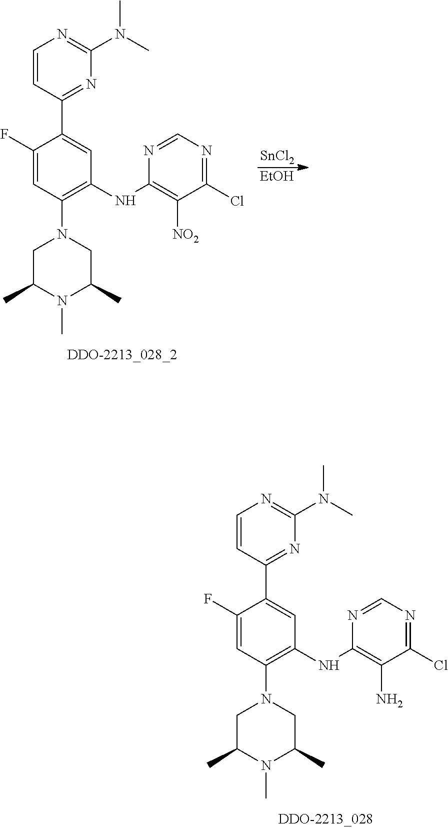

To a mixture of DDO-2213_028_2 (300 mg, 581.43 μmol, 1 eq) in MeOH (5 mL) and H₂O (2 mL) was added SnCl₂·2H₂O (393.59 mg, 1.74 mmol, 3 eq) in one portion. The mixture was stirred at 80° C. for 4 hours. The pH was adjusted to around 8 by progressively adding NaHCO₃ (10 mL). Then the mixture was added DCM (50 mL) and stirred for 5 mins. The mixture was filtered via a filter paper. The combined organic phase was washed with brine (20 mL*2), dried with anhydrous Na₂SO₄, filtered and concentrated in vacuum. The residue was purified by prep. HPLC (Column: Phenomenex Gemini-NX C18 75*30 mm*3 μm; mobile phase: [water (0.04% NH₃H₂O+10 mM NH₄HCO₃)-ACN]; B %: 25%-65%; 11 min). DDO-2213_028 (4.3 mg, 8.71 μmol, 1.50% yield, 98.45% purity) was obtained as a white solid.

¹H NMR (DMSO-d₆, 400 MHz) δ_{H}=8.45 (d, J=8.4 Hz, 1H), 8.38 (d, J=5.2 Hz, 1H), 8.14 (s, 1H), 7.78 (s, 1H), 7.01-6.93 (m, 2H), 5.33 (s, 2H), 3.15 (s, 6H), 3.06 (d, J=10.8 Hz, 2H), 2.47-2.40 (m, 2H), 2.20 (d, J=6.4 Hz, 2H), 2.15 (s, 3H), 0.96 (d, J=6.0 Hz, 6H).

HPLC Rₜ=1.62 min in 8 min chromatography, Ultimate XB-C18 3.0*50 mm, 3 μm, purity 98.45%.

LCMS Rₜ=2.326 min in 4 min chromatography, XBridge Shield RP18, 5 μm, 2.1*50 mm, purity 96.137%, MS ESI calcd. for 485.22 [M+H]⁺496.22, found 486.3.

### Pharmaceutical Compositions

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl cellulose, propylene glycol, isopropyl myristate and purified alcohol USP. The resulting gel mixture is then incorporated into containers, such as tubes, which are suitable for topical administration.

### BIOLOGICAL EXAMPLES

### Example B-1: Enzyme Assay of Inhibition Against MLL1-WDR5 Protein-Protein Interactions

WDR5 TR-FRET Assay Procedure: Stock compounds were transferred to the assay plate by Echo Liquid Handler. Reactions were performed in the assay buffer (1×PBS, 300 mM NaCl, 0.5 mM TCEP, 0.1% CHAPS) containing 5 nM WDR5 protein, 10 nM peptide (Ac-ARTEVHLRKS-[Ahx-Ahx] [C]-Alexa Fluor 488-NH2) and 0.25 nM Tb-anti His antibody (Tb-Ab) in 384-well white plate (PerkinElmer) with a final volume of 20 μl. Compounds were incubated with WDR5 protein for 30 min at room temperature. Plates were covered, protected from light and incubated for 60 min at room temperature after adding the peptide and Tb-Ab. EnVision Multimode Plate Reader (PerkinElmer) was used for the TR-FRET assay with excitation wavelength at 340 nm and emission wavelength at 495 and 520 nm. The ratio of the 520/495 wavelengths were used to assess the degree of the FRET signal. IC₅₀ was calculated by fitting the inhibition data using XLfit software to sigmoidal dose-response model.

Biphenyl compound DDO-2084 was reported to be a small molecule inhibitor that can inhibit MLL1-WDR5 protein-protein interaction, reduce MLL1 enzyme catalytic activity, and downregulate the expression of Hox and Meis-1 genes (Eur. J. Med. Chem. 2016, 124, 480-489). DDO-2084 is used as a positive control compound.

TABLE 5
MLL1-WDR5 PPI inhibitory
activity of representative
compounds disclosed herein.

| | MLL1-WDR5 |
|---|---|
| | PPI inhibitory |
| Compound No. | activity (nM) |
| HYBI_002 | 44.20 |
| HYBI_003 | 15.60 |
| HYBI_010 | 9.62 |
| HYBI_013 | 19.39 |
| HYBI_016 | 20.65 |
| HYBI_018 | 16.79 |
| HYBI_019 | 20.36 |
| HYBI_019A | 17.13 |
| HYBI_020 | 8.23 |
| HYBI_021 | 7.09 |
| HYBI_022 | 62.27 |
| HYBI_023 | 19.78 |
| HYBI_027 | 10.83 |
| HYBI_028 | 5.24 |
| HYBI_037 | 108.63 |
| HYBI_038 | 184.61 |
| HYBI_039 | 131.43 |
| HYBI_040 | 828.28 |
| HYBI_042 | 802.43 |
| HYBI_044 | 902.87 |
| HYBI_045 | 384.48 |
| HYBI_046 | 270.62 |
| HYBI_047 | 465.77 |
| HYBI_050 | 5556.62 |
| HYBI_053 | 19.42 |
| HYBI_082 | 5.32/4.49 (avg. 4.91) |
| HYBI_083 | 3.08 |
| HYBI_084 | 4.48/4.96 (avg. 4.72) |
| HYBI_085 | 3.86 |
| HYBI_086 | 3.99 |
| HYBI_087 | 2.60 |
| HYBI_101 | 3.65 |
| HYBI_110 | 3.00 |

As shown in Table 5, the compounds disclosed herein have relatively strong inhibitory activity against MLL1-WDR5 protein-protein interaction when compared to DDO-2084, the structure of which is shown below.

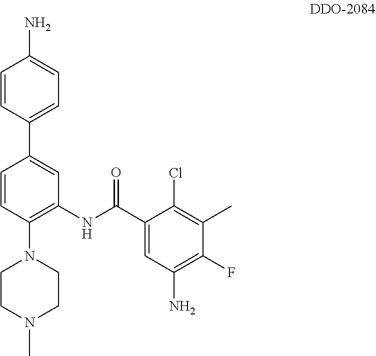 [SMILES: Cc1c(F)c(N)cc(C(=O)Nc2cc(-c3ccc(N)cc3)ccc2N2CCN(C)CC2)c1Cl]

### Example B-2: Enzyme Assay of Inhibition in Leukemia Cell Lines

Procedure: MV-411 or Molm-13 cells were seeded into 384 well plates at 2000 cells/well density in 50 uL according plate map and were allowed to naturally sediment by waiting about 30 min in Clean Bench. Next, plated cells were centrifuged for 1 min at 1000 rpm and the excess cells were transferred into the flasks for further culture. Cells in the assay plates were incubated (at least 4 hrs) at 37° C., 5% CO₂ for next process followed by adding the compounds as the plate map indicated. The tests were performed in duplicates with treatment of compounds at 10 pts 3 fold titration in 384 well plates. Taxol was as positive control while DMSO as negative control. To rule out edge effect, the wells on the edge were not seeded and therefore one 384 well plate holds 13 compounds. Cells viability was measured 72 hrs after incubation with compounds and using CellTiterGlo (promega) viability assay according to manufactory's instruction to check the ATP production in each well.

Experiments on anti-proliferative activity against leukemia cells were conducted with some of the compounds of the invention. Table 6 shows the results of evaluation of the anti-proliferative activity of some of the compounds disclosed herein against acute leukemia cells, wherein MV-411 is human acute monocytic leukemia cell and Molm-13 is human acute myeloid leukemia cell. Table 6 indicates that the compounds of the disclosure are effective in inhibiting the proliferation of various leukemia cells.

TABLE 6
Anti-proliferative activity against leukemia cells of
some of the compounds.

| Compound No. | GI₅₀ μM (MV-411) |
|---|---|
| HYBI_002 | 6.31 |
| HYBI_003 | 3.41 |
| HYBI_010 | 5.95 |
| HYBI_013 | 2.46 |
| HYBI_014 | 2.62 |
| HYBI_016 | 7.10 |
| HYBI_018 | 5.99 |
| HYBI_019 | 27.06 |
| HYBI_019A | 24.85 |
| HYBI_020 | 8.36 |
| HYBI_021 | 25.06 |
| HYBI_022 | 17.15 |
| HYBI_023 | 31.96 |
| HYBI_027 | 20.00 |
| HYBI_028 | 30.87 |
| HYBI_037 | 8.38 |
| HYBI_038 | 17.49 |
| HYBI_039 | 0.1085 |
| HYBI_040 | 0.2105 |
| HYBI_041 | 13.24 |
| HYBI_042 | 4.746 |
| HYBI_043 | 76.04 |
| HYBI_044 | 64.75 |
| HYBI_045 | 4.151 |
| HYBI_046 | 10.52 |
| HYBI_047 | 16.78 |
| HYBI_050 | 10.72 |
| HYBI_053 | 5.69 |
| HYBI_082 | 2.40 |
| HYBI_083 | 40.18 |
| HYBI_084 | 3.35 |
| HYBI_085 | 2.51 |
| HYBI_086 | 19.69 |
| HYBI_087 | >200 |
| HYBI_101 | 10.49 |
| HYBI_110 | 25.01 |
| DDO-2306 | 7.11 |
| DDO-2308 | 9.292 |
| DDO-2312 | 24.53 |
| DDO-2313 | 40.89 |
| DDO-2316 | 25.36 |
| DDO-2315 | 24.48 |

^{a}defined as IC₅₀/μM;
^{b}defined as % inhibition at 22 μM
ND not determined

TABLE 7
Anti-proliferative activity against leukemia cells and inhibitory activity against MLL1-WDR5
protein-protein interaction as observed in some compounds of this disclosure.

| | | WDR5 TR-FRET |
|---|---|---|
| Compound No. | GI₅₀ μM (MV-411) | Assay (IC₅₀ nM ) |
| DDO-2213_001 | 11.59 | 11.07 |
| DDO-2213_002 | 15.58 | 55.53 |
| DDO-2213_003 | 59.94 | 394.97 |
| DDO-2213_004 | 14.82 | 68.84 |
| DDO-2213_005 | 3.37 | 11.06 |
| DDO-2213_006 | 6.09 | 38.61 |
| DDO-2213_007 | 11.49 | 51.26 |
| DDO-2213_008 | 4.76 | 8.63 |
| DDO-2213_009 | 1.69 (n = 2) | 33.89 |
| DDO-2213_010 | 7.93 | 249.60 |
| DDO-2213_012 | 104.16 | 56.38 |
| DDO-2213_014 | 8.26 | 186.15 |
| DDO-2213_015 | 4.14 (n = 2) | 85.54 |
| DDO-2213_016 | 6.31 (n = 2) | 12.15 |
| DDO-2213_017 | 40.45 | 51.65 |
| DDO-2213_018 | 10.83 | 110.03 |
| DDO-2213_019 | 6.98 | 215.24 |
| DDO-2213_020 | 5.21 | 7.44 |
| DDO-2213_021 | 9.01 | 23.43 |
| DDO-2213_022 | 3.68 | 41.22 |
| DDO-2213_023 | 2.79 (n =2) | 3.56 |
| DDO-2213_024 | 18.53 | 15.04 |
| DDO-2213_026 | 12.69 | 18.62 |
| DDO-2213_027 | 6.56 | 31.27 |
| DDO-2213_028 | >200 | 51.76 |
| DDO-2213_029 | 17.21 | 12.94 |
| DDO-2213_030 | 2.43 | 417.61 |

### Example B-3: hERG Assay Results

Procedure: Compounds were prepared and diluted with DMSO to make 0.2 mM and 0.02 mM. Reference compound was diluted with DMSO to make 8-point 4-fold serial dilution, starting at 0.2 mM. One μl of compounds/high control/low control was transferred to the assay plate according to the plate map. Next, and by following the plate map, 100 μl of membrane stocks was dispensed into the plate followed by adding 100 μl of radio ligand. Plates were then sealed and were incubated at RT for 1 hrs. In the meantime, soak the Unifilter-96 GF/C filter plates with 50 μl of 0.5% BSA per well for at least 0.5 hour at room temperature. When binding assays are completed, filter the reaction mixture through GF/C plates using Perkin Elmer Filtermate Harvester, and then wash each plate for 4 times with cold wash buffer. Next, Dry the filter plates for 1 hour at 50 degrees and seal the bottom of the filter plate wells using Perkin Elmer Unifilter-96 backing seal tape. Next, add 50 μl of Perkin Elmer Microscint 20 cocktail. Seal the top of the filter plate with Perkin Elmer TopSeal-A sealing film. Using Perkin Elmer MicroBeta2 Reader count ³H trapped on filter. Finally, analyze the data with GraphPad Prism 5. Calculate the “Inhibition [% Control]” using the equation: % Inh=(1−Background subtracted Assay value/Background subtracted HC value)*100.

The compound of the disclosure were tested in several hERG assays. The results with representative compounds are listed in Table 8.

TABLE 8
hERG assay results.

| Compound No. | IC₅₀ (nM) | Ki (nM) |
|---|---|---|
| 18 | >10000 | Data Not Shown |
| 19 | >10000 | Data Not Shown |
| 21 | >10000 | Data Not Shown |
| 29 | 3605 | 2038 |

Furthermore, several compounds were tested in the hERG channel assay and found to be essentially inactive, with IC₅₀>10.0 μM. These hERG assay results are encouraging as the selectivity ratios (IC₅₀ hERG/EC₅₀ MV-411) are quite high, ˜25 to 42 fold selectivity, so potential cardiotoxicity issues should be minimal.

The compounds disclosed herein have strong inhibitory activity against MLL1-WDR5 protein-protein interaction, can reduce the MLL1 catalytic activity of MLL1 at cellular level, downregulate the expression of Hox and Meis-1 genes and induce apoptosis of leukemia cells. Also, the phenyl triazole compounds of the invention exhibit good water solubility and pharmaceutical safety, and can be used for treating leukemia.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are incorporated by reference in their entirety for all purposes.

1. A compound having a structure of Formula I, Formula II, Formula III, Formula IV, Formula V or Formula VI, or a pharmaceutically acceptable salt, solvate, polymorph or prodrug thereof, as described in the specification.

2. The compound of claim 1, wherein the compound has a structure of Formula I, wherein the compound is selected from Table 1, or a pharmaceutically acceptable salt, solvate, polymorph or prodrug thereof.

3. The compound of claim 1, wherein the compound has a structure of Formula II or Formula III, wherein the compound is selected from Table 1, or a pharmaceutically acceptable salt, solvate, polymorph or prodrug thereof.

4. The compound of claim 1, wherein the compound has a structure of Formula III, wherein the compound is selected from Table 2, or a pharmaceutically acceptable salt, solvate, polymorph or prodrug thereof.

5. The compound of claim 1, wherein the compound has a structure of Formula I or Formula III, wherein the compound is selected from Table 2, or a pharmaceutically acceptable salt, solvate, polymorph or prodrug thereof.

6. The compound of claim 1, wherein the compound has a structure of Formula IV, wherein the compound is selected from Table 3, or a pharmaceutically acceptable salt, solvate, polymorph or prodrug thereof.

7. The compound of claim 1, wherein the compound has a structure of Formula V, wherein the compound is selected from Table 3, or a pharmaceutically acceptable salt, solvate, polymorph or prodrug thereof.

8. The compound of claim 1, wherein the compound has a structure of Formula VI, wherein the compound is selected from Table 3, or a pharmaceutically acceptable salt, solvate, polymorph or prodrug thereof.

9. The compound of claim 1, wherein the compound does not have the structure of DDO-2093.

10. The compound of claim 1, wherein the compound does not have the structure of DDO-2213.

11. The compound of claim 1, wherein the compound has the formula: 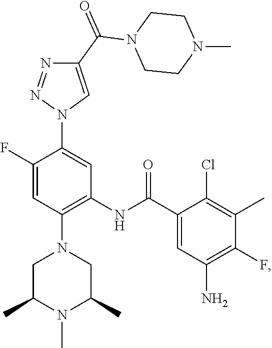 [SMILES: Cc1c(F)c(N)cc(C(=O)Nc2cc(-n3cc(C(=O)N4CCN(C)CC4)nn3)c(F)cc2N2C[C@@H](C)N(C)[C@@H](C)C2)c1Cl]
or a pharmaceutically acceptable salt thereof.

12. A pharmaceutical composition comprising a compound of claim 1, or a pharmaceutically acceptable salt, solvate, polymorph or prodrug thereof, and a pharmaceutically acceptable excipient.

13. A method for the treatment or prevention of acute leukemia in a patient in need thereof, comprising administering to the patient a therapeutically acceptable dose of the compound of claim 1, or the pharmaceutical composition of claim 12.

14. The method of claim 13, wherein the acute leukemia is acute leukemia with MLL1 gene rearrangement.
